(19) United States
(12) Patent Application Publication
(10) Pub. No.: US 2006/0093596 A1
(43) Pub. Date: May 4, 2006

(54) Genomic approach to identification of novel broad-spectrum antimicrobial peptides from bony fish

(76) Inventors: Susan Douglas, Boutilier's Point (CA); Jeffrey Gallant, Halifax (CA); Aleksander Patrzykat, Halifax (CA)

Correspondence Address: DINSMORE & SHOHL, LLP, 1900 CHEMED CENTER, 255 EAST FIFTH STREET, CINCINNATI, OH, 45202, US

(21) Appl. No.: 10/525,126

(22) PCT Filed: Aug. 22, 2003
(86) PCT No.: PCT/CA03/01323

§ 371 Date: Feb. 18, 2005

Related U.S. Application Data
(60) Provisional application No. 60/404,922, filed on Aug. 22, 2002.

(51) Int. Cl. A61K 38/54; C07H 21/04; C12P 21/06; C07K 14/47

(52) U.S. Cl. 424/94.2; 435/69.1; 435/320.1; 435/325; 530/324; 536/23.5

(57) ABSTRACT

There is provided a method of identifying candidate nucleic acid sequences encoding antimicrobial peptides. The method comprises: identifying an initial peptide of interest; identifying genomic DNA encoding the initial peptide; identifying a flanking sequence on each side of the initial peptide; obtaining primers complementary to the flanking sequences; and, screening a wide range of nucleic acid sequences to identify candidate sequences capable of being amplified using the primers from step e). In some instances the antimicrobial peptide is a hepcidin or a pleurocidin.

The present application is a 371 of PCT/CA2003/001323 and claims priority under 35 U.S.C. §119 to U.S. Application Ser. No. 60/404,922 filed Aug. 22, 2002.

### BACKGROUND OF THE INVENTION

Antimicrobial peptides have been isolated from a wide variety of plants and animals, and play an important role in defense against microbial invasion. They fall into three main classes based on secondary structure and amino acid sequence similarities: α-helical structures, highly disulphide-bonded (cysteine-rich) β-sheets and those with a high percentage of single amino acids such as proline or arginine. Most molecules are amphiphilic and contain both cationic and hydrophobic surfaces, enabling them to insert into biological membranes. Although one of the modes of action of antimicrobial peptides has been described as lysis of pathogens, they may also exert their effects by binding to intracellular targets. They have also been reported to exert a number of effects such as mediating inflammation and modulating the immune response.

A small number of natural antimicrobial peptides have been isolated from teleosts including the pleurocidin, from the skin of winter flounder (Cole, Weis et al. 1997), pardaxin from Red Sea Moses sole (Oren and Shai 1996), misgumin from loach (Park, Lee et al. 1997), HFA-1 from hagfish (Hwang, Seo et al. 1999), piscidins from hybrid striped bass eosinophilic granule cells (Silphaduang and Noga 2001), moronecidins from hybrid striped bass (Lauth, Shike et al. 2002), parasin, a cleavage product of histone 2A from catfish (Park, Park et al. 1998) and some uncharacterized mucous secretions from carp (LeMaitre, Orange et al. 1996) and trout (Smith, Fernandes et al. 2000). In addition, a cationic steroidal antibiotic, squalamine, has been isolated from the shark, Squalus acanthias (Moore, Wehrli et al. 1993).

Cysteine-rich antimicrobial peptides of the defensin family have been detected in the fat body of insects and the hemolymph of molluscs and crustaceans. They have also been isolated from various epithelia of mammals as well as circulating cells such as neutrophils and macrophages. Recently, small cysteine-rich peptides exhibiting antimicrobial activity against various fungi, Gram positive and Gram negative bacteria have been isolated from blood ultrafiltrate (Krause, Neitz et al. 2000), the human urinary tract (Park, Valore et al. 2001), and the gill of bacterially challenged hybrid striped bass (Shike et al. 2002). These peptides, referred to as hepcidin or LEAP-1 (liver-expressed antimicrobial peptide), have been proposed to be the vertebrate counterpart of insect peptides induced in the fat body in response to infection (Park, Valore et al. 2001). Antimicrobial peptides have a variety of potential uses. (see for example U.S. Pat. No. 6,288,212 of Hancock)

The conventional approach to identifying antimicrobial peptides involves biochemical purification from tissues or secretions. Fractions are tested for antimicrobial activity, and the purified peptides that exhibit activity are then sequenced. This approach is costly, time consuming, and not well suited to the identification of low abundance or difficult-to-purify antimicrobial peptides.

Thus, it is an object of the invention to provide a method for identifying potential antimicrobial peptides.

### SUMMARY OF THE INVENTION

In one aspect of the invention there is provided a method of identifying candidate nucleic acid sequences encoding antimicrobial peptides, said method comprising:
(a) identifying an initial peptide of interest;
(b) identifying genomic DNA encoding the initial peptide;
(c) identifying a flanking sequence on each side of the initial peptide;
(d) obtaining primers complementary to the flanking sequences; and,
(e) screening a wide range of nucleic acid sequences to identify candidate sequences capable of being amplified using the primers from step d).

According to one aspect of the invention the nucleotide and deduced amino acid sequences of hepcidin-like peptides are provided.

According to another aspect of the invention, the nucleotide and deduced amino acid sequences of pleurocidin-like peptides are provided.

According to another aspect of the invention primers suitable for use in the identification, isolation and/or amplification of nucleic acid sequences encoding novel microbial peptides are provided.

According to another aspect of the invention there is provided a method for the identification of families of nucleic acid sequences encoding antimicrobial peptides.

### BRIEF DESCRIPTION OF THE DRAWINGS

FIG. 1 is a textual and graphical depiction of pleurocidin WF2 cDNA from winter flounder (A), a graphical depiction of a predicted hydrophobicity plot of peptide WF2 (B), and a diagrammatic depiction of a predicted helical structure of WF2 (C).

FIG. 2 is a pictorial depiction of results of amplification of certain hepcidin-like cDNAs.

FIG. 3 is a depiction of certain aligned pleurocidin-like peptide sequences.

FIG. 4 is a pictorial depiction of the results of PCR amplification of certain pleurocidin-like genomic sequences.

FIG. 5 is a depiction of an extended genomic sequence of WF4.

FIG. 6 is a depiction of an alignment of certain pleurocidin-like polypeptide sequences.

FIG. 7 is a pictorial depiction of the results of expression of certain pleurocidin-like genes in different winter flounder tissues.

FIG. 8 is a pictorial depiction of the results of RTPCR of expression of certain pleurocidins during winter flounder development.

FIG. 9 is a pictorial depiction of the results of a study of the expression of certain pleurocidin-like genes during winter flounder development.

FIG. 10 is a pictorial depiction of the results of a Southern analysis of certain pleurocidin genes of winter flounder.

FIG. 11 is a schematic depiction of the genomic organization of certain pleurocidin genes from winter flounder.

FIG. 12 is a schematic depiction of certain transcription factor binding sites located upstream from pleurocidin genes from winter flounder.

FIG. 13 is a graphical depiction of results showing the impact of peptide NRC-15 on bacterial survival.

FIG. 14 is a graphical depiction of results showing the impact of peptide NRC-13 on bacterial survival.

FIG. 15 is a graphical depiction of results showing the impact of peptide NRC-12 on yeast survival.

FIG. 16 is a depiction of nucleotide sequences of an unspliced (A) and partially spliced (B) cDNA encoding a type I hepcidin and a schematic depiction of intron/exon structure of a hepcidin gene in human, mouse and salmon (C).

FIG. 17 is a depiction of certain hepcidin sequences from different species shown in alignment.

FIG. 18 is a depiction of certain aligned 3′ untranslated regions of hepcidin genes from winter flounder (A) and Atlantic salmon (B).

FIG. 19 is a pictorial depiction of the results of Southern hybridization analysis of certain hepcidins from different fish species.

FIG. 20 is a pictorial depiction of the results of an assay of the expression of certain hepcidin and actin genes in various tissues of winter flounder.

FIG. 21 is a pictorial depiction of the results of an assay of the expression of certain Type I(A) and Type 2(B) hepcidin and actin genes in various tissues of control and infected salmon.

FIG. 22 is a pictorial depiction of the results of an assay of expression of certain Type I(A), Type II(B) and Type III(C) hepcidin and actin genes in developing winter flounder larvae.

FIG. 23 is a schematic depiction of steps taken in an embodiment of the method for identifying pleurocidins.

FIG. 24 is a schematic depiction of steps taken in an embodiment of the method for identifying hepcidins.

FIG. 25 is a graphical depiction of experimental results using antimicrobial peptide NRC-13 in the presence of 150 mM NaCe.

### DETAILED DESCRIPTION OF THE INVENTION

The method of the invention builds on the surprising discovery that the flanking sequences around antimicrobial peptides, including without limitation pleurocidins and hepcidins, are conserved. The method of the invention provides a means of identifying nucleotide sequences encoding pleurocidins and hepcidins, and identifying the encoded polypeptide sequences.

In one embodiment, the method provides, generally, a way of identifying members of a family of antimicrobial peptides once a single family member has been identified. The initial family member may be an initial peptide of interest. Initial peptides of interest can be identified based on either known or reported antimicrobial activity or based on sequence similarity to other known antimicrobial peptides. Once an initial peptide has been identified, the genomic DNA encoding it is identified and its flanking sequences are determined.

As used herein, the term “flanking sequences” refers to nucleic acid sequences appearing at or near one or both ends of a target nucleic acid sequence encoding an antimicrobial peptide.

As used herein a nucleic acid sequence is “at or near” the end of a target sequence if a portion of the sequence is within 50 nucleic acids of the end of the gene (whether within the coding region or outside it).

When an initial peptide of interest is identified based on sequence similarity to another peptide with known antimicrobial activity, the initial peptide preferably has an amphipathic structure and a net charge. In some instances the charge will preferably be a net positive charge of at least 2. In some instances, the peptide is at least 75%, 85% or 95% identical in sequence to the peptide having known antimicrobial activity. In some instances the sequence similarity identified may relate to similarity between nucleic acid sequences encoding the known peptide and encoding the peptide of interest. In such instances, the predicted peptide for the peptide of interest will be considered with respect to predicted charge and amphipathic structure.

For example, the prepro-sequences of pleurocidins and hepcidins tend to be conserved. Thus, by employing nucleic acid primers specific for such sequences, one can identify potential pleurocidin- and hepcidin-encoding sequences. Alternatively or additionally, known gene sequences of other classes of antimicrobial peptides can be examined to identify regions which appear to encode conserved prepro-sequences and a similar strategy used to identify other members of this family of peptides. The corresponding antimicrobial peptide encoded by such sequences can be predicted using the general features found in most pleurocidins and hepcidins, such as, for example, a net positive charge of at least 2 and an amphipathic structure.

As used herein with respect to pre-, pro- and prepro sequences of antimicrobial peptides, “pre” and “pro” have the following meaning: “Pre” refers to the signal peptide portion (or a functional portion thereof) of the peptide. “Pro” refers to the propiece. In pleurocidins the propiece is the anionic region at the carboxy terminus. In hepcidins the propiece is the region upstream of the mature peptide. In the non-limiting examples disclosed herein pleurocidin primers were designed based on the pre and pro regions, and hepcidin primers were designed based on the pre region and the 3′ untranslated region (UTR).

PCR can be used to amplify nucleic acid sequences encoding potential pleurocidins or hepcidins. This can be conveniently accomplished by using a pair of PCR primers, one of which recognises a nucleic acid sequence complementary to a polynucleotide sequence encoding an amino-terminal prepro-sequence conserved in the peptide type of interest, and the other complementary to a 3′ conserved region in the nucleotide encoding the peptide-type of interest. It will be appreciated that other prepro-sequences may exist and are specifically contemplated. For example, redundancy in the genetic code allows for multiple nucleic acid sequences encoding a particular amino acid sequence. As discussed with respect to 5′prepro-sequences, other 3′ conserved sequences may exist and are specifically contemplated. When designing primers it is useful to have reference to known codon usage information for the species in which sequence amplification is sought.

In an embodiment of the invention there is provided the use of signal sequence I or a nucleic acid sequence encoding same in identifying or amplifying potential pleurocidins.

Signal Sequence I (SEQ ID NO: 305)
MKFTATFL (X)ₙ (L)ₒ (F)ₚ I (F)_{q} (X)_{y} VLM
(X)_{z} (V)ᵣ (E)ₛ (D)ₜ (P)ᵤ (L)ᵥ G E (C)_{w} (G)ₓ

| | |
|---|---|
| Wherein: | |
| n is 1 to 3 | u is 0 or 1 |
| o is 0 to 2 | v is 0 or 1 |
| p is 0 or 1 | w is 0 or 1 |
| r is 0 or 1 | |
| s is 0 or 1 | x is 0 or 1 |
| t is 0 or 1 | y is 0 or 1 |
| | z is 0 or 1 |
| with the restriction that: | |
| x + o + p = 3, | s + t = 1, |
| u + v = 1, | w + x = 1, and |
| q + = 1. | |

In an embodiment of the invention there is provided the use of one or both sequence PL1 or PL2 in identifying or amplifying potential pleurocidins.

| | |
|---|---|
| PL1 GCCCACTTTGTATTCGCAAG | (SEQ ID NO: 5) |
| PL2 CTGAAGGCTCCTTCAAGGCG | (SEQ ID NO: 6) |

In an embodiment of the invention there is provided the use of an acidic sequence I or a nucleic acid sequence encoding same in identifying or amplifying potential pleurocidins.

Acidic Sequence I (SEQ ID NO: 306)
(Y)ₐ (X)_{b} (X)_{c} (E)_{d} (X)ₑ (Q)_{f}(E)_{g} L (N/D)
KR (A/S) V D (D/E)

| | |
|---|---|
| wherein: | |
| a is 0 or 1 | e is 1 to 3 |
| b is 0 or 1 | f is 0 or 1 |
| c is 1 or 2 | g is 0 or 1 |
| d is 0 or 1 | |
| with the restriction that | |
| a + b = 1, | |
| c + d = 2, and | |
| e + f + g = 3. | |

As used in the sequences herein “X” refers to any amino acid. Nucleic acid sequences encoding signal sequence I and acidic sequence I are specifically contemplated, as are nucleic acid sequences complementary to such nucleic acid sequences. In an embodiment of the invention there is provided the use of signal peptide II, III, IV, V or a nucleic acid encoding same, in the identification or amplification of hepcidins.

| | |
|---|---|
| Signal Peptide II | |
| MKXXXXAXXVXXVL | (SEQ ID NO: 307) |
| Signal Peptide III | |
| MKTFSVAV | (SEQ ID NO: 308) |
| Signal Peptide IV | |
| MKTFSVAVTVAVVLXFICIQQSSA | (SEQ ID NO: 309) |
| Signal Peptide V | |
| MKTFSVAVAV (T/V) (L/V) VLA | (SEQ ID NO: 310) |
| (F)ₙ(V/C) (C/M) (I/F) (Q/I) X | |
| (X)ₘ S (S/T) AV P FXXV, | |

Wherein n is 0 or 1 and m is 0 or 1.

In an embodiment of the invention there is provided the use of prosequence I, Prosequence II or a nucleotide sequence encoding same or complementary to one encoding same in the identification or amplification of hepcidins.

| | |
|---|---|
| Prosequence I | |
| PEVQXLEEAXSXDNAAAEHQE | (SEQ ID NO: 311) |
| Prosequence II | |
| PFXXVX(X)ₙ (L/T) EEV (E/G) (G/S) | (SEQ ID NO: 312) |
| XD (T/S) PV (A/G) XHQ, | |

Wherein n is 0 or 1,

In an embodiment of the invention there is provided the use of HcPA3b3′ and/or HcSa13′ in the identification or amplification of hepcidins.

| | | |
|---|---|---|
| HcPa3b 3′ | 3′ACAACCTCGTCCTTAGG5′ | (SEQ ID NO: 313) |
| HcSal 3′ | 3′ACGCCCGTCCAGGAAT5′ | (SEQ ID NO: 314) |

Non-Limiting Examples Of Uses

Antimicrobial peptides are useful in the treatment and/or prevention of infection in a variety of subjects, including fish, reptiles, birds, mammals, amphibians and insects.

Antimicrobial peptides are also useful for reducing bacterial growth and/or accumulation on surfaces. This is of particular benefit in the food industry where antimicrobial peptides can be used for coating surfaces used in the processing, preparation, and/or packaging of food.

Antimicrobial peptides disclosed herein can be administered in a variety of ways. In some instances, oral administration will be desirable. Some types of oral administration will be improved by encapsulation of the peptides so as to allow their preferential release at a particular stage in digestion. In some instances it will be desirable to include pre and/or pro sequences in the administered peptide (for example to improve stability or modulate activity). The pre and/or pro sequences can be cleaved off by endogenous proteases at the appropriate stage. Peptides may be administered by inhalation where the subject breathes air or by addition to water for gilled subjects. Administration by injection will in some cases be desirable. Peptides may be injected into any number of sites. In some cases intravenous injection will be desired. In some instances injection directly into or adjacent to the site of infection or potential infection will be desired. In some instances topical administration will be desired. Where the presence of the antimicrobial peptide is desired at a remote and specific site, or where the peptide will be desired for a prolonged period of time, gene therapy may be used to provide expression of one or more antibacterial peptides in the tissue(s) of concern.

Where the subject is a cultured or domesticated creature such as a fish, bird or non-human mammal, production of a transgenic variety which expresses one or more antibacterial peptides may be desired. Methods for producing transgenic animals are well known. (See for example Mar. Biotechnol.4: 338,2002).

A variety of antimicrobial peptides are contemplated and fall within the scope of the invention. By way of non-limiting example, peptides comprising the following amino acid sequences or a sequence at least 80% or 90% homologous thereto, and nucleic acid sequences encoding them are specifically contemplated:

| | | |
|---|---|---|
| i) | GW(G/K)XXFXK | (SEQ ID NO: 315) |
| ii) | GXXXXXXXHXGXXIH | (SEQ ID NO: 316) |
| iii) | FKCKFCCGCCXXGVCGXCC | (SEQ ID NO: 317) |
| iv) | CXXCCNCC (K/H) XKGCGFCCKF | (SEQ ID NO: 318) |
| v) | FKCKFCCGCRCGXXCGLCCKF | (SEQ ID NO: 319) |
| vi) | XXXCXXCCNXXGCGXCCKX | (SEQ ID NO: 320) |

Other specific, non-limiting examples of antimicrobial sequences of interest can be found in Tables 4 and 11.

Antimicrobial peptides of the invention may be modified. Such modifications may in some instances improve the peptides′ stability or activity. Examples of modifications specifically contemplated include:
conservative amino acid substitutions (acidic with acidic, basic with basic, neutral with neutral, polar with polar, hydrophobic with hydrophobic, etc.)
addition of positively charged amino acids (lysine, arginine, histidine) at either or both ends
replacement of amino acids with others unlikely to result in structural changes including D-amino acids and peptidomimetics
deletion of one or more amino acids
modifications at C-terminal or N-terminal ends, including methyl esters and amidates
cyclised versions of the peptides (which may result in increased stability without adversely affecting activity)

### EXAMPLES

### Methods

Fish Rearing

Winter flounder larvae were reared as described (Douglas, Gawlicka et al. 1999), the disclosure of which is incorporated herein by reference. Saint John River stock Atlantic salmon (Salmo salar L.) were maintained in single-pass, heated, dechlorinated fresh water at 12° C. in the Dalhousie University Aquatron facility in Halifax, Nova Scotia. All fish were euthanised with an overdose of tricaine methanesulfonate (MS 222, 0.1 g L⁻¹, Argent Chemical Laboratories, Inc., Redmond, Wash., USA) prior to sampling. All animal procedures were approved by the Dalhousie University Committee for Laboratory Animals and the National Research Council—Halifax Local Animal Care Committee.

Bacterial Challenge

Aeromonas salmonicida subsp salmonicida strain A449 (Trust et al. 1983) was cultured to mid-logarithmic growth in Tryptic Soy Broth (TSB) at 17° C. The absorbance at 600 nm of the bacterial suspension was determined and the bacteria were resuspended to approximately 5×10⁷ cfu mL⁻¹ in sterile Hanks Balanced Salt Solution (HBSS). Three salmon (200 g each) were anaesthetised with 50 mg L⁻¹ TMS, injected intraperitoneally with 2.5×10⁶ cfu bacteria in 50 μL HBSS and allowed to recover in fresh water. Uninjected fish from the same cohort were maintained in separate tanks as controls. Three days post-injection, control and infected salmon were euthanised as described above and samples of tissues removed. Blood was drawn from the caudal vein into a heparinised container. To confirm that the fish were positive for A. salmonicida, the posterior kidney of both infected and control fish were swabbed and used to inoculate tryptic soy agar (TSA) that was incubated at room temperature overnight. Atlantic halibut tissue samples were obtained from a bacterial challenge study performed at Bedford Institute of Oceanography, Dartmouth, Nova Scotia.

Sampling

Tissues (oesophagus, stomach, pyloric caecae, liver, spleen, intestine, anterior kidney, posterior kidney, gill, skin, ovary, rectum, heart, muscle and brain) were removed into RNALater (Ambion, Austin, Tex., USA) and kept at −80° C. until used. Samples of winter flounder larvae at different stages and juveniles were rinsed in RNALater (Ambion, Austin, Tex., USA), transferred into 1.5 ml Eppendorf tubes containing 0.5-1.25 ml RNALater, and kept at −80° C. until used.

Pleurocidins

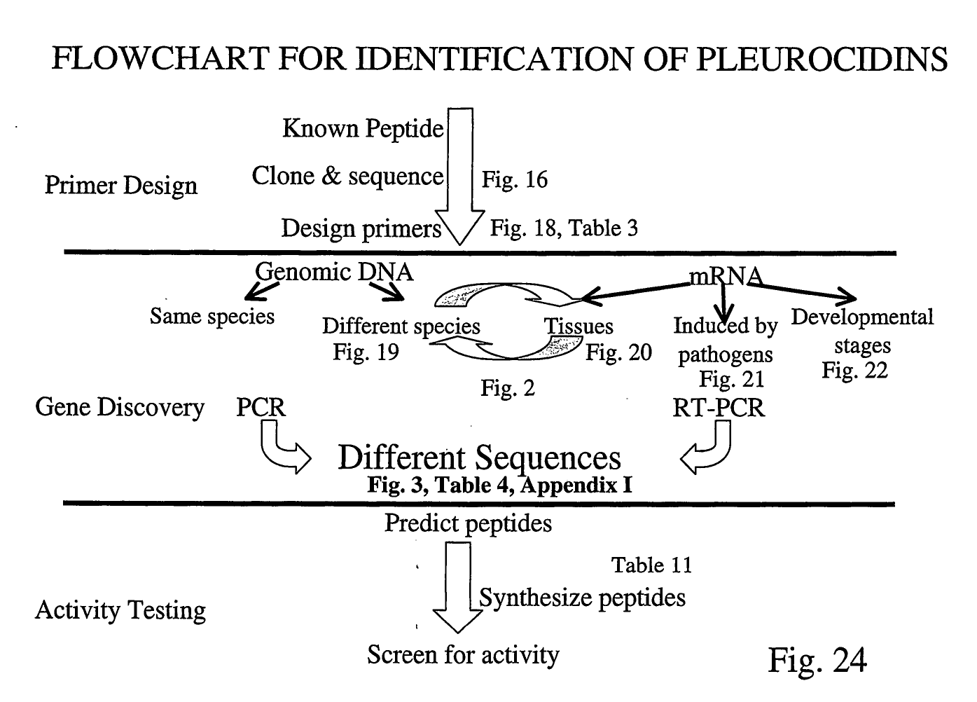

The general approach followed is shown in FIG. 24

Isolation of Pleurocidin cDNA

A cDNA library constructed from winter flounder skin (Gong et al 1996) was screened using degenerate oligonucleotides (PleuroA, PleuroB; Table 1). The library was plated at 80,000 phage/plate and duplicate lifts to HyBond filters were made of each of eight plates. A mixture of radioactively end-labelled PleuroA and PleuroB probes was hybridised with the filters at 50° C. using standard procedures, and the filters were washed in 1×SSC/0.1% SDS at 50° C. for 45 min. Plaques that showed matching hybridization signals on both duplicate filters were picked and the library rescreened until 100% purity of the recombinant plaques was obtained. Two recombinants were completely sequenced using an ABI373 stretch automated sequencer and the AmpliTaqFS Dye Terminator Cycle Sequencing Ready Reaction kit (Perkin-Elmer, Foster City, Calif., USA). Sequence data were analyzed using Sequencher (Gene Codes, Inc., Ann Arbor, Mich., USA) and DNA Strider. The amino-terminal signal sequence was predicted using SignalP (http://www.cbs.dtu.dk/services/SignalP). The Helical Wheel routine of the GCG package (http://www.gcg.com) was used to model the helical structure of the predicted antimicrobial peptide sequences.

Genomic PCR

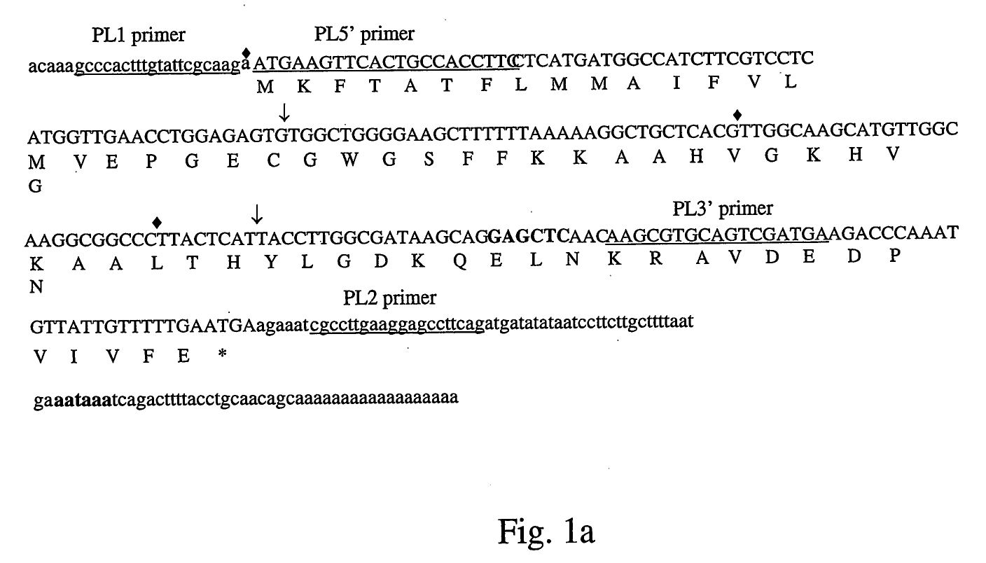

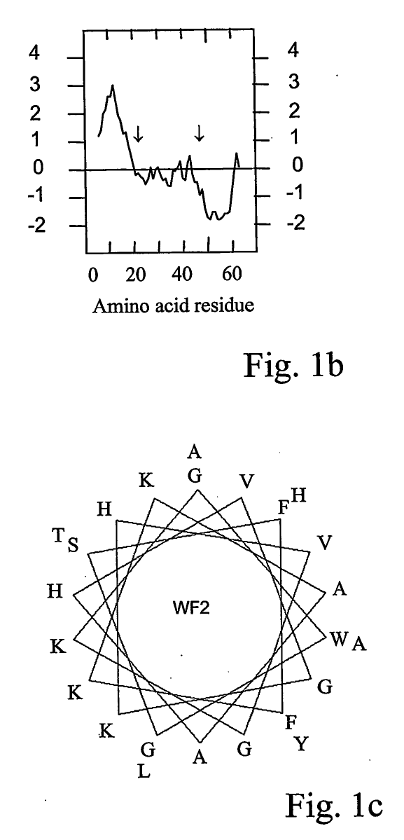

Genomic sequences were amplified using two sets of primers specific to the winter flounder pleurocidin cDNA (PL1/PL2 and PL5′/PL3′; Table 1; FIG. 1). The amplification conditions were: 1 min at 94° C.; 35 cycles of 30 s at 940 C; 30 s at 52° C., 90 s at 72° C.; and 2 min at 72° C., and products were resolved on a 1% agarose gel. Bands were excised from the gel, extracted using Gene-Clean (Bio101, La Jolla, Calif., USA) and cloned into the Topo TA2.1 vector (Invitrogen, Carlsbad, Calif., USA) as recommended by the manufacturers. Several isolates from each transformation were sequenced and analyzed as described above. Intron positions were identified by comparison with the cDNA sequence.

Identification of Additional Winter Flounder Pleurocidin-Like Sequences by RT-PCR

Total RNA was isolated from winter flounder skin and intestine substantially as described in Douglas, Gawlicka et al (1999). Reverse transcription of 2 μg of total RNA was performed using the RETROScript kit (Ambion, Austin, Tex., USA) according to the manufacturer's recommendation. PCR was performed using PL3′ and a primer corresponding to the amino terminus of the precursor polypeptide (PL5′; Table 1). The amplification conditions were: 1 min at 94° C.; 32 cycles of 30 s at 94° C., 30 s at 50° C., 90 s at 72° C.; and 2 min at 72° C. and products were resolved on a 2% NuSeive gel. Bands were excised, cloned and sequenced as described above.

Identification of Additional Pleurocidin-Like Sequences From Different Tissues

Tissue-specific expression of pleurocidin was investigated by northern analysis using polyadenylated RNA (500 ng) from adult skin, liver, ovary, muscle, spleen, pyloric caeca, stomach and intestine. The entire insert from the cDNA clone corresponding to WF2 was radioactively labelled and incubated with the blot overnight at 60° C. in UltraHyb hybridisation solution (Ambion, Austin, Tex., USA). The blot was washed to a stringency of 50° C. in 1×SSC/0.1% SDS for 1 h before exposure to X-ray film. RT-PCR was also employed using primers specific to WF1, WF1a, WF2, WF3, WF4, WFYT and WFX (Table 2) to assay expression of the different pleurocidin-like variants in various tissues. The conditions used were as described in the preceding paragraph except that the annealing temperature was 52° C.

Identification of Additional Pleurocidin-Like Sequences From Different Developmental Stages

Two larval time series were used to assess developmental expression of pleurocidin-like genes. In the first, RNA was isolated from pooled samples of twenty whole larvae (5 and 13 dph), ten whole metamorphosing larvae (20 dph) and newly metamorphosed larvae (27 dph), gut tissue of two juveniles (41 dph), skin from the upper and lower side of adult fish and tissue from adult upper and lower intestine. RNA was isolated as described (Douglas, Gawlicka et al. 1999), the disclosure of which is incorporated herein by reference, and the assays were performed using the primers PL5′ and PL2 and conditions described above for RT-PCR. Amplification of the actin mRNA was performed as previously described (Douglas, Bullerwell et al. 1999), the disclosure of which is incorporated herein by reference, to confirm the steady level of expression of a housekeeping gene and to provide an internal control for pleurocidin expression. In the second larval time series, RNA was isolated from pooled samples of twenty whole larvae (hatch, 5 and 9 dph), ten whole larvae (15, 20, 25, 30 and 36 dph) and gut tissue of two juveniles (41 dph). Assays were performed using primers specific to WF1, WF1a, WF2, WF3, WF4, WFYT and WFX (Table 2) to determine expression of the different pleurocidin-like variants at different stages of development. The conditions used were as described in the preceding paragraph.

Southern Analysis

Southern analysis of BamHI- and SstI-digested genomic DNA from winter flounder, three other flatfish (American plaice Hippoglossoides platessoides Fabricius, Atlantic halibut Hippoglossus hippoglossus L. and yellowtail flounder Pleuronectes ferruginea Storer), haddock (Melanogrammus aeglefinus L.), pollock (Pollachius virens L.) and smelt (Osmerus mordax Mitchill) was performed sequentially using the entire inserts from genomic clones corresponding to WF1, WF2, WF3 and WF4 as probes. Hybridisations were performed overnight at 65° C. as previously described (Douglas, Gallant et al. 1998), the disclosure of which is incorporated herein by reference, and the blots were washed at 65° C. in 0.5×SSC/0.1% SDS for 1 h and exposed to X-ray film. Blots were stripped by incubating twice in boiling 0.5% SDS and checked for residual signal by exposure to X-ray film overnight.

Identification of Additional Pleurocidin-Like Sequences From Other Fish Species

Total RNA was isolated from skin and intestine of yellowtail flounder, witch flounder and Atlantic halibut and reverse-transcribed as described above (RT-PCR analysis). Total genomic DNA was isolated from milt of yellowtail flounder, witch flounder, American plaice, Atlantic halibut and tissue samples of Petrale sole, C—O sole, English sole, Starry flounder, European plaice, Greenland halibut and Pacific halibut. Two sets of primers specific to the winter flounder pleurocidin cDNA (PL1/PL2 and PL5′/PL3′; Table 1; FIG. 1) were used and the amplification conditions were: 1 min at 94° C., 32 cycles of 30 s at 94° C.; 30 s at 50° C., 90 s at 2 min at 72° C. Products were resolved on a 2% NuSeive gel, bands excised, cloned and sequenced as described above.

FIG. 1 is a textual and graphical depiction of WF2 pleurocidin from winter flounder A. Nucleotide sequence of cDNA for pleurocidin from winter flounder isolated from the skin library. The positions of primers used for PCR are underlined and the deduced amino acid sequence is shown in upper case letters below the nucleotide sequence. Arrows indicate the mature 5′ and 3′ termini of the pleurocidin peptide and diamonds indicate the positions of introns. The single SstI restriction endonuclease site (GAGCTC) and the putative polyadenylation site (aataaa) are indicated in boldface. B. Hydrophobicity plot of predicted pleurocidin polypeptide WF2 constructed using the Kyte-Doolittle option of DNA Strider (Marck 1992). The borders of the mature pleurocidin are indicated by vertical arrows. C. Diagrammatic representation of helical structure of predicted pleurocidin polypeptide WF2 constructed using the Helical Wheel routine of GCG. Hydrophobic residues and glycines are boxed and polar residues are not. The first amino acid (G) of the mature polypeptide is found at the top of the wheel.

Identification of Pleurocidin-Like Sequences in the Winter Flounder Genome

A winter flounder genomic λ-GEM library was screened using a radioactively labeled probe for pleurocidin (WF2; Douglas et al., 2001). Four clones were picked and replated until 100% purity was achieved. The clones were mapped using BamHI, SstI, XhoI and Eco RI and two clones (λ1.1 and λ5.1) that differed in restriction pattern were selected for sequencing. Both clones were completely sequenced using an ABI373 stretch automated sequencer and the AmpliTaqFS Dye Terminator Cycle Sequencing Ready Reaction kit (Perkin Elmer, Foster City, Calif., USA. Transcription factor binding sites were identified using WWW Signal Scan (http://bimas.dcrt.nih.gov/molbio/signal/) with the TransFac and TFD databases and promoters were detected using the eukaryotic promoter prediction by neural network software available at the Baylor College of Medicine (http://searchlauncher.bcm.tmc.edu/seq-search/gene-search.html).

Hepcidins

The general approach followed is depicted in FIG. 24

Molecular Characterisation of Hepcidin cDNAs

Eight ESTs showing high similarity to human hepcidin were identified from the winter flounder EST database (Douglas, Gallant et al. 1999) and four from the Atlantic salmon database (Douglas, Tsoi et al. 2002). Using these sequences to screen dbEST, BLASTX analysis revealed two related sequences from Japanese flounder (C23298.1 and C23432.1), one sequence from rainbow trout (AF281354_{—1}) and five identical sequences from medaka (AU178966, AU179222, AU179314, AU179768 and AU180044). Sequence data were analyzed using Sequencher (Gene Codes, Inc., Ann Arbor, Mich., USA) and DNA Strider (Marck 1992). Alignments and similarity matrices were calculated using ClustalW (Thompson, Higgins et al. 1994) and graphically visualised using SeqVu (Garvan 1996). The on-line servers PSORT (http://PSORT.nibb.ac.ip), Compute pI (http://expasy.hcuge.ch/cgi-bin/pi tool), and Network Protein Sequence @nalysis (http://npsa-pbil.ibcp.fr/cpi-bin/secpred_consensus.pl) were used to predict N-terminal signal sequences, pI and secondary structure, respectively. The secondary structure prediction program utilized seven different algorithms (for details, see web site) and provided a consensus prediction based on these results.

Southern Hybridisation

Total genomic DNA was prepared from winter flounder (Pleuronectes americanus), yellowtail flounder (Pleuronectes ferruginea), witch flounder (Glyptocephalus cynoglossus), Japanese flounder (Paralichthys olivaceus), American plaice (Hippoglossoides platessoides), Atlantic salmon (Salmo salar), haddock (Melanogrammus aeglefinus), smelt (Osmerus mordax), hagfish (Eptatretus burgeri), tiger shark (Scyliorhinus torazame) and white sturgeon (Acipenser transmontanus) as previously described (Douglas, Bullerwell et al. 1999), the disclosure of which is incorporated herein by reference. DNA (7.5 □g) was digested with SstI according to the manufacturer's recommendations and the fragments resolved on a 1% agarose gel. A 104 bp probe corresponding to amino acid residues WMENPT . . . GCGFCC (SEQ ID NO: 321 and 322 respectively) of Type I winter flounder hepcidin was labeled using the DIG Labelling Kit (Roche Applied Science, Laval, PQ, Canada) and hybridized to the membrane for 2 h at 42° C. using the Easy Hyb kit (Roche Applied Science, Laval, PQ, Canada). The membrane was washed in 0.2×SSC at 65° C. and signal detected using the DIG Luminescent Detection Kit (Roche Applied Science, Laval, PQ, Canada).

Identification of Additional Hepcidin-Like Sequences by RT-PCR

Primers were designed based on the cDNA sequences determined in this study (Table 3). Amplification of actin mRNA was performed to confirm the steady-state level of expression of a housekeeping gene and provide an internal control for the hepcidin gene expression analyses. Controls were performed using single primers to eliminate single primer artifacts and without reverse transcription to eliminate amplification products arising from contaminating genomic DNA.

Total RNA was isolated from tissues of uninfected adult winter flounder and uninfected and infected adult salmon and halibut using the RNAWiz Kit (Ambion, Austin, Tex., USA) according to the manufacturer's recommendations. Tissues were homogenized using a 7mm generator on a Polytron standard rotor stator homogenizer (Kinematica). In addition, RNA was isolated from pooled samples of twenty whole larvae (hatch, 5 and 9 dph), ten whole larvae (15, 20, 25, 30 and 36 dph), gut tissue of two juveniles (41 dph) and adult winter flounder liver. To eliminate contaminating DNA, the Ambion DNA-free TM protocol was used as directed. Briefly, 4 units of DNase 1 was added to the resuspended RNA and incubated for 1 hour at 37 C. After incubation, DNAse Inactivation Reagent was added to remove the enzyme and RNA concentrations were determined using a Beckman DU-64 Spectrophotometer.

First strand cDNA was synthesized from 1 μg of total RNA using the RetroScript kit (Ambion, Austin, Tex., USA) and aliquots of the reaction products were subjected to PCR using rTaq polymerase (Amersham Pharmacia Biotech AB, Uppsala, Sweden) or the Advantage2 PCR kit (Clontech, Palo Alto, Calif., USA). The primers and annealing temperatures are listed in Table 3. The amplification conditions were: 1 min at 95° C.; 32 cycles of 15 s at 95° C.; 30 s at the annealing temperature, 30 s at 68° C.; hold at 4° C. Amplification products were resolved on a 2% NuSieve agarose gel with a 100 bp ladder as a marker (Gibco BRL, Gaithersburg, Md., USA) and the amount of each product was quantified using a GelDoc 1000 video gel documentation system (BioRad, Mississauga, Ont., Canada) with the Multianalyst software.

Identification of Additional Hepcidin-Like Sequences From Other Fish Species

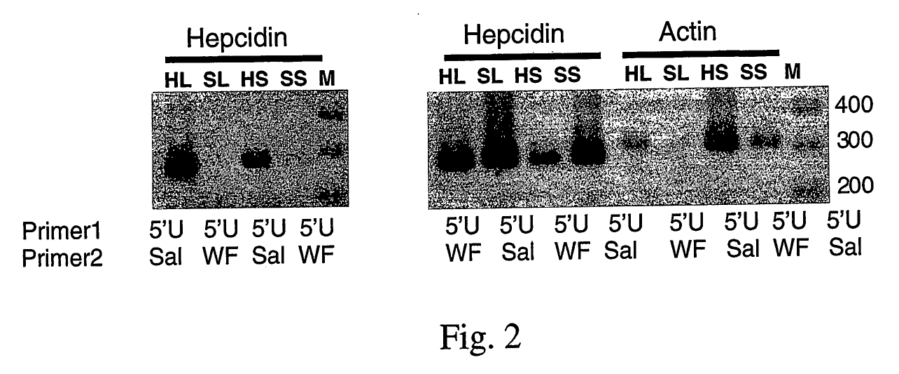

Total RNA was isolated from liver and spleen of bacterially challenged Atlantic halibut and Atlantic salmon and reverse-transcribed as described above (RT-PCR analysis). Two sets of primers were used (see legend, FIG. 2) and the amplification conditions were: 2 min at 94° C.; 32 cycles of 30 s at 94° C.; 30 s at 52° C., 30 s at 72° C.; and 2 min at 72° C. Products were resolved on a 2% NuSeive gel, bands excised, cloned and sequenced as described above.

Bacterial Strains and Candida albicans

All strains used in this study are listed in Table 5. Most non-fish bacterial strains as well as Candida albicans were grown at 37° C. in Mueller-Hinton Broth (MHB; Difco Laboratories, Detroit), while the fish bacteria were maintained at 16° C. in Tryptic Soy Broth (TSB; Difco, 5g/l NaCl). All strains were stored at −70° C. until they were thawed for use and sub-cultured daily. The following strains, Pseudomonas aeruginosa K799 (parent of Z61), Pseudomonas aeruginosa Z61 (antibiotic supersusceptible), Salmonella typhimurium 14028s (parent of MS7953s), Salmonella typhimurium MS7953s (defensin supersusceptible), as well as Staphylococcus epidermidis (human clinical isolates) and methicillin-resistant Staphylococcus aureus (MRSA; isolated by Dr. A. Chow, University of British Columbia) have been kindly donated by Prof R. E. W. Hancock, University of British Columbia.

Escherichia coli strain CGSC 4908 (his-67, thyA43, pyr-37), auxotrophic for thymidine, uridine, and L-histidine (Cohen et al., 1963) was kindly supplied, free of charge, by the E.coli Genetic Stock Centre (Yale University, New Haven, Conn.). MHB supplemented with 5 mg/L thymidine, 10 mg/L uridine and 20 mg/L L-histidine (Sigma Chemical Co., St. Louis, Mo.), was used to grow E.coli CGSC 4908 unless otherwise specified.

Two field isolates of the salmonid pathogen Aeromonas salmonicida are from the IMB strain collection.

Minimum Inhibitory Concentrations

The activities of the antimicrobial peptides were determined as minimal inhibitory concentrations (MICs) using the microtitre broth dilution method of Amsterdam (Amsterdam, 1996), as modified by Wu and Hancock (1999). Serial dilutions of the peptide were made in water in 96-well polypropylene (Costar, Coming Incorporated, Coming, New York) microtiter plates. Bacteria or C. albicans were grown overnight to mid-logarithmic phase as described above, and diluted to give a final inoculum size of 10⁶ cfu/ml. A suspension of bacteria or yeast was added to each well of a 96 well plate and incubated overnight at the appropriate temperature. In the case of E. coli CGSC 4908, supplemented MHB was used. Inhibition was defined as growth lesser or equal to one-half of the growth observed in control wells, where no peptide was added. Three repeats of each MIC determination were performed.

Killing Assays

Survival of bacteria and C. albicans upon exposure to selected peptides applied at their minimal inhibitory concentrations (MICs) and ten times their MICs was measured using standard methodology. The test organisms were grown in MHB and exposed to the peptides. At the specified time intervals equal aliquots were removed from the cultures, plated on MHB plates, and the resulting colonies were counted. Percentage survival was plotted against time on a logarithmic scale. Two repeats of each experiment were performed.

Preparation of a Synthetic Antimicrobial Peptide

Prediction of active cationic peptide sequences.

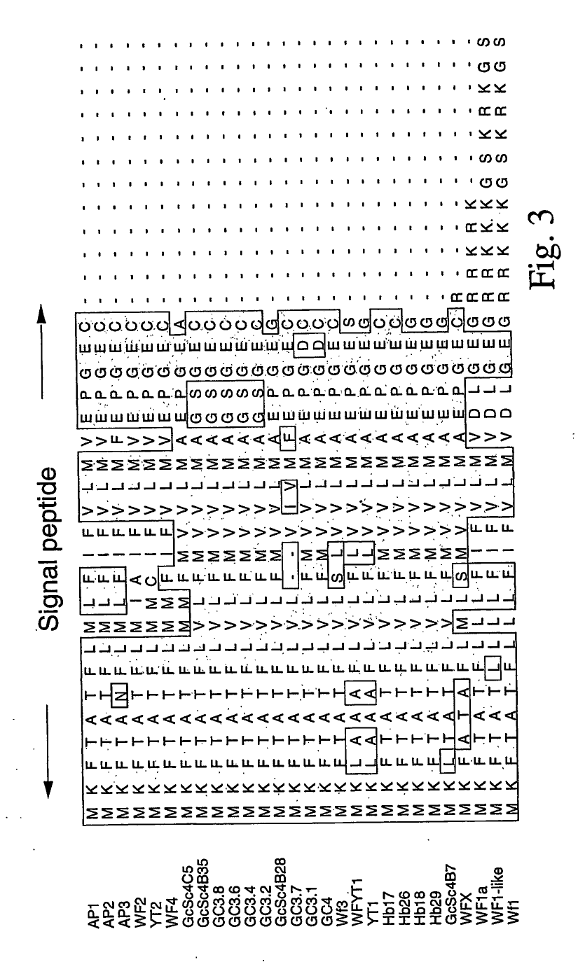

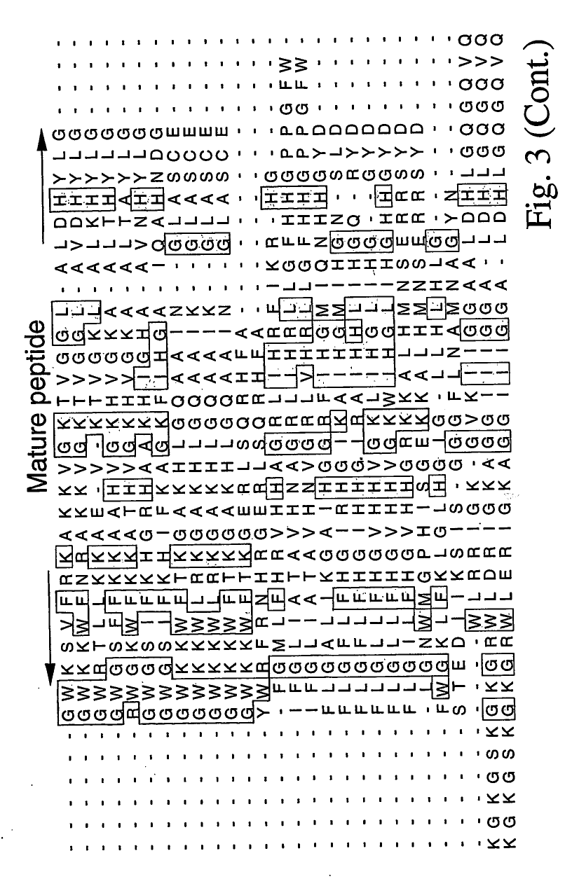

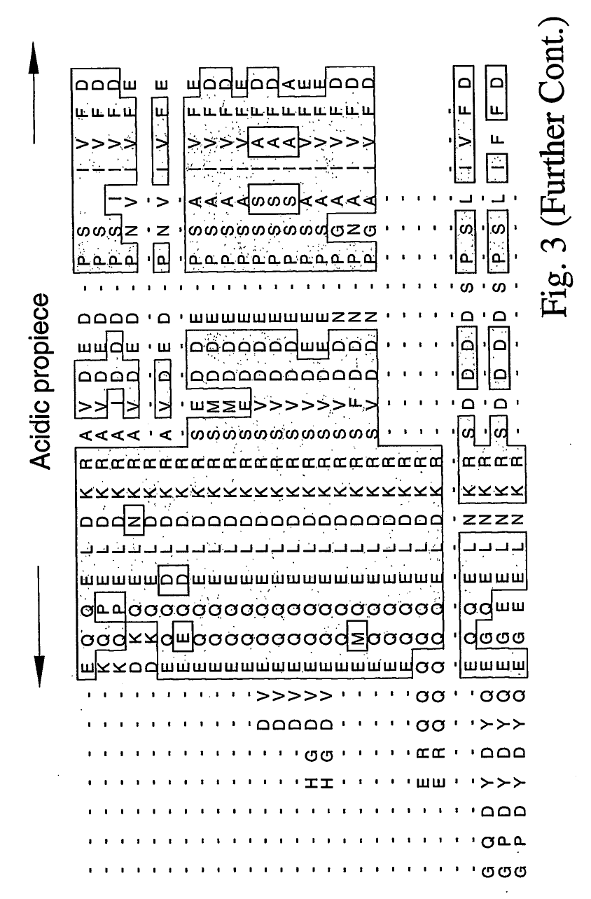

The mature peptide sequences from FIG. 3 (pleurocidin-like peptide sequences deduced from nucleotide sequences of genes and PCR products amplified from fish tissues) constituted the basis of sequence selection.

Upon extensive sequence analysis, sequences were selected for peptides that possessed a net positive charge and had their hydrophilic and hydrophobic residues well separated spatially in models.

Also, generally those peptide genes that were likely to be expressed (possessed promoters, were transcribed, etc.) were produced, although pseudogenes were also included in the panel.

The exact start/end residues were decided upon based on several factors:

a) In most cases the N-terminus of the mature peptide was well-defined, since it followed directly the conserved signal peptide region, and aligned well with other mature peptides.

b) Wherever a straightforward determination on the N-terminal amino acid was not possible, an attempt was made to preserve GW or GF at the N-terminus, as this is frequently encountered among cationic peptides.

c) In addition, two versions of WF1a (NRC-2 and NRC-3) were produced: one contained N-terminal GRRKRK (SEQ ID NO: 323), and the other did not; this was done because it was hypothesized that the presence of the highly positively charged GRRKRK (SEQ ID NO: 323) would improve activity.

d) Although in some cases the C-terminus of the mature peptide was also well defined, since it was followed directly by a conserved acidic propiece, significant ambiguity as to the C-terminal amino acid existed among many peptides. Generally, two rules were followed in deciding upon C-terminal amino acids:

1. wherever glycine appeared at or near the C-terminus, it was considered to be a precursor for carboxy-terminus amidation;

2. large numbers of negatively charged amino acids near the C-terminus were generally considered to be a part of the propiece and not mature active peptide and were not included in the sequence.

In order to estimate the net charge, K and R were assumed to have the value of +1, H of +1/2, D and E of −1, and C-terminal amidation was counted as an additional +1. The EMBOSS Pepwheel and Pepnet internet tools available through an NRC mirror site (http://bioinfo.pbi.nrc.ca:8090/EMBOSS/index.html) were used to analyse the separation of hydrophilic and hydrophobic residues in helical wheel and helical net models.

All antimicrobial peptides used in this study were synthesized by N-(9-fluorenyl) methoxy carbonyl (Fmoc) chemistry at the Nucleic Acid Protein Service (NAPS) unit at the University of British Columbia. Peptide sequences are shown in Table 4. Peptide purity was confirmed by HPLC and mass spectrometry analysis in each case. In the case of NRC-7 further purification by RP-HPLC was performed until homogeneity of the sample was obtained.

Peptides produced according to the above steps are screened for antimicrobial activity in vitro by standard means. Those peptides showing in vitro antimicrobial activity are useful as antimicrobial peptides for use in vivo and for the treatment of surface, etc.

### EXAMPLES

### Results

Pleurocidins

cDNA sequence

The two clones isolated from the winter flounder skin cDNA library were identical in sequence to each other and to the genomic PCR product WF2 after introns were removed (see below). They contain 356 bp and encode an open reading frame of 68 amino acids (FIG. 1A). There is a 5′-untranslated region of 26 bp and a 3′-untranslated region of 84 bp, excluding the polyA tail. A canonical polyadenylation signal AATAAA is found 22 bp upstream of the polyA tail. The first 22 amino acids of the open reading frame form a highly hydrophobic domain (FIG. 1B) predicted to be a signal peptide with a cleavage site that precisely matches the amino terminus of the mature pleurocidin. The predicted amino acid sequence of residues 23-47 exactly matches the published amino acid sequence of mature pleurocidin (arrows, FIG. 1A). The mature peptide can assume an amphipathic helix that contains a predominance of positively charged amino acids on one face and hydrophobic amino acids on the other (FIG. 1C). The carboxy-terminal 21 amino acids form a negatively charged domain that is not present in the mature pleurocidin, confirming the recent report of Cole et al. (2000).

Genomic PCR

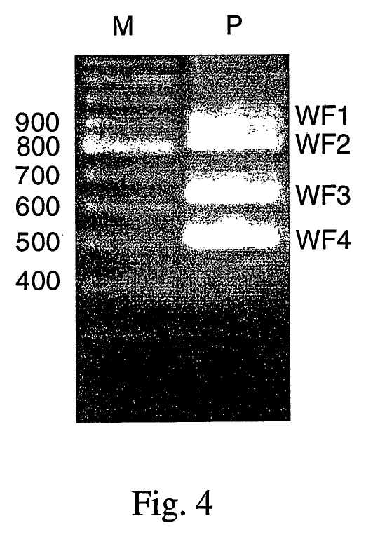

Four distinct bands (WF1-4) were amplified using primers PL5′ and PL3′ (FIG. 4). Sequence analysis of each product was consistent with the sizes of the bands and verified that each amplification product was different (Table 6). Two distinct bands were amplified using primers PL1 and PL2 that corresponded to WF2 and WF4 containing additional upstream and downstream sequence (data not shown). When the intron sequences were removed, the sequence of WF2 exactly matched that of the pleurocidin cDNA clone isolated from the skin library (FIG. 1A).

FIG. 4 is a depiction of the results of PCR amplification of pleurocidin-like sequences from winter flounder genomic DNA. Amplification products (P) were resolved on a 1% agarose gel using the 100 bp ladder as molecular weight markers (M). Products visible as distinct bands are labeled WF1 (00 bp), WF2 (810 bp), WF3 (650 bp) and WF4 (510 bp).

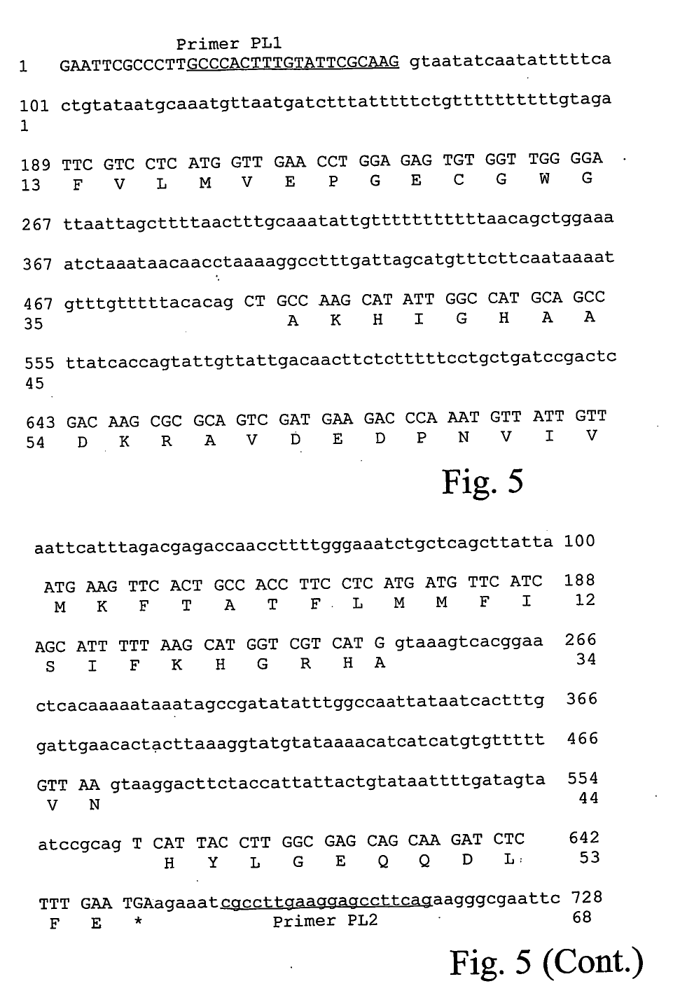

All four of the pleurocidin-like genes contained two introns within the coding sequence and three of the genes showed identical intron locations (WF1, WF2 and WF4). However, the position of the second intron in WF3 occurred upstream of those of the other genes, resulting in a shorter second exon and longer third exon. The sizes and sequences of the introns varied among the four pleurocidin genes (Table 6). Evidence from the two more extensive genomic sequences of WF2 and WF4 obtained using primers PL1 and PL2 indicates that a third intron immediately upstream of the initiation codon is also a feature of this gene family (FIG. 5). This was also noted for the genomic sequence reported by Cole et al (Cole, Darouiche et al. 2000).

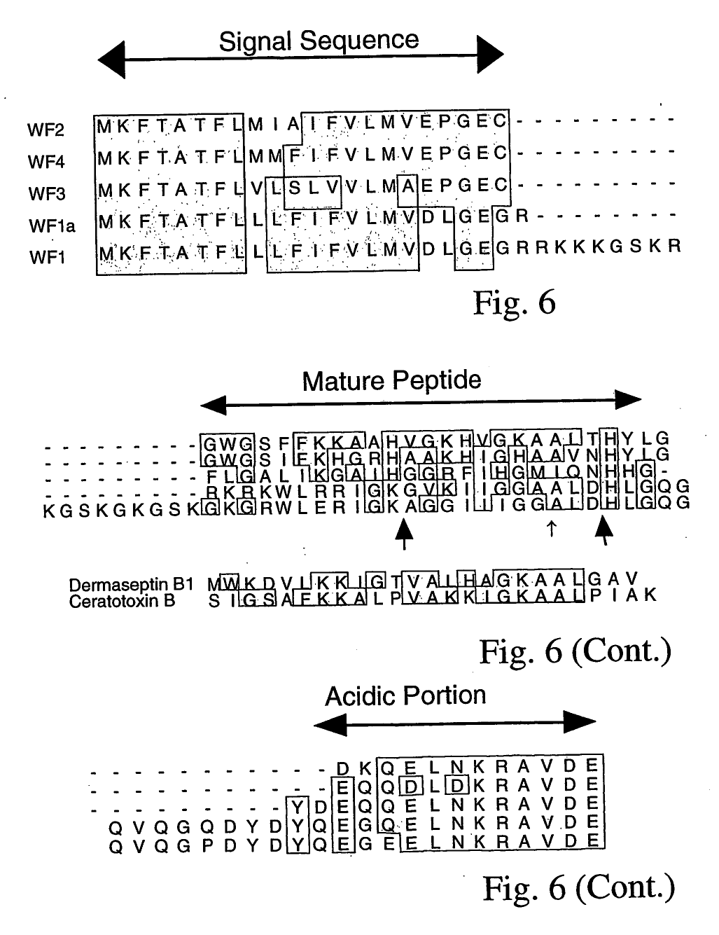

An alignment of the predicted amino acid sequences is shown in FIG. 6. The positions of the introns (indicated by vertical arrows) were determined by comparison with the corresponding RT-PCR and cDNA-derived sequences. The positions of the mature peptide were determined by comparison with the published amino acid sequence of pleurocidin (Cole, Weis et al. 1997). All of the predicted mature polypeptides could assume amphipathic α-helical structures similar to that shown in FIG. 1C, although the positively charged portions were not as striking in WF1 and WF3 as in WF2 and WF4 (data not shown).

FIG. 5 describes extended genomic sequence of WF4 obtained by PCR using primers PL1/PL2. Introns are indicated in lower case and coding sequence in upper case The positions of the primers PL1 and PL2 used for PCR are underlined.

FIG. 6 describes Alignment of predicted polypeptide sequences of five winter flounder pleurocidin family members. Large vertical arrows indicate the positions where introns were found in the genomic sequences. The second intron of WF3, indicated by a small vertical arrow, is found more upstream than those of the other genes. The predicted polypeptide sequences of dermaseptin B1 (Amiche et al. 1994) and ceratotoxin B (Marchini et al. 1995) are shown below the pleurocidin family members. Boxed amino acids are shared by half of the sequences.

Identification of Additional Pleurocidin-Like Sequences From Different Tissues

Northern analysis was only able to detect pleurocidin transcripts in skin (data not shown). However, the more sensitive RT-PCR assay indicated that pleurocidin was also expressed in other tissues, particularly gill and gut. Using primers PL5′ and PL3′, two bards were obtained from winter flounder skin (265 and 175 bp) and two from intestine (215 and 175 bp). Sequence analysis of several clones of each size showed that the 265 bp winter flounder skin clones corresponded to the genomic sequence of WF1 when intron sequences were removed (Table 7). Five of the 175 bp clones from skin and two of the 175 bp clones from intestine corresponded to the genomic sequence of WF2. This is consistent with results of northern analysis using the cDNA clone corresponding to the WF2 probe that showed hybridisation only to 200-nucleotide mRNA from the skin (data not shown). On the other hand, nine of the 175 bp clones from intestine and four of the 175 bp clones from skin corresponded to the genomic sequence of WF3. No RT-PCR products were obtained that corresponded to WF4. All seven of the 215 bp intestine clones corresponded to a novel family member (WF1a) not represented by any of the winter flounder genomic sequences determined in this study.

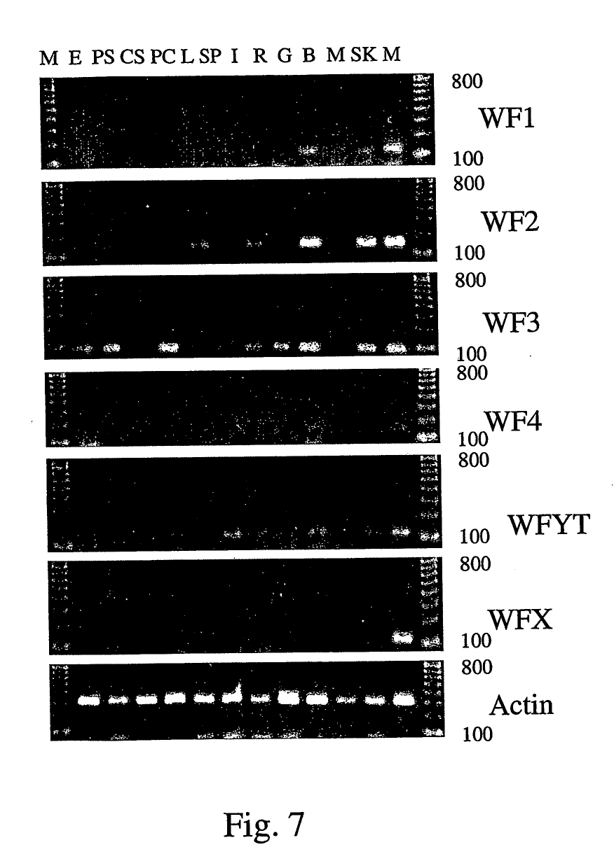

Using primers specific to each of the pleurocidin-like variants reported above, as well as to additional pleurocidin-like variants identified on Lambda clones, we were able to demonstrate that different variants were expressed in different tissues (FIG. 7). WF2, WF3 and WFYT showed the expression in the widest distribution of tissues, whereas WF1 and WF4 were expressed in mainly in the gill and skin, and WFX was only expressed in the skin. Transcripts of WF 1 a could not be detected in any tissue.

FIG. 7 describes the expression of specific pleurocidin-like genes in different tissues of winter flounder. Tissues were esophagus (E), pyloric stomach (PS), cardiac stomach (CS), pyloric caeca (PC), liver (L), spleen (SP), intestine (I), rectum (R), gill (G), brain (B) and skin (SK). Markers (M) were the 100 bp ladder. Primers were specific to each pleurocidin variant (Table 2)

Identification of Additional Pleurocidin-Like Sequences From Different Developmental Stages

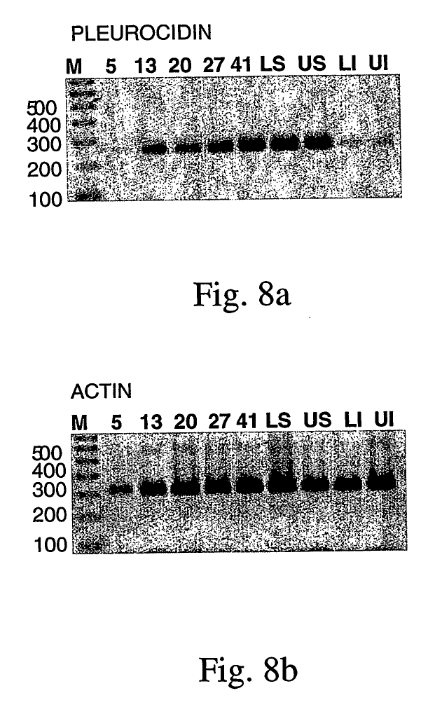

Using primers PL5′ and PL2 (Table 1) from highly conserved regions of the pleurocidin-like peptides, low levels of transcripts were evident at 5 dph and increased during development (FIG. 8). Strong signals were obtained from adult skin and weak signals from intestinal tissue. Expression of the housekeeping gene, actin, was relatively constant throughout development.

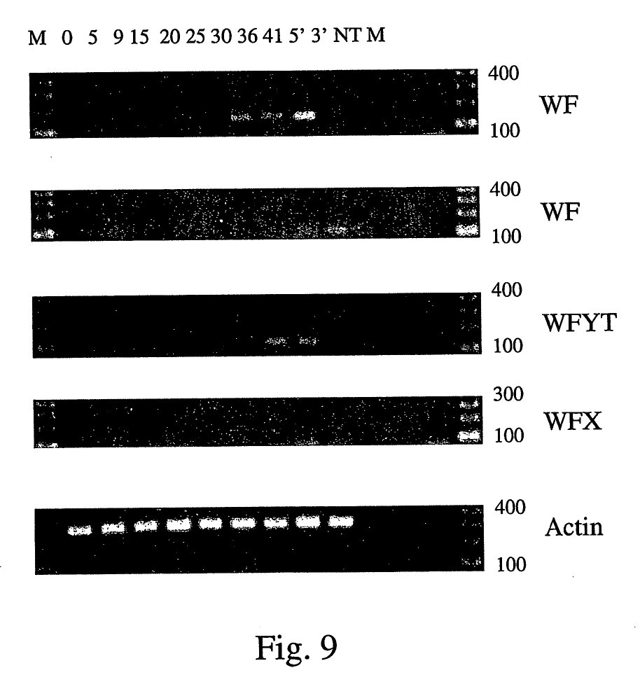

Using primers specific to each of the pleurocidin-like variants reported above, as well as to additional pleurocidin-like variants identified on Lambda clones, it was demonstrated that different variants were expressed at different times during development (FIG. 9). WFX transcripts were only detectable at 20 dph, and WF2, WF3 and WFYT were detectable in premetamorphic larvae and metamorphic juveniles. No expression of WF1 and WF4 was detectable at any stage of development.

FIG. 8 describes Reverse transcription-polymerase chain reaction assay of pleurocidin expression. Samples are from larvae (5 and 13 dph), metamorphosing larvae (20 dph), newly metamorphosed larvae (27 dph), juveniles (41 dph), skin from the lower (LS) and upper side (US) of the fish and tissue from the lower (LI) and upper (UI) intestine. Primers specific for pleurocidin (panel A) and actin (panel B) were used.

FIG. 9 describes Expression of specific pleurocidin-like genes during winter flounder larval development. Samples are from larvae (5, 9 and 15 dph), metamorphosing larvae (20 dph), newly metamorphosed larvae (25, 30 and 36 dph) and juveniles (41 dph). Controls using the 5′ or 3′ primers alone and with no template (NT) are also shown. Primers were specific to each pleurocidin variant (Table 2).

Southern Analysis

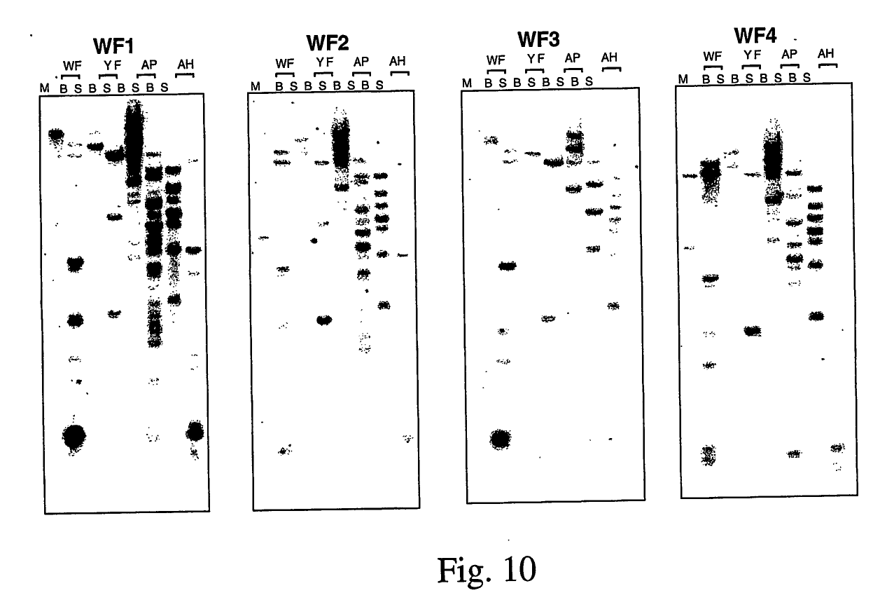

Positive signals were specific to flatfish DNA using the WF1, WF2, WF3 and WF4 genomic probes (FIG. 10). No signals were detected with haddock, pollock or smelt DNA (data not shown). All four probes showed hybridisation to common SstI and BamHI bands from the DNAs of all four flatfish, indicating that the genes are clustered on these genomes. The sizes of the hybridising fragments from the winter flounder digest are given in Table 8.

FIG. 10 describes Southern analysis of pleurocidin genes of winter flounder (WF), yellowtail flounder (YF), American plaice (AP) and Atlantic halibut (AH). Total genomic DNA (7.5 μg) was digested with BamHI (B) or SstI (S) and the fragments resolved on a 1.0% agarose gel. The blot was hybridized successively with probes corresponding to WF1, WF2, WF3, and WF4. Markers (M) are lambda DNA digested with StyI (24.0, 7.7, 6.2, 3.4, 2.7, 1.9, 1.4, 0.9 Kb).

Identification of Additional Pleurocidin-Like Sequences From Other Fish Species

An alignment of the deduced amino acid sequences of pleurocidin-like peptides from American plaice, yellowtail flounder, witch flounder and Atlantic halibut is shown in FIG. 3. Sequences were obtained from genomic DNA of Petrale sole, C—O sole, English sole, starry flounder, European plaice, Greenland halibut and Pacific halibut. High conservation is present in the signal peptide and acidic propiece regions, whereas the portion corresponding to the mature peptide shows much more variability.

FIG. 3 describes Alignment of pleurocidin-like peptide sequences deduced from nucleotide sequences of genes and PCR products amplified from skin and/or intestine of the following species: winter flounder (WF), yellowtail flounder (YF), witch flounder (GC), American plaice (AP) and Atlantic halibut (AH). Specific non-limiting examples of pleurocidin-like sequences identified are shown in Table 4. Non-limiting examples of cDNA and/or genomic sequences are provided in Appendix I.

Identification of Pleurocidin-Like Sequences in the Winter Flounder Genome

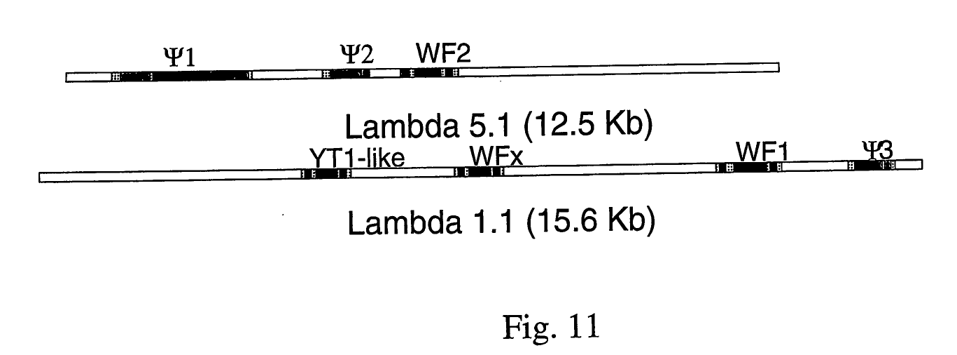

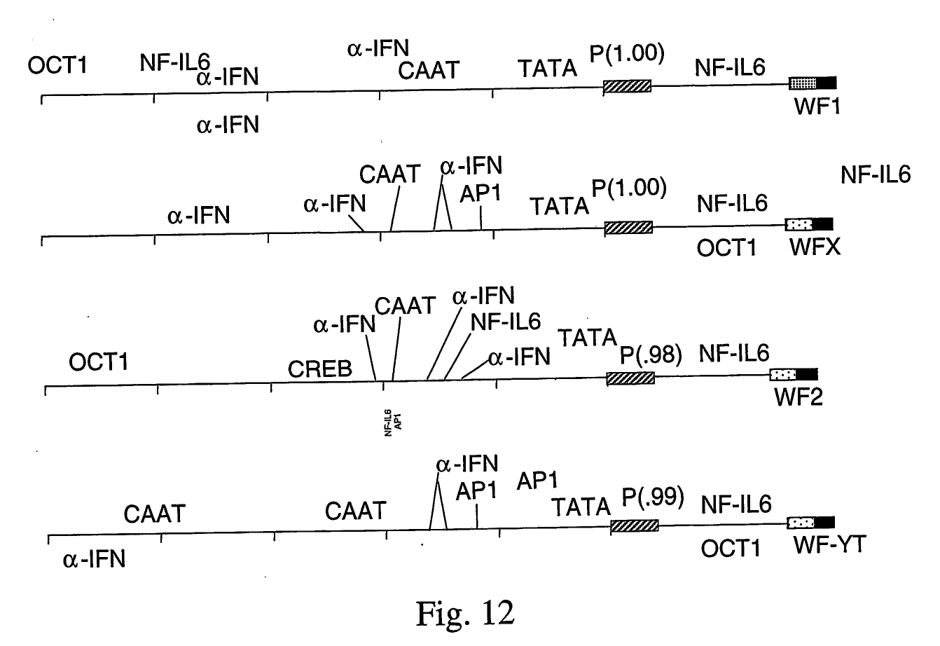

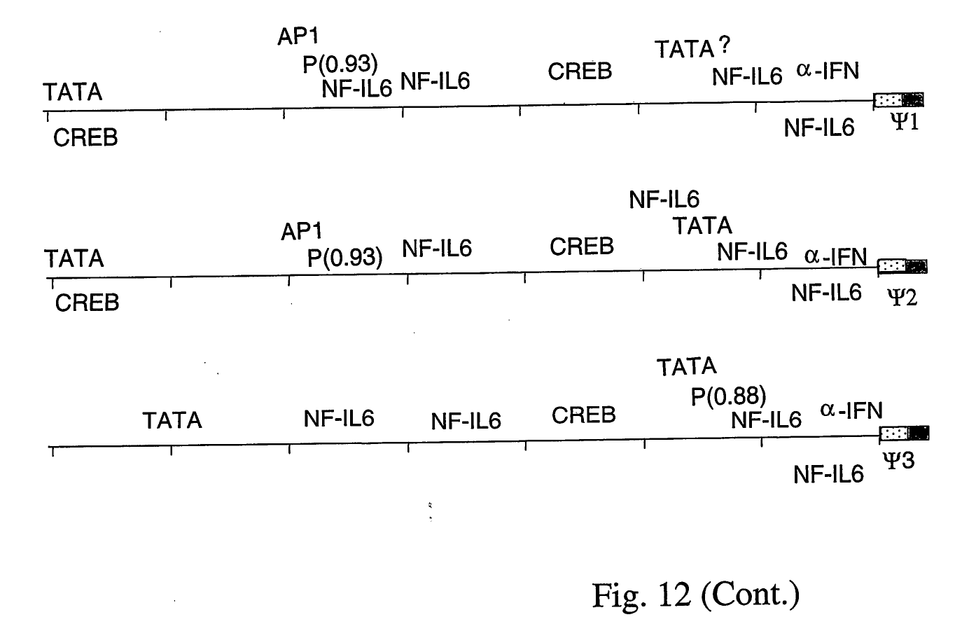

Two clones containing fragments of 12.5 and 15.6 kb, respectively, were isolated from a genomic library from winter flounder. The 12.5 kb fragment encoded the gene corresponding to WF2 and two pseudogenes. The 15.6 kb fragment encoded the gene corresponding to WF1, one pseudogene and two previously undescribed pleurocidin-like sequences referred to as WFX and WFYT. A schematic of the gene arrangement is shown in FIG. 11. Scanning of the sequences upstream of the coding sequence revealed a canonical eukaryotic promoter, TATA and CAAT boxes as well as highly conserved sites for several transcriptions factors including NF-IL6, API and α-interferon (FIG. 12). No promoter sequences were identified upstream of pseuodgenes.

FIG. 12 describes Locations of transcription factor binding sites upstream of pleurocidin genes and pseudogenes. Promoters are indicated by hatched boxes, introns by solid boxes and genes and exons by stippled boxes.

Prediction and Assessment of Antimicrobially Active Peptide Sequences

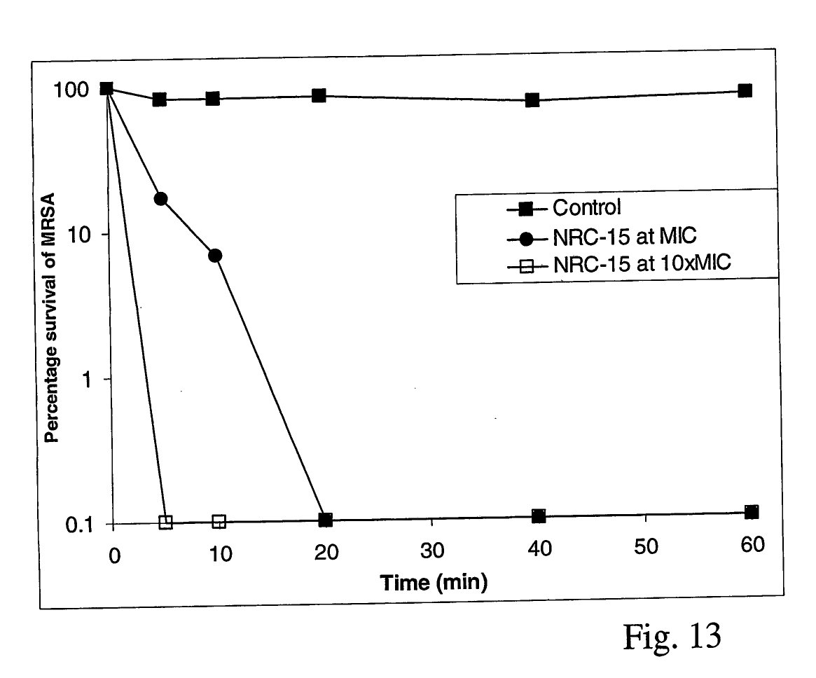

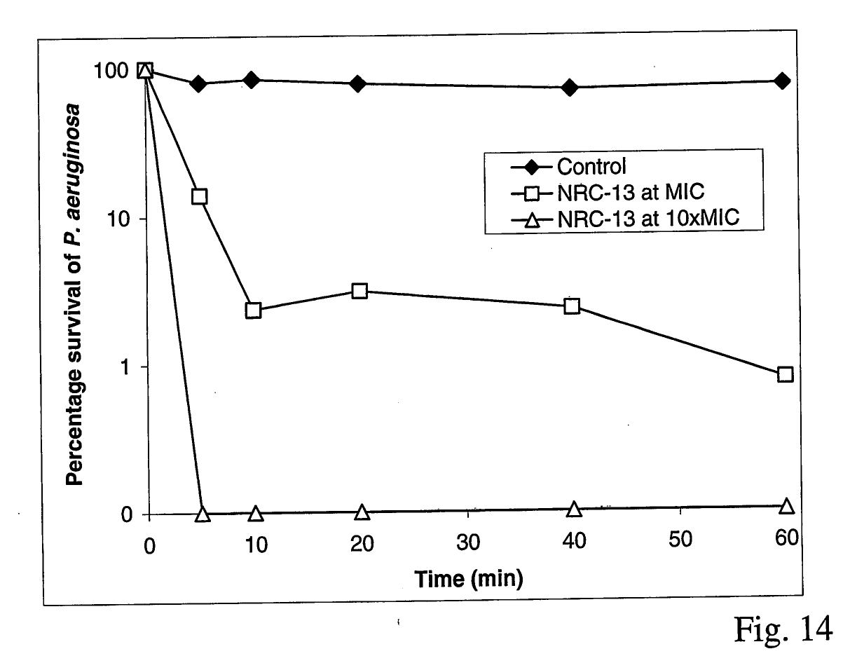

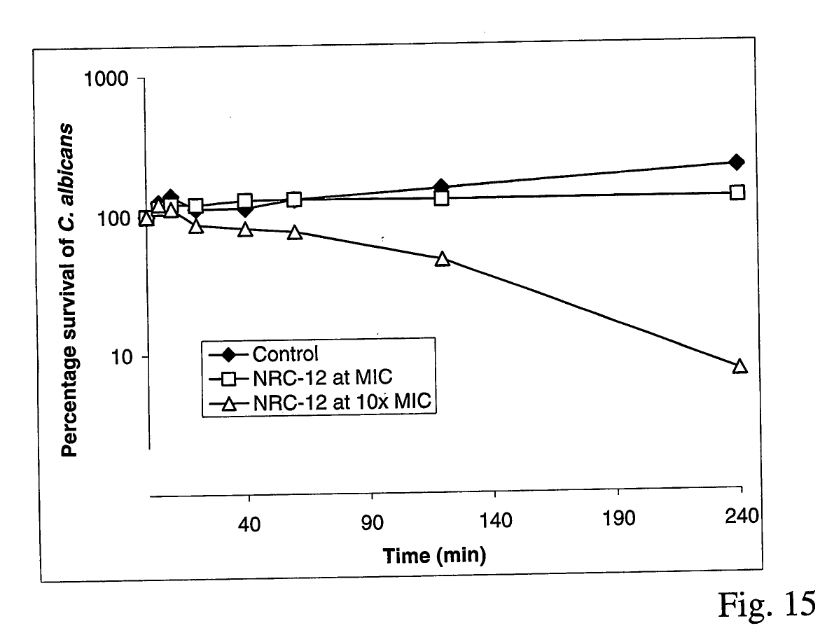

The minimal inhibitory concentrations of the chemically produced peptides against a wide range of baterial pathogens and C. albicans were determined and are shown in Table 9. Generally speaking many peptides showed the ability to inhibit the growth of a broad spectrum of bacterial pathogens and C. albicans. Particularly good examples of peptides with a broad spectrum of antimicrobial activity are the three peptides derived from American plaice (NRC-11, NRC-12, and NRC-13) and three peptides derived from witch flounder (NRC-15, NRC-16, and NRC-17). Of those, NRC-15, NRC-13, and NRC-12 showed ability to kill methicillin-resistant S. aureus (FIG. 13), P. aeruginosa (FIG. 14) and C. albicans (FIG. 15), respectively.

FIG. 13 describes Survival of a Gram-positive bacterium (methicillin-resistant Staphylococcus aureus—MRSA) upon exposure to NRC-15 at its minimal inhibitory concentration (MIC) and ten times its MIC. S. aureus was grown in Mueller-Hinton broth and exposed to NRC-15 at its MIC and ten times its MIC. At the specified intervals equal aliquots were removed from the culture, plated on MHB plates, and the resulting colonies were counted.

FIG. 14 describes Survival of a Gram-negative bacterium (Pseudomonas aeruginosa) upon exposure to NRC-13 at its minimal inhibitory concentration (MIC) and ten times its MIC. P. aeruginosa was grown in Mueller-Hinton broth and exposed to NRC-13 at its MIC and ten times its MIC. At the specified intervals equal aliquots were removed from the culture, plated on MHB plates, and the resulting colonies were counted.

FIG. 15 describes Survival of a yeast (Candida albicans) upon exposure to NRC-12 at its minimal inhibitory concentration (MIC) and ten times its MIC. C. albicans was grown in Mueller-Hinton broth and exposed to NRC-12 at its MIC and ten times its MIC. At the specified intervals equal aliquots were removed from the culture, plated on MHB plates, and the resulting colonies were counted.

In addition to demonstrating that pleurocidin-like peptides are active against a wide range of bacteria as well as C. albicans, the results indicate which factors should preferably be considered in selecting antimicrobially active peptides from genomic sequences.

Firstly, a notable group of peptides with poor or no observed activities were peptides derived from pseudogenes (NRC-8, NRC-9, NRC-10). These results indicate that peptides capable of being expressed in the host organism may be better candidates for antimicrobials.

Secondly, the previously described N-terminal GRRKRK in WF1a (FIG. 2) proved to be a determinant of antimicrobial activity in NRC-3 as shown by the fact NRC-2 (identical to NRC-3 but missing the aforementioned fragment) was only marginally active (Table 9). This result stresses the importance of carefully selecting the start/end residues in the mature peptide, wherever these are not apparent in the original pre-pro-sequence.

Thus in an embodiment of the invention there is provided a group of pleurocidin-related antimicrobial peptides having the amino acid sequence GRRKRK. It will be appreciated that pleurocidin-like antimicrobial peptides lacking this sequence also exist and are specifically contemplated herein.

The previously described principles of: selecting positively charged peptides with good separation of hydrophilic and hydrophobic residues in helical wheel models, preserving GW or GF at the N-terminus, amidating the C-terminus where glycine was present, and cropping off clusters of acidic C-terminal amino acids were successful in selecting antimicrobially active peptides.

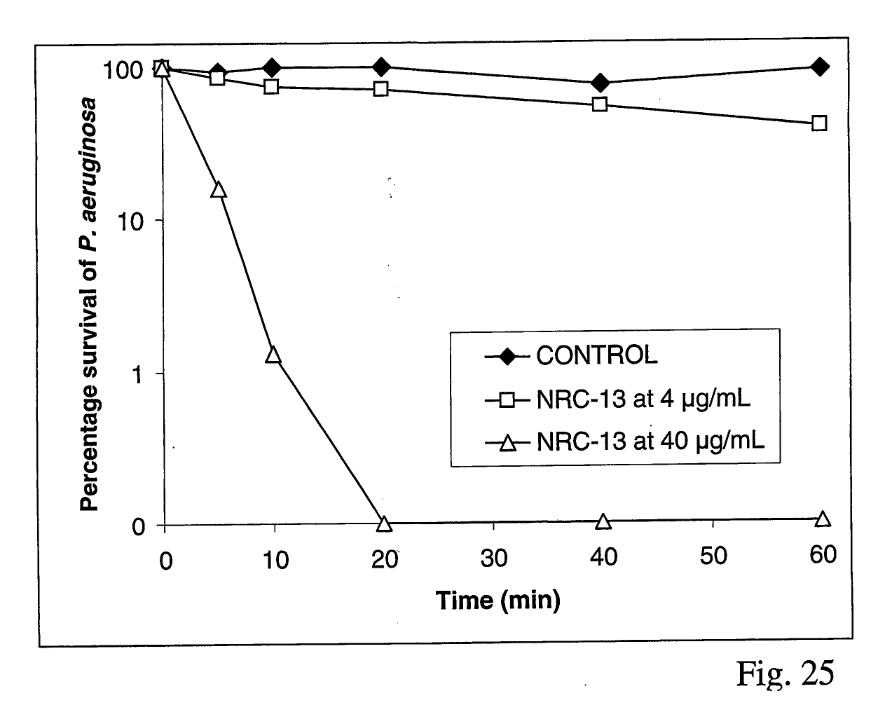

Peptides of the invention can be used at a range of pH's, salt concentrations, and temperatures. These peptides are useful against pathogens grown in biofilms or under any other conditions for pathogen growth or culture. See for example FIG. 25 in which the ability of NRC-13 to kill P. aeruginosa K799 in 50 mM NaCl is shown. NRC-13 was added to a culture of P. aeruginosa supplemented with 150 mM NaCl to a final concentration of 4 μg/ml (□) or 40 μg/ml (Δ), representing the MIC and 10×MIC, respectively. A control with no peptide added is also shown (♦).

Peptides may be used alone or in combination with one or both of their pre-and pro-sequences.

Peptides of the invention have many uses, including as antibacterial, antifungal, antiviral, anti-cancer, and antiparasitic agents, including in combination with other antibiotics, anti-infectives, and chemotherapeutants as well as with each other.

Peptides can be used as immunomodulatory agents such as for wound healing, tissue regeneration, anti-sepsis, immune promoters, etc. including in combination with other agents.

The peptides can be delivered topically (including e.g., aerosols-especially for respiratory tract infections in CF patients, ointments, lotions, rinses, eyewashes, etc.), systemically (including e.g. IV, IP, IM, subcutaneously, intracavity or transdermally) and, orally (e.g. pills, liquid medication, capsules, etc.).

Delivery via encapsulation, including in liposomes, proteinoids is contemplated, as is delivery in transgenic systems involving agricultural animals and/or plants.

Peptides can be used as protective coatings on medical devices (including catheters, etc, food preparation machinery and packaging.

Examples of antibiotics which can be used together with peptides disclosed herein in aquaculture operations include: Terramycin Aqua (oxytetracycline), Romet, (sulfadimethoxine and ormetroprim), and Tribrissen (trimethoprim and sulfadiazine. In the hatchery, dipping in formaldehyde can be used together with peptides disclosed herein. Peptides can be used in combination with each other and/or in combination with conventional antibiotics for any of the uses described herein.

Bacterial Challenge

Three days post-injection, the infected Atlantic salmon were lethargic and anorexic. On sampling, the posterior kidneys of the injected fish were positive for A. salmonicida whereas those of the control fish were not.

Molecular Characterisation of Hepcidin cDNAs

Although the winter flounder EST database contains sequences from liver, ovary, stomach, intestine, spleen and pyloric caecae cDNA libraries and the Atlantic salmon EST database contains sequences from liver, head kidney and spleen, hepcidin-like sequences were only detected in spleen and liver cDNA libraries of both fish. Four of 135 ESTs (3.0%) in the winter flounder liver library and two of 281 ESTs (0.7%) in the winter flounder spleen library encoded hepcidins. Three of 982 (0.3%) ESTs in the Atlantic salmon liver library encoded hepcidins. Five hepcidin sequences were also found in subtracted spleen (1.8%) and three in subtracted liver (0.6%) Atlantic salmon cDNA libraries that were enriched in transcripts up-regulated during infection with Aeromonas salmonicida. Unfortunately, since these are subtracted libraries, the inserts are only portions of the complete transcripts.

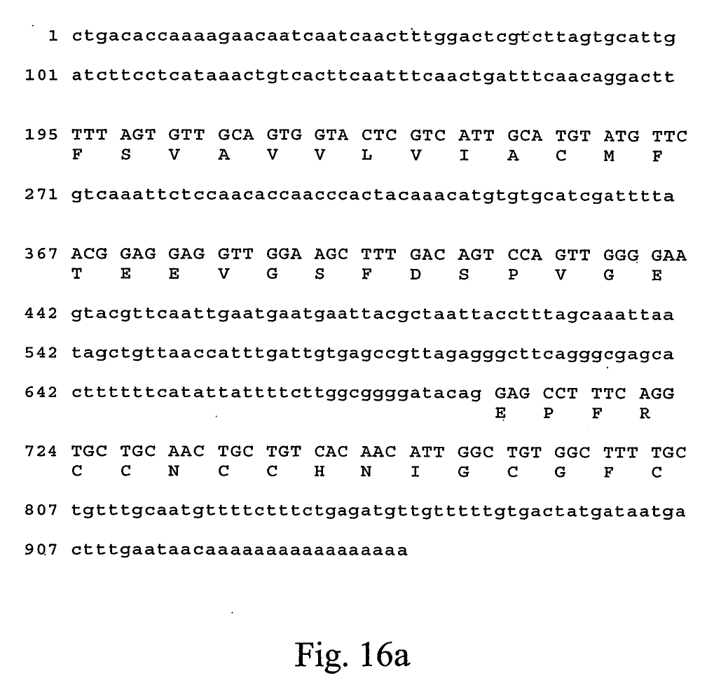

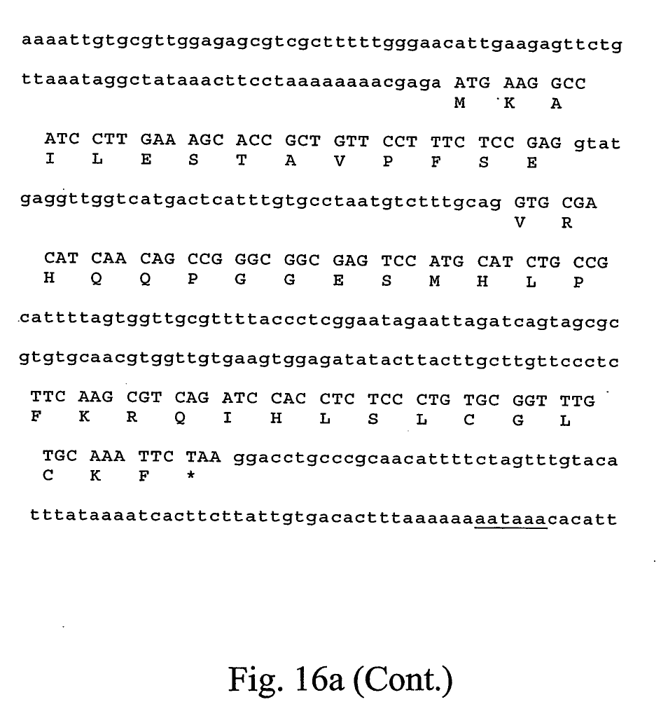

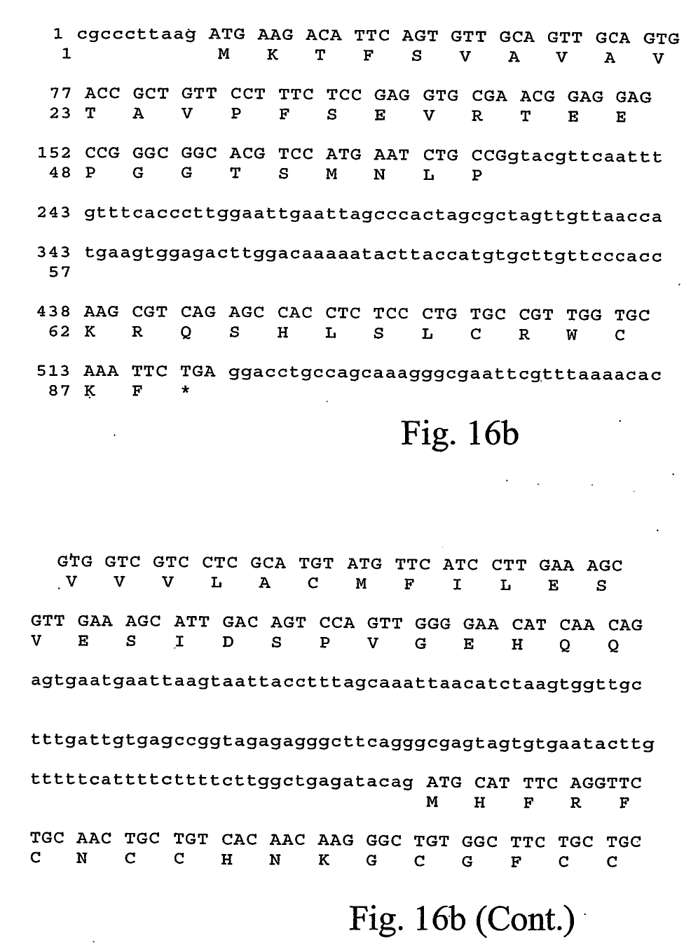

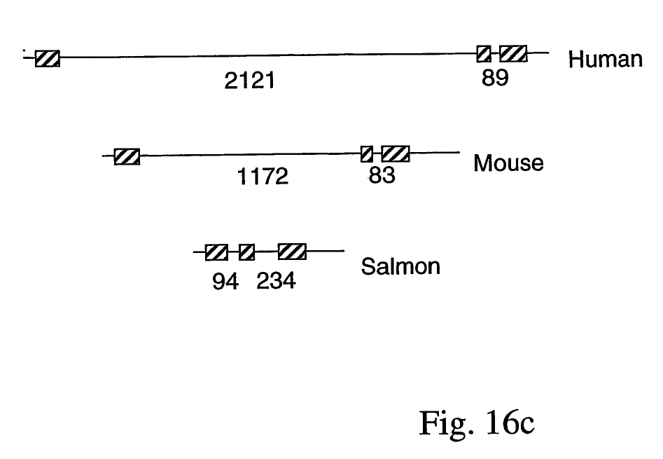

Analysis of the nucleotide sequences of Atlantic salmon hepcidin cDNAs revealed that one salmon EST (SL1-0412) was approximately 300 nucleotides longer than the other two. Furthermore, the hepcidin coding sequence was incomplete. Complete sequencing of this clone revealed the presence of two introns with standard GT/AG splice junctions (FIG. 16A). When removed, an open reading frame encoding a complete hepcidin-like peptide was obtained. Similarly, an incompletely spliced halibut transcript was amplified that still retained the second intron (FIG. 16B). Compared to mammals, the introns of salmon and probably halibut are in similar locations but of shorter length (FIG. 16C). In addition to these incompletely spliced cDNAs, we identified a winter flounder EST (WF4) that contains a large deletion relative to the other sequences that corresponded closely to the second exon of salmon and human hepcidin. Assuming the intron positions are conserved among vertebrates, this deletion could correspond to the removal of exon 2, and resulted in a peptide that differed from WF3a and WF3b in only five amino acid positions of the remaining peptide.

FIG. 16 describes a Nucleotide sequence of unspliced liver cDNA encoding Type I salmonid hepcidin. Exon sequences are indicated in upper case letters and the deduced amino acid sequence is shown below the nucleotide sequence. The gt/ag intron/exon boundaries are highlighted in boldface and the polyadenylation signal (aataaa) is underlined. B. Nucleotide sequence of partially spliced cDNA from halibut spleen encoding Type I salmonid hepcidin. C. Comparison of intron/exon structure in human, mouse and salmon. Exons are represented by hatched boxes and introns by a single line (sizes in bp shown beneath).

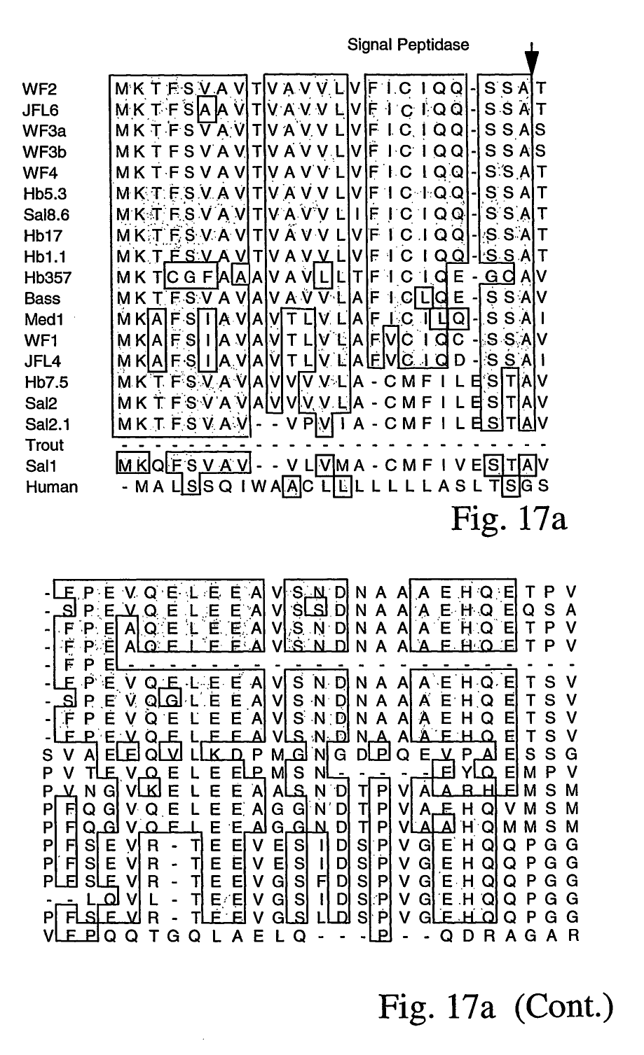

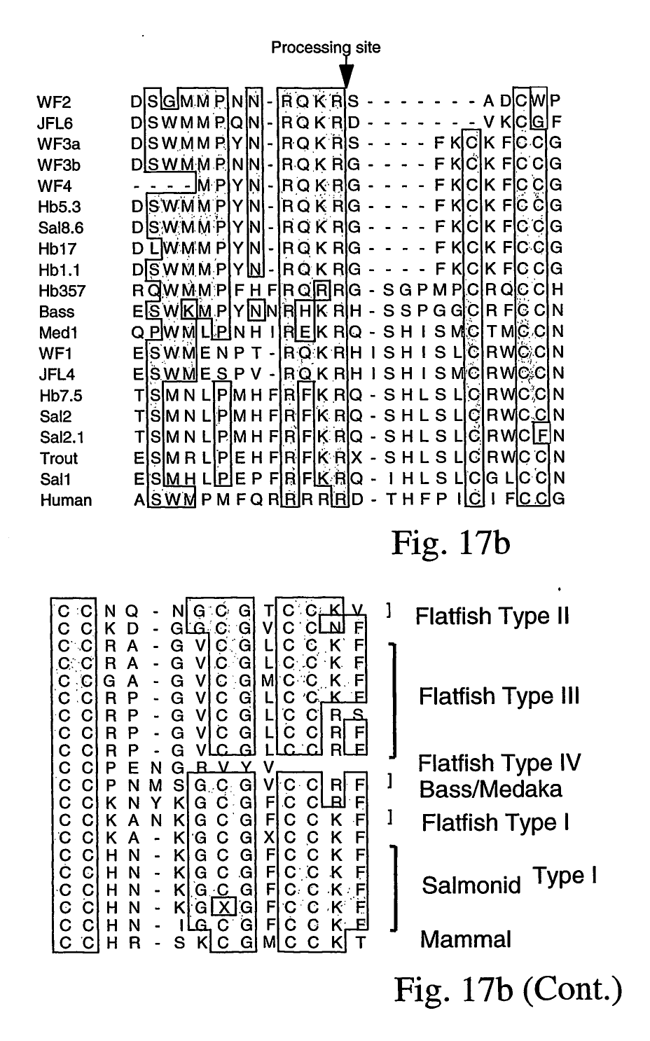

The deduced amino acid sequences of five different winter flounder hepcidin cDNAs and two different Atlantic salmon hepcidins were aligned for comparison purposes with those extracted from dbEST corresponding to Japanese flounder (two), medaka (one) and rainbow trout (one), as well as the recently reported hepcidin from hybrid striped bass (Shike et al. 2002) and two from Atlantic halibut (Hb 17 and Hb 357). The sequences obtained from spleen and liver of Atlantic salmon (Sal2.1 and Sal8.6) and Atlantic halibut (Hb1.1, Hb5.3 and Hb7.5) by PCR are also included (FIG. 17). Human hepcidin was included as a representative of the mammals. The position of cleavage by signal peptidase was predicted by PSORT and the RX(K/)R motif typical of propeptide convertases (Nakayama 1997) was identified (vertical arrows; FIG. 17). The signal peptide sequence is 22-24 amino acids and is highly conserved among all of the fish sequences. The anionic propiece is 38-40 amino acids, depending on the particular hepcidin variant. The processed hepcidins contain 19-27 amino acids and all are positively charged at neutral pH except WF2 (Table 10). Types I and III hepcidin from flatfish as well as salmon type hepcidin contain eight cysteine residues in the mature peptide, which have been proposed to form four disulphide bonds. Type II winter flounder hepcidin is missing two cysteine residues, indicating that a maximum of three disulphide bonds could form. Hb357 contains only five cysteine residues and is quite different from the remaining hepcidin-like sequences. Results of secondary structure prediction methods indicated that the consensus structure of fish hepcidins was mostly random coil, although short stretches of extended strand were predicted by some methods.

FIG. 17 describes Alignment of winter flounder (WF1, WF2, WF3a, WF3b, WF4), Atlantic halibut (Hb1.1, Hb5.3, Hb7.5, Hb17, Hb357) and Atlantic salmon (Sal1, Sal2, Sal2.1, Sal8.6) hepcidins with those of Japanese flounder (JFL4, JFL6), medaka, hybrid striped bass and human. A partial sequence from rainbow trout (GenBank accession AF281354_{—1}) is also shown. The predicted positions of signal peptidase and pre-protein cleavages are indicated by arrows.

From FIG. 17, it is apparent that all of the flatfish-type hepcidins have very similar signal peptides, which differ somewhat from the salmonid type and human hepcidin. Other novel features identified included different groups of hepcidins based on (1) number of cysteines, (2) unique insertion FKC in flatfish Type III, (3) two other locations that may contain unique insertions (4) a truncated version (Flatfish Type IV), (5) longer versions at the amino terminus.

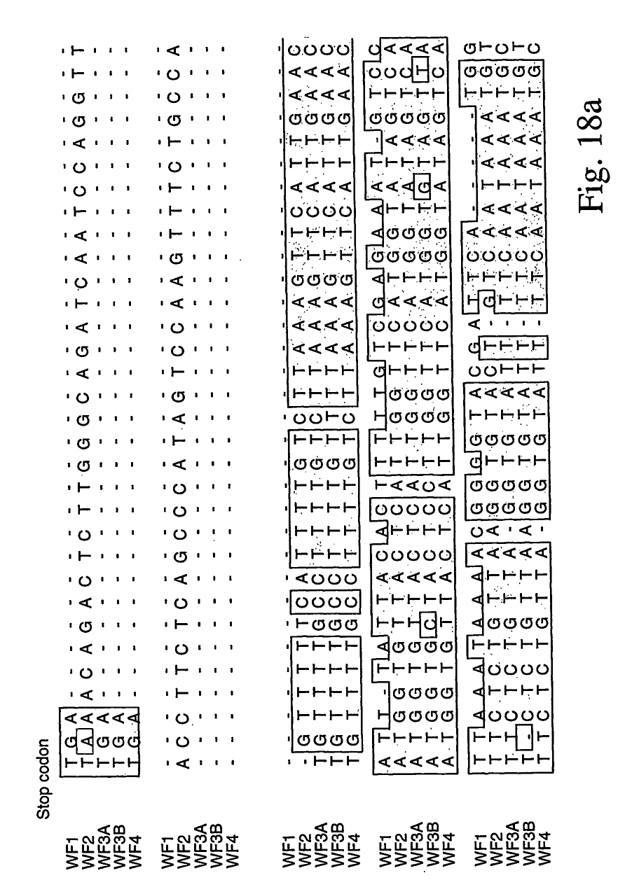

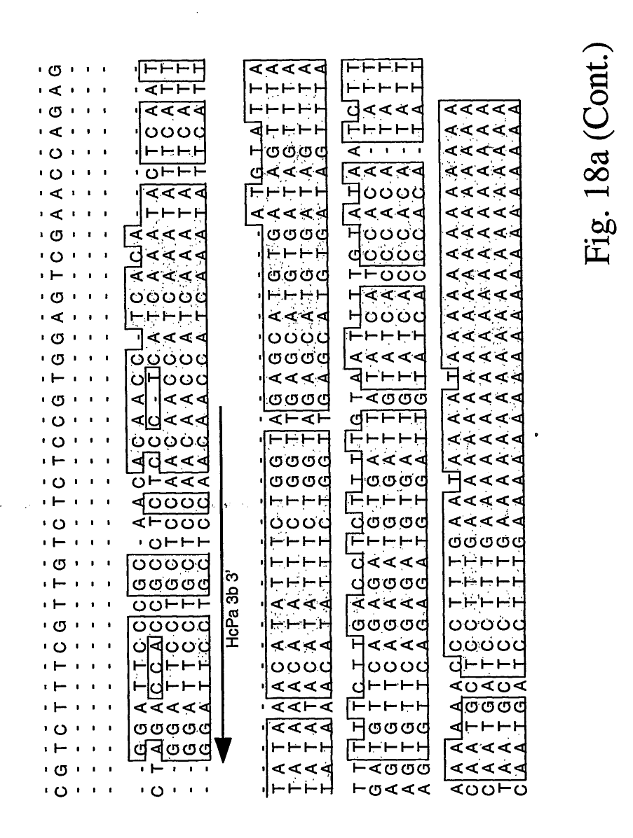

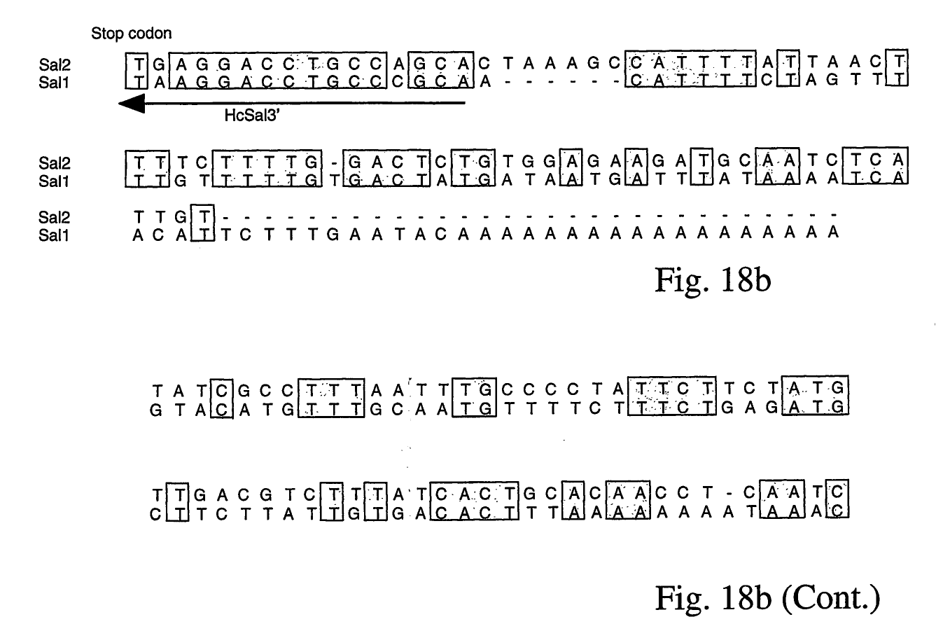

Based on the alignment, it is apparent that there are at least three different groups of flatfish hepcidins distinguishable by shared insertions and deletions. WF2 and JFL6 (Flatfish Type II) share a deletion of seven amino acids near the KR cleavage site resulting in a processed peptide of 19 amino acids, whereas WF3a, WF3b, WF4, Hb1.1, Hb17, Hb5.3 and Sal8.6 (Flatfish Type III) exhibit a deletion of only four amino acids (excluding the portion corresponding to the missing exon of WF4) resulting in processed peptides of 22 amino acids. WF1 and JFL4 (Flatfish Type I) do not contain this deletion but do contain an insertion relative to all other reported hepcidins at a position adjacent to the signal peptidase cleavage site. In addition, WF1, bass and medaka share an insertion of one amino acid within the mature peptide relative to all other reported hepcidins, giving a peptide of 26-27 amino acids. WF3a and WF3b differ from each other by only one amino acid although they contain several silent substitutions and differences in the 5′ and 3′ untranslated regions. Hb357 represents a possible fourth class of flatfish hepcidins. The 3′ untranslated regions of WF2 and WF1 are very different from those of the other hepcidin transcripts, WF2 containing a long additional portion relative to the others and WF1 being shorter and less highly conserved (FIG. 18A).

The salmonid hepcidin-like peptides fall into one group; the four reported sequences all share two deletions and differ from each other by four amino acids in the mature peptide and four amino acids in the upstream pre-protein portion. The 3′ untranslated regions of the salmon hepcidins are only moderately conserved (FIG. 18B).

FIG. 18 describes Alignment of 3′ untranslated regions of (A) winter flounder (WF1, WF2, WF3a, WF3b, WF4) and (B) Atlantic salmon (Sal1, Sal2) hepcidin cDNAs. Conserved nucleotides are boxed. The positions of the primers used to amplify hepcidin homologs from halibut and salmon are indicated by arrows.

Genomic Organisation of Winter Flounder Hepcidin Genes

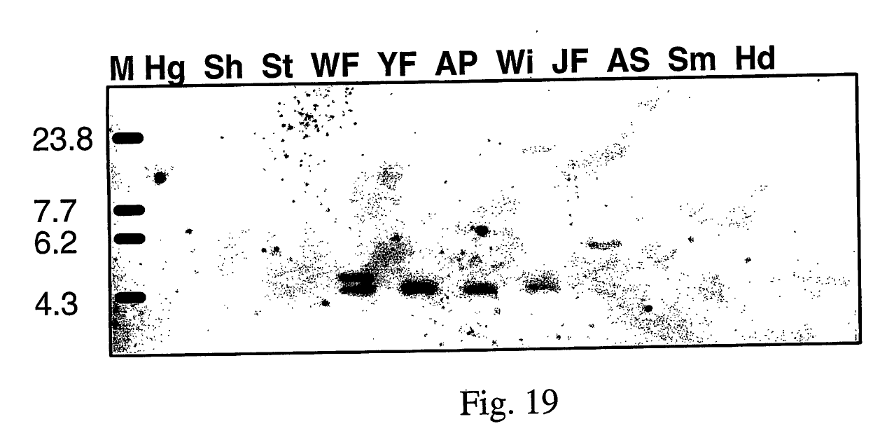

Southern hybridization analysis of genomic DNA from a wide variety of fish with a probe corresponding to Type I hepcidin identified bands in all flatfish tested but none of the other fish species (FIG. 19). In winter flounder, two fragments of 4.3 and 4.5 kb hybridized with the probe. Two fragments of yellowtail flounder of identical size hybridized (4.3 kb) and two fragments of witch flounder genomic DNA also hybridized (4.3 and 20 kb), whereas only one fragment (4.3 kb) of the American plaice and one fragment (5.5kb) of the Japanese flounder genomic DNA hybridized.

FIG. 19 describes Southern hybridization analysis of hepcidin in different fish species. SstI digests of genomic DNA (7.5 μg) from hagfish (Hg), shark (Sh), white sturgeon (St), winter flounder (WF), yellowtail flounder (YF), American plaice (AP), witch flounder (Wi), Japanese flounder (JF), Atlantic salmon (AS), smelt (Sm) and haddock (Hd) were hybridized with Type I hepcidin from winter flounder. Size markers (M) are Lambda DNA digested with StyI.

Identification of Hepcidin-Like Sequences by RT-PCR

FIG. 2 describes amplification of hepcidin cDNAs from halibut and salmon liver and spleen. RNA was prepared from tissues of fish infected with a bacterial pathogen to induce expression of antimicrobial peptide genes, reverse-transcribed and subjected to PCR using the primers listed below. Actin was run as a control to show expression of a house-keeping gene. The labelling on the figure is as follows: HL−halibut liver; SL—salmon liver; HS—halibut spleen; SS—salmon spleen; M—markers. For the primers 5′U is the Universal 5′ primer used in all reactions, Sal is Hc Sal (below) and WF is HcPA3b (below).

(SEQ ID NO: 324)

| | |
|---|---|
| HepUniversal 5′: | AAGATGAAGACATTCAGTGTTGCA |

(SEQ ID NO: 325)

| | |
|---|---|
| HcPA3 3′B2: | GTTGTTGGAGCAGGAATCC |

(SEQ ID NO: 326)

| | |
|---|---|
| Hc Sal: | TGCTGGCAGGTCCTCAGAATTTGC |

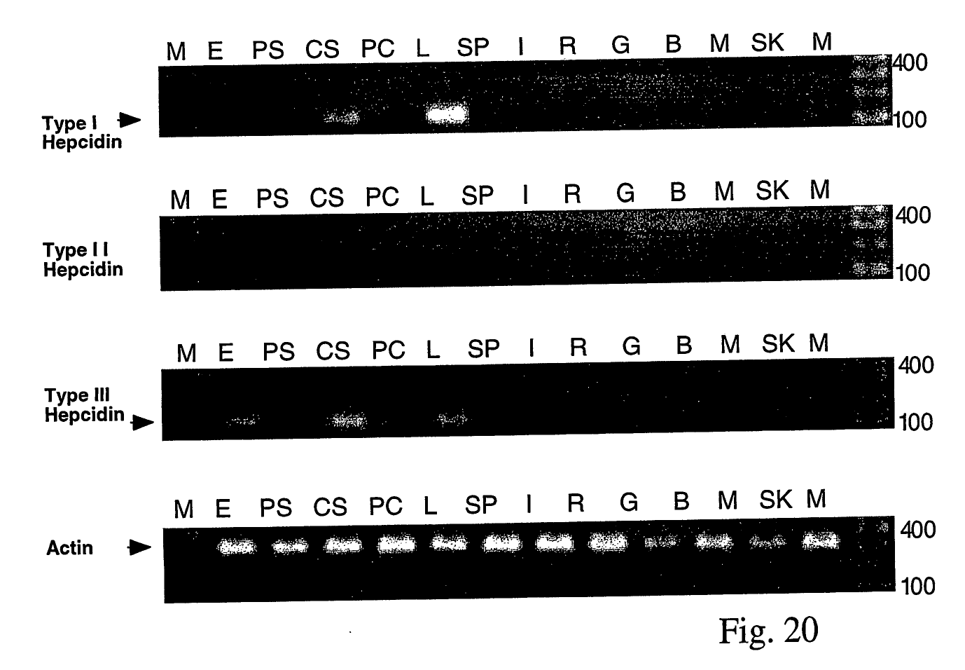

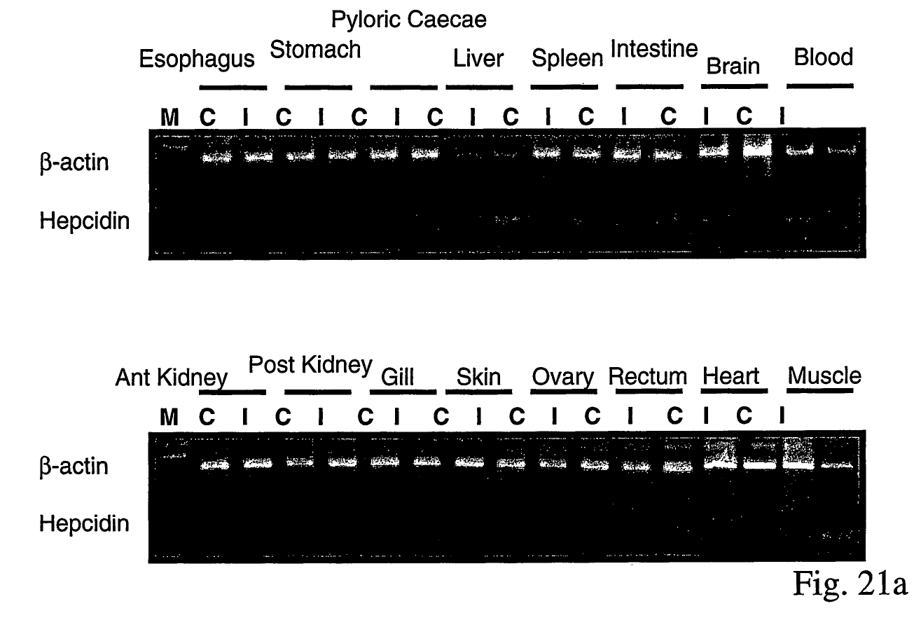

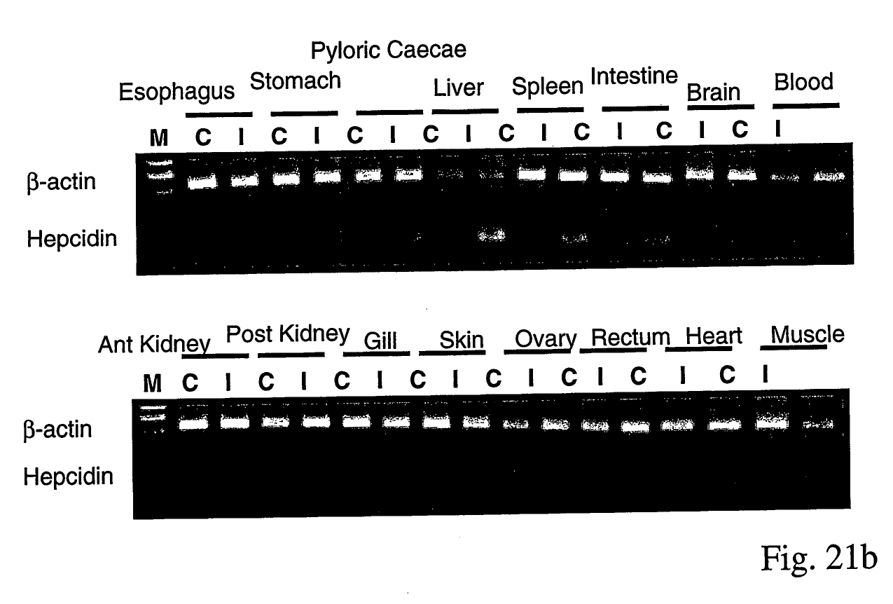

The results of RT-PCR assays of tissue-specific expression of the three winter flounder hepcidins are shown in FIG. 20. Type I hepcidin was abundantly expressed in the liver and, to a lesser extent, in the cardiac stomach. Type II hepcidin could not be detected in any tissues, whereas Type III hepcidin was moderately expressed in the esophagus, cardiac stomach, and liver. In uninfected Atlantic salmon, Type I hepcidin was expressed at quite high levels in the liver, blood and muscle, at low levels in gill and skin, and at barely detectable levels in anterior and posterior kidney (FIG. 21A, Table 10). Type II hepcidin was expressed at barely detectable levels in the gill and skin only (FIG. 21B). However, fish infected with Aeromonas salmonicida showed expression of both types of hepcidin in most tissues tested (see below).

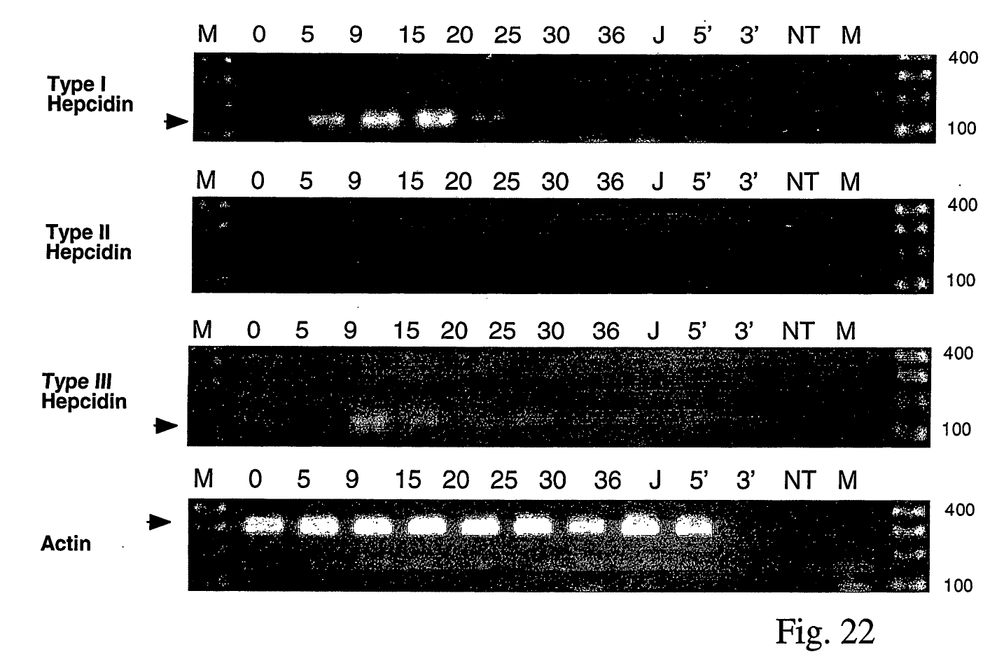

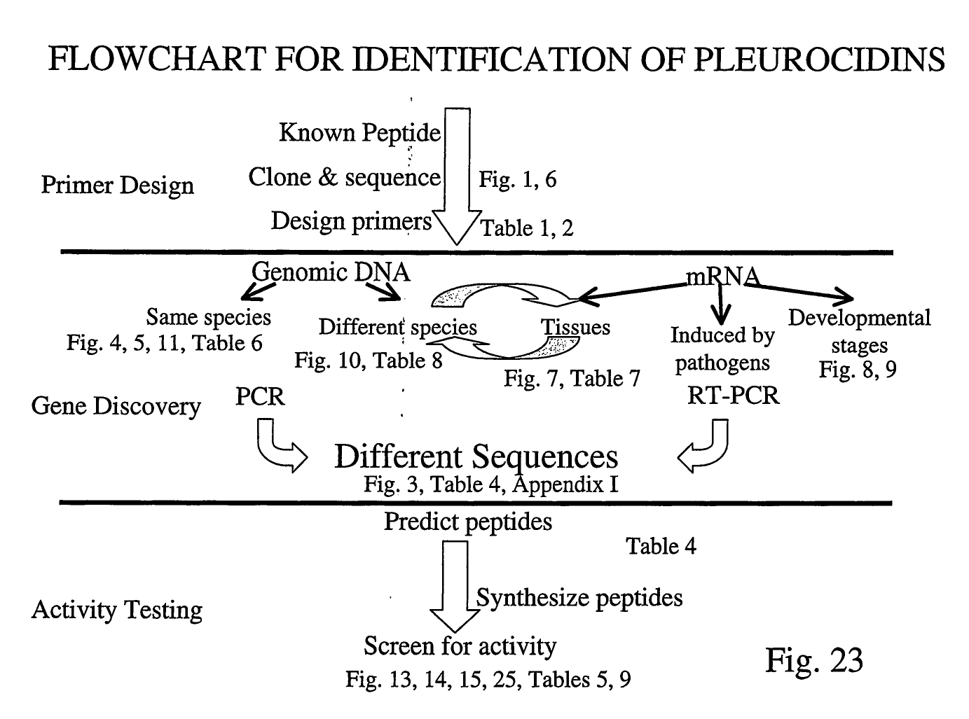

RT-PCR analysis of hepcidin gene expression in winter flounder larvae of different ages is shown in FIG. 22. Transcripts of Type II hepcidins could not be detected at any stage of development, whereas Type I and Type III hepcidins were detectable in pre-metamorphic larvae. Type I hepcidin was more abundantly expressed than Type II hepcidin and was also expressed at an earlier time (5 dph vs. 9 dph.).

FIG. 20 describes Reverse transcription-PCR assay of hepcidin and actin gene expression in different tissues of winter flounder. Amplification products from adult winter flounder were amplified using gene-specific primers for Flatfish Type I (panel A), Type II (panel B) and Type III (panel C) hepcidins and for actin (310 bp) and resolved by electrophoresis on a 2% agarose gel. Markers (M) are the 100 bp ladder (BRL)

FIG. 21 describes Reverse transcription-PCR assay of hepcidin and actin gene expression in different tissues of control Atlantic salmon (C) and those infected with Aeromonas salmonicida (I). Amplification products from reactions using gene-specific primers for Salmonid Type I (panel A) and Type II (panel B) hepcidins (163 bp) and for actin (400 bp) were resolved by electrophoresis on a 2% agarose gel. Markers (M) are the 100 bp ladder (BRL).

FIG. 22 describes Reverse transcription-PCR assay of hepcidin and actin expression in developing winter flounder larvae. Samples were larvae at 5 dph (lane 1), 12 dph (lane 2), 19 dph (lane 3), 27 dph (lane 4), 41 dph (lane 5) and adult (lane 6). Amplification products from reactions using gene-specific primers for Flatfish Type I (panel A), Type II (panel B) and Type III (panel C) hepcidins and for actin (400 bp) were resolved by electrophoresis on a 2% agarose gel using a 100 bp ladder (Pharmacia) as markers (lane M).

Identification of Additional Hepcidin-Like Sequences From Other Fish Species

Using a primer based on highly conserved sequences in the signal peptide of all reported hepcidins (Hep Universal 5′) in combination with primers based on highly conserved sequences in the 3′ UTR of salmon (HcSal 3′) and flatfish (HcPA3b 3′), it was possible to amplify hepcidin-like sequences from the liver and spleen of halibut and salmon (FIG. 2). An alignment of the deduced amino acid sequences of hepcidin-like peptides from winter flounder, Atlantic halibut and Atlantic salmon is shown in FIG. 17. Interestingly, flatfish-type hepcidin could be amplified from salmon (S8.6) and salmon-type hepcidin could also be amplified from a flatfish (Hb7.5). Additional sequences were obtained from genomic DNA of Petrale sole, C—O sole, English sole, starry flounder, European plaice, Greenland halibut and Pacific halibut.

FIG. 17 depicts an alignment of certain winter flounder (WF1, WF2, WF3a, WF3b, WF4) Atlantic halibut (Hb1.1, Hb5.3, Hb7.5, Hb17, Hb357) and Atlantic salmon (Sal1, Sal2, Sal2.1, Sal8.6) hepcidins with those of Japanese flounder (JFL4, JFL6, medaka, hybrid striped bass and human. A partial sequence from rainbow trout (Genbank Accession AF281354_{—1}) is also shown. The predicted positions of signal peptidase and pre-protein cleavages are indicated by arrows.

Specific non-limiting examples of hepcidin sequences identified are shown in Table 11. Examples of cDNA or genomic sequences are shown in Table 13.

Pleurocidins

Most antimicrobial peptides, including cecropins and dermaseptins, are encoded by multigene families that have probably arisen by sequential gene duplications. We have demonstrated that the winter flounder, and probably other flatfish, possess a gene family encoding antimicrobial compounds similar to pleurocidin. Comparison of the genomic amplification products obtained using PL1/2 with the cDNA sequence (FIG. 1A) showed that WF2 and WF4 contain three introns, the first of which occurs only 1 bp upstream from the initiator methionine. The second and third introns both occur within the mature peptide. The genes for GLa, xenopsin, levitide and caerulein—all skin peptides from Xenopus laevis—also contain an intron 1 bp upstream from the initiator methionine (Kuchler et al 1989). The intron positions are conserved in all but WF3 (FIG. 6), but they differ dramatically in size (Table 5), indicating that a considerable period of evolutionary time has elapsed since the duplication events occurred, or that the intron sequences are relatively free to drift.

Southern analysis shows that WF1-4 probes hybridise to other flatfish DNAs, including yellowtail flounder, Atlantic halibut and American plaice, but not to haddock, smelt or pollock. This hybridisation could be due to the highly conserved signal sequence and anionic portion which we have shown to be conserved in sequences isolated from these flatfish. Flatfish may provide a rich reservoir of potential therapeutants for the aquaculture industry. The probes for the different pleurocidin family members often recognise the same restriction fragments in winter flounder DNA, indicating that they may be clustered at a single locus on the genome. Complete sequencing of two Lambda clones hybridizing to pleurocidin confirms that such clustering does in fact occur (FIG. 11). Clustering of antimicrobial peptide genes has also been noted for insect cecropins (Gudmundson et al. 1991) and apidaecins (Casteels-Jossen et al. 1993), among others.

FIG. 11 describes an embodiment of a Schematic of genomic organization of pleurocidin-like genes and pseudogenes (ψ) from winter flounder. Introns are represented by solid boxes and exons by stippled boxes.

All of the members of the pleurocidin family are encoded as prepropolypeptides consisting of an amino-terminal signal sequence followed by the active peptide and ending with an acidic portion. The deduced amino acid sequences of the signal and acidic sequences are very highly conserved whereas those of the predicted mature antimicrobial peptides are more variable (FIG. 6). All, however, appear to fold into amphipathic α-helices. This sequence conservation has allowed us to use a genomic approach to identify many different members of the pleurocidin gene family, not only from winter flounder but also from a variety of other flatfish (FIG. 3, Table 4, Appendix I).

The structure of the pleurocidin prepro polypeptides bears certain resemblances to the frog dermaseptin precursors, which also contain a signal sequence of similar length (22 amino acids) and an acidic portion of 16-25 amino acids. From the full-length cDNA clone (FIG. 1A), the acidic portion of pleurocidin was shown to contain 21 residues. A major difference between the pleurocidin and dermaseptin prepolypeptides is the position of the acidic portion—downstream of the mature peptide in pleurocidin and upstream of the mature peptide in dermaseptins. The acidic proparts of defensins have been proposed to prevent interaction of the antimicrobial peptide with the membrane by neutralising the cationic charges (Valore et al. 1996) and this may also be its function in pleurocidin. This feature can be of practical significance for delivering peptides that are inactive until specifically cleaved.

The signal sequences and acidic carboxy-terminal sequences of the pleurocidin family members are extremely highly conserved. The former, and possibly the latter, are presumed to target the precursor molecules to the cell membrane for secretion. Gene families for antimicrobial peptides that contain highly conserved signal peptides (often encoded by the first exon) followed by end products with different biological activities have been described from the dermaseptin family (Valore et al. 1996) and the GLa, xenopsin, levitide and caerulein, all of which are skin peptides from Xenopus laevis (Kuchler et al. 1989). These authors proposed that this modular gene structure allows targeting for secretion to be achieved for markedly different peptides using a common pathway. In the pleurocidin gene family, a modular structure is also present with exon 2 encoding the signal sequence and first half of the antimicrobial peptide, exon 3 encoding the next ten amino acids of the antimicrobial peptide, and exon 4 encoding the last three amino acids of the antimicrobial peptide and the acidic carboxy terminus.

The mature peptides encoded by WF2 and WF4 are 60% identical to each other (FIG. 6) and somewhat less similar to dermaseptin B I and ceratotoxin B (Cole et al. 1997). WF1 is 64% identical to WFla but contains a remarkably cationic stretch of 18 amino acids between the signal sequence and the mature peptide that is not present in WF1a. Whether or not this potentially antimicrobial 18-mer peptide arises when pleurocidin WF1 processing occurs remains to be determined. Both WF1 and WF1a contain an additional 10-11 amino acids relative WF2, WF3 and WF4 between the mature peptide and the acidic carboxy terminus. WF3 shares similarities with both WF2/4 and WF1/1a. Synthetic pleurocidin identical to the central portion of WF2 has been shown to protect Coho salmon against infection by Vibrio anguillarum, as have hybrid peptides based on pleurocidin, dermaseptin and ceratotoxin (Jia et al. 2000).

The tissue-specific expression of the pleurocidin genes was assessed using northern blot analysis and RT-PCR. Northern analysis proved to be not sufficiently sensitive for detecting the low level of transcripts present in winter flounder mRNA. Transcripts were present only in skin in sufficient quantities to be detected by this method, so the more sensitive RT-PCR assay was used. Pleurocidin transcripts were found in both skin and intestine using this method, in agreement with the recently reported ultrastructural localisation of pleurocidin in these tissues (Cole, Darouiche et al. 2000) and supporting the role of pleurocidin in mucosal immunity. The transcript size (approximately 200 bp) is consistent with the size of products obtained by RT-PCR (Table 7), showing that the pleurocidin genes are transcribed separately.

RT-PCR analysis showed that the genes for the different pleurocidin-like peptides are expressed in a tissue-specific manner with WF2 being expressed predominantly in the skin and gill and to a lesser extent in the muscle, intestine, stomach and liver whereas WF1 and WF4 are detected predominantly in the gill and skin (FIG. 7). WF3 and WFYT are expressed in most of the tissues sampled, WFX is detected solely in the skin and WF I a was not expressed in any of the tissues sampled. Possibly, the different antimicrobial peptides are required to control the growth of different bacterial populations in the two tissues. Since no RT-PCR products were detected for WF4, it is possible that this gene is expressed only at low levels in adult skin or intestine or that it is expressed at a different life stage or in a different tissue.

Using primers that did not discriminate between the transcripts of the various pleurocidin-like genes, expression was first detected at 5 dph and showed a progressive increase towards adulthood. However, recent experiments using primers specific for WF1, WF1a, WF2, WF3, WF4, WFX and WFYT, transcripts were detected at different developmental stages (FIG. 9). WFX was only detectable at 20 dph, whereas WFYT, WF3 and WF2 were detectable at 5 dph and at higher levels between 25-36 dph. Interestingly, WF1 was not detectable at any larval stage and may only be expressed under specific environmental conditions in response to specific bacterial pathogens, as has been shown for Drosophila (Rivas and Ganz 1999). This is the first demonstration of developmental expression of an antimicrobial peptide in fish and shows that at least this component of innate immunity is present in early larval stages of winter flounder. Larval mortality prior to metamorphosis is of great concern and although the reasons for such mortality are not yet known, high bacterial load in the gut has been proposed (Padros, Minkoff et al. 1993). The adaptive immune systems of flatfish have been shown to develop later than those of other teleosts (Padros, Sala et al. 1991). Thus, the ability of larvae to produce antimicrobial peptides during this period may be crucial to survival, and the identification of factors that increase the production of such compounds would be of great benefit to aquaculturalists.

These results of testing synthetic peptides against a variety of bacterial pathogens as well as the fungal pathogen, Candida albicans, show promising candidates with broad-spectrum antimicrobial activities. Of particular interest is the ability of the peptides NRC-13 and NRC-15 to inhibit the growth of methicillin-resistant S. aureus at concentrations as low as 4 μg/ml. NRC-13 is also capable of inhibiting the growth of C. albicans at 4 μg/ml, P. aeruginosa at 1 μg/ml (and killing P. aeruginosa at this concentration), and A. salmonicida at 2 μg/ml. This means that NRC-13 is highly active against a fish pathogen, a Gram-negative human bacterium, a drug-resistant Gram-positive human bacterium, and a yeast. The example of NRC-13 demonstrates the range of potential targets and applications for cationic antimicrobial peptides.

These results also validate the process we used for selecting antimicrobially active peptides from a large amount of sequence data. The ability to accurately predict which peptides are likely to be active is a crucial link between genomics and therapeutics. While much work remains to be done in this area, we have clearly demonstrated that judicious application of the principles described earlier will aid in selecting active peptides.

Thus, a variety of cDNA and genomic sequences encoding the precursors of antimicrobial peptides identical to or similar to pleurocidin from a variety of flatfish species have been isolated. Northern hybridisation and sequence analysis of RT-PCR products showed that expression was tissue-specific. Most importantly, the timing of expression of different pleurocidin variants in developing larval winter flounder was determined, allowing an estimate of the onset of the innate immune system in this fish. These assays of pleurocidin expression are useful in directing the screening strategy for isolating novel peptide sequences expressed during specific tissues and/or developmental stages. Environmental parameters affecting the production of pleurocidin can also be assayed.

This work paves the way to further studies aimed at the over-expression of pleurocidin as a therapeutant for aquacultured fish and the production of disease-resistant fish through transgenic technology as has been demonstrated in transgenic tobacco expressing antimicrobial peptides (Jach et al. 1995) and proposed for fish (Jia et al. 2000). Furthermore, because many fish live in a saline environment, the properties of their antimicrobial peptides may be different from those produced by terrestrial animals and have application in unique situations. For instance, the pulmonary mucosa of patients with cystic fibrosis contain elevated NaCl concentrations, which inhibit the natural cationic peptides secreted by the lung (Goldman et al. 1997). Salt-adapted cationic peptides from marine fish may have application in the treatment of lung infections in these patients.

Hepcidins

Sequence analysis of one salmon EST (SL1-0412) and one halibut clone (Hb7.5), revealed the presence of unspliced transcripts and allowed the positions of some of the introns to be determined (FIG. 16). Similar to mouse, human and hybrid striped bass, the salmon hepcidin is composed of three exons and two introns (Park, Valore et al. 2001; Shike et al. 2002; Pigeon, Ilyin et al. 2001). The position of the first intron of salmon and bass are identical and correspond to a position two amino acids 5′ to those of mouse and human. However, the second salmon intron and the second halibut intron of Hb7.5 correspond to a position two amino acids 3′ to those of mouse and human and several amino acids 5′ to that of the bass. This is probably due to “intron sliding” whereby the positions of introns have shifted by several nucleotides over the course of evolution. Interestingly, the deletion in WF4 corresponds precisely to the position of the first salmon intron and the second mouse/human intron, indicating an intermediate intron/exon structure.

Mouse contains two hepcidin genes that are clustered on the genome (Pigeon, Ilyin et al. 2001) but in human (Park, Valore et al. 2001) and striped bass (Shike et al. 2002) only one hepcidin gene has been identified. Although the number of hepcidin genes in winter flounder and Atlantic salmon remains to be determined, there are at least five in winter flounder, five in Atlantic halibut and four in Atlantic salmon. Since there are no SstI sites within the hepcidin probe used in the Southern hybridization analysis, it is highly probable that the five winter flounder hepcidin genes reported here are clustered on two genomic fragments. Multiple genes for pleurocidin also exist (Douglas, Gallant et al. 2001) and are clustered on the genome (FIG. 11). Interestingly, all of the small flounders tested from the Atlantic exhibited a similar hybridizing band of 4.3 kb, indicating that they share similarity at the genomic level. Japanese flounder, found in the Pacific, exhibited a single hybridizing band of 5.5 kb.

The deduced amino acid sequences of the fish prepro-hepcidins can be aligned with those from mammals throughout their length but only show high similarity in the portion corresponding to the processed peptides (FIG. 17). However, within the fish, the signal peptide and the propiece are also very highly conserved. Conservation of these segments has also been noted in the pleurocidin family (Douglas, Gallant et al. 2001). The amino-termini of the processed peptides were assigned based on the amino acid sequence of human hepcidin (Krause, Neitz et al. 2000; Park, Valore et al. 2001) and the proximity to the RX(K/R)R motif characteristic of processing sites (Nakayama 1997). The molecular weights of the processed hepcidins from winter flounder and Atlantic salmon range from 1992 Da (WF2) to 3066 (WF1), comparable to hepcidins isolated from mouse, human and bass. With the exception of WF2, which has an acidic pI (5.54), the pIs of hepcidins are between 7.73 and 8.76.

Like pleurocidins, the amino acid sequences of the hepcidin variants are highly similar within species, suggesting relatively recent duplication of an ancestral gene. It is possible that the aquatic environment in which fish live necessitates the existence of a more diverse suite of antimicrobial peptides than in terrestrial mammals. In addition, this component of the innate immune system plays a more major role in fish than in mammals, which have a more highly evolved adaptive immune system.

The human hepcidin molecule has been proposed to form a secondary structure containing a series of β-turns, loops and distorted β-sheets (Park, Valore et al. 2001). Consensus secondary structure prediction of fish hepcidins show that they contain mostly random coil structure with some extended strand structure. With the exception of WF2, JFL6 and Hb357, all hepcidins reported thus far contain eight cysteine residues which are proposed to form four disulphide bonds (Krause, Neitz et al. 2000; Park, Valore et al. 2001) in the following linkage pattern: 1-4, 2-8, 3-7, 5-6 (Park, Valore et al. 2001). The loss of cysteine residues 1 and 3 from WF2 suggests that at least one disulphide bond cannot form.

Using gene-specific primers, we were able to demonstrate that different hepcidin genes are expressed in different tissues of both winter flounder (FIG. 20) and Atlantic salmon (FIG. 21). In Atlantic salmon, hepcidin was detectable in normal uninfected fish predominantly in liver, blood and muscle (Type I) and to a lesser extent in gill and skin (both types). This is consistent with the presence of three ESTs for Type I hepcidin in cDNA libraries constructed from uninfected livers, and the absence of ESTs for Type II hepcidin in cDNA libraries constructed from uninfected liver, spleen and head kidney. Type II hepcidin expression appears be confined to external epithelial surfaces in contact with the aqueous environment, whereas Type I hepcidin expression is more widespread, being expressed in liver, blood and muscle as well as external epithelial surfaces. In uninfected winter flounder, no transcripts of Type II hepcidin could be detected in any tissue but transcripts of Types I and III hepcidin were present in the liver and cardiac stomach. Type III hepcidin transcripts were also present in the esophagus.

Mouse hepcidin was also reported to be predominantly expressed in liver, and weakly in stomach, intestine, colon, lungs, heart and thymus by Northern analysis using one of the mouse hepcidin sequences as probe (Pigeon, Ilyin et al. 2001). However, this study did not discriminate between the two hepcidin genes and it is not known whether or not the two mouse genes are differentially expressed in tissues of mouse. Similarly, dot-blot analysis of human tissues and cell lines using the human hepcidin cDNA as probe revealed strong expression in adult and fetal liver and weaker expression in adult heart, fetal heart and adult spinal cord (Pigeon, Ilyin et al. 2001). An earlier study using RealTime quantitative RT-PCR (Krause, Neitz et al. 2000) revealed strong expression of hepcidin in human liver, heart and brain and weak expression in a variety of other tissues. Interestingly, we could not detect either Type I or Type II hepcidin expression in the brain of normal Atlantic salmon or winter flounder, or heart of normal Atlantic salmon. However, in infected animals, Type II hepcidin was expressed in both tissues, indicating that this form is the predominant one produced under conditions of stress.

It is intriguing that we detected transcripts of Type I hepcidin that were constitutively expressed in blood cells of Atlantic salmon. Constitutively expressed non-enzymic antimicrobial molecules have been reported only rarely in blood of fish; a small hydrophobic cationic peptide was found in mucus of rainbow trout (Smith et al., 2000) and moronecidin, an antimicrobial peptide from bass, was expressed in blood of uninfected animals (Lauth et al. 2002). Interestingly, expression of neither hepcidin increased in blood of infected salmon relative to the uninfected control animals. Possibly, hepcidin is fulfilling a role in iron homeostasis in control animals as well as an antimicrobial role. Its presence in circulating blood cells of uninfected animals may be a precautionary measure against impending infection.

Type I and II hepcidins from Atlantic salmon were up-regulated during infection with Aeromonas salmonicida, but to different extents in various tissues. While Type I hepcidin was noticeably up-regulated in the esophagus, stomach, pyloric caecae, liver, spleen, intestine, posterior kidney, rectum and muscle and to a lesser extent in anterior kidney and skin, Type II hepcidin showed a more dramatic increase in stomach, pyloric caecae, liver, spleen, intestine, brain, heart and muscle. Weaker up-regulation was present in esophagus, anterior and posterior kidney, skin and rectum. These results are consistent with those reported for bacterially challenged hybrid striped bass where up-regulation was most dramatic in liver, but was also demonstrated in skin, gill, intestine, spleen, anterior kidney and blood (Shike et al. 2002). It is not known whether there are multiple hepcidins in hybrid striped bass and, if so, whether they are differentially expressed as in Atlantic salmon and winter flounder.

Studies with mice have shown a 4.3-fold increase in hepcidin expression in livers of mice injected with LPS and a 7-fold increase in primary hepatocytes exposed to LPS (Pigeon, Ilyin et al. 2001). These studies were based on Northern analysis using only one of the mouse hepcidin sequences as probe, and were therefore unable to distinguish possible differential expression of the two mouse variants. Similar increases were noted in livers of mice subjected to iron overload, but not for primary hepatocytes exposed to iron citrate, possibly due to the differentiation status of the cultured hepatocytes. The fact that both iron overload and LPS exposure increase hepcidin expression indicates the importance of these two factors in the host response to pathogens.

During infection, iron is removed from the system by various mechanisms so that it is unavailable for use by invading pathogens. It has been proposed that recently discovered transferrin receptor2 mediates iron uptake by hepatocytes and increases their expression of hepcidin (Fleming and Sly 2001; Nicolas, Bennoun et al. 2001). Hepcidin, in turn, increases iron accumulation in macrophages and increases dietary iron absorption in duodenal crypt cells via β2 microglobulin, HFE and transferrin receptor1. These crypt cells differentiate into enterocytes with reduced amounts of iron transport proteins, thereby decreasing dietary iron uptake. Hepcidin thus appears to play a crucial role in iron homeostasis during inflammation as well as acting as an antimicrobial peptide. It is also possible that hepcidin could modulate expression of liver-derived acute phase proteins and exhibit synergistic effects with other components of the immune system.

Antimicrobial peptides have been shown to modulate gene expression in mouse macrophages (Scott, Rosenberger et al. 2000), and it is possible that they may exert similar effects in fish macrophages or hepatocytes. The presence of a functional nuclear localization signal (four K/R residues in a row) within prohepcidin of mouse and human indicates that hepcidin could act as a signaling molecule involved in maintenance of iron homeostasis in these organisms (Pigeon, Hlyin et al. 2001). Interestingly, the nuclear localization signal also contains the recognition signal for processing of prohepcidin, indicating that nuclear localization would occur only prior to removal of the propiece, or that the propiece itself is localized to the nucleus. Teleost hepcidins contain only 3 out of 4 K/R residues, which may not be sufficient for nuclear localization; a role for hepcidin in intracellular signaling awaits testing with synthetic or in vitro-expressed peptide.

In conclusion, the sequences of new hepcidin-like peptides from different fish species and the presence of related sequences in several flatfish species by Southern hybridization have been determined. Furthermore, it has been shown that the various types of fish hepcidins are differentially expressed in a tissue-specific manner in normal fish, as a result of bacterial infection, and during larval development, thus providing a strategy for identifying additional sequences for novel peptides. Apparently in fish, different tissues produce hepcidins in a constitutive or inducible manner, indicating that hepcidin variants may have different functions under different circumstances. Given their role in iron homeostasis in mammals, it is possible that fish hepcidin variants may fulfill this role as well as that of killing specific pathogens. In vitro expression of hepcidin variants will allow their spectrum of antimicrobial activity to be determined as well as their effect on the innate immune response.

Thus, there has been provided a method for identifying potential antimicrobial peptides.

Tables

Table 1. Nucleotide sequences of oligonucleotides used for isolating pleurocidin-like sequences (SEQ ID NOS: 1-10, left to right, in order of appearance)

Table 2. Nucleotide sequences of oligonucleotides used for assay of pleurocidin-like gene expression in different tissues and at different stages of development of winter flounder (SEQ ID NOS: 11-34, left to right, in order of appearance)

Table 3. Nucleotide sequences of primers used in RT-PCR assays to analyse hepcidin gene expression. (SEQ ID NOS: 35-61, left to right, in order of appearance) The amino acid sequence on which the 5′ primer was based is shown. The 3′ primers were within the 3′ untranslated region (3′ UTR). The annealing temperatures used in the PCR reactions and the sizes of the amplification products are listed.

Table 4. One-letter amino acid sequences for pleurocidins based on genomic and expression data (SEQ ID NOS: 62-81, respectively, in order of appearance)

Table 4a. Bacterial and Candida strains used in this study

Table 5. Sizes of introns (in bp) in genomic sequences amplified using primers PL5′ and PL3′

Table 6. RT-PCR products from skin and intestine corresponding to different pleurocidin genes

Table 7. Sizes of bands (in kb) hybridising to pleurocidin probes in BamHI and SstI digests of winter flounder DNA

Table 8. Minimal inhibitory concentrations of pleurocidin-like cationic antimicrobial peptides against a wide spectrum of bacterial pathogens and Candida albicans.

Table 9. Characteristics of winter flounder and Atlantic salmon hepcidin-like peptides

Table 10. Results of PCR analysis of hepcidin expression

Table 11. One-letter amino acid sequences for certain hepcidins based on genomic and expression data, including NRC reference numbers (SEQ ID NOS: 174-211, respectively, in order of appearance)

Table 12. Nucleotide sequences of pleurocidin-like peptides of Table 4

Table 13. Nucleotide sequences of hepcidin-like peptides of Table 11.

### REFERENCES

The mention of a reference is not an admission or suggestion that it is relevant to the patentability of anything disclosed herein.

Amsterdam, D. 1996. Susceptibility Testing of Antimicrobials in Liquid Media. In V. Lorian (ed.), Antibiotics in Laboratory Medicine. Williams and Wilkins, Baltimore.

Casteels-Jossen, K., T. Capaci, et al. (1993). “Apidaecin multipeptide precursor structure: a putative mechanism for amplification of the insect antibacterial response.” EMBO J. 12: 1569-78.

Cohen, S., M. Skiguchi, J. Stern, and H. Barner. 1963. The synthesis of messenger RNA without protein synthesis in normal and phage-infected thymineless strains of Escherichia coli. Proc. Natl. Acad. Sci. U.S.A Biochem. 49:699-706.

Cole, A. M., R. O. Darouiche, et al. (2000). “Characterization of a fish antimicrobial peptide: gene expression, subcellular localization, and spectrum of activity.” Antimic. Ag Chemotherapy. 44: 2039-45.

Cole, A. M., P. Weis, et al. (1997). “Isolation and characterization of pleurocidin, an antimicrobial peptide in the skin secretions of winter flounder.” J. Biol. Chem. 272(18): 12008-12013.

Douglas, S. E., C. E. Bullerwell, et al. (1999). “Molecular investigation of aminopeptidase N expression in the winter flounder, Pleuronectes americanus.” J. Appl. Ichthyol 15: 80-86.

Douglas, S. E., J. W. Gallant, et al. (1999). “Winter flounder expressed sequence tags: establishment of an EST database and identification of novel fish genes.” Mar. Biotechnol 1: 458-464.

Douglas, S. E., J. W. Gallant, et al. (1998). “Isolation of cDNAs for trypsinogen from the winter flounder, Pleuronectes americanus.” J. Mar. Biotechnol. 6: 214-9.

Douglas, S. E., J. W. Gallant, et al. (2001). “Cloning and developmental expression of a family of pleurocidin-like antimicrobial peptides from winter flounder, Pleuronectes americanus (Walbaum).” Dev. Comp. Immunol. 25: 137-147.

Douglas, S. E., A. Gawlicka, et al. (1999). “Ontogeny of the stomach in winter flounder: characterisation and expression of the pepsinogen and proton pump genes and determination of pepsin activity.” J. Fish Biol. 55: 897-915.

Douglas, S. E., S. C. M. Tsoi, et al. (2002). Expressed sequence tags—a snapshot of the fish genome. A Step Toward the Great Future of Aquatic Genomics, Tokyo, Japan.

Fleming, R. E. and W. S. Sly (2001). “Hepcidin: A putative iron-regulatory hormone relevant to hereditary hemochromatosis and the anemia of chronic disease.” Proc. Natl. Acad. Sci. USA 98(15): 8160-8162.

Garvan, J. (1996). SeqVu. Sydney, Australia, The Garvan Institute of Medical Research.

Goldman, M. J., G. M. Anderson, et al. (1997). “Human beta-defensin-1 is a salt-sensitive antibiotic in lung that is inactivated in cystic fibrosis.” Cell. 88: 553-60.

Gong, Z., K. V. Ewart, et al. (1996). “Skin antifreeze protein genes of the winter flounder, Pleuronectes americanus, encode distinct and active polypeptides without the secretory signal and prosequences.” J. Biol. Chem. 271: 4106-12.

Gudmundsson, G. H., D. A. Lidholm, et al. (1991). “The cecropin locus. Cloning and expression of a gene cluster encoding three antibacterial peptides in Hyalophora cecropla.” J. Biol. Chem. 166: 11510-7.

Hwang, E.-Y., J.-K. Seo, et al. (1999). “Purification and characterization of a novel antimicrobial peptide from the skin of the hagfish, Eptatretus burgeri.” J. Food Sci. Nutr. 4(1): 28-32.

Jach, G., B. Gornhardt, et al. (1995). “Enhanced quantitative resistance against fungal disease by combinatorial expression of different barley antifungal proteins in transgenic tobacco.” Plant J. 8: 97-109.

Jia, X., A. Patrzykat, et al. (2000). “Antimicrobial peptides protect coho salmon from Vibria anguillarium infections.” Appl. Environ. Microbiol. 66: 1928-32.

Krause, A., S. Neitz, et al. (2000). “LEAP-1, a novel highly disulfide-bonded human peptide, exhibits antimicrobial activity.” FEBS Lett. 480: 147-150.

Kuchler, K., G. Kreil, et al. (1989). “The genes for the frog skin peptides GLAa, xexopsin, levitide, and caerulin contain a homologous export exon encoding a signal sequence and part of an amphiphilic peptide.” Eur. J. Biochem. 179: 281-5.

Lauth, X., H. Shike, et al. (2002). “Discovery and characterization of two isoforms of moronecidin, a novel antimicrobial peptide from hybrid striped bass.” J. Biol. Chem. 277: 5030-5039.

LeMaitre, C., N. Orange, et al. (1996). “Characterization and ion channel activities of novel antibacterial proteins from the skin mucosa of carp (Cyprinus carpio).” Eur. J. Biochem. 240: 143-149.

Marck, C. (1992). DNA Strider Version 1.2. Service de Biochimie—Bat 142, Centre d'Etudes Nucleares de Saclay,. Gif-sur-Yvette, France.

Moore, K. S., S. Wehrli, et al. (1993). “Squalamine: an aminosterol antibiotic from the shark.” Proc; NatI. Acad. Sci. USA. 90: 1354-1358.

Nakayama, K. (1997). “Furin: a mammalian subtilisin/Kex2p-like endoprotease involved in processing of a wide variety of precursor proteins.” Biochemical J. 327: 625-635.

Nicolas, G., M. Bennoun, et al. (2001). “Lack of hepcidin gene expression and severe tissue iron overload in upstream stimulatory factor 2 (USF2) knockout mice.” Proc. Natl. Acad. Sci. USA. 98(15): 8780-8785.

Oren, Z. and Y. Shai (1996). “A class of highly potent antibacterial peptides derived from pardaxin, a pore-forming peptide isolated from Moses sole fish Pardachirus marmoratus.” Eur. J. Biochem. 237(1): 303-310.

Padros, F., G. Minkoff, et al. (1993). “Histopathological events throughout the development of turbot (Scophthalmus maximus L.).” J. Comp. Pathol. 109: 321-4.

Padros, F., R. Sala, et al. (1991). Organogenesis in turbot, Scophthalmus maximus, larvae related to the main developmental stages: in Larvi'91. Fish and Crustacean Larviculture Symposium. Ghent, Belgium: European Aquaculture Society.

Park, C. B., J. H. Lee, et al. (1997). “A novel antimicrobial peptide from the loach, Misgurnus anguillicandatus.” FEBS Lett. 411: 173-178.

Park, C. H., E. V. Valore, et al. (2001). “Hepcidin, a urinary antimicrobial peptide synthesized in the liver.” J. Biol. Chem. 276(11): 7806-7810.

Park, I. Y., C. B. Park, et al. (1998). “Parasin 1, an antimicrobial peptide derived from histone H2A in the catfish, Parasilurus asotus.” FEBS Lett 437(3): 258-262.

Pigeon, C., G. Ilyin, et al. (2001). “A new mouse liver-specific gene, encoding a protein homologous to human antimicrobial peptide hepcidin, is overexpressed during iron overload.” J. Biol. Chem. 276(11): 7811-7819.

Rivas, L. and T. Ganz. (1999). “Eukaryotic antibiotic peptides: not only a membrane business.” Drug Discovery Today. 4: 254-6.

Scott, M. G., C. M. Rosenberger, et al. (2000). “An α-helical cationic antimicrobial peptide selectively modulates macrophage responses to lipopolysaccharide and directly alters macrophage gene expression.” J. Immunol. 165: 3358-3365.

Shike H, Lauth X, Westerman M E, Ostland V E, Carlberg J M, Van O1st JC, Shimizu C, Bums J C (2002). “Bass hepcidin is a novel antimicrobial peptide induced by bacterial challenge.” Eur J Biochem: 269:2232-2237.

Silphaduang, U. and E. J. Noga (2001). “Peptide antibiotics in mast cells of fish.” Nature 414: 268-9.

Smith, V. J., J. M. O. Fernandes, et al. (2000). “Antibacterial proteins in rainbow trout, Oncorhynchus mykiss.” Fish Shellfish Immunol. 10: 243-260.

Thompson, J., D. Higgins, et al. (1994). “CLUSTAL W: improving the sensitivity of progressive multiple sequence alignment through sequence weighting, position specific gap penalties and weight matrix choice.” Nucleic Acids Res. 22: 4673-4680.

Trust T. J,. Ishiguro, E. E., Chart, H. and Kay W. W. (1983) Virulence properties of Aeromonas salmonicida. J. World Maricul. Soc.14:193-200.

Valore, E. V., E. Martin, et al. (1996). “Intramolecular inhibition of human defensin HNP-1 by its propiece.” J. Clin. Invest. 97: 1624-9.

Wu, M., E. Maier, R. Benz, and R. E. W. Hancock. 1999. Mechanism of interaction of different classes of cationic antimicrobial peptides with planar bilayers and with the cytoplasmic membrane of Escherichia coli. Biochem. 38:7235-7242.

TABLE 1

Nucleotide sequences of oligonucleotides used for
isolating pleurocidin-like sequences

| | | |
|---|---|---|
| | Amino Acid | |
| Primer | Sequence | Nucleotide Sequence (5′ => 3′) |

Screening cDNA library

| | | |
|---|---|---|
| PleuroA | FFKKAAHVGKH | TTCTTCAAGAAGGCYGCYCAYGT[C/G]GG |
| | | [C/A]AAGCA |
| PleuroB | HVGKAALTHYL¹ | CAYGT[C/G]GG[C/A]AAGGCYGCYCT[C/G] |
| | | AA[C/T/A]CAYTACCT |

Genomic PCR and RT-PCR

| | | |
|---|---|---|
| PL1 | 5′ untranslated | GCCCACTTTGTATTCGCAAG |
| PL2 | 3′ untranslated | CTGAAGGCTCCTTCAAGGCG |
| PL5′ | MKFTATF | ATGAAGTTCACTGCCACCTTC |
| PL3′ | KRAVDE¹ | TCATCGACTGCGCGCTT |

¹complement

TABLE 2

Nucleotide sequences of oligonucleotides used for
assay of pleurocidin-like gene expression in
different tissues and at different stages of
development of winter flounder

| | | Amino Acid | |
|---|---|---|---|
| Gene | Primer | Sequence | Nucleotide Sequence (5′ => 3′) |
| WF1 | RTWF1 | KGRWLER | AAGGGCAGGTGGTTGGAAAGG |
| | RTWF1/3′ | YQEGEE¹ | CCCTCCCCCTCCTGGTA |
| WF1a | RTWF1a | RKRKWLR | CGTAAGAGAAAGTGGTTGAGA |
| | RTWF1a/3′ | YQEGEE¹ | CCCTCCCCCTCCTGGTA |
| WF2 | RTWF2 | KAAHVG | AAGGCTGCTCACGTTGGC |
| | PL2 | 3′ untranslated | CTGAAGGCTCCTTCAAGGCG |
| WF3 | RTWF3 | FLGALIK | TTCTTAGGAGCCCTTATCAAA |
| | RTWF3/3′ | YDEQQE¹ | CTCCTGCTGCTCGTCATA |
| WF4 | RTWF4 | HGRHAA | CATGGTCGTCATGCTGCC |
| | PL2 | 3′ untranslated | CTGAAGGCTCCTTCAAGGCG |
| WFYT | RTWFYT | GFLFHG | GGGATTTCTTTTTCATGG |
| | RTWFYT/3′ | SFDDNP¹ | GGGTTGTCATCGAATGAG |
| WFX | RTWFX | RSTEDI | CGTTCTACAGAGGACATC |
| | RTWFX/3′ | DDDDSP¹ | GGGGCTGTCATCATCATC |

TABLE 3

Nucleotide sequences of primers used in RT-PCR assays to analyse
hepcidin gene expression. The amino acid sequence on which the
5′ primer was based is shown. The 3′ primers were within the 3′
untranslated region (3′ UTR). The annealing temperatures used in
the POR reactions and the sizes of the amplification products
are listed.

| | | | | | |
|---|---|---|---|---|---|
| Type | | | | | |
| (size) | Primer | Amino acid | Nucleotide sequence | Annealing | |
| (bp) | Product | aequence | (5′ => 3′) | temperature | size |

Winter flounder

| | | | | |
|---|---|---|---|---|
| Type I | HcPA1 5′ | WMENPT | TGGATGGAGAATCCCACC | 50° C. |
| | 137 | | | |
| | HcPA1b 3′ | 3′ UTR | GTGAGGTTGTGTTGCGGG | |
| Type II | HcPA2 5′ | GMMPNN | GGGATGATGCCAAACAAC | 50° C. |
| | 180 | | | |
| | HcPA2b 3′ | 3′ UTR | ACTTGGACTATGGGCTGAG | |
| Type III | HcPA3 5′ | WMMPNN | TGGATGATGCCATACAAC | 50° C. |
| | 118 | | | |
| | HcPA3b 3′ | 3′ UTR | GTTGTTGGAGCAGGAATCC | |
| Actin | ActF (WF) | AALVVD | TCGCTGCCCTCGTTGTTGAC | 50° C. |
| | 312 | | | |
| | ActR (WF)* | VLLTEAP* | GGAGCCTCGGTCAGCAGGA | |
| | Actin F1 | VFPSIV | GTGTTCCATCCATCGTC | 50° C. |
| | 194 | | | |
| | Actin R1 | HTFYNEL | GAGCTCGTTGTAGAAGGTGT | |

Atlantic salmon

| | | | | |
|---|---|---|---|---|
| Type I | HCSS 5′ | MHLPEP | ATGCATCTGCCGGAGCCT | 55° C. |
| | 163 | | | |
| | Hep Liv R | 3′ UTR | CATTGCAAACATGTACAAACTAG | |
| Type II | Hep Sp F | MNLPMH | ATGAATCTGCCGATGCA | 52° C. |
| | 163 | | | |
| | Hep Sp R | 3′ UTR | GGGCAAATTAAAGGCG | |
| Actin | Act400F | IVGRPRHQ | TCGTCGGTCGTCCCAGGCATCAG | 52° C. |
| | 400 | | | |
| | Act400R | GYALPHAI | ATGGCGTGGGGCAGAGCGTAACC | |

*complement

TABLE 4

Sequences of pleurocidin-like peptides used for activity
testing. Final peptide sequences and patterns of
C-terminal amidation were selected based on the analysis
of translated nucleotide sequences and on principles
described in the text.

| | | |
|---|---|---|
| Origin | Amino acid sequence | Code |

| | | |
|---|---|---|
| Winter Flounder (1) | GKGRWLERIGKAGGIIIGGALDHL-NH₂ | NRC-01^{a} |
| Winter Flounder (1a) | WLRRIGKGVKIIGGAALDHL-NH₂ | NRC-02^{a,d} |
| Winter Flounder (1a-l) | GRRKRKWLRRIGKGVKIIGGAALDHL-NH₂ | NRC-03^{a,d} |
| Winter Flounder (2) 2.1 | GWGSFFKKAAHVGKHVGKAALTHYL-NH₂ | NRC-04^{a} |
| Winter Flounder (3) | FLGALIKGAIHGGRFIHGMIQNHH-NH₂ | NRC-05^{a} |
| Winter Flounder (4) 1.1 | GWGSIFKHGRHAAKHIGHAAVNHYL-NH₂ | NRC-06^{a} |
| Yellowtail Flounder YT2 | RWGKWFKKATHVGKHVGKAALTAYL-NH₂ | NRC-07^{b} |
| Winter Flounder X | RSTEDIIKSISGGGFLNAMNA-NH₂ | NRC-08^{b,c} |
| Winter Flounder Y | FFRLLFHGVHHGGGYLNAA-NH₂ | NRC-09^{b.c} |
| Winter Flounder Z | FFRLLFHGVHHVGKIKPRA-NH₂ | NRC-10^{b,c} |
| American Plaice AP1 | GWKSVFRKAKKVGKTVGGLALDHYL-NH₂ | NRC-11^{b} |
| American Plaice AP2 | GWKKWFNRAKKVGKTVGGLAVDHYL-NH₂ | NRC-12^{b} |
| American Plaice AP3 | GWRTLLKKAEVKTVGKLALKHYL-NH₂ | NRC-13^{b} |
| Witch Flounder GcSc4C5 | AGWGSIFKHIFKAGKFIHGAIQAHND-NH₂ | NRC-14^{b} |
| Witch Flounder GcSc4B7 | GFWGKLFKLGLHGIGLLHLHL-NH₂ | NRC-15^{b} |
| Witch Flounder GC3.8-t | GWKKWLRKGAKHLGQAAIK-NH₂ | NRC-16^{b} |
| Witch Flounder GC3.8 | GWKKWLRKGAKHLGQAAIKGLAS | NRC-17^{b} |
| Witch Flounder GC3.2 | GWKKWFTKGERLSQRHFA | NRC-18^{b} |
| Halibut Hb26 | FLGLLFHGVHHVGKWIHGLIHGHH-NH₂ | NRC-19^{b} |
| Halibut Hb18 | GFLGILFHGVHHGRKKALHMNSERRS | NRC-20^{b} |

^{a}Peptide predicted from expressed tag and/or expression confirmed by RT-PCR and/or by in situ hybridization.
^{b}Peptide predicted from genomic sequence
^{c}Pseudogenes
^{d}NRC-2 and NRC-3 are both derived from the same sequences with the latter including an additional N-terminal fragment.

TABLE 4a
Bacterial and Candida strains used in this study.

| Species | Code ID | Comments |
|---|---|---|
| Escherichia coli | C498, UB1005 | Parent of DC2 |
| Escherichia coli | C500, DC2 | Outer membrane-permeable mutant |
| Escherichia coli | C786, CGSC4908 | Triple auxotroph (thy, uri, L-his) |
| Salmonella enterica s. Typhimurium | C587, 14028S | Parent of C610 |
| Salmonella enterica s. Typhimurium | C610, MS4252S | Supersusceptible strain |
| Pseudomonas aeruginosa | H187, K799 | Parent of H188 |
| Pseudomonas aeruginosa | H188, Z61 | Supersusceptible strain |
| Enterococcus faecalis | C625, ATCC29212 | Standard strain (ATCC) |
| Staphylococcus aureus | C622, ATCC25923 | Standard strain (ATCC) |
| Staphylococcus aureus | C623, SAP017 | MRSA clinical isolate (from Tony Chow - VGH) |
| Staphylococcus epidermidis | C960, ATCC14990 | Standard strain (ATCC) |
| Staphylococcus epidermidis | C621 | Clinical isolate (from David Speert - Children's) |
| Bacillus subtilis | C971, ATCC6633 | Standard strain (ATCC) |
| Aeromonsa salmonicida | 99-1, A449 | Field isolate being sequenced at IMB |
| Aeromonas salmonicida | 97-4 | Field isolate |
| Candida albicans | C627, CALB105 | Yeast test strain |

TABLE 5
Sizes of introns (in bp) in genomic sequences amplified
using primers PL5′ and PL3′

| Gene | Exon 1 | Intron 1 | Exon 2 | Intron 2 | Exon3 | Total |
|---|---|---|---|---|---|---|
| WF1 | 154 | 539 | 31 | 95 | 82 | 901 |
| WF1a¹ | 103 | ? | 31 | ? | 82 | ? |
| WF2² | 100 | 525 | 31 | 108 | 49 | 813 |
| WF3 | 100 | 374 | 19 | 97 | 64 | 654 |
| WF4² | 100 | 230 | 31 | 101 | 49 | 511 |

¹Intron sizes could not be determined as this sequence is only represented by an RT-PCR product
²Sequences were also amplified using primer PL1 and PL2

TABLE 6
RT-PCR products from skin and intestine corresponding to
different pleurocidin genes

| Skin | Intestine | Size | Band |
|---|---|---|---|
| 4 | n/d¹ | 265 bp | WF1 |
| 5 | 2 | 175 bp | WF2 |
| 4 | 9 | 175 bp | WF3 |
| n/d¹ | n/d¹ | — | WF4 |
| n/d¹ | 7 | 215 bp | n/d² |

¹not detected
²not detected by genomic PCR (corresponds to WF1a)

TABLE 7
Sizes of bands (in kb) hybridising to pleurocidin
probes in BamHI and SstI digests of winter flounder DNA

| Probe | BamHI | SstI |
|---|---|---|
| WF1 | >24, 6 | 19, 17, 4.5, 4.4, 3.0, 2.9, 2.2, 1.3, x |
| WF2 | 6 | 19, 17, 4.5, 4.4, 2.9, x 1.3, x |
| WF3 | >24 | 19, 17, 4.5, x 2.9, x 2.2, 1.3, x |
| WF4 | 17, 6 | 19, 17, 4.5, 4.4, 2.9, x 2.2, 1.3, 1.2 |

x = no hybridising band evident

TABLE 8
Minimal inhibitory concentrations of pleurocidin-like cationic antimicrobial peptides against a wide
spectrum of bacterial pathogen and Candida albicans. Pathogers were grown in Mueller-Hinton broth
and exposed to a range of concentrations of the specified peptide. The lowest peptide concentration
which inhibited bacterial growth by at least 50% was recorded as the minimal inhibitory concentration.

| | A. sal | A. sal | S. typh | S. typh | P. aeru | P. aeru | E. coli | E. coli | E. coli | S. epi | MRSA | C. alb |
|---|---|---|---|---|---|---|---|---|---|---|---|---|
| | 99-1 | 97-4 | MS4252s | 14028s | K799 | Z61 | C786 | UB1005 | DC2 | C621 | C623 | C627 |
| NRC-1 | 64 | 64 | 16 | >64 | >64 | 32 | 32 | 32 | 32 | >64 | >64 | 64 |
| NRC-2 | >128 | 128 | 64 | >64 | 64 | 32 | 64 | 64 | 64 | >64 | >64 | >64 |
| NRC-3 | 2 | 4 | 2 | 8 | 2 | 1 | 2 | 8 | 2 | 8 | 8 | 4 |
| NRC-4 | 2 | 2 | 2 | 16 | 8 | 4 | 2 | 4 | 2 | 8 | 8 | 8 |
| NRC-5 | >64 | >64 | 64 | >64 | >64 | 32 | 64 | 64 | >64 | 32 | 32 | >64 |
| NRC-6 | 4 | 4 | 4 | 64 | 16 | 4 | 4 | 4 | 2 | >64 | 32 | 32 |
| NRC-7 | N/A | N/A | N/A | N/A | N/A | N/A | N/A | N/A | N/A | N/A | N/A | N/A |
| NRC-8 | >64 | >64 | >64 | >64 | >64 | >64 | >64 | >64 | >64 | >64 | >64 | >64 |
| NRC-9 | >64 | >64 | 64 | >64 | >64 | 64 | 64 | >64 | >64 | >64 | >64 | >64 |
| NRC-10 | >64 | 32 | 16 | >64 | 32 | 8 | 32 | 32 | 32 | 32 | 64 | >64 |
| NRC-11 | 8 | 8 | 4 | 32 | 32 | 4 | 4 | 16 | 4 | 64 | >64 | 32 |
| NRC-12 | 2 | 2 | 2 | 8 | 4 | 1 | 2 | 8 | 2 | 8 | 16 | 4 |
| NRC-13 | 4 | 2 | 2 | 8 | 4 | 1 | 2 | 4 | 2 | 4 | 4 | 4 |
| NRC-14 | 32 | 16 | 16 | >64 | 32 | 8 | 16 | 16 | 16 | 16 | 16 | >64 |
| NRC-15 | 8 | 16 | 4 | 16 | 8 | 4 | 8 | 8 | 8 | 4 | 4 | 16 |
| NRC-16 | 2 | 1 | 0.5 | 16 | 4 | 1 | 1 | 2 | 0.5 | 16 | 32 | 8 |
| NRC-17 | 2 | 1 | 1 | 8 | 4 | 2 | 1 | 4 | 1 | 32 | 16 | 8 |
| NRC-18 | >64 | 128 | 32 | >64 | >64 | 64 | 64 | 64 | 64 | >64 | >64 | >64 |
| NRC-19 | 64 | >64 | 16 | 64 | 32 | 8 | 32 | 16 | 32 | 8 | 8 | 64 |
| NRC-20 | >64 | >64 | >64 | >64 | >64 | 64 | >64 | >64 | >64 | >64 | >64 | >64 |

TABLE 9
Characteristics of winter flounder and Atlantic salmon hepcidin-like
peptides

| | Total | Total | Molecular | |
|---|---|---|---|---|
| Name | Amino Acids | Cysteines | Weight | pI |
| WF1 | 27 | 8 | 3066 | 8.75 |
| WF2 | 19 | 6 | 1992 | 5.54 |
| WF3 | 22 | 8 | 2367 | 8.74 |
| WF4 | 22 | 8 | 2256 | 8.52 |
| Hb5.3 | 22 | 8 | 2363 | 8.75 |
| Sal8.6 | 22 | 8 | 2331 | 8.76 |
| Hb17 | 22 | 8 | 2391 | 8.76 |
| Hb1.1 | 22 | 8 | 2391 | 8.76 |
| Hb357 | 22 | 5 | 2397 | 7.84 |
| Hb7.5 | 25 | 8 | 2881 | 8.53 |
| Sal2.1 | 25 | 7 | 2925 | 8.60 |
| Sal1 | 25 | 8 | 2720 | 7.73 |
| Sal2 | 25 | 8 | 2881 | 8.53 |

TABLE 10
Semi-quantitative RT-PCR analysis of hepcidin expression
in Atlantic salmon during bacterial challenge

| | Type I Hepcidin | | | Type II Hepcidin | | |
|---|---|---|---|---|---|---|
| Tissue | Control | Infected | Ratio | Control | Infected | Ratio |
| Esophagus | nd | 0.08 | ↑ | nd | 0.09 | ↑ |
| Stomach | nd | 0.09 | ↑ | nd | 0.27 | ↑↑ |
| Pyloric caecae | nd | 0.14 | ↑ | nd | 0.37 | ↑↑ |
| Liver | 1.19 | 2.36 | 2 | nd | 1.45 | ↑↑↑ |
| Spleen | nd | 0.18 | ↑ | nd | 0.41 | ↑↑ |
| Intestine | nd | 0.21 | ↑ | nd | 0.33 | ↑↑ |
| Brain | nd | nd | 0 | nd | 0.50 | ↑↑ |
| Blood | 0.82 | 0.84 | 1 | nd | nd | ˜ |
| Anterior kidney | 0.06 | 0.07 | 1.2 | nd | 0.08 | ↑ |
| Posterior kidney | 0.07 | 0.14 | 2 | nd | 0.11 | ↑ |
| Gill | 0.13 | 0.12 | 1 | 0.08 | 0.07 | 1 |
| Skin | 0.14 | 0.18 | 1.3 | 0.07 | 0.09 | 1.3 |
| Ovary | nd | nd | 0 | nd | nd | 0 |
| Rectum | 0.07 | 0.13 | 2 | nd | 0.08 | ↑ |
| Heart | nd | nd | 0 | nd | 0.43 | ↑↑ |
| Muscle | 0.38 | 0.8 | 2.1 | nd | 0.60 | ↑↑ |

Pixel densities obtained by densitometry are expressed relative to the actin signal. The ratio of infected: control was calculated where numerical values were obtained for both conditions.
nd, not detected;
↑ weakly up-regulated;
↑↑ strongly up-regulated.

[image] [image] TABLE 12
Nucleotide sequences encoding pleurocidin-like peptides of Table 4.

NRC-01 Winter Flounder WF1 (SEQ ID NO: 82)
ATGAAGTTCACTGCCACCTTCCTCCTGTTGTTCATCTTCGTCCTCATGGTTGATCTCGGAGAGGGTCGTCGTAAGA
AAAAGGGGTCGAAGAGAAAGGGGTCCAAGGGAAAGGGGTCCAAGGGAAAGGGCAGGTGGTTGGAAAGGATTGGTAA
AGGTAGAGTCACGGAATTAATTTGCTTTTTACATTGCAAATATTTTTCATATAACATTGCTGGAAAATCACAAAAA
TAAGTAGTCAATATATTTGGCCAAATAGAATCACTTTGATTTCAATAATAATCAAAATAACAACCTAAAAGGCCTT
TGATTAGCATGTTCCTTCAATGAAATGGACATTGTAATTTACTTTGATTCTCACATGCTACGACCTGCTGCAGCAA
CATTTGAAAATAAATTTGTCCCAGAAGATTTTAAAGTACATTGTTATAGGCGATTTATCTTTCTATTACTCAGATA
TTTGTTCAAACCAATAGAATAACTGGATCTCTATGCTAAAATAATAAAACACACATTCAGATGTTACCAGTCAAGA
TTGAACGCTGTTTAAAAGTAAGTATGAAACATCCTCTGTATGTATAATTGTTTAACTGGTAACTTATAGTCCTAAT
AATTGCGTTATGGAAATGTATTAATTGTCATTTAATATAATTTGCTGGAATTTATCACTGTGTGTTTTTGTTTGTT
TTTACACAGCTGGCGGGATAATTATCGGGGGGGCCCTTGAGTAAGGACTTCTACCATCATTACTGTGTAATATTTA
TAGTTATGATCAGTACAGTTATTAACAACTTCTCTTGTCTCGCTGAACTTCTCCATCAGTCACCTCGGGCAGGGGC
AGGTGCAGGGGCCGGATTACGACTACCAGGAGGGGGAGGAGCTCAACAAGCGCGCAGTCGATGAA
//
NRC-02 and NRC-03 Winter Flounder WF1A (SEQ ID NO: 83)
ATGAAGTTCACTGCCACCTTCCTCCTGTTGTTCATCTTCGTCCTCATGGTTGATCTCGGAGAGGGTCGTCGTAAGA
GAAAGTGGTTGAGAAGGATTGGTAAAGGTGTCAAGATAATTGGCGGGGCGGCCCTTGATCACCTCGGGCAGGGGCA
GGTGCAGGGGCAGGATTACGACTACCAGGAGGGGCAGGAGCTCAACAAGCGCGCAGTCGATGAAA
//
NRC-04 Winter Flounder WF2 (SEQ ID NO: 84)
GCCCACTTTGTATTCGCAAGGTAATATTGATATTTTTCATATTCATTTAGACAAATGTGCTCAGCTTGTTACTGTA
TAATGCAAAAGTTAATGATCTTTATTTTTCTGTTTTTTTTTGTAGAATGAAGTTCACTGCCACCTTCCTCATGATT
GCCATCTTCGTCCTCATGGTTGAACCTGGAGAGTGTGGCTGGGGAAGCTTTTTTAAAAAGGCTGCTCACGGTAGAG
TCACAGAATTAATTAGCTTTTTGCTTTGCAAATATTTTTTTTATAACAGCTGGAAAATCACAAAAATAAATAGTAT
ATATATTTGGCCAATAAAATCACTTTGATTTCAATAATAATCTAAATAACCAACCTAAAAGGCCTTTGATTAGCAT
GTTCCTTCAATGAAATGTACGTTGAGGTTTATTTTGATTCTCACAAGCACCAACCTGCTGCGTCAACAATTGAATT
CAAATTTGTCCCAAAGGAATTCAAAGTAAATTTTTCTAGGCGATTTAATCTTTCCATTACTCTGATTTGTTTTAAA
AATATAGAATAACTCAATCTCTATGATAAAACAATTACACATACATTCAGATTTTTATAGGACAAGATTGAAAACT
TCTTACAAGTATGTATAAAACATCATCTGTATGTATAATTGTTTAACATGTAACAACTAGTCCTACTAATTGTGTT
AAATTGTCATTTAATATCAATTGCTTGAGTTTATCATTATGTGTTTTGTTTTTTTTTACACAGTTGGCAAGCATGT
TGGCAAGGCGGCCCTTACGTAAGGACTTCTACCATTTTACTGTATAATTTTGATAGTGTTATCACCAGTACTGTTT
TTGACAACTTCTCTATTCCTGCTGACTCTCTCCATCCGACTCATCCGCAGTCATTACCTTGGCGATAAGCAGGAGC
TCAACAAGCGTGCAGTCGATGAAGACCCAAATGTTATTGTTTTTGAATGAAGAAAT
//
NRC-05 Winter Flounder WF3 (SEQ ID NO: 85)
ATGAAGTTCACTGCCACCTTCCTGGTGCTGTCCCTGGTCGTCCTAATGGCTGAGCCTGGAGAGTGTTTCTTAGGAG
CCCTTATCAAAGGGGCCATACATGGTAGAGTCAAGGAATTAATTAGATTTTTACATGTCAAATAATGTAGTAGAAC
ATATATAAGTAGTCAATATATTTGACCAAGTAGAATCATTTTGATTTCAATAATAATCAAAATAACAATCTCCAGG
CGATTTAATATTTGCAATAATTGGATTTTATAGAATACGGAACAACTGGATCTTAATGCTAAAATAATCCAACATA
CATTCTGATTTTGCCAGGCAAAATTAAACACTACTTTAAAGTATGTATAAAACATAATCTGTATGTTATAACAAAT
ACTCCAAGCAATTGTGTGATGGAAATGTATTCATTGTCATTTAATATAATTTGCTTGAGTTTATCATCTTGTGTTT
TTGTTTGTTTTTTCACAGGTGGCAGGTTTATCCATGGGTAAGGACTTCTACCATCATGACTGTGTATTTTTAATAT
TATTATCATCAGTACTGTTATTGACAACTTCACTTGTCTCGCTGACTCTCTCCATCAGAATGATCCAAAACCATCA
CGGTTATGACGAGCAGCAGGAGCTCAACAAGCGCGCAGTCGATGAA
//
NRC-06 Winter Flounder WF4 (SEQ ID NO: 86)
GCCCACTTTGTATTCGCAAGGTAATATCAATATTTTTCAAATTCATTTAGACGAGACCAACCTTTTGGGAAATCTG
CTCAGCTTATTACTGTATAATGCAAATGTTAATGATCTTTATTTTTCTGTTTTTTTTTTGTAGAATGAAGTTCACT
GCCACCTTCCTCATGATGTTCATCTTCGTCCTCATGGTTGAACCTGGAGAGTGTGGTTGGGGAAGCATTTTTAAGC
ATGGTCGTCATGGTAAAGTCACGGAATTAATTAGCTTTTAACTTTGCAAATATTGTTTTTTTTTTTAACAGCTGGA
AACTCACAAAAATAAATAGCCGATATATTTGGCCAATTATAATCACTTTGATCTAAATAACAACCTAAAAGGCCTT
TGATTAGCATGTTTCTTCAATAAAATGATTGAACACTACTTAAAGGTATGTATAAAACATCATCATGTGTTTTTGT
TTGTTTTTACACAGCTGCCAAGCATATTGGCCATGCAGCCGTTAAGTAAGGACTTCTACCATTATTACTGTATAAT
TTTGATAGTATTATCACCAGTATTGTTATTGACAACTTCTCTTTTTCCTGCTGATCCGACTCATCCGCAGTCATTA
CCTTGGCGAGCAGCAAGATCTCGACAAGCGCGCAGTCGATGAAGACCCAAATGTTATTGTTTTTGAATGAAGAAAT
//
NRC-07 Yellowtail Flounder YT2 (SEQ ID NO: 87)
ATGAAGTTCACTGCCACCTTCCTCATGATGTGCATCTTCGTCCTCATGGTTGAACCTGGAGAGTGTCGTTGGGGGA
AATGGTTTAAAAAGGCCACACACGGTAGAGTCACAGAATTAATTAGCTTTTTGCTTTGCAAATATTTTTTTATAAC
AGCTGGAAAATCACAAAAATAAATAGTCTATATATTTGGCCAATTAGAATCACTTTGCTTTCAATAAAAATCTAAA
TAACAACCTAAAAGTCCTTTGATTAGCATTTTCCATCAATGAAATGGACGTTGAGGTTTATTTTGATTCTCACATG
CACCGACCTGGTATGTCAACAATTGAATACAAATTTGTCCCAGAGGAATTCAAAGGAAATTTTTCTAGGCGATCTA
ATCTTTCCATTACTCGGATTTGTTTTTAAATATATAGAATAACTCAATCTCTATGATAAAATAATAACACATACGT
AAAGATTTTTACAAGACAAGATTGAAAACTTCTTAAAAGTACGTATAAAACATCATCTGTATTTATAATTGTTTAA
CATTTAACAAATAGCCCTACTAATTGTGTTATGGAAATGTATAAATTGTCATTTAACATAACTTGTTTGAGTTTAT
CATTATTTGTTTTTGTTTGTTTTTACACAGTTGGCAAGCATGTTGGCAAGGCGGCCCTTACGTAAGGACTTCTACC
ATCATTACTGTATAATTTTGATAGTATTATCACCAGTACTGTTATTGACAACTTCTCTTGTCCTGCTGACTCTCTC
CATCCGACTCATCCATAGTGCTTACCTTGGCGACAAGCAAGAACTCGACAAGCGCGCAGTCGATGA
//
NRC-08 Winter Flounder WFX (SEQ ID NO: 88)
TAATAAAACTAATGTGTAAAGTCTTCCACTTTTTTTACTGTATTTACTTAAACAGAAAATTATTCTCACGATTCTG
GAGCTGCAGCCACTAAGTGTTGCTTCATGAAGTGAATACACAATTGTTCTAACAACCACTCACCCAATTAACCAGA
ATCTACAAAGTGAGGAAGTGAGAGGAGTCGTCCTGTGTTTTCAAATTTTTTGAATGATCTACCACTATGTGAGCTC
CTCCTGTTATAGCTCTAAATGTTACACAATGAATGTGAAGTCAGTTCTGTGTATATAAAGAGTTGCCTCTGTAGAG
CATACAACAGATTTCACCTTTGAATCTCACAAACCTCACTTTGTATTCGACAGGTAAGATCGATATTTTTCAAACT
CATTTAGACGAGACCAAGTATTTGGGAAATGTGCTCAGCTTGTCAATGTATAATGCAAATGTTAACAATCGTTTTG
TTCTTATGTTGTGTTTGTAGGATGAAGTTCGCTACTGCCTTCCTGATGTTGTCCATGGTCGTCCTCATGGCTGAAC
CTGGAGAGTGTCGTTCTACAGAGGACATCATCAAGTCTATCTCGGGTAGAGTCCAGGAATTAATTATTATCAATAA
CAATGAAATAACAACCAAAAGGCCTCTGATTAGCATGTTCCTTCAATGAAATGGTCGTTTTTTATCTATTTTGATT
CTCACATGCAACGACCTGCTGCGGCAACATTTGAAAATCAATCTTTTTTACACAAATTCAAAGTACATTGATTTAT
TCGATTTAATCTTAACATTAATCAGATTTGTTTTTGTTTAAATATATCGAATAACTGGATCTCTATGATAAAATAA
TTAAACATACATTCTTATTTTACCAATCAAGATTGAACACTTCTTAAAAGTACGTATAAAACATCATCTGTATGTA
TAATTGTTTGATTGTTAAGTAATATTTCCAATAATTGTGTAATGGAAATGTATTAATTGTCATTTAATATAATTTG
CTTGAATTTATCACCATGTGTTTTTTGTTTGTTTTTAAACAGGTGGAGGTTTTCTCAATGCGTAAGGACTTCTATC
ATCATTACTGTGTAATTTTTATAGTATTATCATCAGTACTGTTATTAACAGCTTCTCTTGTCTCACTGACTCTCTC
CATCAGAATGAACGCCGGTTACAATGAGCAGCAGGAGCTCAACAAGCGCTCAGATGATGATGACAGCCCCAGTCTT
ATTGTTTTTGACTGAAGAAGTCGCCCTGAAGGAGCCTTCAGATGATATATTATGCTTCTTGCTCTTCATTGAAATA
AATCAAAC
//
NRC-09 and NRC-b Winter Flounder WFY and WFZ (alternative splice products
from the same pseudogene) (SEQ ID NO: 89)
GAGCTCGATCAAACCAGACAAAGTTGCCTTCCTTCACAACAATAGAGTGGAAGAGAAAACAGGAGAGGACTTGTAT
CCTCCTGATGCTGAGAAGAAGAAATAAGAAAGACTTGCAGCATTGATACTTTTACTTATACAGAAAACCTATAAAC
ATGACGGGAGCATAAGTTAAAGTCACAATACAGAAGAGAACCAGAAGCCAAACTGCAGCAAATTTACTGGTATTCA
TATGATACTGGAGCCAAAGCAACGCAGAGACTCAGCAGCAGTGAACCAAAGAGTTTAACTGTACTTGTGTCCAGGT
TGAATGAAAGTATTGAATAAAAAAAACCAAGACAGAACATGCATATTTTTTTGGAATGGAATATAAGTCAGGAGAA
TATGTGTTGTTGTGGTGGCAGGATCCATCACTCTGTCAAGTTAACACAAGAACTTTTAGAAACATAGATACGATCT
CAAGTAAACTTCCATTTACTATTTGACTTTTTTTAAATACTTACAAATTATATTTTAAAAAGCAACAATAAATCAG
AGATAACTTCATGGAGAAGTCTATATTCATATTTGTGAGCTGAACATTCATGCTGCCTGTTCTATCACATCTGAGT
GTGGAGGCCACTGACGTTTACTGACCTCAACGTCTACCGCTCTAATGCATTTGGAGTTAAAGGTAAGCATTTTGTT
ATTTGTCTTCACTGTATTGATACTAAATATACAGGGTTACAAATACAGTTAAAACAAGAGAGACGAGGTGTCGAAA
GCTTCAGCATCAATGTGCTGATCGCTGATAGCTGATCTTACCCGACACCGGTGACATGGCATCAAAATGACCACCT
CTTTTTTCTTCTCTTTTTTTTGTAGGACGAAGTTCGCTGCCGCCTTCCTCGTGTTGTTCATGGTCATCGTCATGTT
TGAACCTGGAGAGTGTTTTTTTAGATTGCTTTTTCACGGGGTCCACCATGGTAGGGTCCCGGAAGTAATTTGATTA
TTACATGCCAAATATTTTAATGAAACATACCTTATGAGTAGTTGTATTATTTGGACAAGTAGAATCTCTATGATTT
CAGTAGTAATTAGAATAACAATCAAAAAGGCCTTTGATTAGCATGTTTCTTCAATGAAATGGACATTGAGGTTTAT
TTTGATTCTCACATGCTACAGCAACAATTGAAATCAAATTTTTCGCAGAAGAAACTTAATTAACATTGTTGTGCAA
TAGTGCTTAAAAAGTGTTACCATGGAATGGTGTGCGTTTAGGCACTCAATAAATTTTGGTTATCAAATTAAATTAA
AAAAATTAATATTTAAAATATTAATATTAAATCATAACTTTAATTGTTTAAAGTTCTCGCGGGGAACCACCCTTCT
TCTGAAGGTAAAGGATAGCCAATTTATTGATTAAGATCAGTCTCATTTAGATCTAGTTCAAATAGAAATCTCAATA
TTTTACCATCGAAGATTTTATAATGAACAGTGAAGGTTATGGAGTTCTAAACAGTGTAACAGTTGGCAAAGTTCAC
TATTGCAATATTAATGACAGACCATTTGTGAAAGAAGAACATTTATTATGAGCATAATAAAGTATGAAAGCACGAA
TTACTAAACAATCAAAGCTAACTAACAAGGACGTGTGTGGGTGTGTGTGTGAATGTAAATAAGGGGGGGGCTCAAA
CTGGTGGCCTACAAGAAGAGCCTTAAGATAGCAACCACAAGGGCTGTACCATAAATGTTGTAGTAAAAAGAGTTAT
TAAAATGAGTTAGAATAACTAATGACTAATTAGTAGACAAACTAGTAGACAAACTAAACAACTAACAATAACAAGG
AAGTGTGTGTGAGTGTGTTTGTGTGTAAATGTTAATTAGGGGCTCTCAAACTGGTGTCTTACCAGAAGAGTAAGAT
AACAATTCCCCCCCTTCTTCTGAGGTTGTTTTACGACTGTTGCTTTATGGCCGTGAGGGAAGGTTTAACTCGGTGA
CATGCTATACGTGTCTGTGTAGATGTTAATCAGAGAATGCCAGAGTCAGAGAGACCTACGGAGGAAGTCTGTGAAG
GGCCTATCTAACATTAGCTTTCCTTTAACTTATAACACAATATCAGAAACACATATCAACCTTATAAACACACACA
GAATCAAATAAACAGTCTTGCTTAGCATGTATAATTATTAAGCCCAGATTATGTTACCAGTCCGAGGGAAAGAGTT
CAGTTGCAGTTCTGTGACGTCTCCTGGCTTTGTGGTCGTAGAGTTCTGCATTCGCGATTCTGTCGAGCCGTGTGCT
CAGATGCAGGTTGAAGTTCTCCTGCAGGACATCGCGTCGCTGCGAGGATTTTGTAGAGCTTGAAGGGCGAGGAGAT
TTCCTTGAGTGGTGAGCTGGAAGCTGGACCTCTGACCTCTGGTTGTTGGTTGGAAGAGAAGAAAGCTGGAGCGGCG
TGGTTTCTCCCTCTAGCCGATGCAGGAGGAGAAGCCGGCAGCCCCACTCCTTGAAGAGTTGTGGAGAGAGATGGGA
GCAAAGAGCTAGATTTTGGGGAGACCTCTCCTTATATTGGCCCCGATGACCTCACAGGCCTTGGAACGGAGTGACC
AATAGGAGTTGACCCTGGTAATTCTTGACACCTTTGTGGGACATTGTCAAGACCCCAGGACATGCAGCATCCTGTT
ACAATCTGGGAGACGGAGTTCCTTGACTGTCTCAGAACAATGAGAACCTGTGGCATCTTGGGGGATTGAGTCCACT
CGAGCACATGCGGCATGTTTGTTCCAAGTTTGACTGAAAGGAGGCCTGTGGTTTGCACAAAAACCATGTCCCAACA
ACATTTTCTAGGCGATTTAATCTTTACATAAATTGGATTTGTTTTAAAAAATATATAGAATAACTCGATCTTTCTG
CGTAAATAATAAAAAATAAATTCAAATTTGACCAGTCAAGATTGAACACTAATGAAAAGTACCTATAAAACATAAT
CTGTATGTATAGTTGTTTGACTGTTAAATAGTAGTCCTAACAATTGTGTAATGGAAATGTATTCATTGTCTTTTAA
TACTATTTGCTTATCATAATGTGTTTGTTTGTTTTTTAGCAGGTGGAGGTTATCTCAATGCGTAAGGACTTCTACC
ATCATTACTGTGTAATTGTATTAGTTTTATCATCAGTACTGTTATTGACAACGTCTCTTGTCTTGCTGACTTGACT
CTCTTCATCAGATTAAACCCAGGGCCGGTTACAATGAGCAGCAGGAGCTCGACAAGCGCGCAGTCGATGACAACCT
CAGTGCTATTGTTTTTTACTGAAGAAGTCGACCTGAAGAATCTTTTGAAATGATATGAAATGTTTGCCTTTCAATG
AAATAAATCAAACATGACTGGATATTTGTTCTTTTGCATTGATGTATTGTTGAGTGACAGTTGAATAATTTTGGAA
AACTTATAACAGATCTCAATTTTAGGATGTCAAATCATTTCTCTGTGTCTTATTCAAATATGAGATTTAACAATGA
CAAT
//
NRC-11 American Plaice AP1 (SEQ ID NO: 90)
GCCCACTTTGTATTCGCAAGGTAAGATCAATATTTTTCAAATTCATTTAGACGAGACCAACCGTTTGCGAAATGTG
CTCAGCTTGTTATTGTATAATAACAAAGTTAACGATCTTTATTTTTCTGTTTTTTTGTAGAATGAAGTTCACTGCC
ACCTTCCTGATGTTGTTCATCTTCGTCCTCATGGTTGAACCTGGAGAGTGTGGATGGAAAAGTGTGTTTCGTAAGG
CTAAGAAAGGTAGAGTCACGGAATTAATTAGCTTTTTACATTGCAAATAGATTTTTTATAACAGCTGGAAAATCAC
AAAAATAAATAGTCGATATATTTGGCCAATTAGAATCACTTTAATTTCAATAATAATCTAAATAACAACCTAAAAG
GCCTTTGATTAGCATGTTTCTTCAATGAAATGGACATTGAGGTTTATTTTGATTCTCACATGCACCGACCTGTGCG
GCAACCATTGAATTCAGATTTGTCCCAGAAGAATTCAAAGTACATTTTTCCAGGCGATTAAATCTTTCCATTACTC
AGATTCAAAAATAAATAAATGGAATAATTGAAGCACTATGATAAAATAATTACACATTCACTCTGACTTTACAAGT
CAAGATTGAACACTATTAAAAAGTGTGTATAAAACAACATCTGTATGCATAATTGTTTAACTGTTAATAGTCCTAA
TAATTGTTTTATGGAAATGTATTAATTTACATTTAATATTATTTGCTTGAGTTTACCATCATGTGTTTTTGTTTGT
TTTTACACAGTTGGCAAGACTGTTGGCGGCTTGGCCCTTGAGTAAGGACTTCTACCATCATTACTGTATAATTTTG
ATAGTATTATCACCAGTACTGTTATTAACTACTTCTCTTGTCTGCTGACTCTCTCCATCCGACTCATCTGCAGTCA
TTACCTTGGCGAGCAGCAGGAGCTTGACAGCGCGCAGTCGATGAGGACCCCAGTGCTATTGTCTTTGACTGAAGAA
GTCGCCTTGAAGGAG
//
NRC-12 American Plaice AP2 (SEQ ID NO: 91)
ACTTTGTATTCGCAAGGTAAGATCAATATTTTTCAAATTCATTTAGACGAGACCAACCGTTGGCGAAATGTGCTCA
ACTTGTTATTGTATAATAACAAAGTTAACGATCTTTATTTTTCTGTTTTTTTGTAGAATGAAGTTCACTGCCACCT
TCCTGATGTTGTTCATCTTCGTCCTCATGGTTGAACCTGGAGAGTGTGGATGGAAAAAATGGTTTAATAGGGCTAA
GAAAGGTAGAGTCACGGAATTAATTAGCTTTTTACATTGCAAATAGATTTTTTATAACAGCTGGAAAATCACAAAA
ATAAATAGTCGATATATTTGGCCAATTAGAATCACTTTAATTTCAATAATCTAAATAACAACCTAAAAGGCCTTTG
ATTAGCATGTTTCTTCAATGAAATGGACATTGAGGTTTATTTTGATTCTCACATGCACCGACCTGTGCGGCAACCA
TTGAATTCAGATTTGTCCCAGAAGAATTCAAAGTACATTTTTCCAGGCGATTAAATCTTTCCATTACTCAGATTCA
AAAATAAATAAATAGAATAATTGAAGCACTATGATAAAATAATTACACATTCACTCTGATTTTACAAGTCAAGATT
GAACACTATTAAAAACTGTGTATAGAACATCATCTGTATGTGTAATTGTTTAACTGTTAATAGTCCTAATAATTGT
TTTATGGAAATGTATTAATTTACATTTAATATTATTTGCTTGAGTTTACCATCATGTGGTTTTGTTTGTTTTTACA
CAGTTGGCAAGACTGTTGGCGGCTTGGCCGTTGAGTAAGGACTTCTACCATCATTACTGTATAATTTTGATAGTAT
TATCACCAGTACTGTTATTAACTACTTCTCTTGTCTCGCTGACTCTCTCCATCCGACTCCTCTGCAGTCATTACCT
TGGCAAGCAGCCGGAGCTCGACAAGCGCGCAGTCGATGAGGACCCCAGTGCTATTGTCTTTGACTGAAGAAGTCGC
CTTGAAGGAGCCTTCAGAA
//
NRC-13 American Plaice AP3 (SEQ ID NO: 92)
TTGCCCACTTTGTATTCGCAAGGTAAGATCAATATTTTTCAAATTCATTTAGACGAGACCAACCATTTGGGAAATG
TGCTCAGCTTGTTACTGTATAATGCAAAAGTTAAGTATCTTTATTTTTCTGTTTTTTTTTGTAGAATGAAGTTCAC
TGCCAACTTCCTCATGTTGTTCATCTTCGTCCTCATGTTTGAACCTGGAGAGTGTGGTTGGCGAACATTGCTTAAA
AAAGCTGGTCACGGAATTAATACGCTTTTTACATTGCAAATAGATTTTTTATAACAGCTGGAAAATGACAAAAATA
AATAGTCGATATATTTGGCCAATTAGAATTATTTTGATTTCAATAATAATCTAAATAACAACCTAAAAGGTCTTTG
ATTAGCATGTTTCTTCAATGAAATGGACATTGAGGTTTATTTTGATTCTCACATGACCGACCTGCTGCGGCAACAA
TTGAATTCAGATTTGTCCCAGAAGAATTCAAAGTAAATTTTCCAGGGGATTAAATCTTTCCATTACTCGGATTTAA
AAAAAAAAAAAATAGAATAACTGAATTGCCATGAAAAAATAATTACACATACTGTCTGATTTTACAAGTCAAGATT
GAACACTACTTAAAAGTATGTATAAAACATCATCTGTATGTATAATTGTTTAACTGTTAACAAATAGTCCAAATAA
TTGTGTTATGGAAATGTATTAATTGTCATTAAATATAATTTGCTTGAGTTTATCATCATGTGTTTTTTTTTTTTTT
TTACACAGAGGTTAAGACTGTTGGCAAGTTGGCCCTTAAGTAAGGACTTCTACCATCATTACTGTATAATTTTGAT
AGTATTATCACCAGTACTGTAGTACTGACAACTTCTCTCTCCACCCAACTCATCCGCAGACATTACCTTGGCAAGC
AGCCGGAGCTCGACAAGCGCGCAATTGATGACGACCCCAGTATTATTGTTTTTGACTGAAGAAGTCGCCTTGAAGG
AGCCTTCAGAA
//
NRC-14 Witch Flounder GcSc4C5 (SEQ ID NO: 93)
ATGAAGTTCACTGCCACCTTCCTCATGATGTTCATGGTCGTCCTCATGGCTGAACCCGGAGAGGCTGGTTGGGGAA
GTATTTTCAAACATATTTTCAAAGCTGGAAAGTTCATCCATGGTGCGATCCAGGCACACAATGACGGCGAGGAGCA
GGATCTCGACAAGCGCGCAGTCGATGA
//
NRC-15 Witch Flounder GcSc4B7 (SEQ ID NO: 94)
ATGAAGTTCACTGCCACCTTCCTGGTGTTGTTCATGGTCGTCCTCATGGCTGAACCTGGAGAGGGTTTTTGGGGAA
AGCTTTTGAAATTGGGCATGCATGGAATCGGGCTGCTCCATCAGCATTTGGGTGCTGACGAGCAGCAGGAGCTCGA
CGAGCGCTCAGAGGAGGACGAGCCCAATGTTATTGTTTTTGAATGAAGAAGTCGCATTGAAGGAGCCTTCAG
//
NRC-16 and NRC-17 Witch Flounder GC3.8 (SEQ ID NO: 95)
ATGAAGTTCACTGCCACCTTCCTGGTGTTGTTCATGGTCGTCCTCATGGCTGGATCCGGAGAGTGTGGTTGGAAAA
AGTGGCTCCGTAAAGGTAGAGTCATGGATTTAATTTGCTTTTTACATTGCAAATACTTTAATATAACATAGTTGGA
AAACCACAAAAATAAGTAGTCGATATATTTGGCCATATAGAATCACTTTGATTTCAATAATAATCAAAACAACAAT
CAAAAAGCCCATTGATTAGCATGTCCCTTCACTAAAATGGACATTGTAATTTATTTTGATTCTCACAGGCACCAAC
CTGCTGCGGCAACAATTGAAATCAAATTTGTCTCAGAAGAATTCAAAGTACATTGTTCTAGGCGATTTAATCTTTC
CATTCATCGGATCTGTTTTTAAAAATATAGAATAACTGGATCTCTATGTTAAAATAATAAAACACACATTCTGATT
TTACCTGTCAAGATTGAACACGACTTAAAAGTATGTATAAAACATCATCTGTATGTATAATTGTTTAACTGTCAAC
TAATAGTCCAAATAATTGTGTTATGGAAATGTATTCATTGTCATATAATATCATTTGCTTGAATTTATCACCATGT
GTTTTTGTTTGTTTTTACACAGGTGCCAAGCACCTTGGCCAGGCGGCCATTAAGTAAGGACTTCTACCATCATTAC
TGTGTAATTTTAACAGTATTATCATCAGTACTGTTATTGACAACTACTCTTGTCTCTGTTACTCTCTCCAGGGGTT
TGGCCTCTTGCGAAGAGCAGCAGGAGCTCGACAAGCGCTCAATGGATGACGAGCCCAGTGCTATTGTTTTTGACTG
AAGAAGTCGCCTTGAAGGAGCCTTCA
//
NRC-18 Witch Flounder GC3.2 (SEQ ID NO: 96)
ATGAAGTTCACTGCCACCTTCCTGGTGTTGTTCATGGTCGTCCTCATGGCTGGATCCGGAGAGTGTGGTTGGAAAA
AGTGGTTCACTAAAGGTAGAGTCATGGATTTAATTTGCTTTTTACATTGCAAATACTTTAATATAACATAGCTGGA
AAATCACAAAAATAAGTAGTCGATATATTTGGCCATATAGAATCACTTTGATTTCAATAATAATCAAAACAATAAT
CAAAAAGCCTATTGATTAGCATGTTCCTTCACTAAAATGGACATTGTAATTTATTTTGATTCTCACAGGCACCAAC
CTGCTGTGGCAACAATTGAAATCAAATTTGTCTCAGAAGAATTCAAAGTACATTGTTCTAGGCGATTTAATCTTTC
CATTCATCGGATTTGTTTTCAAAAATATAGAATAACTGGATCTCTATGTTAAAATAATAAAACACATTCTGATTTT
ATCTGTCAAGATTGAACACGACTTAAAAGTATGAATAAAACATCATCTGTATGTATAATTTTTTAACTGTCAACTA
ATAGTCCAAATAATTGTGTTATGGAAATGTATTCATTGTCATATAATATCATTTGCTTGAATTTATCACCATGTGT
CTTTGTTTGTTTTTACACAGGTGAAAGGTTATCCCAGAGGTAAGGACTTCTACCATCATTACTGTATAATTTTAAT
AGTATTATCATCAGTACTGTTATTGATAACTTCTCTTGTCTCGCTGACTCTCTCCATCAGGCATTTCGCTGACGTC
GAGCAGCAGGAGCTCGACAAGCGCTCAGTGGATGACGAGCCCAGTTCTATTGCTTTTGACTGAAGAAGTCGCCTTG
AAGGAGCCTTCAG
//
NRC-19 Halibut HB26 (SEQ ID NO: 97)
TTATGAAGTTCACTGCCACCTTCCTGGTGTTGTTCATGGTCGTCCTCATGGCTGAGCCTGGAGAGTGTTTTTTGGG
ATTGCTTTTTCACGGGGTCCACCATGGTAGGGTCACGGAAGTAATTCGATTTTTACATGGCAAATATTTTAAGATA
ACACACCATATGAGTAGTCGATATATTTGACCAATTAGAATCACTTTAATTTCAATAATAATCACAATAACAATCT
CTAGGCCATTTAATCTTTCCATTAATCGGATTTGTTTTTTTAAATATAGAATAACTGGATCTCTATGTTAAAATAA
TAAAACATACATTCTGATTTTACCAGTCAAGATTGTACGCTACTTAAAAGTATGTATAAAACATCATCTGTATGTA
TAATTGTTTAACTGTTAACTAATAGTCCAAATAATTGTGTAATGGAAATGTATTAATTGTCATTTAATATCATTTG
CTTGAATTTATCACCATGTGTTTTTGTTTGTTTTTACACAGTTGGAAAGTGGATCCATGGGTAAGGACTTCTACCA
TCATTACTGTGTATTTTTAATAGTATTATCATCAGTACTGTTATTGATATTTTCTCTTGTCTCGCTGACTCTCTCC
ATCAGACTCATCCATGGGCATCACGGTTACGACGAGCAGCAGGAGCTCGACAAGCGCGCAGTCGATGAAA
//
NRC-20 Halibut HB18 (SEQ ID NO: 98)
TTATGAAGTTCACTGCCACCTTCCTGGTGTTGTTCATGGTCGTCCTCATGGCTGAACCTGGAGAGGGTTTTTTGGG
AATTCTTTTTCACGGGGTCCACCATGGTAGAGTCACGGAATTAATTCGATTTTTACATGGCAAATATTTTAAGATA
ACACACCATATGAGTAGTCGATATATTTGACCAATTAGATTCACTTTAATTTCAATAATAATCACAATAACAATCT
CTAGGCCATTTAATCTTTCCATTAATCGGATTTGTTTTTTTAAATATAGAATAACTGGATCTCTATGTTAAAATAA
TAAAACATACATTCTGATTTTACCAGTCAAGATTGAACACTACTTAAAAGTATGTATAAAACATCATCTGTATGTA
TAATTGTTTAACTGTTAACAATAGTCCAAATAATTGTGTTATGGAAATGTATTAATTGTCATTTAATATCATTTGC
TTGAATTTATCACCATGAGTTTTTTGTTTGTTTTTACACAGGTAGAAAGAAGGCCTTGCAGTAAGGACTTCTACCA
TCATTACTTTGTAATTTTTATAGTATTATCATCAGTACTGTTATTGACAACTTCTCTTGTCTCGCTGACTCTCTCC
ATCAGGATGAACTCAGAGCGTCGCAGTTACGACGAGCGGCAGCAGCAGCAGCAGGAGCTCGACAAGCGCGCAGTCG
ATGAAA
//
NRC-101 Yellowtail Flounder YT1 (SEQ ID NO: 99)
GCCCACTTTGTATTCGCAAGGTAAGATCGATATTTTTCAAACTCATTTAGACGAGACCAAGCATTTGTTGAAATGT
GATAAGCTTCTAACTTTATAATGCAAATGTTAACAATCTTITTGTTCTGTTGTTTTTGTAGGATGAAGTTGGCTGC
CGCCTTCCTGGTGCTGTTCCTGGTCGTCCTCATGGCTGAACCTGGAGAGGGTTTCTTGGGATTTCTTTTTCACGGT
ATCCACCATGGTAAAGTCACTCATTTAATACATTTTTACATGGCAAATATTTGAATATAACATACTATATGAGTTG
TCAATATATGTGGCCAAGTAGAAGCACTTTGATTTCAATAATAATCAAAATAACAATCACTAAGCCATTTAATAAT
TGAATTAATTACATTTGTTTTAAAAAAATATAGAATAACTGGATCTTTATGCTAAAATAATTAAACCTAAATTCAG
ATTTTACCACTCAAGATTGAACACTACTTAAAAGTATGTAAAAAAAACATCATCTGTATGTATAATTAAATACTAG
TCCAGTTAATTGTTTTATGGAAATGTGTTAATTGACATATATCATTTGCTTGAACTTATAATGTGCTTTGTTTGTT
TTTACACAGGTATCAGGGCGATCCATCAGTAAGGACTTCTACCATCATGACTGTGTATTTTTAATAGTATTATCAT
CAGTACTTTTATTAACAACTTCTCTTGTCTCGCTGACTCTCTCCATCAGTCTCATCCATGGTCAAAGATACGACGA
GCAGCAGGAGCTTGACAAGCGCTCAGTCGATGACAACCCCGGTGCTATTGTTTTTGACTGAAGACGTCGCCTTGAA
GGAGCCTTCAG
//
NRC-102 Yellowtail Flounder YT3 (SEQ ID NO: 100)
ATGAAGTTCACTGCCACCTTCCTGGTGTTGTCCATGGTCGTCCTCATGGCTGAACCTGGAGAGGGTTTCTTTGGAG
CCCTTATCAAAGGGGCCATCCATGGTGGCAAGTTGCTCCATAAACTCATCAAAAAAAAACATGAACATCACGGTTA
TGGCAAGCATTGGGGGCTTGACAAGCGCGCAGTCGATGA
//
MRC-103 Winter Flounder WF-YT (SEQ ID NO: 101)
TTGAAAGTGAGGAAGTGAGAGGAGGACTAGGTCCTGTGTTTTCAGTCGTTGAATTATCTAACACTATCTGAGCCCC
TCCTGCAATAACTCTAAATGTTACACAGTGACTAGGAAGTCAGTCCTGTGTATATAAAGAGTTGCATCTGTTGTTA
TCAGTAGACAACAGATTACACCTTTGAATCTCACAAAGCTCATTTTGTATTCGACAGGTAAGATCGATATGTTTCA
AACTCATTTAGATGAGACCAAGCATTTGGGAAATGTGCTCAGCTTCTAACTGTATGATGCAAATGTTAACAATCTT
TTTGTTCTGTTGTTTTGTAGGATGAAGTTGGCTGCCGCCTTCCTGGTGCTGTTCCTGGTCGTCCTCATGGCTGAAC
CTGGAGAGAGTTTTTTGGGATTTCTTTTTCATGGTATCCGCCATGGTAGGGTCACTGAATTGATACATTTTTACAT
GGCAAATATTTGAATGTAACATACTATATGAGTTGTCAATATATGTGGCCAAGTAGAAGCACTTTGATTTCAGTAA
TAATCAAAATAACAATCACTAGGCCATTTAATAATTGCATTAATTACACTTGTTTTTATATAGAATATAGAATAAC
TGGATCTTTATGCTAAAATTAATAAACATGAATTCAGATTTTAAGATTTTTCAAGATTGAAAACTACTTAAAAGTA
TGTAAAAAAACATCATCTGTATGTATAATTAAATACTTGTCCAGATAATTGTGTTGTGGAAATGTGTTAATTGACA
TATATCATTTGCTTGAATTTATCATTATCTGCTTTGTTTGTTTTTACACAGGTATCAAGGCGATCCATGGGTAAGG
ACTTCTACCTTCATGACTGTGTATTTTTAATAGTATTATATTCAGTACTGTTATTGAAAACTTCTCTTGTCTCGCT
GACTCTCTCCATCAGAATGATCCATGGTAACAGTTTAGACGAGATGCAGGAGCTCGACAAGCGCTCATTCGATGAC
AACCCCAACGCAATTGTTTTTGACTGAAGAAGTCGCCCTGAAGGAGCCTTCAGATGATATATAATGCTTCTTGCTT
TTCAATGAAATAAATTGAATAATTACCCGCAACAGC
//
NRC-104 Winter Flounder WF1-like (SEQ ID NO: 102)
TACTTTTATCTACCACTATGTGAGCTCCTCCTGTTATAACTCTAAATGTTACACAATGAAGATGAGGTCAATTCTG
TGTATATAAAGAGTTGCCTCTGTATAGTAGACAACATATTTCACCTTTGAATCCCACAAAGCTCACTTTGTACTCA
ACAGGTAAGATCGATATTTAAAAACTAATTTAGACGAAACCAAGCATTTTGGGGAATTTGCTCAACTTCTAAATGT
ATGATACAAATGTTAACAATCTTTTATTTCTGTTGTTGTTTTTTGTAGGATGAAGTTCACTGCCACCCTCCTCCTG
TTGTTCATCTTCGTCCTCATGGTTGATCTCGGAGAGGGTCGTCGTAAGAAAAAGGGGTCGAAGAGAAAGGGGTCCA
AGGGAAAGGGGTCCAAGGGAAAGGGCAGGTGGTTGGACAGGATTGGTAAAGGTAGAGTCACGGAATTAATTTGCTT
TTTACATTGCAAATATTTTTCATATAACATTGCTGGAAAATCACAAAAATAAGTAGTCAATATATTTGGCCAAATA
GAATCACTTTGATTTCAATAATAATCAAAATAACAACCTAAAAGGCCTTTGATTAGCATGTTCCTTCAATGAAATG
GACATTGTAATTTACTTTGATTCTCACATGCTACGACCTGCTGCAGCAACATTTGAAAATAAATTTGTCCCAGAAG
ATTTTAAAGTACATTGTTATAGGCGATTTATCTTTCTATTACTCAGATATTTGTTCAAACCAATAGAATAACTGGA
TCTCTATGCTAAAATAATAAAACACACATTCAGATGTTACCAGTCAAGATTGAACGCTGTTTAAAAGTAAGTATGA
AACATCCTCTGTATGTATAATTGTTTAACTGGTAACTTATAGTCCTAATAATTGCGTTATGGAAATGTATTAATTG
TCATTTAATATAATTTGCTGGAATTTATCACTGTGTGTTTTTGTTTGTTTTTACACAGCTGGCGGGATAATTATCG
GGGGGGCCCTTGAGTAAGGACTTCTACCATCATTACTGTGTAATATTTATAGTTATGATCAGTACAGTTATTAACA
ACTTCTCTTGTCTCGCTGAACTTCTCCATCAGTCACCTCGGGCAGGGGCAGGTGCAGGGGCCGGATTACGACTACC
AGGAGGGGGAGGAGCTCAACAAGCGCTCAGACGATGATGACAGCCCCAGTCTTATTTTTTTTGACTGAAGAAGTCG
CCCTGAAGGAGCCTTCAGATGATATATAATGCTTCTGGCTTTTCATTGAAATAAATAATACGTTTACCTGCAACAG
CAACCATG
//
NRC-105 Halibut Hb29 (SEQ ID NO: 103)
TTATGAAGTTCACTGCCACCTTCCTGGTGTTGTTCATGGTCGTCCTCATGGCTGAACCTGGAGAGGGTTTGGGAAA
TTGGATGGGGCCCCATATCAGCGGTAGAGTCACGGAATTAATTTGCTTTTTCCATTGCAAATATTTTAATATTGCA
TAGCTGGAAAATCACGAAATAAGTAGTCGATATATTTGGCCAAATAGAATCACTTTGATTTCAATAATAATCAAAA
TAACAATCAAAAAGGCCTTTGATTAGCATGTTCCTTCAATAAAATGGACATTGAAGTTTATTTTGATGCTCACATG
CACCGACCTGCTGCGGCAACAATTGAAATCAAATTTGTCTCAGAATTTAAAGTACATTTTTCTAGGTGATTTAATC
TTTCCATTAACTTGATTTGTTTTTATAAATATAGAATAACTGGATCTTTATGCCAAAATAATAAAACACACATTCT
GATTTTACCAGTCAAGATTGAACACTACTTAAAAGTAATATAAAACATCATCTGTATGTATAATTGTTTAACTGTT
AACAAAAGTCCAAATAATTGTGTTATGGAAATGTATTAATTGTCATTTAATATCATTTGCTTGAATTCATCACCAT
GTGTTTTTTGTTTGTTTTTACACAGGTGAAAAGAAGGCCTTGCAGTAAGGACTTCTACCATCATTACTTTGTAATT
TTTATAGTATTATCATCAGTACTGTTATTGACAACTTCTCTTGTCTCGCTGACTCTCTCCATCAGGATGAACTCAG
AGCGTCGCAGTTACGACGAGCGGCAGCAGCAGCAGCAGGAGCTCGACAAGCGCGCAGTCGATGA
//
NRC-106 Halibut HbSc1A13 (SEQ ID NO: 104)
ATGAAGTTCACTGCCACCTTCCTGGTGTTGTTCATGGTCGTCCTCATGGCTGAACCTGGAGAGGGTTTGGGAAATT
GGATCGTGCGCCCTATCGGAGGTGAAAAGAAGGCCTTGCAGATGAACTCAGAGCGTCGCAGTTACGACGAGCGGCA
GCAGCAGCAGCAGGAGCTCGACAAGCGCGCAGTCGATGAAA
//
NRC-107 Halibut HbSc1A24 (SEQ ID NO: 105)
ATGAAGTTCACTGCCACCTTCCTGGTGTTGTTCATGGTCGTCCTCATAGCTGAACCTGGAGAGAGTCTTTTTGGAA
AGTTCCTCAAGAAAGTTGTCCATGCTGGCACGTCAATTGGCGAGACAGCCTTGCATGTCGCCGCAGAGCATCACGG
GCTTCATGCGCATCACGGGTGTCACGGGCGTCACGGGGGTCACAGGCGTCACGGGGGTCACAGGCGTCACGGGCGT
CGCGGTTACGACGAGCAGCAGCAGGAGGAGCTCGACAAGCGCGCATTCGATGA
//
NRC-108 Halibut HbSc1B34 (SEQ ID NO: 106)
TATGAAGTTCACTGCCACCTTCCTGGTGTTGTTCATGGTCGTCCTCATGGCTGAACCTGGAGAGGGTTTGGGAAAT
TGGATGGGGCCCCATATCAGCGGTAGAAAGAAGGCCTTGCACATGAACTCAGAGCGTCGCAGTTACGACGAGCGGC
AGCAGCAGCAGCAGGAGCTCGACAAGCGCGCAGTCGATGAAA
//
NRC-109 Halibut Hb17 (SEQ ID NO: 107)
ATGAAGTTCACTGCCACCTTCCTGGTGTTGTTCATGGTCGTCCTCATGGCTGAACCTGGAGAGTGTTTTTTGGGAT
TGCTTTTTCACGGGGTCCACCATGGTAGGGTCACGGAAGTAATTCGATTTTTACATGGCAAATATTTTAAGATAAC
ACACCATATGAGTAGTCGATATATTTGGCCAATTAGAATCACTTTGATTTCAATAATAATCAAAATAACAATCTCT
AGGCGATTTAATATTTGCATTAATTGGATTTGTTTTTAAAAATATAGAATAACTGGATCTTTATGGTAAAATAATT
AAACATACATTCTGATTTTACCAGTCAAGATTGAACACTACTTAGAAGTATGTATAAAACATCATCTGTATGTATA
ATTGTTTAACTGTTAACGAATAGTCCAAATAATTGTGTTATGGAAATGTATTAATTGTCATTTAATATCATTTGCT
TGAATTTATCACCATGTGTTTTTGTTTGTTTTTACACAGTTGGAAAGTTGATCCATGGGTAAGGACTTCTACCATC
ATTACTGTGTATTTTTAATAGTATTATCATCAGTACTATTATTGACAACTTCTCTTGTCTCGCTGACTCTCTCCAT
CAGACTCATCCATGGCGGTTACGACGAGCAGCAGGAGCTCGACAAGCGCGCAGTCGATGAA
//
NRC-110 Witch Flounder GC1.2 (SEQ ID NO: 108)
GCCCACTTTGTATTCGCAAGGTAAGAGCGATATATTTCAAATTCATTCGGATGAGACCAAGCATTTGGGAAATGTG
CTCAGCTTGTTACTGTTTAATGCAAATGTTAACAATATCCTTTTTCTGTTGTTTTTGTAGAATGAAGTTCGCTGCC
GCCTTCCTCATGATGTTCATGGTCGTCCTCATGGCTGAACCCGGAGAGGCTCGTTGGGGAACGTTCTTCAAACATA
TTTTCAAAGGTAGAGTCACAGAATTAATTTGCTTTTTACATTGCAAATATTTTCATATAACATAGCTGGAAAATCA
CAAAAATAAGGGCTTGATATATTTGGCAAAGTAGAATCCCTTTGATTTCAATAATAATCAAAATAAAAATCAGAAA
GGCCTTTGATTAGCATGTTCCTTCAATAAAATGGACATTGTAGTTTATTTTGATTCTCAAATGCACCAACCTGCTG
CGGCAACAATTGAAATCAAATTTGTCTCCGAAACATTTAAAGTACATTTTTCGAGGCAATTTAATCTTTCCTTTGA
TCGAATTCGTTTTTAAAAATATAGAATAACTGGATCTTTATGCTAAAATAATAAATCATACATTCTGATTTTACCA
GTCAAGATTGAACGCTACTTAAAAGTATGTATAAAACATCATCTGTATGTATAATTGTTTAACTTTTAACTAATAG
TCCTAATAATTGTGTTATGGAAATGTATTCATTGTCATTTAATATCATTTGCTTGAATTTATCACCATGTGTTTTT
GTTTGTTTTTACACAGCTGGAAGGTTCATCCATGGGTAAGGACTTCTACCATCATTACTGTGTATTTTTAATAGTA
TTATCATCAGTACTGTTATTGATAACTTCTCTTGTCTCGCTGACTCTCTCCATCAGTGCGATCCAGGCACACAATG
ACGGCGAGCAGCAGGATCTCGACAAGCGCTCAGTGGATGATGAGCCCAGTGTTATTGTTTTTGAATGAAGAAGTCG
CCTTGAAGGAGCCTTCAG
//
NRC-111 Witch Flounder GC1.3 (SEQ ID NO: 109)
GCCCACTTTGTATTCGCAAGGTAAGAGCAATATATTTCAAATTCATTTAGACGAGACCAAGCATTTGGGATCTGTG
CTCAACTTGTAACTGTATAATGCAAATGTTAACAATATTCTTTTTCTGTTGTTTTTGTAGAATGAAGTTCGCTGCC
GCCTTCCTCATGATGTTCATGGTCGTCCTCATGGCTGAACCCGGAGAGGGTGCTTGGATACCTGCCTTGAATAGGA
TCTATCATGGTAGAGTCACAGAGTTAATTTGCTTTTTACATTGCAAATATTTTAATATAACATGGCTGGAAAATCA
CAAAAATGAGTACTCGATATATTTGGCAAAGTAGAATCCCTTTGATTTCAATAATAATCAAAAACACAATCAAAAA
GGCCATTGATTAGCATGTTCCTTCAATGAAATGGACATTGTAGTTTATTTTGATTCTGACATGCACCAACTTGCTG
CGGCAACAATTGAATTCAAATTTGTCTCAGAAAAATTTAAAGTACATTTTTCTTTCCATTAGTCGGATTTGTTTTA
AAAAATACAGAATAACTGGATCTTTATGCTAAAATAATAAATCATACATTCTGATTTTACCAGTCAAGATTGAACG
CTACTTAAAAGTATGTATAAAACATCATCTGTATTGATAATTGTTTAACTTTTAACTAATAGTCCTAATAATTGTG
TTATGGAAATGTATTCATTGTCATTTAATATCATTTGCTTGAATTTATCACCATGTGTTTTTGTTTGTTTTTACAC
AGCTCTACTGAGGATCAATCGGTAAGGACTTCTACCATCATTACTGTGTAATTTTAATAGTATTATCATCAGTACT
GTTATTGATAACTTCTCTTGTCTTGCTGGCTCTCTCCATCAGCCAAATGGTGTATTATCGTCGGCACTGGCACGGT
GACGTCGAGCAGCAGGCTCTCGACAAGCGCTCAGTGGAGGACCAGCCCAGTTCTATTGCTTCTGCCTGAAGAAGTC
GCCTTGAAGGAGCCTTCAG
//
NRC-112 Witch Flounder GC1.4 (SEQ ID NO: 110)
GCCCACTTTGTATTCGCAAGGTAAGAGCAATATATTTCAAATTCATTTAGACGAGACCAAGCATTTGGGATCTGTG
CTCAACTTGTAACTGTATAATGCAAATGTTAACAATATTCTTCTTCTGTTGTTTTTGTAGAATGAAGTTCGCTGCC
GCCTTCCTCATGATGTTCATGGTCGTCCTCATGGCTGAACCCGGAGAGGGTGCTTGGATGCCTGCCTTGAATAGGA
TCTATCATGGTAGAGTCACAGAGTTAATTTGCTTTTTACATTGCAAATATTTTAATATAACATGGCTGGAAAATCA
CAAAAATGAGTACTCGATATATTTGGCAAAGTAGAATCCCTTTGATTTCAATAATAATCAAAAACACAATCAAAAA
GGCCATTGATTAGCATGTTCCTTCAATGAAATGGACATTGTAGTTTATTFTGATTCTGACATGCACCAACTTGCTG
CGGCAACAATTGAATTCAAATTTGTCTCAGAAAAATTTAAAGTACATTTTTCTTTCCATTAATCGGATTTGTTTTA
AAAAATACAGAATAACTGGATCTTTATGCTAAAATAATAAATCATACATTCTGATTTTACCAGTCAAGATTGAACG
CTACTTAAAAGTATGTATAAAACATCATCTGTATTGATAATTGTTTAACTTTTAACTAATAGTCCTAATAATTGTG
TTATGGAAATGTATTCATTGTCATTTAATATCATTTGCTTGAATTTATCACCATGTGTTTTTGTTTGTTTTTACAC
AGCTCTACTGAGGATCAATCGGTAAGGACTTCTACCATCATTACTGTGTAATTTTAATAGTATTATCATCAGTACT
GTTATTGATAACTTCTCTTGTCTTGCTGACTCTCTCCATCAGCCAAATGGTGTATTATCGTAGGCACTGGCACGGT
GACGTCGAGCAGCAGGCTCTCGACAAGCGCTCAGTGGAGGACCAGCCCAGTTCTATTGCTTCTGCCTGAAGAAGTC
GCCTTGAAGGAGCCTTCAG
//
NRC-113 Witch Flounder GcSc4B35 (SEQ ID NO: 111)
ATGAAGTTCACTGCCACCTTCCTGGTGTTGTTCATGGTCGTCCTCATGGCTGGATCCGGAGAGTGTGGTTGGAAAA
AGTGGTTCACTAAAGGTGCCAAGCACCTTGGCCAGGCGGCCATTAACGGTTTGGCCTCTTGCGAAGAGCAGCAAGA
GCTCGACAAGCGCTCAGAGGATGACGAGCCCAGTGCTATTGTTTTTGAA
//
NRC-114 Witch Flounder GC3.6 (SEQ ID NO: 112)
ATGAAGTTCACTGCCACCTTCCTGGTGTTGTTCATGGTCGTCCTCATGGCTGGATCCGGAGAGTGTGGTTGGAAAA
AGTGGCTCCGTAAAGGTAGAGTCATGGATTTAATTTGCTTTTTACATTGCAAATACTTTAATATAACATAGTTGGA
AAATCACAAAAATAAGTAGTCGATATATTTGGCCATATAGAATCACTTTGATTTCAATAATAATCAAAACAACAAT
CAAAAAGCCCATTGATTAGCATGTTCCTTCACTAAAATGGACATTGTCATTTATTTTGATTCTCACAGGCACCAAC
CTGCTGCGGCAACAATTGAAATCAAATTTGTCTCAGAAGAATTCAAAGTACATTGTTCTAGGCGATTTAATCTTTC
CATTCATCGGATTTGTTTTTAAAAATATAGAATAACTGGATCTCTATGTTAAAATAATAAAACACACATTCTGATT
TTACCTGTCAAGATTGAACACGACTTAAAAGTATGTATAAAACATCATCTGTATGTATAATTGTTTAACTGTCAAC
TAATAGTCCAAATAATTGTGTTATGGAAATGTATTCATTGTCATATAATATCATTTGCTTGAATTTATCACCATGT
GTTTTTGTTTGTTTTTACACAGGTGCCAAGCACCTTGGCCAGGCGGCCATTAAGTAAGGACTTCTACCATCATTAC
TGTGTAATTTTAACAGTATTATCATCAGTACTGTTATTGACAACTACTCTTGTCTCTGTGACTCTCTCCAGGGGTT
TGGCCTCTTGCGAAGAGCAGCAGGAGCTCGACAAGCGCTCAATGGATGACGAGCCCAGTGCTATTGTTTTTGACTG
AAGAAGTCGCCTTGAAGAGCCTTCAG
//
NRC-115 Witch Flounder GC2.2 (SEQ ID NO: 113)
GCCCACTTTGTATTCGCAAGGTAAGAGCGATATATTTCAAACTCATATAGACGAGACCAAGCATTTGGGAAATGTG
CTCAGCTTGTTACTGTATAATGCAAATGTTAACAATGTTTTTGTTCTGTTGTTTTTGCAGAATGAAGCTCGCTGCT
GCCTTCCTGGTGTTGTTCATGGTCGTCCTCATGGCTGAACATGGAGAGGGTTTTGGGGATTTCTATATGAAGCCTG
GTAGAGTCACGGAATTAATTCGATTTTAACATGGCAAATATTTTACTATAACATACCATATGAGTAGTCGATTAAT
TAATTGGATTTGTTTTTAAAAATATAGAATAATTGGATCTTTATGCTAAAATAATTAAACATACATTCTGATTTTA
CCAGTTAAGATTGAACGCTACTTAAAAGTATGTATAAAACATCATCTGTACATATAATTGTTTAACTGTTAACCAA
TAGTCCAAATAATTGTGTTGTGGAAATGTATTAATTGTCATTTAATATCATTTGCTTGAATTTGTCACCATGTGTT
GTTGTTTGTTTTTACACAGGTAGAAAGATTTCCCATGGGTAAGGACTTCTACCATCATTACTGTGTATTTTTAGCA
GTATTATCATCAGTACTGTTATTGATAACTTCTCTTGTCTCGCTGACTCTCTACAGGTACATCAGAAGTCCTTATG
GTTACGACGAGCAGCAGGAGGTCGACAAGCGCTCAGTCGATGACAACCCCAGTGCCATTGCTTCTGACTGAAGAAG
TCGCCTTGAAGGAGCCTTCAGA
//
NRC-116 Witch Flounder GcSc4B28 (SEQ ID NO: 114)
ATGAAGTTCACTGCCACCTTCCTGGTGTTGTTCATGGTCGTCCTCATGGCTGAACCTGGCGAGGGTTATTGGCGCT
TCCGCAACCACCGTGGTGAAAGGTTATCCCAGAGGCATTTCGCTGACGTCGAGCAGCAGGAGCTCGACAAGCGCTC
AGTGGATGACGAGCCCAGTTCTATTGCTTTTGA
//
NRC-117 Witch Flounder GC3.7 (SEQ ID NO: 115)
ATGAAGTTCACTGCCACCTTCCTCGTGTTGTTCATCGTCATGTTTGAACCTGGAGAGTGTTTTTGGAATGCTTTTT
CACCGGGTCCACCATGGTCGGGTCACGGAAGTAGTTCGATTTTTACATGGCAAATATTTAAATGAAACATACCATA
TGAGTAGTCGATATATTTGGCCAAGTAGAATCACTTTGACTTCAATAATAATCAAAAACATAATCAAAAAGCCCAT
TGATTAGCATGTTCCTTCAATGAAATGGACATTGAGGTTTATTTTGATTCTCACAGGCACCAACCTGCTGCGGCAA
CAATTGCATTCAAATTTGTCCCAAAGAAACTTAATTAACATTTTCTGGCGATTTAATCTTTGCATAAATTGGATTT
GTTTTTAAAAATATAGAATAACTGGATCTTTATGCTCAAATAATTAATCATACATTCTTATTTTATCAGTCAAGAT
TGAACGCTACTTAAAAGTATGTATAAAACATCATCTGTATGTATAATTGTTTAACTTTTAACTAAAAGTCCTAATA
ATTGTGTTATGGAAATGTATTAATTGTCATTTAATATCATTTCCTTGAATTTATCACCATGTGTTTTTGTTTGGTT
TTTACACAGCTGGAAGGTTGATCCATAGGTAAGGACTTCTACCATCATTACTGTATAATGTTAATAATAGCATTAT
CATCAGTACTGTTATTGATAACTTCTCTTGTCTCGCTGACTCTCTCCATCAGATTCATCAAACGTCACGGTGACGT
CGAGCAGCAGGAGCTCGACAAGCGCTCAGTGGATGACGAGCCCAGTTCTATTGCTTTTGCCTGAAGAAGTCGCCTT
G
//
NRC-118 Witch Flounder GC3.1 (SEQ ID NO: 116)
ATGAAGTTCACTGCCACCTTCCTGGTGTTGTTCATGGTCGTCCTCATGGCTGAACCTGGAGACTGTATTTTTGGAT
TGATTGCGACTGCGGTCCACAATGGTAAGTCAAGGAATTAATTCGATTTTTACGTGGCAAATATTTTAGTATAACA
TACCTTATGAGTAGTCGATATATTTGACCAAGTAGAATCATTTTGACTTCAATAATAATCAAAATAACAATCTCTA
GGCAATTTAATATTTGCATTAATTGGATTTGTTTTTAAAAATATAGAATAACTGGATCTTAATGCTAAAATAATTA
AACATACATTCTGATATTACCAGTCAAGATTGAACGCTACTTAAAAGTATGTATAAAACATCATCTGTATGTATAA
TTGTTTAACTGTCGACTAATAGTCCTAATAATTGTGTTATGGAAATGTATTCATTGTCATATAATATCATTTGCTT
GAATTTATCACCATGTGTTTTTGTTTGTTTTTACACAGCTGGAAGGTTGATCCATAGGTAAGGACTTCTACCATCA
TTACTGTATAATTTTAAGAGCATTATCATCAGTACTGTTATTGATAACTTCTGTTGTCTCGCTGACTCTCTCCATC
AGACTACTCGGCTTTCATCATGGGCCTCCCGGGTTCTGGCACGGTGACGTCGAGCAGCAGGAGCTCGACAAGCGCT
CAGTGGATGAGGAGCCCAGTTCTATTGCTTTTGACTGAAGAAGTCGCCTTGAAGGAGCCTTCAG
//
NRC-119 Witch Flounder GC4.1 (SEQ ID NO: 117)
ATGAAGTTCACTGCCACCTTCCTGGTGTTGTTCATGGTCGTCCTCATGGCTGAACCTGGAGACTGTATTTTTGGAT
TGATTGCGACTGCGGTCCACAATGGTAAGTCAAGGAATTAATTCGATTTTTACTTGGCAAATATTTTAGTATAACA
TACCTTATGAGTAGTCGATATATTTGACCAAGCAGAATCATTTTGATTTCAATAATAATCAAAATAACAATCTCTA
GGCAATTTAATATTTGCATTAATTGGATTTGTTTTTAAAAATATAGAATAACTGGATCTTAATGCTAAAATAATTA
AACATACATTCTGATATTACCAGTCAAGATTGAACGCTACTTAAAAGTATGTATAAAACATCATCTGTATGTATAA
TTGTTTAACTGTCGACTAATAGTCCTAATAATTGTGTTATGGAAATGTATTCATTGTCATATAATATCATTTGCTT
GAATTTATCACCATGTGTTTTTGTTTGTTTTTACACAGTTGGAAGGTTGGTCCATGGGTAAGGACTTCTACCATCA
TTACTGTATAATTTTAAGAGCATTATCATCAGTACTGTTATTGATAACTTCTCTTGTCTCGCTGACTCTCTCCATC
AGACTACTCGGCTTTCATCATGGGCCTCCCGGGTTCTGGCACGGTGACGTCGTGCAGCAGGAGCTCGACAAGCGCT
CAGTGGATGAGGAGCCCAGTGCTATTGTTTTTGAATGAAGAAGTCGCCTTGAAGGAGCCTTCAG
//
NRC-120 Witch Flounder GC4.4 (SEQ ID NO: 118)
ATGAAGTTCACTGCCACCTTCCTGGTGTTGTTCATGGTCGTCCTCATGGCTGAACCTGGAGACTGTATTTTTGGAT
TGATTGCGACTGCGGTCCACAATGGTAAGTCAAGGAATTAATTCGATTTTTACGTGGCAAATATTTTAGTATAACA
TACCTTATGAGTAGTCGATATATTTGACCAAGTAGAATCATTTTGGTTTCAATAATAATCAAAATAACAATCTCTA
GGCAATTTAATATTTGCATTAATTGGATTTGTTTTTAAAAATATAGAATAACTGGATCTTAATGCTAAAATAATTA
AACATACATTCTGATATTACCAGTCAAGATTGAACGCTACTTAAAAGTATGTATAAAACATCATCTGTATGTATAA
TTGTTTAACTGTCGACTAATAGTCCTAATAATTGTGTTATGGAAATGTATTCATTGTCATATAATATCATTTGCTT
GAATTTATCACCATGTGTTTTTGTTTGTTTTTACACAGTTGGAAGGTTGGTCCATGGGTAAGGACTTCTACCATCA
TTACTGTATAATTTTAAGAGCATTATCATCAGTACTGTTATTGATAACTTCTCTTGTCTCGCTGACTCTCTCCATC
AGACTACTCGGCTTTCATCATGGGCCTCCCAGGTTCTGGCACGGTGACGTCGAGCAGCAGGAGCTCGACAAGCGCT
CAGTGGATGAGGAGCCCAGTGCTATTGTTTTTGAATGAAGAAGTCGCCTTGAAGGAGCCTTCAG
//
NRC-121 Petrale sole 02A (3) (SEQ ID NO: 119)
ATGAAGTTCACTGCCACCTTCCTCGTGTTGTTCATGGTCATCGTCATGTTTGAACCTGGAGAGTGTTTTTTTGGAA
TGCGTTTTCACGGGGTCCACCATGGTAGGGTCACAAAAGTGATTTGATTATTACATGCCAAATATGTTAATGAAAC
ATACCATATGAGCAGTCGTATTATTTGGACAAGTAGAATCACTTTGATTTCAATAGTAATTAAAATAACAATCAAA
AAGGCCTTTGATTAGCATGTTCCTTCAATGAAATGGACATTGAGGTTTATTTTGATTCTCACCTGCATCGACCTGC
TGCGGCAACTATTGAAATCAAATTTGTCCCAGAAGAAACTAAATTAACATTTTCTAGGCCATCTAATCTTTGCATG
AATTGGATTTGCTTTCAAAAATATAGAATAACTGGATATTTATGCTAAAATAATAAAAACACACATTCTGATTTTA
CCAGTCAAGATTGAACACTACTTAAAAGTACGTATAAAACATCATCTGTATGTATAATTGTTTGACTTTTAACAAA
TAGTCAAAATGATTGTTATGGAAATGCATTAATTGTCATTTAATATCATTTACTTGAATTTATCACCATGTGTTTG
TTTGTTTTTTAGCAGGTGGAGGTTTTCTCAATGCGCAAGGACTTCTACCATCATTACTGTGTAATTTTAATAGTAT
TATCATCAGTACTCTTATTGACAACGTCTCTTGTCTCGCTGACTCTCTCTATCAGATTAAACCCAGGGTATCGCGG
TTACGACGAGCAGCAGGAGCTCGACAAGCGCGCAGTCGATGA
//
NRC-122 Petrale sole 02B (SEQ ID NO: 120)
ATGAAGTTCACTGCCACCTTCCTGGTGTTGTCCTTGGTCGTCCTCATGGCTGAACCTGGAGAGGGTTTCTTTGGAG
CCCTTCTCAAAGGTAGAGTCACGGAATTAATTTGATTGTTACATGGCAAATAATTTTGTATAACATATCATATGAG
CAGTCGATGTATTTGACCAAGAAGAATCATTTTGATTTCAATAATAATCAAAATAACAATCTCTTGGAGATTATAT
ATTTGCAATAATTGGATTTTATAAAATATAGAACAACTGGATCTTAATGCTAAAATAATTTAACATACATTCTGAT
TTTACCAGTCAAAATTAACCACTACTTTAAAGTATGTATAAAACATCATCTGTATGTTTAATTGTTTAACTTTTAA
CAAATAGTCCAAATAATTGTGTAATGGAAATGTATTCATTGTCATATAATATAGTTTGCTTGACTTTATCACCGTG
TGTTTTTGTTTGTTTTTTCACAGGTGCCCAGGCGCTCCATGGGTAAGGACTTCTACCATCATGACTGTGTAAGTTT
AATAATATTATCATCAGTACTGTTATTAACGACTTCTCTTGTCTCGCTGACTCTCTCCATCAGAATCATCCACAAT
GCTCGTCACGGTTACGACGAGCAGCAGGAACTCAACAAGCGCGCAGTCGATGA
//
NRC-123 Petrale sole PL1/2/2.1 (SEQ ID NO: 121)
GCCCACTTTGTATTCGCAAGGTAAGATCAATATTTTTCAAATTCATTTAGACGAGACCAACCGTTTGCGAAATGTG
CTCAGCTTGTTATTGTATAATAACAAAGTTAACGATCTTTATTTTTCTGTTTTTTTGTAGAATGAAGTTCACTGCC
ACCTTCCTGATGTTGTTCATCTTCGTCCTCATGGTTGAACCTGGAGAGTGTGGTTGGAAAGATTGGTTTCGTAAGG
CTAAGAAAGGTAGAATCACGGAATTAATTAGCTTTTTACATTGCAAATAGATTTTTTATAACAGCTGGAAATCACA
AAAATAAATAGTCGATATATTTGGCCAATTAGAATCACTTTAATTTCAATAATAATCTAAATAACAACCTAAAAGG
CCTTTGATTAGCATGTTCCTTCAATGAAAAGGACATTGAGGTTTATTTTGATTCTCACATGCACCGACCTGTGCGG
CAACAATTGAATTCAGATTTGTCCCAGAAGAATTCAAAGTACATTTTTCCAGGCGATTAAATCTTTCCATTACTCG
GATTTAAAAATAAATAAATAGAATAACTGAAGCGCTATGATAAAATAATTACACATTCATTCTGATTTTACAAGTC
AAGATTGAACACTATTAAAAAGTGTGTATAAAACATCATCTGTATGTATAATTGTTTAACTGTTAATAGTCTTAAT
AATTGTGTTATGGAAATGTATTAATTTACATTTAATATCATTTGCTTGAGTTTACCATCATGTGTTTTTGTTTGTT
TTTACACAGTTGGCAAGACTGTTGGCGGCTTGGCCCTTAAGTAAGAACTTCTACCATCATTACTGTATAATTTTGA
TAGTATTATCACCAGTACTGTTATTAACTACTTCTCTTGTCTCGCTGACTCTCTCCATCCGACTCATCCGCAGTCA
TTACCTTGGCGAGCAGCAGGAGCTTGCCAAGCGCGCAGTCGATGACGACCCCAGTGTTATTGTCTTTGACTGAAGA
AGTCGCCTTGAAGGAGCCTTCAG
//
NRC-124 English sole 05A (SEQ ID NO: 122)
ATGAAGTTCACTGCCACCTTCCTCATGATTTTAATCTTCGTCCTCATGGTCGAACCTGGAGAGTGTGGTATTAGGA
AATGGTTTAAAAAGGCTGCTCACGGTAAAGTCACGGAATTAATTTGCTTTTTGCTTTACAAATATTTTTTTATAGC
AGCTGGAAAATCACAAAAATAAATAGTCGATGTATTTGGCCAATTAGAATCACTTTGATTTCAAATAATAATCTAA
ATAGCAACCTAAAAGGCCTTTGATTAGCATGTTCCTTCAATGAAATGGATGTTGAGGTTTATTTTGATTCTCACAT
GCACCGACCTGCTGCGGCAACAATTGAATTCAAATTTGTCCCAAAGGAATTCAAAGTAAACTTTTCTAGATGATTT
AATCTTTCCATAACTCGGCTTTGTTTTTAAAAATATATAATAACTCAATCACTATGATAAAATAATAACACATACA
TTCTGATTTATACAAGACAAGATTGAAAACTTCTTAAAAGTATGTATAAAACATCATCTGTTTGTATAATTGTTTA
TCATTTCACAAAAAGTCCAACTAATTGTGTTATGGAATTGTATAAATTGTCATTTAATATAATTTTTTTGAGTTTA
TCAATATGTGTTTTTGTTTGTTTTACACAGTTGGCAAGGAAGTTGGCAAGGTGGCCCTTAAGTAAGGACTTCTACC
ATTATTACTGTATAATTTTGATAGTATTATCACCCGTACTGTTATTGACAACTTCTCTTTTCCTGCTGACTCTCTC
CATCTGACTCATCTGCAGTGCTTGCCTTGACAAGCAGCAGCAGCTCGACAAGCGCGCAGTCGATGA
//
NRC-125 English sole PL1/2/5 (SEQ ID NO: 123)
GCCCACTTTGTATTCGCAAGGTAATATCGATATTTTTCAAACTCATTTAGACGAGACCAAGCATTTGGGAAATGTG
CTAAGGTTGTTACTGTATAATGCAAAATTAATGATCTTTATTTTTCTGTTTTTTTTTGCAGAATGAAGTTCACTGC
CACCTTCCTCATGATTTTAATCTTCGTCCTCATGGTCGAACCTGGAGAGTGTGGTTTGAAGAAATGGTTTAAAAAG
GCTGTTCACGGTAGAGTCACGGAATTAATTTGCTTTTTGCTTTACAAATATTTTTTTATAGCAGCTGGAAAATCAC
AAAAATAAATAGTCGATGTATTTGGCCAATTAGAATCACTTTGATTTCAATAATAATCTAAATAGCAACCTAAAAG
GCCTTTGATTAGCATGTTCCTTCAATGAAATGGATGTTGAGGTTTATTTTGATTCTCACATGCACCGACCTGCTGC
GGCAACAATTGAATTCCAATTTGTCCCAAAGGAATTCAAAGTAAACTTTTCTAGGCGATTTAATCTTTCCATAACT
CGGCTTTGTTTTTAAAAATATATAATAACTCAATCCCTATGATAAAATAATAACACATACATTCTGATTTATACAA
GACAAGATTGAAAACTTCTTGAAAGTATGTATCAAACATCATCTGTTTGTATAATTGTTTAACAGTTCACAAAAAG
TCCAACTAATTGTGTTATGGAATTGTATAAATTGTCATTTAATATAATTTTTTTGAGTTTATCAATATGTGTTTTT
GTTTGTTTTACACAGTTGGCAAGAAAGTTGGCAAGGTGGCCCTTAAGTAAGGACTTCTACCATTATTACTGTGTAA
TTTTGATAGTATTATCACCAGTACTGTTATTGACAACTTCTCTTTTCCTGCTGACTCTCTCCATCCGACTCATCTG
CAGTGCTTACCTTGGCGAGCAGCAGCAGCTCGACAAGCGTGCAGTCGATGAAGAGCCCAGTGTTATTGCTTTTGAC
TGAAGAAGTCGCCTTGAAGGAGCCTTCAG
//
NRC-126 Starry flounder 09A (SEQ ID NO: 124)
ATGAAGTTCACTGCCACCTTCCTCATGATGTTCATCTTCGTCCTCATGGTTGAACCTGGAGAGTGTGGTTGGAGGA
AATGGATTAAAAAGGCTACTCACGGTAAAGTCACGGAATTAATTCGTTTTTTGCTTTGCAAATATTTTTTTTATAA
CAGCTGGAAAGTCACAAAAATAAATAGTCAATATATTTGGCCAATTAGAATCACTTTGAGTTCAATAATAATCTAA
ATAACAACCAAAAAGGCCTTTCCTTTAATGAAATGTACGTTGAAGTTTATTTTGAATCTCACATGCACCGACCTGC
TGCGGCAACAATTGAATTCAAATTTCTCCCAGAGGAATTCAAAGTAAATTTTTCTAGGCGATTTAATCTTTCCATT
ACTCTGATTTGTTTTAAATATATAGAATGACTCAATTGCTATGATAAAATAATAAGCCATACATTCTGATTTTTAC
AAGACAAGATTGAAAACTTCTTAAAAGTACGTATAAAACATCATCTGTATTTATAATTGTTTAACATTTAACAAAT
TGTCCTACTAATTGTGTTATGGAAATGTATAAATTGTCATTTAATATCATTTGCTTGAGTTTATCATTATTTGTTT
TTGTTTGTTTTTACACAGTTGGCAAGCATATTGGCAAGGCGGCCCTTGAGTAAGAACTTCTACCATCATTACTGTA
TAATTTTGATAGTATTATCACCAGTACTGTTATTGACAACTTCTCTTGTCCTGATGACTCTGTTCATCCAACTCAT
CTGCAGTGCTTACATTGGCGGGAAGCAAGAACTCGACAAGCGCGCAGTCGATGA
//
NRC-127 (SEQ ID NO: 327)
ATGAAGTTCACTGCCACCTTCCTCATGATTTTAATCTTCGTCCTCATGGTCGAACCTGGAGAGTGTGGTTGTAAGA
AATGGTTTAAAAAGGCTGCTCACGGTAGAGTCACGGAATTAATTTGCTTTTTGCTTTACAAATATTTTTTTATAGC
AGCTGGAAAATCACAAAAATAAATAGTCGATGTATTTGGCCAATTAGAATCACTTTCATTTCAATAATAATCTAAA
TAGCAACCTAAAAGGCCTTTGATTAGCATGTTCCTTCAATGAAATGGATGTTGAGGTTTATTTTGATTCTCACATG
CACCGACCTGCTGCGGCAACAATTGAATTCCAATTTGTCCCAAAGGAATTCAAAGTAAACTTTTCTAGGCGATTTA
ATCTTTCCATAACTCGGCTTTGTTTTTAAAAATATATAATAACTCAATCCCTATGATAAAATAATAACACATACAT
TCTGATTTATACAAGACAAGATTGAAAACTTCTTGAAAGTATGTATCAAACATCATCTGTTTGTATAATTGTTTAA
CATTTCACAAAAAGTCCAACTAATTGTGTTATGGAATTGTATAAATTGTCATTTAATATAATTTTTTTGAGTTTAT
CAATATGTGTTTTTGTTTGTTTTACACAGTTGGCAAGAACGTTGGCAAGGTGGCCCTTAAGTAAGGACTTCTACCA
TTATTACTGTATAATTTTGATAGTATTATCACCAGTACTGTTATTGACAACTTCTCTTTTCCTGCTGACTCTCTCC
ATCCGACTCATCTGCAGTGCTTACCTTGGTGAGCAGCAGCAGCTCGACAAGCGTGCAGTCGATGAAGAGCCCAGTG
TTATTGCTTTTGACTGAAGAAGTCGCCTTGAAGGAGCCTTCAG
//
NRC-128 (SEQ ID NO: 129)
GCCCACTTTGTATTCGCAAGGTAATATCGATATTTTTCAAACTCATTTAGACGAGACCAAGCATTTGGGAAACGTG
CTAAGGTTGTTACTGTATAATGCAAAATTAATGATCTTTATTTTTCTGTTTTTTTTTGCAGAATGAAGTTCACTGC
CACCTTCCTCATGATTTTAATCTTCGTCCTCATGGTCGAACCTGGAGAGTGTGGTATTAGGAAATGGTTTAAAAAG
GCTGCTCACGGTAAAGTCACGGAATTAATTTGCTTTTTGCTTTACAAAATATTTTTTTATAGCAGCTGGAAAATCA
CAAAAATAAATAGTCGATGTATTTGGCCAATTAGAATCACTTTGATTTCAATAATAATCTAAATAGCAACCTAAAA
GGCCTTTGATTAGCATGTTCCTTCAATGAAATGGATGTTGAGGTTTATTTTGATTCTCACATGCACCGACCTGCTG
CGGCAACAATTGAATTCAAATTTGTCCCAAAGGAATTCAAAGTAAACTTTTCTAGGCGATTTAATCTTTCCATAAC
TCGGGCTTTGTTTTTAAAAATATATAATAACTCAATCCCTATGATAAAATAATAACACATACATTCTGATTTATAC
AAGACAAGATTGAAAACTTCTTGAAAGTATGTATCAAACATCATCTGTTTGTATAATTGTTTAACATTTCACAAAA
AGTCCAACTAGTTGTGTTATGGAATTGTATAAATTGTCATTTAATATAATTTTTTTGAGTTTATCAATATGTGTTT
TTGTTTGTTTTACACAGTTGGCAAGAAAGTTGGCAAGGTGGCCCTTAAGTAAGGACTTCTACCATTATTACTGTAT
AATTTTGATAGTATTATCACCAGTACTGTTATTGACAACTTCTCTTTTCCTGCTGACTCTCTCCATCCGACTCATC
TGCAGTGCTTACCTTGGCGAGCAGCAGCAGCTCGACAAGCGTGCAGTCGATGAAGAGCCCAGTGTTATTGCTTTTG
ACTGAAGAAGTCGCCTTGAAGGAGCCTTCAG
//
NRC-129 (SEQ ID NO: 130)
AATGAAGTTCACTGCCACCTTCCTCATAGAATGGTTCATCTTCGTCCTCAATGGGTTGAAACCTGAAGAAGTGTGG
TTGGAAAGAAAGTGGTTTAAAAAGGCTACTCACGGTAAAGTCACGGAATTAATTAGCATTTTTCTTTGCAAATATT
TTTTTTATACAGCTCGAAAATTCACAAAAATAAATAGTCGATATATTTGGCCAATTAGAATCACTTTGATTTCAAT
AATAATCTAAATAACAACCTAAAAGGCCTTTGATTAGCATGTTCCTTCAATGAAATGGACGTTGAGGTTTATATTG
ATTCTCACATGCACCGACCTGCTGCGTCAACAATTGAATTCAAATTTGAGAGGAATTCAGCGTAAATTTTTCTAGG
CGATTTAATCTTTCCATTACTCGGATTTGTTTTTAAATATATAGAATAACTCAATTGCTATGATAAAATAATAACA
CATACATTCAGATTTTTACAAGACAAGATTGAAAACTTCTTAAAGGTACGTATAAAACATCATCTGTATTTATAAT
TGTTTAACATTTAACAAATAATCCTACTAATTGTGTTATGGAAATGTATAAATTGTAATTTAATATAATTTGCTTT
AGTTTATCATTATTTGTTTTTGTTTGTTTTTACACAGTTGGCAAGCATGTTGGCAAGGCGGCCCTTGAGTAAGAAC
TTCTACCATCATTACTGTATAATTTTGATAGTGTTATCACCAGTACTGTTATTGACAACTTCTCTTGTCCTGCTGA
CTCTCTCCATCCGACTCATCCGCAGTGCTTACCTCGGCGAGAAGCAAGAACTCGACAAGCGCGCAGTCGATG
//
NRC-130 Greenland halibut 12B (SEQ ID NO: 131)
ATGAAGTTCACTGCCACCTTCCTGGTGTTGTTCATGGTCGTCCTCATGGCTGAACCTGGAGAGGGTTTTTTCGGAT
TGCTTTTTCACGGGATCCACCATGGTAGGGTCACGGAATTAATTAGATGTTTACATGGCAAATATTTTAAGATAAC
ACACCATATGAGTAGTCGATATATTTGACCAATTAGAATCACTTTAATTTCAATAATAATCACAATAACAATCTCT
AGGCCATTTAATCTTTCCATTAATCGGATTTGTTTTTTTAAATATAGAATAACTGGATCTTTATGCTAAAATAATG
AAACATACATTCTGATTTTACCAGTCAAGATTGAACGTTACTTAAAAGTATGTTTAAAACATCATCTGTATGTATA
ATTGTTTAGCTGTAAACAAATAGTCCAAATAATTGTGTTATGGAAATGTATTAATTGTCATATAATATAATTTGCT
TGAATTTATCACCATGTGTTTTTGTTTGTTTTTTAACACAGCTGGAAAGTTGATCCATGGGTAAGGACTTCTACCA
TCATTACTGTGTATTTTTAATAGTATTATCATCAGTACTGTTATTAACAACTTCTCTTCTATCGCTGACTCTCTCC
ATCAGACTCATCCATCATGGTTACGACGAGCAGCAGGAGCTCGACAAGCGCGCAGTCGATGA
//
NRC-131 Pacific Halibut 15A (SEQ ID NO: 132)
ATGAAGTTCACTGCCACCTTCCTGGTGTTGTTCATGGTCGTCCTCATGGCTGAACCTGGAGAGGGTTTGGGAAATT
GGATGGGGCCCCATATCAGCGGTAGAGTCACGGAATTAATTTGCTTTTTCCATTGCAAATATTTTAATATTGCATA
GCTGGAAAATCACGAAATAAGTAGTCGATATATTTGGCCAAATAGAATAACTTTGATTTCAATAATAATCAAAATT
ACAATCAAAAAGGCCTTTGATTAGCATGTTCCTTCAATAAAATGGACATTGAAGTTTATTTTGATGCTCACATGCA
CCGACCTGCTGCGGCAACAATTGAAATCAAATTTGTCTCAGAATTTAAAGTACATTTTTCTAGGTGATTTAATCTT
TCCATTCATCTGATTTATTTTATAAATATAGAATAACTGGATCTTTCTGCTAAAATAATAAAACACACATTCTGAT
TTTACCAGTCAAGATTGAACACTACTTAAAAGTATGTATAAAACATCATCTGTATGTATAATTGTTTAACTGTTAA
CAATAGTCCAAATAATTGTGTTAAGGAAATGTATTAATTGTCATTTAATATCATTTGCTTGAATTTATCACCATGA
GTTTTTTGTTTGTTTTTACACAGGTAGAAAGAAGGCCTTGCAGTAAGGACTTCTACCATCATTACTTTGTAATTTT
TATAGTATTATCATCAGTACTGTTATTGACAACTTCTCTTGTCTCGCTGACTCTCTCCATCAGGATGAACTCAGAG
CGTCGCAGTTACGACGAGTAGCAGCAGAAGCTCGACAAGCGCGCAGTCGATGA
//
NRC-132 Pacific Halibut 15B (SEQ ID NO: 133)
ATGAAGTTCACTGCCACCTTCCTGGTGTTGTTCATGGTCGTCCTCATGGCTGAACCTGGAGAGTGTTTTTTGGGAT
TGCTTTTTCACGGGGTCCACCATGGTAGGGTCACGGAAGTAATTCGATTTTTACATGGCAAATATTTTAAGATAAC
ACACCATATGAGTAGTCGATATATTTGATATATTAGAATCACTTTGATTTCAATAATAATCAAAATAACAATCTCT
AGGCGATTTAATATTTGCATTAATTGGATTTGTTTTTAAAAATATAGAATAACTGGATCTTTATGGTAAAATAATT
AAACATACATTCTGATTTTACCAGTCAAGATTGAACACTACTTAGAAGTATGTATAAAACATCATCTGTATGTATA
ATTGTTTAACTGTTAACTAATAGTCCAAATAATTGTGTTATGGAAATGTATTAATTGTCATTTAATATCATTTGCT
TGAATTTATCACCATGTGTTTTTGTTTGTTTTTACACAGTTGGAAATTTGATCCATGGGTAAGGACTTCTACCATC
ATTACTGTGTATTTTTAATAGTATTATCATCAGTACTGTTATTGACAACTTCTCTTGTCTCGCTGACTCTCTCCAT
CAGACTCATCCATCACGGTTACGACGAGCAGCAGGAGCTCGACAAGCGCGCAGTCGATGA
//
NRC-133 C 0 sole PL1/2/6 (SEQ ID NO: 134)
GCCCACTTTGTATTCGCAAGGTAATATCGATATTTTTCAAACTCATTTAGACGAGACCAGGCATTTGGGAAACGTGC
TAAGGTTGTTACTGTATAATGCAAAATTAATGATCTTTATTTTTCTGTTTTTTTTTGCAGAATGAAGTTCACTGCCA
CCTTCCTCATGATTTTAATCTTCGTCCTCATGGTCGAACCTGGAGAGTGTGGTATTAGGAAATGGTTTAAAAAGGCT
GCTCACGGTAAAGTCACGGAATTAATTTGCTTTTTGCTTTACAAATATTTTTTTACAGCAGCTGGAAAATCACAAAA
ATAAATAGTCGATGTATTTGGCCAATTAGAATCACTTTGATTTCAATAATAATCTAAATAGCAACCTAAAAGGCCTT
TGATTAGCATGTTCCTTCAATGAAATGGGTGTTGAGGTTTATTTTGATTCTCACATGCACCGACCTGCTGCGGCAAC
AATTGAATTCAAATTTGTCCCAAAGGAATTCAAAGTAAACTTTTCTAGGCGATTTAATCTTTCCATAACTCGGCTTT
GTTTTTAAAAATATATAATAACTCAATCGCTATGATAAAATAATAACACATACATTCTGATTTATACAAGACAAGAT
TGAAAACTTCTTGAAAGTATGTATCAAACATCATCTGTTTATATAATTGTTTAACATTTCACAAAAAGTCCAACTAA
TTGTGTTATGGAATTGTATAAATTGTCATTTAATATAATTTTTTTGAGTTTATCAATATGTGTTTTTGTTTGTTTTA
CACAGTTGGCAAGAAAGTTGGCAAGGTGGCCCTTAAGTAAGGACTTCTACCATTATTACTGTATAATTTTGATAGTA
TTATCACCAGTACTGTTATTGACAACTTCTCTTTTCCTGCTGACTCTCTCCATCCGACTCATCTGCAGTGCTTACCT
TGGCGAGCAGCAGCAGCTCGACAAGCGTGCAGTCGATGAAGAGCCCAGTGTTATTGCTTTTGACTGAAGGAGTCGCC
TTGAAGGAGCCTTC
//

TABLE 13
Nucleotide sequences encoding hepcidin-like peptides of Table 11.

NRC201 (SEQ ID NO: 135)
CGCCCTTAAGATGAAGACATTCAGTGTTGCAGTTGCAGTGGTGGTCGTCCTCGCATGTATGTTCATCCTTG
AAAGCACCGCTGTTCCTTTCTCCGAGGTGCGAACGGAGGAGGTTGAAAGCATTGACAGTCCAGTTGGGGAA
CATCAACAGCCGGGCGGCACGTCCATGAATCTGCCGGTACGTTCAATTTAGTGAATGAATTAAGTAATTAC
CTTTAGCAAATTAACATCTAAGTGGTTGCGTTTCACCCTTGGAATTGAATTAGCCCACTAGCGCTAGTTGT
TAACCATTTGATTGTGAGCCGGTAGAGAGGGCTTCAGGGCGAGTAGTGTGAATACTTGTGAAGTGGAGACT
TGGACAAAAATACTTACCATGTGCTTGTTCCCACCTTTTTCATTTTCTTTTCTTGGCTGAGATACAGATGC
ATTTCAGGTTCAAGCGTCAGAGCCACCTCTCCCTGTGCCGTTGGTGCTGCAACTGCTGTCACAACAAGGGC
TGTGGCTTCTGCTGCAAATTCTGAGGACCTGCCAGCAAAGGGCGAATTCGTTTAAAACAC
//
NRC202 (SEQ ID NO: 136)
AGATGAAGACATTCAGTGTTGCAGTTGCAGTGGTGGTCGTCCTCGCATGTATGTTCATCCTTGAAAGCACC
GCTGTTCCTTTCTCCGAGGTGCGAACGGAGGAGGTTGAAAGCATTGACAGTCCAGTTGGGGAACATCAACA
GCCGGGCGGCACGTCCATGAATCTGCCGATGCATTTCAGGTTCAAGCGTCAGAGCCACCTCTCCCTGTGCC
GTTGGTGCTGCAACTGCTGTCACAACAAGGGCTGTGGCTTCTGCTGCAAATTCTGAGGACCTGCCAGCA
//
NRC203 (SEQ ID NO: 137)
ACGAGGTCCCTCATCCGCTGACACCAAAAGAACAATCAATCAACTTTGGACTCGTCTTAGTGCATTGAAAA
TTGTGCGTTGGAGAGCGTCGCTTTTTGGGAACATTGAAGAGTTCTGATCTTCCTCATAAACTGTCACTTCA
ATTTCAACTGATTTCAACAGGACTTTTAAATAGGCTATAAACTTCCTAAAAAAAACGAGAATGAAGGCCTT
TAGTGTTGCAGTGGTACTCGTCATTGCATGTATGTTCATCCTTGAAAGCACCGCTGTTCCTTTCTCCGAGG
TGCGAACGGAGGAGGTTGGAAGCTTTGACAGTCCAGTTGGGGAACATCAACAGCCGGGCGGCGAGTCCATG
CATCTGCCGGAGCCTTTCAGGTTCAAGCGTCAGATCCACCTCTCCCTGTGCGGTTTGTGCTGCAACTGCTG
TCACAACATTGGCTGTGGCTTCTGCTGCAAATTCTAAGGACCTGCCCGCAACATTTTCTAGTTTGTACATG
TTTGCAATGTTTTCTTTCTGAGATGTTGTTTTTGTGACTATGATAATGATTTATAAAATCACT
TCTTATTGTGACACTTTAAAAAAAATAAACACATTCTTTGAATACAAAAAAAAAAAAAAAAAA
//
NRC204 (SEQ ID NO: 138)
CGAACGGAGGAGGTTGAAAGCATTGACAGTCCAGTTGGGGAACATCAACAGCCGGGCGGCACGTCCATGAA
TCTGCCGATGCATTTCAGGTTCAAACGTCAGAGCCACCTCTCCCTGTGCCGTTGGTGCTGCAACTGCTGTC
ACAACAAGGGCTGTGGCTTCTGCTGCAAATTCTGAGGACCTGCCAGCACTAAAGCCATTTTATTAACTTAT
CGCCTTTAATTTGCCCCTATTCTTCTATGTTTCTTTTGGACTCTGTGGAGAAGATGCAATCTCATTGACGT
CTTTATCACTGCACAACCTCAATCTTGT
//
NRC205 (SEQ ID NO: 139)
AAGATGAAGACATTCAGTGTTGCAGTGGTACCCGTCATTGCATGTATGTTCATCCTTGAAAGCACCGCTGT
TCCTTTCTCCGAGGTGCGAACGGAGGAGGTTGGAAGCTTTGACAGTCCAGTTGGGGAACATCAACAGCCGG
GCGGCACGTCCATGAATCTGCCGATGCATTTCAGGTTCAAGCGTCAGAGCCACCTCTCCCTGTGCCGTTGG
TGCTTCAACTGCTGTCACAACAAAGGCTGTGGCTTCTGCTGCAAATTCTGAGGACCTGCCAGCA
//
NRC206 (SEQ ID NO: 140)
TAAGATGAAGCAATTCAGTGTGGCAGTGGTACTCGTCATGGCATGTATGTTCATCGTGGAAAGCACCGCTG
TTCCTTTCTCCGAGGTGCGAACGGAGGAGGTTGGAAGCTTGGACAGTCCAGTTGGGGAACATCAACAGCCG
GGCGGCGAGTCCATGCATCTGCCGGAGCCTTTCAGGTTCAAGCGTCAGATCCACCTCTCCCTGTGCGGTTT
GTGCTGCAACTGCTGTCACAACATTGGCTGTGGCTTCTGCTGCAAATTCTGAGACTGCCAGCA
//
NRC207 (SEQ ID NO: 141)
ACGAGGCACACGCTGACCAGGGGGTCACCACAACTTCTGAAGAGACCCAGGTTCCTAGAGAGCCACTAGAG
AATCACCCGGGAGCCCGAAGAACACAGGACGCTGCGGTGCTCGTCGGTGGCCGGACACCCATGAGACAGAA
GACCTACAAGCCTCTCAGCTTCAGAAGGATTTCCTGACTCAGCATCTAAAACCTCCCTCAAAATGAAGGCA
TTCAGCATTGCAGTTGCAGTGACACTCGTGCTCGCCTTTGTTTGCATTCAGTGCAGCTCTGCCGTCCCATT
CCAAGGGGTGCAGGAGCTGGAGGAGGCCGGGGGCAATGACACTCCAGTTGCGGAACATCAAGTGATGTCAA
TGGAATCCTGGATGGAGAATCCCACCAGGCAGAAGCGCCACATCAGCCACATCTCCCTGTGCCGCTGGTGC
TGCAACTGCTGCAAGGCCAACAAGGGCTGTGGCTTCTGCTGCAAGTTCTGAGGATTCCCGCAACACAACCT
CACAATGTATTAATTTATTACACTTTTTGTCGAGAAATGTCCTTTTTCTTGACCTCTTTTGTAATTTTGTA
TAATCTTTTAAATAAAACGGGGTACGATTCATGGAAAAAACCCTTTGAATAAAATAAAAAAAAAAAAAAAA
AAAAAAAC
//
NRC208 (SEQ ID NO: 142)
AAGATGAAGACATTCAGTGTTGCAGTTGCAGTGACACTCGTGCTCGCCTTTGTTTGCATTCAGGACAGCTC
TGCCGTCCCATTCCAGGGGGTAAGAACGCAACTTTAACTCGCTTCATTTGCTTATTAGCCATAAATGTTTT
GTCAGGATGCTGAGACACGGCTCCTAAATGTGTATAATTCATTAACAGGTGCAGGAGCTGGAGGAGGCAGG
GGGCAATGACACTCCAGTTGCGGCACATCAAATGATGTCAATGGAATCGTGGATGGTATGTTCAATCTGTT
CAATCGACTGGATGAATTAAGCCAATTACTGTGAGCGCGTTAACATTTAAGTGGCTGTGTTCCAGCCCGGT
GGTGTAGGGAATAAAACCCCTCGTTCATGTGTCTTGTCCGTCCACAGGAGAGTCCCGTCAGGCAGAAGCGT
CACATCAGCCACATCTCCATGTGCCGCTGGTGCTGCAACTGCTGCAAGGCCAAGGGCTGTGGCCCCTGCTG
CAAATTCTGAGGACCTGCCCAGCA
//
NRC209 (SEQ ID NO: 143)
AAGATGAAGACATTCAGTGTTGCAGTCACAGTGGCCGTCGTGCTCGTCTTTATTTGTATCCAGCAGAGCTC
TGGCACCTTTCCTGAGGTAAGCTCCTGACTTCAGATCGTTTCATTTTGCTTGTTATCCATGAATCTCTCAT
CAACAGACTGAGACTTGATTCCTTCTTTATCAGGTACAAGAGCTGGAGGAGGCAGTGAGCAATGACAATGC
AGCTGCTGAACATCAGGAGACATCAGTGGACTCATGGATGGTAGOTTCAGTTCACTGAATGGATCAAACCA
ATTCACATCAGACCTTTCAGATGGAAGTGAATGTGTTTTAGTCTCAAAGOTGCCCTGAAGCTCAGTTTACA
CAAGCAGTGAAAACAAACACAGAAAGTTATGATGATGCTGATGAACTTCTCCTCATGTCTCATGTCTCTCA
CACAGATGCCATACAACAGACAGAAGCGTGCCTTCAAGTGTAAGTTCTGCTGCGGCTGCTGCAGAGCTGGT
GTCTGTGGACTGTGCTGCAAGTTCTGAGGATTCCTGCTCCAACAAC
//
NRC210 (SEQ ID NO: 144)
ACGAGCTGACAGGAGCTGACAGGAGTCACCAGCAGAGTCAAAGAACTAAACAACTTAACTCAGTCAAACTC
TCAAAGATGAAGACATTCAGTGTTGCAGTCACAGTGGCCGTCGTCCTCGTCTTTATTTGTATCCAGCAGAG
CTCTGCCTCCTTTCCTGAGGCACAAGAGCTGGAGGAGGCAGTGAGCAATGACAATGCAGCTGCTGAGCATC
AGGAGACACCAGTGGACTCGTGGATGATGCCATACAACAGACAGAAGCGTAGCTTTAAGTGTAAGTTCTGC
TGCGGCTGCTGCAGAGCTGGTGTCTGTGGACTGTGGTGCAAGTTCTGAGGATTCCTGCTCCAACAACCATC
AAATATTCATTTGTTTTGCCTTTTGTCTTAAAGTTCATTGAACTATAAACATATTTCTGGTTGAGCATGTG
ATAGTTTAATGGTGTTACTCATTGGTTCATGGTATAGTCAAGTGTTCAGAGATGTGATTGTATCACCCACA
TATTTTCTCTGTTAGGTGTATTTTCAATAAATGCCAATGATCCTTTGAAAAAAAAAAAAAAAAAAAAAAAA
AAAAAAAAAAAAA
//
NRC211 (SEQ ID NO: 145)
ACGAGCGGCACGAGGTGAACTGACAGGAGCTGACAGGAGTCACCAGCAGAGTCAAAGAACTAAACAACTTA
ACTCAGTCAAACTCTCAAAGATGAAGACATTCAGTGTTGCAGTCACAGTGGCCGTCGTGCTCGTCTTTATT
TGTATCCAGCAGAGCTCTGCCTCCTTTCCTGAGGCACAAGAGCTGGAGGAGGCAGTGAGCAATGACAATGC
AGCTGCTGAACATCAGGAGACACCAGTTGACTCGTGGATGATGCCAAACAACAGACAGAAGCGTGGCTTTA
AGTGTAAGTTCTGCTGCGGCTGCTGCAGAGCTGGTGTCTGTGGACTGTGCTGCAAGTTCTGAGGATTCCTG
CTCCAACAACCATCAAATATTCATTTGTTTTGCCTTTTGTTTTAAAGTTCATTGAACTATATACATATTTC
TGGTAGAGCATGTGATAGTTTAATGGTGCTACTCCTTGGTTCATGGTGTAGTTAAGTGTTCAGAGATGTGA
TTGTATCACCCACATATTTCTCTGTTAAGGTGTATTTTCAATAAATGTTAATGCTCCTTTGAAAAAAAAAA
AAAAAAAAAAAAA
//
NRC212 (SEQ ID NO: 146)
ACGAGACTGACAGGAGCTGACAGGAGTCACCAGCAGAGTCAAAGAACTAAACAACTTAACTCAGTCAAACT
CTCAAAGATGAAGACATTCAGTGTTGCAGTCACAGTGGCCGTCGTGCTCGTCTTTATTTGTATCCAGCAGA
GCTCTGCCACCTTTCCTGAGATGCCATACAACAGACAGAAGCGTGGCTTTAAGTGTAAGTTCTGCTGCGGC
TGCTGCGGAGCTGGTGTCTGTGGAATGTGCTGCAAGTTCTGAGGATTCCTGCTCCAACAACCATCAAATAT
TCATTTGTTTTGCCTTTTGTCTTAAAGTTCATTGAACTATAAACATATTTCTGGTTGAGCATGTGATAGTT
TAATGGTGTTACTCATTGGTTCATGGTATAGTCAAGTGTTCAGAGATGTGATTGTATCACCCACATATTTT
CTCTGTTAGGTGTATTTTCAATAAATGCCAATGATCCTTTGAAAAAAAAAA
//
NRC213 (SEQ ID NO: 147)
AAGATGAAGACATTCAGTGTTGCAGTCACAGTGGCCGTCGTGCTCGTCTTTATTTGTATCCAGCAGAGCTC
TGCCTCCTTTCCTGAGGTAAGCACCTGACTTCAGATCGTTTCATTTGCTTGTTATCCATGAATCTCTCATC
ATCATACTGAGACTTGATTCCTTCTTTATCAGGCACAAGAGCTGGAGGAGGCAGTGAGCAATGACAATGCA
GCTGCTGAGCATCAGGAGACACCAGTGGACTCCAGGAGTGAATGTGTTTTAGTCAcAAAAGTGCCCTGAAG
CTCAGTTTACACAAGCAGAGAAAACAAACAGAGTAAGTTATGATGATGCTGATGAAGGTCTCCTCATGTCT
CATGTCTCTCACACAGATTCCATACAACAGACAGAAGCGTAGCTTTAAGTGTAAGTTCTGCTGCGGCTGCT
GCAGAGCTGGTGTCTGTGGACTGTGCTGCAAGTTCTGAGGATTCCTGCTCCAACAAC
//
NRC214 (SEQ ID NO: 148)
AGATGAAGACATGCAGTGTTGCAGTCACAGTGGCCGTCGTGCTCGTCTTTATTTGTATCCAGCAGAGCTCT
GCCTCCTTTCCTGAGGTAAGCACCTGACTTCAGATCGTTTCATTTGCTTGTTATCCATGAATCTCTCATCA
TCATACTGAGACTTGATTCCTTCTTTATCAGGTACAAGAGCTGGAGGAGGCAGTGAGCAATGACAATGCAG
CTGCTGAACATCAGGAGACACCAGTTGACTCGTGGATGGTAGGTTCAGTTCACTGAATGGATCAATCCATT
TCACATCAGATCTTTCAGATGGAAGTGAATGTGTTTTAGTCACAAAAGTGCCCCTGAAGCTCAGTTTACAC
AAGCAGAGAAAACAAACAGAGTAAGTTATGATGATGCTGATGAAGGTCTCCTCATGTCTCATGTCTCTCAC
ACAGATGCCAAACAACAGACAGAAGCGTGGCTTTAAGTGTAAGTTCTGCTGCGGCTGCTGCAGAGCTGGTG
TCTGTGGACTGTGCTGCAAGTTCTGAGGATTCCTGCTCCGGACAA
//
NRC215 (SEQ ID NO: 149)
AAGATGAAGACAATCAGTGTTGCAGTCACAGTGGCCGTCGTCCTCGTCTTTATTTGTATCCAGCAGAGCTC
TGCCTCCTTTCCTGAGGTAAGCACCTGACTTCAGATCGTTTAATTTGCTTGTTATCCATGAATCTCTCATC
AACATACTGAGACTTGATTCCTTCTTTATCAGGCACAAGAGCTGGAGGAGGCAGTGAGCAATGACAATGCA
GCTGCTGAGCATCAGGAGACACCAGTG
GACTCAGGGATGGTAGGTTCAGTTCACTGAATGGATCAATCCATTTCACATCAGATCTTTCAGATTGAAGT
GAATGTGTTTTAGTCACAAAAGTGCCCTGAAGCTCAGTTTACACAAGCAGAGAAAACAAACAGAGTAAGTT
ATGATGATGCTGATGAAGGTCTCCTCATGTCTCATGTCTCTCACACAGATTCCATACAACAGACAGAAGCG
TAGCTTTAAGTGTAAGTTCTGCTGCGGCTGCTGCAGAGCTGGTGTCTGTGGACTGTGCTGCAAATTCTGAG
GACCTGCCAGCA
//
NRC216 (SEQ ID NO: 150)
AAGATGAAGACATTCAGTGGTGCAGTCACAGTGGCCGTCGTGCTCGTCTTTATTTGTATCCAGCAGAGCTC
TGCCTCCTTTCCTGAGGTAAGCACCTGACTTCAGATCGTTTCATTTGCTTGTTATCCATGAATCTCTCATC
ATCATACTGAGACTTGATTCCTTCTTTATCAGGTACAAGAGCTGGAGGAGGCAGTGAGCAATGACAATGCA
GCTGCTGAACATCAGGAGACACCAGTTGACTCGTGGATGGTAGGTTCAGTTCACTGAATGGATCAATCCAT
TTCACATCAGATCTTTCAGATGGAAGTGAATGTGTTTTAGTCACAAAAGTGCCCTGAAGCTCAGTTTACAC
AAGCAGAGAAAACAAACAGAGTAAGTTATGATGATGCTGATGAAGGTCTCCTCATGTCTCATGTCTCTCAC
ACAGATGCCAAACAACAGACAGAAGCGTGGCTTTAAGTGTAAGTTCTGCTGCGGCTGCTGCAGAGCTGGTG
TCTGTGGACTGTGCTGCAAATTCTGAGGACCTGCCAGCA
//
NRC217 (SEQ ID NO: 151)
AAGATGAAGACATCAGTGGTTGCAGTCACAGTGGCCGTCGTGCTCGTCTTTATTTGTATCCAGCAGAGCTC
TGCCACCTTTCCTGAGGTAAGCACCTGACTTCAGATAGCTTCATTTGCTTGTTATCCATGAATCTCTCATC
AACATACTGAGACTTTATTCCTTCTTTATCAGGTACAAGAGCTGGAGGAGGCAGTGAGCAATGACAATGCA
GCTGCTGCGCATCAGGAGACATCAGTGGACTCGTGGATGGTAGGTTCAGTTCACTCAATGGATCAAACCAA
TTCACATCAGATCTTTCAGATGGAAGCGAATGTGTTTTAGTCAAAAAAGTGACCTGATGCTCAGTTTACAC
AAGCAGAGAAAACAAGCAGAGTAAGTTATGATGATGCTGATGAACGTGTCCTCATGTCTCATGTCTCTCAC
ACAGATGCCATACAACAGACCGAAGCGTAGCTTTAAGTGTAAGTTCTGCTGCGGCTGCTGCAGAGCTGGTG
TCTGTGGACTGTGCTGCAAATTCTGAGGATTCCTGCTCCAACAAC
//
NRC218 (SEQ ID NO: 152)
AAGATGAAGACATTCAGTGTGGCAGTCACAGTGGCCGTCGTGCTCGTCTTTATTTGTATCCAGCAGAGCTC
TGCCACCTTTCCTGAGGTAAGCACCTGACTTCAGATAGCTTCATTTGCTTGTTATCCATGAATCTCTCATC
AACATACTGAGACTTGATTTCTTCTTTATCAGGTACAAGAGCTGGAGGAGGCAGTGAGCAATGACAATGCA
GCCGCTGAACATCAGGAGACATCAGTGGACTCGTGGATGGTAGGTTCAGTTCACTCAATGGATCAAACCAA
TTCACATCAGATCTTTCAGATGGAAGTGAATGTGTTTTAGTCACAGAAGTGCCCTGATGCTCAGTTTACAC
AAGCAGAGAAAACAAGCAGAGTAAGTTATGATGATGCTGATGAACGTGTCCTCATGTCTCATGTCTCTCAC
ACAGATGCCATACAACAGACCGAAGCGTAGCTTTAAGTGTAAGTTCTGCTGCGOCTGCTGTAGAGCTGGTG
TCTGTGGACTGTGCTGCAAATTCTGAGGATTCCTGCTCCAACAAC
//
NRC219 (SEQ ID NO: 153)
AAGATGAAGACATTCGTGGTTGCAGTCACAGTGGCCGTCGTGCTCGTCTTTATTTGTATCCAGCAGAGCTC
TGCCACCTTTCCTGAGGTAAGCACCTGACTTCAGATAGCTTCATTTGCTTGTTATCCATGAATCTCTCATC
AACATACTGAGACTTGATTCCTTCTTTATCAGGTACAAGAGCTGGAGGAGGCAGTGAGCAATGACAATGCA
GCCGCTGAACATCAGGAGACATCAGTGGACTCGTGGATGGTAGGTTCAGTTCACTCAATGGATCAAACCAA
TTCACATCAGATCTTTCAGATGGAAGTGACTGTGTTTTAGTCACAAAAGTGCCCTGATGCTCAGTTTACAC
AAGCAGAGAAAACAAGCAGAGTAAGTTATGATGATGCTGATGAACGTCTCCTCATGTCTCATGTCTCTCAC
ACAGATGCCATACAACAGACAGAAGCGTAGCTTTAAGTGTAAGTTCTGCTGCGGCTGCTGCAGAGCTGGTG
TCTGTGGACTGTGCTGCAAATTCTGAGGATTCCTGCTCCAACAAC
//
NRC220 (SEQ ID NO: 154)
AAGATGAAGACATCAGTGGTTGCAGTCACAGTGGCCGTCGTGCTCGTCTTTATTTGTATCCAGCAGAGCTC
TGCCACCTTTCCTGAGGTAAGCACCTGACTTCAGATAGCTTCATTTGCTTGTTATCCATGAATCTCTCATC
AACATACTGAGACTTTATTCCTTCTTTATCAGGTACAAGAGCTGGAGGAGGCAGTGAGCAATGACAATGCA
GCTGCTGCACATCAGGAGACATCAGTGGACTCGTGGATGGTAGGTTCAGTTCACTCAATGGATCAAACCAA
TTCACATCAGATCTTTCAGATGAAGTGACTGTGTTTTAGTCACAAAAGTGCCCTGATGCTCAGTTTACACA
AGCAGAGAAAACAAGCAGAGTAAGTTATGATGATGCTGATGAACGTGTCCTCATGTCTCATGTCTCTCACA
CAGATGCCATACAACAGACATAAGCGTAGCTTTAAGTGTAAGTTCTGCTGCGGCTGCTGCAGAGCTGGTGT
CTGTGGACTGTGCTGCAAATTCTGAGGATTCCTGCT
//
NRC221 (SEQ ID NO: 155)
AAGATAAGACATTCAGTGTTGCAGTCACAGTGGCCGTCGTGCTCGTCTTTATTTGTATCCAGCAGAGCTCT
GCCACCTTTCCTGAGGTAAGCACCTGACTTCAGATCGTTTCATTTGCTTGTTAGCCTTGAATCTCTCATCA
ACATACTGAGACTTGATTTCTTCTTTATCAGGTACAAGAGCTGGAGGAGGCAGTGAGCAATGACAATGCAG
CTGCTGAACATCAGGAGACATCAGTGGACTTGTGGATGGTAGGTTCAGTTCACTGAATGGATCAAACCAAT
TCACATCAGATCTTTCAGATGGAAGTGAATGTGTTTTAGTCACAAAAGTGCCCTGAAGCTCAGTTTACACG
AGCAGAGAAAACCAACACAGTAAGTTATGATGATGCTGATGAACGTCTCCTCATGTCTCATGTCTCTCACA
CAGATGCCATACAACAGACAGAAGCGTGGCTTTAAGTGTAAGTTCTGCTGCGGCTGCTGCAGCCCTGGTGT
CTGTGGACTTTGCTGCAGATTCTGAGGATTCCTGCTCCAACAAC
//
NRC222 (SEQ ID NO: 156)
AAGATGAAGACATTCAGTGTTGCAGTCGCAGTGGCCGTCGTGCTCATCTTTATTTGTATCCAGCAGAGCTC
TGCCACCTTTCCTGAGGTAAGCACCTGACTTCAGATAGTTTCATTTGCTTGTTATCCATGAATCTCTCATC
AACATACTGAGACTTTATTCCTTCTTTATCAGGTACAAGAGCTGGAGGAGGCAGTGAGCAATGACAATGCA
GCTGCTGAACATCAGGAGACATCATTGGACTCATGGATGGTAGGTTCAGTTCACTCAATGGATCAAACCAA
TTCACATCAGATCTTTCAGATGGAAGTGACTGTGTTTTAGTCACAAAAGTGCCCTGATGCTCAGTTTACAC
AAGCAGAGAAAACAAGCAGAGTAAGTTATGATGATGCTGATGAACGTGTCCTCATGTCTCATGTCTCTCAC
ACAGATGCCATACAACAGACAGAAGCGTGGCTTTAAGTGTAAGTTCTGCTGCGGCTGCTGCAGAGCTGGTG
TCTGTGGACTGTGCTGCAAATTCTGAGGACCTGCCAGCA
//
NRC223 (SEQ ID NO: 157)
AAGATGAAGACATTCAGTGTTGCAGTCACAGTGGCCGTCGTGCTCGTCTTTATTTGTATCCAGCAGAGCTC
TGCCACCTTTCCTGAGGTAAGCACCTGACTTCAGATAGTTTCATTTGCTTGTTATCCATGAATCTCTCATC
AACATACTGAGACTTTATTCCTTCTTTATCAGGTACAAGAGCTGGAGGAGGCAGTGAGCAATGACAATGCA
GCTGCTGAACATCAGGAGACATCATTGGACTCATGGATGGTAGGTTCAGTTCACTCAATGGATCAAACCAA
TTCACATCAGATCTTTCAGATGGAAGTGAATGTGTTTTAGTCACAAAAGTGCCCTGATGCTCAGTTTACAC
AAGCAGAGAAAACAAGCAGAGTAAGTTATGATGATGCTGATGAACGTGTCCTCATGTCTCATGTCTCTCAC
ACAGATGCCATACAACAGACATAAGCGTAGCTTTAAGTGTAAGTTCTGCTGCGGCTGCTGCAGAGCTGGTG
TCTGTGGACTGTGCTGCAAATTCTGAGGACCTGCCAGCA
//
NRC224 (SEQ ID NO: 158)
AGATGAAGACATTCAGTGTTGCAGTCACAGTGGCCGTCGTGCTCGTCTTTATTTGTATCCAGCAGAGCTCT
GCCACCTTTCCTGAGGTAAGCACCTGACTTCAGATAGTTTCATTTGCTTGTTATCCATGAATCTCTCATCA
ACATACTGAGACTTGATTTCTTCTTTATCAGGTACAAGAGCTGGGGGAGGCAGTGAGCAATGACAATGCAG
CCGCTGAACATCAGGAGACATCAGTGGACTCGTGGATGGTAGGTTCAGTTCACTCAATGGATCAAACCAAT
TCACATCAGATCTTTCAGATGGAAGTGAATGTGTTTTAGTCACAAAAGTGCCCTGATGCTCAGTTTACACA
AGCAGAGAAAACAAGCAGAGTAAGTTATGATGATGCTGATGAACGTGTCCTCATGTCTCATGTCTCTCACA
CAGATGCCATACAACAGACCGAAGCGTAGCTTTAAGTGTAAGTTCTGCTGCGGCTGCTGCAGAGCTGGTGT
CTGTGGACTGTGCTGCAAATTCTGAGGACCTGCCAGCA
//
NRC225 (SEQ ID NO: 159)
AAGATGAAGACATTCAGTGTTGCAGTCACAGTGGCCGTCGTGCTCATCTTTATTTGTATCCAGCAGAGCTC
TGCCACCTCTCCTGAGGTACAAGGGCTGGAGGAGGCAGTGAGCAATGACAATGCAGCTGCTGAACATCAGG
AGACATCAGTGGACTCGTGGATGATGCCATACAACAGACAGAAGCGTGGCTTTAAGTGTAAGTTCTGCTGC
GGCTGCTGCAGGCCTGGTGTCTGTGGACTTTGCTGCAGATCCTGAGGATTCCTGCTCCAACAAC
//
NRC226 (SEQ ID NO: 160)
AAGATGAAGACATTCAGTGTTGCAGTCACAGTGGCCGTCGTGCTCGTCTTTATTTGTATCCAGCAGAGCTC
TGCCACCTTTCCTGAGGTAAGCACCTGACTTCAGATCGTTTCATTTGCTTGTTAGCCTTGAATCTCTCATC
AACATACTGAGACTTGATTTCTTCTTTATCAGGTACAAGAGCTGGAGGAGGCAGTGAGCAATGACAATGCA
GCTGCTGAACATCAGGAGACATCAGTGGACTTGTGGATGGTAGGTTCAGTTCACTGAATGGATCAAACCAA
TTCACATCAGATCTTTCAGATGGAAGTGAATGTGTTTTAGTCACAAAAGTGCCCTGAAGCTCAGTTTACAC
GAGCAGAGAAAACCAACACAGTAAGTTATGATGATGCTGATGAACGTCTCCTCATGTCTCATGTCTCTCAC
ACAGATGCCATACAACAGACAGAAGCGTGGCTTTAAGTGTAAGTTCTGCTGCGGCTGCTGCAGACCTGGTG
TCTGTGGACTTTGCTGCAGATTCTGAGGATTCCTGCTCCAACAAC
//
NRC227 (SEQ ID NO: 161)
AAGATGAAGACATTCAGTGTTGCAGTCACAGTGGCCGTCGTGCTCGTCTTTATTTGTATCCAGCAGAGCTC
TGCCACCTTTCCTGAGGTAAGCACCTGACTTCAGATCGTTTCATTTGCTTGTTAGCCTTGAATCTCTCATC
AACATACTGAGACTTGATTTCTTCTTTATCAGGTACAAGAGCTGGAGGAGGCAGTGAGCAATGACAATGCA
GCTGCTGAACATCAGGAGACATCAGTGGACTTGTGGATGGTAGGTTCAGTTCACTGAATGGATCAAACCAA
TTCACATCAGATCTTTCAGATGGAAGTGAATGTGTTTTAGTCACAAAAGTGCCCTGAAGCTCAGTTTACAC
GAGCAGAGAAAACCAACACAGTAAGTTATGATGATGCTGATGAACGTCTCCTCATGTCTCATGTCTCTCAC
ACAGATGCCATACAACAGACAGAAGCGTGGCTTTAAGTGTAAGTTCTGCTGCGGCTGCTGCAGTCCTGGTG
TCTGTGGAGTTTGCTGCAGATTCTGAGGATTCCTGCTCCAAC
//
NRC228 (SEQ ID NO: 162)
AAGATGAAGACATTCAGTGTTGCAGTCACAGTGGCCGTCGTGCTCGTCTTTATTTGTATCCAGCAGAGCTC
TGCCACCTTTCCTGAGGTAAGCACCTGACTTCAGATCGTTTCATTTGCTTGTTAGCCTTGAATCTCTCATC
AACATACTGAGACTTGATTTCTTCTTTATCAGGTACAAGAGCTGGAGGAGGCAGTGAGCAATGACAATGCA
GCTGCTGAACATCAGGAGACATCAGTGGACTCGTGGATGGTAGGTTCAGTTCACTGAATGGATCAAACCAA
TTCACATCAGATCCTTCAGATGGAAGTGAATGTGTTTTAGTCACAAAAGTGCCCTGAAGCTCAGTTTACAC
GAGCAGAGAAAACAAACACAGTAAGTTATGATGATGCTGATGAACGTCTCCTCATGTCTCATGTCTCTCAC
ACAGATGCCATACAACAGACAGAAGCGTGGCTTTAAGTGTAAGTTCTGCTGCGGCTGCTGCAGTCCTGGTG
TCTGTGGACTTTGCTGCAAATTCTGAGGACCTGCCAGCA
//
NRC229 (SEQ ID NO: 163)
AAGATGAAGACATTCAGTGTTGCAGTCACAGTGCCCGTCGTGCTCGTCTTTATTTGTATCCAGCAGAGCTC
TGCCACCTTTCCTGAGGTAAGCACCTGACTTCAGATCGTTTCATTTGCTTGTTAGCCTTGAATCTCTCATC
AACATACTGAGACTTGATTTCTTCTTTATCAGGTACAAGAGCTGGAGGAGGCAGTGAGCAATGACAATGCA
GCTGCTGAACATCAGGAGACATCAGTGGACTCGTGGATGGTAGGTTCAGTTCACTGAATGGATCAAACCAA
TTCACATCAGATCTTTCAGATGGAAGTGAATGTGTTTTAGTCACAAAAGTGCCCTGAAGCTCAGTTTACAC
GAGCAGAGAAAACAAACACAGTAAGTTATGATGATGCTGATGAACGTCTCCTCATGTCTCATGTCTCTCAC
ACAGATGCCATACAACAGACAGAAGCGTGGCTTTAAGTGTAAGTTCTGCTGCGGCTGCTGCAGACCTGGTG
TCTGTGGACTTTGCTGCAAATTCTGAGGACCTGCCAGCA
//
NRC230 (SEQ ID NO: 164)
AAGATGAAGACATTCAGTGTTGCAGTCACAGTGGCCGTCGTGCTCGTCTTTATTTGTATCCAGCAGAGCTC
TGCCACCTTTCCTGAGGTACAAGAGCTGGAGGAGGCAGTGAGCAATGACAATGCAGCTGCTGAACATCACG
AGACATCAGTGGACTCGTGGATGATGCCATACAACAGACAGAAGCGTGGCTTTAAGTGTAAGTTCTGCTGC
GGCTGCTGCAGACCTGGTGTCTGTGGACTTTGCTGCAAATTCTGAGGACCTGCCAGCA
//
NRC231 (SEQ ID NO: 165)
AAGATGAAGACATTCAGTGTTGCAGTCACAGTGGCCGTCGTGCTCGTCTTTATTTGTATCCAGCAGAGCTC
TGCCACCTTTCCTGAGGTACAAGAGCTGGAGGAGGCAGTGAGCAATGACAATGCAGCTGCTGAACATCAGG
AGACATCAGTGGACTCGTGGATGATGCCATACAACAGACAGAAGCGTGGCTTTAAGTGTAAGTTCTGCTGC
GGCTGCTGCAGGCCTGGTGTCTGTGGACTTTGCTGCAGATTCTGAGGATTCCTGCTCCAACAAC
//
NRC232 (SEQ ID NO: 166)
AAGATGAAGACATTCAGTGTTGCAGTCACAGTGGCCGTCGTGCTCGTCTTCATTTGTATCCAGCAGAGCTC
TGCCACCTTTCCTGAGGTAAGCACCTGACTTCAGATCGTTTCATTTGCTTGTTAGCCTTGAATCTCTCATC
AACATACTGAGACTTGATTTCTTCTTTATCAGGTACAAGAGCTGGAGGAGGCAGTGAGCAGTGACAATGCA
GCTGCTGAACATCAGGAGACATCAGTGGACTCGTGGATGGTAGGTTCAGTTCACTGAATGTGTTTTAGTCA
CAAAAGTGCCCTGAAGCTCAGTTTACACAAGCAGAGAAAACAAACAGAGTAAGTTATGATGATGCTGATGA
ACGTCTCCTCATGTCTCATGTCTCTCACACAGATGCCATACAACAGACAGAAGCGTAGCTTTAAGTGCAAG
TTCTGCTGCGGCTGCTGCAGACGTGGTGTCTGTGGACTGTGCTGCAAATTCTGAGGATTCCTGCTCCAACA
AC
//
NRC233 (SEQ ID NO: 167)
AAGATGAAGACTATCAGTGTTGCAGTCACAGTGGCCGTCGTGCTCCTCTTCATTTGTACCCAGCAGAGCTC
TGCCACCTTTCCTGAGGTAAGCACCTGACTTCAGATCGTTTCATTTGCTTGTTAGCCTTGAATCTCTCATC
AACATACTGAGACTTGATTTCTTCTTTATCAGGTACAAGAGCTGGAGGAGGCAGTGAGCAGTGACAATGCG
GCTGCTGAACATCAGGAGACATCAGTGGACTCGTGGATGGTAGGTTCAGTTCACTGAATGGATCAAACCAA
TTCACATCAGATCTTTCAGATGOAAGTGAATGTGTTTTAGTCACAAAAGTGCCCTGAAGCTCAGTTTACAC
AAGCAGAGAAAACAAACACAGTAAGTTATGATGATGCTGATGAACGTCTCCTCATGTCTCATGTCTCATGT
CTCTCACACAGATGCCATACAACAGACAGAAGCGTGGCTTTAAGTGCAAGTTCTGCTGCGGCTGCCGCTGT
GGTGCTCTCTGTGGACTGTGCTGCAAATTCTGAGGATTCCTGCTCCAACAAC
//
NRC234 (SEQ ID NO: 168)
AAGATGAAGACATTCAGTGTTGCAGTCACAGTGGCCGTCGTGCTCGTCTTCATTTGTATCCAGCAGAGCTC
TGCCACCTTTCCTGAGGTAAGCACCTGACTTCAGATCGTTTCATTTGCTTGTTAGCCTTGAATCTCTCATC
AACGTACTGAGACTTGATTTCTTCTTTATCAGGTACAAGAGCTGGAGGAGCCAGTGAGCAGTGACAATGCA
GCTGCTGAACATCAGGAGACATCGGTGGACTCGTGGATGGTAGGTTCAGTTCACTGAATGGATCAAACCAA
TTCACATCAGATCTTTCAGATGGAAGTGAATGTGTTTTAGTCACAAAAGTGCCCTGAAGCTCAGTTTACAC
AAGCAGAGAAAACAAACACAGTAAGTTATGATGATGCTGATGAACGTCTCCTCATGTCTCATGTCTCATGT
CTCTCACACAGATGCCATACAACAGACAGAAGCGTGGCTTTAAGTGCAAGTTCTGCTGCGGCTGCCGCTGT
GOTGCTCTCTGTGGACTGTGCTGCAAATTCTGAGGACCTGCCAGCA
//
NRC235 (SEQ ID NO: 169)
AAGATGAAGACATTCAGTGTTGCAGTCACAGTGGCCGTCGTGCTCGTCTTTATTTGTTCCAGCAGAGCTCT
GCCACCTTTCCTGAGGTAAGCACCTGACTTCAGATCGTTTCATTTGCTTGTTAGCCTTGAATCTCTCATCA
ACATACTGAGACTTGATTTCTTCTTTATCAGGTACAAGAGCTGGAGGAGGCAGTGAGCAGTGACAATGCAG
CTGCTGAACATCAGGAGACATCAGTGGACTCGTGGATGGTAGGTTCAGTTCCCTGAATGGATCAAACCAAT
TCACATCAGATCTTTCAGATGGAAGTGAATGTGTTTTAGTCACAAAAGTGCCCTGAAGCTCAGTTTACACA
AGCAGAGAAAACAAACACAGTAAGTTATGATGATGCTGATGAACATCTCCTCATGTCTCATGTCTCATGTC
TCTCACACAGATGCCATACAACAGACAGAAGCGTGGCTTTAAGTGCAAGTTCTGCTGCGGCTGCCGCTGTG
GTGCTCTCTGTGGACTGTGCTGCAAATTCTGAGGACCTGCCAGCA
//
NRC236 (SEQ ID NO: 170)
ACGAGCTGACAGGAGCTGACAGGAGTCACCAGCAGAGTCAAAGAACTAAACAACTTAACTCAGTCAAACTC
TCAAAGATGAAGACATTCAGTGTTGCAGTCACAGTGGCCGTCGTGCTCGTCTTTATTTGTATCCAGCAGAG
CTCTGCCACCTTTCCTGAGGTACAAGAGCTGGAGGAGGCAGTGAGCAATGACAATGCAGCTGCTGAGCATC
AGOAGACACCAGTGGACTCAGGGATGATGCCAAACAACAGACAGAAGCGCAGCGCCGATTGTTGGCCATGT
TGCAATCAAAATGGCTGTGGAACTTGCTGCAAGGTCTAAACAGACTCTTGGGCAGATCAATCCAGGTTCGT
CTTTCGTTGTCTCTCCGTGGAGTCGAACCAGAGACCTTCTCAGCCCATAGTCCAAGTTTCTGCCACTAGAC
CACCGCCTCTCCCTCATCAAATACTCAATGTTTTTCATTTTGTCTTAAAGTTCATTGAACTATAAACATAT
TTCTGGTAGAGCATGTGATAGTTTAATGGTGTTACTCATTGGTTCATGGTATAGTCAGATGTTCAGAGATG
TGATTATATCATCCACATATTTTCTCTGTTAAGGTGTACTGTCAATAAATGTCAATGCTCCTTTGAAAAAA
AAAAAAAAAAAAAAAC
//
NRC237 (SEQ ID NO: 171)
CGTGCTCGTCTTTATTTGTATCCAGCAGAGCTCTGCCACCTTTCCTGAGGTGAGCTCCTGACTTCAGATCG
TTTCATTTAGCTTGTTATCCATGAATCTCTCATCAACATACTGAGACTTGAATCCTTCTTTATCAGGTACA
GGAGCTGGAGGAGGCAGTGAGCAATGACAATGCAGCTGCTGAACATCAGGAGACATCAGTGGACTCATGGA
TGGTATGTTCAGTTCACTGAATGGATCAAACCAATTCACATCAGATCTTTCAGATGGAAGTGAATTTGTTT
TAGTCCCAAAAGTGCCCTGAAGCTCAGTTTACACAAGCAGAGAAAAACAAAACACAGTAAGTTATGATGAT
GCTGATGAACGTCTCCTCATGTCTCATGTCTCTCACACAGATGCCATACAACAGACAGAAGCGCAGCGCCG
AGTGTAGCTTCTGCTGCAATGAATCTGGCTGTGGAATTTGCTGCAAATTCTGAGGATTCCTGCTCCAACAA
CAAGGGCGAATTC
//
NRC238 (SEQ ID NO: 172)
AAGATGAAGACATTCAGTGTTGCAGTCACAGTGGCCGTCGTGCTCGTCTTTATTTGTATCCAGCAGAGCTC
TGCCACCTTTCCTGAGGTGAGCTCCTGACTTCAGATCGTTTCATTTAGCTTGTTATCCATGAATCTCTCAT
CAACATACTGAGACTTGAATCCTTCTTTATCAGGTACAGGAGCTGGAGGAGGCAGTGAGCAATGACAATGC
AGCTGCTGAACATCAGGAGACATCAGTGGACTCATGGATGGTATGTTCAGTTCACTGAATGGATCAAACCA
ATTCACATCAGATCTTTCAGATGGAAGTGAATTTGTTTTAGTCCCAAAAGTGCCCTGAAGCTCAGTTTACA
CAAGCAGAGAAAAACAAAACACAGTAAGTTATGATGATGCTGATGAACGTCTCCTCATGTCTCATGTCTCT
CACACAGATGCCATACAACAGACAGAAGCGCAGCGCCGAGTGTAGCTTCTGCTGCAATGAATCTGGCTGTG
GAATTTGCTGCAAATTCTGAGGACCTGCCAGCA
//
NRC239 (SEQ ID NO: 173)
GTGGAGGAGCCAGTGAGCAGTGAGAATGGAGCAAATGAACACACATAAGATCTTTCGGATGGAAGTGTATG
TGTTTTAGTCACATGAGTGGCTCGAAGCTCAGTACACACGAGCAGAGAGAACGAACACAGTGTGTTTTATT
CTGCTTGTGTAAACTGAGCTTCAGTTTACACAAGCAGAGAAAACAAACACAGTAAGTTATGATGATGCTGA
TGAACGTCTCCTCATGTCTCATATCTCTCACACAGATGCCAAACAACAGACAGAAGCGTGGCTCTAATTGC
AAACCATGCTGCAATCATAATGGCTGTGGAACGTGCTGCGAAGTCTGAGGATTCCTGCTCCACA
//

1-18. (canceled)

19-42. (canceled)

43. A method of screening a test nucleic acid sequence to identify a candidate nucleic acid sequence encoding an antimicrobial peptide, said method comprising:
(a) identifying an initial peptide of interest;
(b) identifying a DNA sequence from a first fish species containing a nucleotide sequence encoding the initial peptide;
(c) identifying within the DNA sequence a flanking nucleotide sequence on each side of the peptide-encoding sequence;
(d) obtaining a primer oligonucleotide sequence complementary to each flanking sequence; and
(e) screening a test nucleic acid sequence from a fish species other than the first fish species to determine whether it is capable of being amplified by PCR using the primers from step (d); amplification indicating that the test nucleic acid sequence is a candidate nucleic acid sequence encoding an antimicrobial peptide.

44. The method of claim 43 wherein the initial peptide has a net positive charge of at least 2 and has an amphipathic structure.

45. The method of claim 43 wherein the initial peptide is selected from the group consisting of a hepcidin, a pleurocidin, a pardaxin, a misgurin, HFA-1, a piscidin, a moronecidin, and a cleavage product of histone 2A from catfish.

46. The method of claim 45 wherein the cleavage product of histone 2A is a parasin.

47. The method of claim 43 comprising a further step (f) of predicting the amino acid sequence encoded by the candidate sequence and selecting nucleic acid sequences which are predicted to encode peptides having an amphipathic structure and a net charge.

48. The method of claim 47 comprising a further additional step of obtaining a peptide corresponding to the candidate nucleic acid sequence and assaying the peptide sequence for antimicrobial activity.

49. The method of claim 43 comprising a further step (a′) of confirming that the initial peptide has antimicrobial activity.

50. The method of claim 43 wherein the initial peptide is a pleurocidin.

51. The method of claim 50 wherein at least one of the flanking sequences is selected from the group consisting of a nucleotide sequence encoding signal sequence I (SEQ ID NO: 305), a nucleotide sequence encoding Acidic Sequence I (SEQ ID NO: 306),

| | |
|---|---|
| GCCCACTTTGTATTCGCAAG | (SEQ ID NO: 5) |
| and | |
| CTGAAGGCTCCTTCAAGGCG. | (SEQ ID NO: 6) |

52. The method of claim 43 wherein the initial peptide is a hepcidin.

53. The method of claim 52 wherein at least one flanking sequence is selected from the group consisting of a nucleotide sequence encoding signal peptide II (SEQ ID NO: 307), a nucleotide sequence encoding signal peptide III (SEQ ID NO: 308), a nucleotide sequence encoding signal peptide IV (SEQ ID NO: 309), a nucleotide sequence encoding signal peptide V (SEQ ID NO: 310), a nucleotide sequence encoding prosequence I (SEQ ID NO: 311), a nucleotide sequence encoding prosequence II (SEQ ID NO: 312),

| | |
|---|---|
| ACAACCTCGTCCTTAGG | (SEQ D NO: 313) |
| and | |
| ACGCCCGTCCAGGAAT. | (SEQ ID NO: 314) |

54. An isolated nucleic acid sequence identifiable using the method of claim 43.

55. The nucleic acid sequence of claim 54 wherein the sequence is selected from the group consisting of SEQ ID NOS: 82-124, 129-173 and 327.

56. An isolated polypeptide capable of being encoded by the nucleic acid sequence of claim 54.

57. A kit comprising:
a. a first nucleic acid sequence at least 95% identical to a first flanking sequence, located at or near a 5′ end of a target sequence encoding an antimicrobial peptide;
b. a second nucleic acid sequence at least 95% identical to a second flanking sequence located at or near a 3′ end of a target sequence encoding an antimicrobial peptide; and
c. instructions for carrying out the method of claim 43.

58. The method of claim 43, wherein at least one sequence selected from the group consisting of signal sequence I, acidic sequence I, signal peptide II, signal peptide III, signal peptide IV, signal peptide V, prosequence I, prosequence II, nucleic acid sequences encoding them, and nucleic acid sequences substantially complementary to such encoding nucleic acids, is identified.

59. An isolated antimicrobial peptide at least 80% homologous to one of peptide a, b, c or d:

| | |
|---|---|
| Peptide a | GW(G/K)XXFXK |
| Peptide b | GXXXXXXXHXGXXIH |
| Peptide c | FKCKFCCGCCXXGVCGXCC |
| Peptide d | CXXCCNCC(K/H)XKGCGFCCKF |
| Peptide e | FKCKFCCGCRCGXXCGLCCKF |
| Peptide f | XXXCXXCCNXXGCGXCCKX |

60. The antimicrobial peptide of claim 59 which is at least 90% homologous to one of peptide a, b, c or d.

61. The antimicrobial peptide of claim 59 which is one of peptide a, b, c or d.

62. A method of screening a test nucleic acid sequence to identify a candidate nucleic acid sequence encoding an antimicrobial peptide, said peptide comprising:
a) identifying a nucleic acid sequence encoding an initial peptide of interest;
(b) identifying a DNA sequence from a first fish species containing a nucleotide sequence encoding the initial peptide;
(c) identifying within the DNA sequence a flanking nucleotide sequence on each side of the peptide-encoding sequence;
(d) obtaining a primer oligonucleotide sequence complementary to each flanking sequence; and
(e) screening a test nucleic acid sequence from a fish species other that the first fish species to determine whether it is capable of being amplified by PCR using the primers from step (d); amplification indicating that the test nucleic acid sequence is a candidate nucleic acid sequence encoding an antimicrobial peptide.

63. An isolated antimicrobial peptide selected from the group consisting of:

| | |
|---|---|
| (a) | WLRRIGKGVKIIGGAALDHL; |
| (b) | GRRKRKWLRRIGKGVKIIGGAALDHL; |
| (c) | RWGKWFKKATHVGKHVGKAALTAYL; |
| (d) | RSTEDIIKSISGGGFLNAMNA; |
| (e) | FFRLLFHGVHHGGGYLNAA; |
| (f) | FFRLLFHGVHHVGKIKPRA; |
| (g) | GWKSVFRKAKKVGKTVGGLALDHYL; |
| (h) | GWKKWFNRAKKVGKTVGGLAVDHYL; |
| (i) | GWRTLLKKAEVKTVGKLALKHYL; |
| (j) | AGWGSIFKHIFKAGKFIHGAIQAHND; |
| (k) | GFWGKLFKLGLHGIGLLHLHL; |
| (l) | GWKKWLRKGAKHLGQAAIK; |
| (m) | GWKKWLRKGAKHLGQAAIIKGLAS; |
| (n) | GWKKWFTKGERLSQRHFA; |
| (o) | FLGLLFHGVHHVGKWIHGLIHGHH; |
| (p) | GFLGILFHGVHHGRKKALHMNSERRS; |
| (q) | FLGFLFHGIHHGIRAIHLIHG; |
| (r) | FFGALIKGAIHGGKLLHKLIKKKHEHHGYGKHWG; |
| (s) | FLGFLFHGIRHGIKAIHGMIHG; |
| (t) | GKGRWLERIGKAGGIIIGGALDHLG; |
| (u) | GLGNWMGPHISGEKKALHMNSERRS; |
| (v) | GLGNWIVRPIGGEKKALQMNSERRS; |
| (w) | LFGKFLKKVVHAGTSIGETALHVAAEHHGLHAHHG; |
| (x) | GLGNWMGPHISGRKKALHMNSERRS; |
| (y) | FLGLLFHGVHHVGKLIHGLIHG; |
| (z) | ARWGTFFKIFKAGRFIHGAIQAHNDG; |
| (aa) | AWIPALNRIYHGALLRINRQMVYYRRHWHG; |
| (ab) | AWMPALNRIYHGALLRINRQMVYYRRHWHG; |
| (ac) | GWKKWFTKGAKHLGQAAINGLAS; |
| (ad) | GWKKWLRKGAKHLGQAALKGLAS; |
| (ae) | FGDFYMKPGRKISHGYIRSPYG; |
| (af) | GYWRFRNHRGERLSQRHFA; |
| (ag) | FGMLFHRVHHAGRLIHRFIKRHG; |
| (ah) | IFGLIATAVHNAGRLIHRLLGFHHGPPGFWHG; |
| (ai) | IFGLIATAVHNVGRLVHGLLGFHHGPPGFWHG; |
| (aj) | IFGLIATAVHNVGRLVHGLLGFHHGPPRFWHG; |
| (ak) | FFGMRFHGVHHAGGGFLNAQGLLPSLLLNPGYRG; |
| (al) | FFGALLKGAQALHGIIHNARHG; |
| (am) | GWKDWFRKAKKVGKTVGGLALNHYLG; |
| (an) | GIRKWFKKAAHVGKEVGKVALNACL; |
| (ao) | GLKLKWFKKAVHVGKKVGKVALNAYLG; |
| (ap) | GWRKWIKKATHVGKHIGKAALDAYIG; |
| (aq) | GCKKWFKKAAHVGKNVGKVALNAYLG; |
| (ar) | GIRKWFKKAAHVGKKVGKVALNAYLG; |
| (as) | WLERKWFKKATHVGKHVGKAALDAYLG; |
| (at) | FFGLLFHGIHHAGKLIHGLIHHG; |
| (au) | LGNWMGPHISGRKKALQMNSERRS; |
| (av) | FLGLLFHGVHHVGNLIHGLIHHG; |
| (aw) | GIRKWFKKAAHVGKKVGKVALNAYLG; |

(ax) a C-terminally amidated or otherwise C-terminally or N-terminally modified peptide of (a) to (z) or (aa) to (aw);
(ay) a C-terminally amidated peptide of (a) to (z) or (aa) to (aw) where modification replaces C-terminal G; and
(az) a peptide of (a) to (z) or (aa) to (aw) comprising at least one conservative amino acid substitution or deletion of an amino acid residue thereof.

64. An isolated nucleotide sequence encoding a peptide of claim 63.

65. An isolated antimicrobial peptide selected from the group consisting of:

| | |
|---|---|
| (a) | MKTFSVAVAVVVVLACMFILESTAVPFSEVRTEEVESIDSPVGE- |
| | HQQPGGTSMNLPMHFRFKRQSHLSLCRWCCNCCHNKGCGFCCKF; |
| (b) | MKTFSVAVAVVVVLACMFILESTAVPFSEVRTEEVESIDSPVGE- |
| | HQQPGGTSMNLPMHFRFKRQSHLSLCRWCCNCCHNKGCGFCCKF; |
| (c) | MKAFSVAVVLVIACMFILESTAVPFSEVRTEEVGSFDSPVGEHQ- |
| | QPGGESMHLPEPFRFKRQIHLSLCGLCCNCCHNIGCGFCCKF; |
| (d) | RTEEVESIDSPVGEHQQPGGTSMNLPMHFRFKRQSHLSLCRWCC- |
| | NCCHNKGCGFCCKF; |
| (e) | MKTFSVAVVPVIACMFILESTAVPFSEVRTEEVGSFDSPVGEHQ- |
| | QPGGTSMNLPMHFRFKRQSHLSLCRWCFNCCHNKGCGFCCKF; |
| (f) | MKQFSVAVVLVMACMFIVESTAVPFSEVRTEEVGSLDSPVGEHQ- |
| | QPGGESMHLPEPFRFKRQIHLSLCGLCCNCCHNIGCGFCCKF; |
| (g) | MKAFSIAVAVTLVLAFVCIQCSSAVPFQGVQELEEAGGNDTPVA- |
| | EHQVMSMESWMENPTRQKRHISHISLCRWCCNCCKANKGCGFCC- |
| | KF; |
| (h) | MKTFSVAVAVTLVLAFVCIQDSSAVPFQGVQELEEAGGNDTPVA- |
| | AHQMMSMESWMESPVRQKRHISHISMCRWCCNCCKAKGCGPCCK- |
| | F; |
| (i) | MKTFSVAVTVAVVLVFICIQQSSGTFPEVQELEEAVSNDNAAAE- |
| | HQETSVDSWMMPYNRQKRAFKCKCFCCGCCRAGVCGLCCKF; |
| (j) | MKTFSVAVTVAVVLVFICIQQSSASFPEAQELEEAVSNDNAAAE- |
| | HQETPVDSWMMPYNRQKRSFKCKFCCGCCRAGVCGLCCKF; |
| (k) | MKTFSVAVTVAVVLVFICIQQSSASFPEAQELEEAVSNDNAAAE- |
| | HQETPVDSWMMPNNRQKRGFKCKFCCGCCRAGVCGLCCKF; |
| (l) | MKTFSVAVTVAVVLVFICIQQSSATFPEMPYNRQKRGFKCKFCC- |
| | GCCGAGVCGMCCKF; |
| (m) | MKTFSVAVTVAVVLVFICIQQSSASFPEAQELEEAVSNDNAAAE- |
| | HQETPVDSRIPYNRQKRSFKCKFCCGCCRAGVCGLCCKF; |
| (n) | MKTCSVAVTVAVVLVFICIQQSSASFPEVQELEEAVSNDNAAAE- |
| | HQETPVDSWMMPNNRQKRGFKCKFCCGCCRAGVCGLCCKF; |
| (o) | MKTISVAVTVAVVLVFICIQQSSASFPEAQELEEAVSNDNAAAE- |
| | HQETPVDSGMIPYNRQKRSFKCKFCCGCCRAGVCGLCCKF; |
| (p) | MKTFSGAVTVAVVLVFICIQQSSASFPEVQELEEAVSNDNAAAE- |
| | HQETPVDSWMMPNNRQKRGFKCKFCCGCCRAGVCGLCCKF; |
| (q) | MKTSVVAVTVAVVLVFICIQQSSATFPEVQELEEAVSNDNAAAA- |
| | HQETSVDSWMMPYNRPKRSFKCKFCCGCCRA-GVCGLCCKF; |
| (r) | MKTFSVAVTVAVVLVFICIQQSSATFPEVQELEEAVSNDNAAAE- |
| | HQETSVDSWMMPYNRPKRSFKCKFCCGCCRAGVCGLCCKF; |
| (s) | MKTFVVAVTVAVVLVFICIQQSSATFPEVQELEEAVSNDNAAAE- |
| | HQETSVDSWMMPYNRQKRSFKCKFCCGCCRAGVCGLCCKF; |
| (t) | MKTSVVAVTVAVVLVFICIQQSSATFPEVQELEEAVSNDNAAAA- |
| | HQETSVDSWMMPYNRQKRSFKCKFCCGCCRAGVCGLCCKF; |
| (u) | MKTFSVAVTVAVVLVFICIQQSSATFPEVQELEEAVSNDNAAAE- |
| | HQETSVDLWMMPYNRQKRGFKCKFCCGCCSPGVCGLCCRF; |
| (v) | MKTFSVAVAVAVVLIFICIQQSSATFPEVQELEEAVSNDNAAAE- |
| | HQETSLDSWMMPYNRQKRGFKCKFCCGCCRAGVCGLCCKF; |
| (w) | MKTFSVAVTVAVVLVFICIQQSSATFPEVQELEEAVSNDNAAAE- |
| | HQETSLDSWMMPYNRHKRSFKCKFCCGCCRAGVCGLCCKLF; |
| (x) | MKTFSVAVTVAVVLVFICIQQSSATFPEVQELGEAVSNDNAAAE- |
| | HQETSVDSWMMPYNRPKRSFKCKFCCGCCRAGVCGLCCKF; |
| (y) | MKTFSVAVTVAVVLIFICIQQSSATSPEVQGLEEAVSNDNAAAE- |
| | HQETSVDSWMMPYNRQKRGFKCKFCCGCCRPGVCGLCCRS; |
| (z) | MKTFSVAVTVAVVLVFICIQQSSATFPEVQELEEAVSNDNAAAE- |
| | HQETSVDLWMMPYNRQKRGFKCKFCCGCCRPGVCGLCCRF; |
| (aa) | MKTFSVAVTVAVVLVFICIQQSSATFPEVQELEEAVSNDNAAAE- |
| | HQETSVDL-WMMPYNRQKRGFKCKFCCGCCSPGVCGLCCRF; |
| (ab) | KTFSVAVTVAVVLVFICIQQSSATFPEVQELEEAVSNDNAAAEH- |
| | QETSVDS-WMMPYNRQKRGFKCKFCCGCCSPGVCGLCCKF; |
| (ac) | MKTFSVAVTVAVVLVFICIQQSSATFPEVQELEEAVSNDNAAAE- |
| | HQETSVDS-WMMPYNRQKRGFKCKFCCGCCRPGVCGLCCKF; |
| (ad) | MKTFSVAVTVAVVLVFICIQQSSATFPEVQELEEAVSNDNAAAE- |
| | HQETSVDSWMMPYNRQKRGFKCKFCCGCCRPGVCGLCCKF; |
| (ae) | MKTFSVAVTVAVVLVFICIQQSSATFPEVQELEEAVSNDNAAAE- |
| | HQETSVDSWMMPYNRQKRGFKCKFCCGCCRPGVCGLCCRF; |
| (af) | MKTFSVAVTVAVVLVFICIQQSSATFPEVQELEEAVSSDNAAAE- |
| | HQETSVDSWMMPYNRQKRSFKCKFCCGCCRRGVCGLCCKF; |
| (ag) | MKTISVAVTVAVVLLFICTQQSSATFPEVQELEEAVSSDNAAAE- |
| | HQETSVDSWMMPYNRQKRGFKCKFCCGCRCGALCGLCCKF; |
| (ah) | MKTFSVAVTVAVVLVFICIQQSSATFPEVQELEEPVSSDNAAAE- |
| | HQETSVDSWMMPYNRQKRGFKCKFCCGCRCGALCGLCCKF; |
| (ai) | MKTFSVAVTVAVVLVFICIQQSSATFPEVQELEEAVSSDNAAAE- |
| | HQETSVDSWMMPYNRQKRGFKCKFCCGCRCGALCGLCCKF; |
| (aj) | MKTFSVAVTVAVVLVFICIQQSSATFPEVQELEEAVSNDNAAAE- |
| | HQETPVDSGMMPNNRQKRSADCWPCCNQNGCGTCCKV; |
| (ak) | MKTFSVAVTVAVVLVFICIQQSSATFPEVQELEEAVSNDNAAAE- |
| | HQETSVDSWMMPYNRQKRSAECSFCCNESGCGICCKF; |
| (al) | MKTFSVAVTVAVVLVFICIQQSSATFPEVQELEEAVSNDNAAAE- |
| | HQETSVDSWMMPYNRQKRSAECSFCCNESGCGICCKF; |
| (am) | MPNNRQKRGSNCKPCCNRNGCGTCCEV; |
| (an) | a C-terminally amidated peptide (a) to (z) or |
| | (aa) to (am); and |
| (ao) | a peptide of (a) to (z) or (aa) to (am) com- |
| | prising at least one conservative amino |
| | acid substitution of an amino acid |
| | residue thereof. |

66. An isolated nucleotide sequence encoding a peptide of claim 65.


SEQUENCE LISTING (329 sequences)

SEQ ID NO: 1 - protein, 11 aa, Artificial Sequence
  Description of Artificial Sequence Synthetic peptide
FFKKAAHVGKH

SEQ ID NO: 2 - DNA, 32 bp, Artificial Sequence
  Description of Artificial Sequence Primer
ttcttcaagaaggcygcycaygtsggmaagca

SEQ ID NO: 3 - protein, 11 aa, Artificial Sequence
  Description of Artificial Sequence Synthetic peptide
HVGKAALTHYL

SEQ ID NO: 4 - DNA, 32 bp, Artificial Sequence
  Description of Artificial Sequence Primer
caygtsggmaaggcygcyctsaahcaytacct

SEQ ID NO: 5 - DNA, 20 bp, Artificial Sequence
  Description of Artificial Sequence Primer
gcccactttgtattcgcaag

SEQ ID NO: 6 - DNA, 20 bp, Artificial Sequence
  Description of Artificial Sequence Primer
ctgaaggctccttcaaggcg

SEQ ID NO: 7 - protein, 7 aa, Artificial Sequence
  Description of Artificial Sequence Synthetic peptide
MKFTATF

SEQ ID NO: 8 - DNA, 21 bp, Artificial Sequence
  Description of Artificial Sequence Primer
atgaagttcactgccaccttc

SEQ ID NO: 9 - protein, 6 aa, Artificial Sequence
  Description of Artificial Sequence Synthetic peptide
KRAVDE

SEQ ID NO: 10 - DNA, 17 bp, Artificial Sequence
  Description of Artificial Sequence Primer
tcatcgactgcgcgctt

SEQ ID NO: 11 - protein, 7 aa, Pleuronectes americanus
KGRWLER

SEQ ID NO: 12 - DNA, 21 bp, Pleuronectes americanus
aagggcaggtggttggaaagg

SEQ ID NO: 13 - protein, 6 aa, Pleuronectes americanus
YQEGEE

SEQ ID NO: 14 - DNA, 17 bp, Pleuronectes americanus
ccctccccctcctggta

SEQ ID NO: 15 - protein, 7 aa, Pleuronectes americanus
RKRKWLR

SEQ ID NO: 16 - DNA, 21 bp, Pleuronectes americanus
cgtaagagaaagtggttgaga

SEQ ID NO: 17 - protein, 6 aa, Pleuronectes americanus
KAAHVG

SEQ ID NO: 18 - DNA, 18 bp, Pleuronectes americanus
aaggctgctcacgttggc

SEQ ID NO: 19 - DNA, 20 bp, Pleuronectes americanus
ctgaaggctccttcaaggcg

SEQ ID NO: 20 - protein, 7 aa, Pleuronectes americanus
FLGALIK

SEQ ID NO: 21 - DNA, 21 bp, Pleuronectes americanus
ttcttaggagcccttatcaaa

SEQ ID NO: 22 - protein, 6 aa, Pleuronectes americanus
YDEQQE

SEQ ID NO: 23 - DNA, 18 bp, Pleuronectes americanus
ctcctgctgctcgtcata

SEQ ID NO: 24 - protein, 6 aa, Pleuronectes americanus
HGRHAA

SEQ ID NO: 25 - DNA, 18 bp, Pleuronectes americanus
catggtcgtcatgctgcc

SEQ ID NO: 26 - DNA, 20 bp, Pleuronectes americanus
ctgaaggctccttcaaggcg

SEQ ID NO: 27 - protein, 6 aa, Pleuronectes americanus
GFLFHG

SEQ ID NO: 28 - DNA, 18 bp, Pleuronectes americanus
gggatttctttttcatgg

SEQ ID NO: 29 - protein, 6 aa, Pleuronectes americanus
SFDDNP

SEQ ID NO: 30 - DNA, 18 bp, Pleuronectes americanus
gggttgtcatcgaatgag

SEQ ID NO: 31 - protein, 6 aa, Pleuronectes americanus
RSTEDI

SEQ ID NO: 32 - DNA, 18 bp, Pleuronectes americanus
cgttctacagaggacatc

SEQ ID NO: 33 - protein, 6 aa, Pleuronectes americanus
DDDDSP

SEQ ID NO: 34 - DNA, 18 bp, Pleuronectes americanus
ggggctgtcatcatcatc

SEQ ID NO: 35 - protein, 6 aa, Pleuronectes americanus
WMENPT

SEQ ID NO: 36 - DNA, 18 bp, Pleuronectes americanus
tggatggagaatcccacc

SEQ ID NO: 37 - DNA, 18 bp, Pleuronectes americanus
gtgaggttgtgttgcggg

SEQ ID NO: 38 - protein, 6 aa, Pleuronectes americanus
GMMPNN

SEQ ID NO: 39 - DNA, 18 bp, Pleuronectes americanus
gggatgatgccaaacaac

SEQ ID NO: 40 - DNA, 19 bp, Pleuronectes americanus
acttggactatgggctgag

SEQ ID NO: 41 - protein, 6 aa, Pleuronectes americanus
WMMPNN

SEQ ID NO: 42 - DNA, 18 bp, Pleuronectes americanus
tggatgatgccatacaac

SEQ ID NO: 43 - DNA, 19 bp, Pleuronectes americanus
gttgttggagcaggaatcc

SEQ ID NO: 44 - protein, 6 aa, Pleuronectes americanus
AALVVD

SEQ ID NO: 45 - DNA, 20 bp, Pleuronectes americanus
tcgctgccctcgttgttgac

SEQ ID NO: 46 - protein, 7 aa, Pleuronectes americanus
VLLTEAP

SEQ ID NO: 47 - DNA, 19 bp, Pleuronectes americanus
ggagcctcggtcagcagga

SEQ ID NO: 48 - protein, 6 aa, Pleuronectes americanus
VFPSIV

SEQ ID NO: 49 - DNA, 17 bp, Pleuronectes americanus
gtgttccatccatcgtc

SEQ ID NO: 50 - protein, 7 aa, Pleuronectes americanus
HTFYNEL

SEQ ID NO: 51 - DNA, 20 bp, Pleuronectes americanus
gagctcgttgtagaaggtgt

SEQ ID NO: 52 - protein, 6 aa, Salmo salar
MHLPEP

SEQ ID NO: 53 - DNA, 18 bp, Salmo salar
atgcatctgccggagcct

SEQ ID NO: 54 - DNA, 23 bp, Salmo salar
cattgcaaacatgtacaaactag

SEQ ID NO: 55 - protein, 6 aa, Salmo salar
MNLPMH

SEQ ID NO: 56 - DNA, 17 bp, Salmo salar
atgaatctgccgatgca

SEQ ID NO: 57 - DNA, 16 bp, Salmo salar
gggcaaattaaaggcg

SEQ ID NO: 58 - protein, 8 aa, Salmo salar
IVGRPRHQ

SEQ ID NO: 59 - DNA, 23 bp, Salmo salar
tcgtcggtcgtcccaggcatcag

SEQ ID NO: 60 - protein, 8 aa, Salmo salar
GYALPHAI

SEQ ID NO: 61 - DNA, 23 bp, Salmo salar
atggcgtggggcagagcgtaacc

SEQ ID NO: 62 - protein, 24 aa, Artificial Sequence
  Description of Artificial Sequence Synthetic pleurocidin-like peptide
GKGRWLERIGKAGGIIIGGALDHL

SEQ ID NO: 63 - protein, 20 aa, Artificial Sequence
  Description of Artificial Sequence Synthetic pleurocidin-like peptide
WLRRIGKGVKIIGGAALDHL

SEQ ID NO: 64 - protein, 26 aa, Artificial Sequence
  Description of Artificial Sequence Synthetic pleurocidin-like peptide
GRRKRKWLRRIGKGVKIIGGAALDHL

SEQ ID NO: 65 - protein, 25 aa, Artificial Sequence
  Description of Artificial Sequence Synthetic pleurocidin-like peptide
GWGSFFKKAAHVGKHVGKAALTHYL

SEQ ID NO: 66 - protein, 24 aa, Artificial Sequence
  Description of Artificial Sequence Synthetic pleurocidin-like peptide
FLGALIKGAIHGGRFIHGMIQNHH

SEQ ID NO: 67 - protein, 25 aa, Artificial Sequence
  Description of Artificial Sequence Synthetic pleurocidin-like peptide
GWGSIFKHGRHAAKHIGHAAVNHYL

SEQ ID NO: 68 - protein, 25 aa, Artificial Sequence
  Description of Artificial Sequence Synthetic pleurocidin-like peptide
RWGKWFKKATHVGKHVGKAALTAYL

SEQ ID NO: 69 - protein, 21 aa, Artificial Sequence
  Description of Artificial Sequence Synthetic pleurocidin-like peptide
RSTEDIIKSISGGGFLNAMNA

SEQ ID NO: 70 - protein, 19 aa, Artificial Sequence
  Description of Artificial Sequence Synthetic pleurocidin-like peptide
FFRLLFHGVHHGGGYLNAA

SEQ ID NO: 71 - protein, 19 aa, Artificial Sequence
  Description of Artificial Sequence Synthetic pleurocidin-like peptide
FFRLLFHGVHHVGKIKPRA

SEQ ID NO: 72 - protein, 25 aa, Artificial Sequence
  Description of Artificial Sequence Synthetic pleurocidin-like peptide
GWKSVFRKAKKVGKTVGGLALDHYL

SEQ ID NO: 73 - protein, 25 aa, Artificial Sequence
  Description of Artificial Sequence Synthetic pleurocidin-like peptide
GWKKWFNRAKKVGKTVGGLAVDHYL

SEQ ID NO: 74 - protein, 23 aa, Artificial Sequence
  Description of Artificial Sequence Synthetic pleurocidin-like peptide
GWRTLLKKAEVKTVGKLALKHYL

SEQ ID NO: 75 - protein, 26 aa, Artificial Sequence
  Description of Artificial Sequence Synthetic pleurocidin-like peptide
AGWGSIFKHIFKAGKFIHGAIQAHND

SEQ ID NO: 76 - protein, 21 aa, Artificial Sequence
  Description of Artificial Sequence Synthetic pleurocidin-like peptide
GFWGKLFKLGLHGIGLLHLHL

SEQ ID NO: 77 - protein, 19 aa, Artificial Sequence
  Description of Artificial Sequence Synthetic pleurocidin-like peptide
GWKKWLRKGAKHLGQAAIK

SEQ ID NO: 78 - protein, 23 aa, Artificial Sequence
  Description of Artificial Sequence Synthetic pleurocidin-like peptide
GWKKWLRKGAKHLGQAAIKGLAS

SEQ ID NO: 79 - protein, 18 aa, Artificial Sequence
  Description of Artificial Sequence Synthetic pleurocidin-like peptide
GWKKWFTKGERLSQRHFA

SEQ ID NO: 80 - protein, 24 aa, Artificial Sequence
  Description of Artificial Sequence Synthetic pleurocidin-like peptide
FLGLLFHGVHHVGKWIHGLIHGHH

SEQ ID NO: 81 - protein, 26 aa, Artificial Sequence
  Description of Artificial Sequence Synthetic pleurocidin-like peptide
GFLGILFHGVHHGRKKALHMNSERRS

SEQ ID NO: 82 - DNA, 901 bp, Pleuronectes americanus
atgaagttcactgccaccttcctcctgttgttcatcttcgtcctcatggttgatctcggagagggtcgtcgtaagaaaaaggggtcgaagagaaaggggtccaagggaaaggggtccaagggaaagggcaggtggttggaaaggattggtaaaggtagagtcacggaattaatttgctttttacattgcaaatatttttcatataacattgctggaaaatcacaaaaataagtagtcaatatatttggccaaatagaatcactttgatttcaataataatcaaaataacaacctaaaaggcctttgattagcatgttccttcaatgaaatggacattgtaatttactttgattctcacatgctacgacctgctgcagcaacatttgaaaataaatttgtcccagaagattttaaagtacattgttataggcgatttatctttctattactcagatatttgttcaaaccaatagaataactggatctctatgctaaaataataaaacacacattcagatgttaccagtcaagattgaacgctgtttaaaagtaagtatgaaacatcctctgtatgtataattgtttaactggtaacttatagtcctaataattgcgttatggaaatgtattaattgtcatttaatataatttgctggaatttatcactgtgtgtttttgtttgtttttacacagctggcgggataattatcgggggggcccttgagtaaggacttctaccatcattactgtgtaatatttatagttatgatcagtacagttattaacaacttctcttgtctcgctgaacttctccatcagtcacctcgggcaggggcaggtgcaggggccggattacgactaccaggagggggaggagctcaacaagcgcgcagtcgatgaa

SEQ ID NO: 83 - DNA, 217 bp, Pleuronectes americanus
atgaagttcactgccaccttcctcctgttgttcatcttcgtcctcatggttgatctcggagagggtcgtcgtaagagaaagtggttgagaaggattggtaaaggtgtcaagataattggcggggcggcccttgatcacctcgggcaggggcaggtgcaggggcaggattacgactaccaggaggggcaggagctcaacaagcgcgcagtcgatgaaa

SEQ ID NO: 84 - DNA, 968 bp, Pleuronectes americanus
gcccactttgtattcgcaaggtaatattgatatttttcatattcatttagacaaatgtgctcagcttgttactgtataatgcaaaagttaatgatctttatttttctgtttttttttgtagaatgaagttcactgccaccttcctcatgattgccatcttcgtcctcatggttgaacctggagagtgtggctggggaagcttttttaaaaaggctgctcacggtagagtcacagaattaattagctttttgctttgcaaatattttttttataacagctggaaaatcacaaaaataaatagtatatatatttggccaataaaatcactttgatttcaataataatctaaataaccaacctaaaaggcctttgattagcatgttccttcaatgaaatgtacgttgaggtttattttgattctcacaagcaccaacctgctgcgtcaacaattgaattcaaatttgtcccaaaggaattcaaagtaaatttttctaggcgatttaatctttccattactctgatttgttttaaaaatatagaataactcaatctctatgataaaacaattacacatacattcagatttttataggacaagattgaaaacttcttacaagtatgtataaaacatcatctgtatgtataattgtttaacatgtaacaactagtcctactaattgtgttaaattgtcatttaatatcaattgcttgagtttatcattatgtgttttgtttttttttacacagttggcaagcatgttggcaaggcggcccttacgtaaggacttctaccattttactgtataattttgatagtgttatcaccagtactgtttttgacaacttctctattcctgctgactctctccatccgactcatccgcagtcattaccttggcgataagcaggagctcaacaagcgtgcagtcgatgaagacccaaatgttattgtttttgaatgaagaaat

SEQ ID NO: 85 - DNA, 654 bp, Pleuronectes americanus
atgaagttcactgccaccttcctggtgctgtccctggtcgtcctaatggctgagcctggagagtgtttcttaggagcccttatcaaaggggccatacatggtagagtcaaggaattaattagatttttacatgtcaaataatgtagtagaacatatataagtagtcaatatatttgaccaagtagaatcattttgatttcaataataatcaaaataacaatctccaggcgatttaatatttgcaataattggattttatagaatacggaacaactggatcttaatgctaaaataatccaacatacattctgattttgccaggcaaaattaaacactactttaaagtatgtataaaacataatctgtatgttataacaaatactccaagcaattgtgtgatggaaatgtattcattgtcatttaatataatttgcttgagtttatcatcttgtgtttttgtttgttttttcacaggtggcaggtttatccatgggtaaggacttctaccatcatgactgtgtatttttaatattattatcatcagtactgttattgacaacttcacttgtctcgctgactctctccatcagaatgatccaaaaccatcacggttatgacgagcagcaggagctcaacaagcgcgcagtcgatgaa

SEQ ID NO: 86 - DNA, 684 bp, Pleuronectes americanus
gcccactttgtattcgcaaggtaatatcaatatttttcaaattcatttagacgagaccaaccttttgggaaatctgctcagcttattactgtataatgcaaatgttaatgatctttatttttctgttttttttttgtagaatgaagttcactgccaccttcctcatgatgttcatcttcgtcctcatggttgaacctggagagtgtggttggggaagcatttttaagcatggtcgtcatggtaaagtcacggaattaattagcttttaactttgcaaatattgtttttttttttaacagctggaaactcacaaaaataaatagccgatatatttggccaattataatcactttgatctaaataacaacctaaaaggcctttgattagcatgtttcttcaataaaatgattgaacactacttaaaggtatgtataaaacatcatcatgtgtttttgtttgtttttacacagctgccaagcatattggccatgcagccgttaagtaaggacttctaccattattactgtataattttgatagtattatcaccagtattgttattgacaacttctctttttcctgctgatccgactcatccgcagtcattaccttggcgagcagcaagatctcgacaagcgcgcagtcgatgaagacccaaatgttattgtttttgaatgaagaaat

SEQ ID NO: 87 - DNA, 826 bp, Pleuronectes ferruginea
atgaagttcactgccaccttcctcatgatgtgcatcttcgtcctcatggttgaacctggagagtgtcgttgggggaaatggtttaaaaaggccacacacggtagagtcacagaattaattagctttttgctttgcaaatatttttttataacagctggaaaatcacaaaaataaatagtctatatatttggccaattagaatcactttgctttcaataaaaatctaaataacaacctaaaagtcctttgattagcattttccatcaatgaaatggacgttgaggtttattttgattctcacatgcaccgacctgctatgtcaacaattgaatacaaatttgtcccagaggaattcaaaggaaatttttctaggcgatctaatctttccattactcggatttgtttttaaatatatagaataactcaatctctatgataaaataataacacatacgtaaagatttttacaagacaagattgaaaacttcttaaaagtacgtataaaacatcatctgtatttataattgtttaacatttaacaaatagccctactaattgtgttatggaaatgtataaattgtcatttaacataacttgtttgagtttatcattatttgtttttgtttgtttttacacagttggcaagcatgttggcaaggcggcccttacgtaaggacttctaccatcattactgtataattttgatagtattatcaccagtactgttattgacaacttctcttgtcctgctgactctctccatccgactcatccatagtgcttaccttggcgacaagcaagaactcgacaagcgcgcagtcgatga

SEQ ID NO: 88 - DNA, 1300 bp, Pleuronectes americanus
taataaaactaatgtgtaaagtcttccactttttttactgtatttacttaaacagaaaattattctcacgattctggagctgcagccactaagtgttgcttcatgaagtgaatacacaattgttctaacaaccactcacccaattaaccagaatctacaaagtgaggaagtgagaggagtcgtcctgtgttttcaaattttttgaatgatctaccactatgtgagctcctcctgttatagctctaaatgttacacaatgaatgtgaagtcagttctgtgtatataaagagttgcctctgtagagcatacaacagatttcacctttgaatctcacaaacctcactttgtattcgacaggtaagatcgatatttttcaaactcatttagacgagaccaagtatttgggaaatgtgctcagcttgtcaatgtataatgcaaatgttaacaatcgttttgttcttatgttgtgtttgtaggatgaagttcgctactgccttcctgatgttgtccatggtcgtcctcatggctgaacctggagagtgtcgttctacagaggacatcatcaagtctatctcgggtagagtccaggaattaattattatcaataacaatgaaataacaaccaaaaggcctctgattagcatgttccttcaatgaaatggtcgttttttatctattttgattctcacatgcaacgacctgctgcggcaacatttgaaaatcaatcttttttacacaaattcaaagtacattgatttattcgatttaatcttaacattaatcagatttgtttttgtttaaatatatcgaataactggatctctatgataaaataattaaacatacattcttattttaccaatcaagattgaacacttcttaaaagtacgtataaaacatcatctgtatgtataattgtttgattgttaagtaatatttccaataattgtgtaatggaaatgtattaattgtcatttaatataatttgcttgaatttatcaccatgtgttttttgtttgtttttaaacaggtggaggttttctcaatgcgtaaggacttctatcatcattactgtgtaatttttatagtattatcatcagtactgttattaacagcttctcttgtctcactgactctctccatcagaatgaacgccggttacaatgagcagcaggagctcaacaagcgctcagatgatgatgacagccccagtcttattgtttttgactgaagaagtcgccctgaaggagccttcagatgatatattatgcttcttgctcttcattgaaataaatcaaac

SEQ ID NO: 89 - DNA, 3500 bp, Pleuronectes americanus
gagctcgatcaaaccagacaaagttgccttccttcacaacaatagagtggaagagaaaacaggagaggacttgtatcctcctgatgctgagaagaagaaataagaaagacttgcagcattgatacttttacttatacagaaaacctataaacatgacgggagcataagttaaagtcacaatacagaagagaaccagaagccaaactgcagcaaatttactggtattcatatgatactggagccaaagcaacgcagagactcagcagcagtgaaccaaagagtttaactgtacttgtgtccaggttgaatgaaagtattgaataaaaaaaaccaagacagaacatgcatatttttttggaatggaatataagtcaggagaatatgtgttgttgtggtggcaggatccatcactctgtcaagttaacacaagaacttttagaaacatagatacgatctcaagtaaacttccatttactatttgactttttttaaatacttacaaattatattttaaaaagcaacaataaatcagagataacttcatggagaagtctatattcatatttgtgagctgaacattcatgctgcctgttctatcacatctgagtgtggaggccactgacgtttactgacctcaacgtctaccgctctaatgcatttggagttaaaggtaagcattttgttatttgtcttcactgtattgatactaaatatacagggttacaaatacagttaaaacaagagagacgaggtgtcgaaagcttcagcatcaatgtgctgatcgctgatagctgatcttacccgacaccggtgacatggcatcaaaatgaccacctcttttttcttctcttttttttgtaggacgaagttcgctgccgccttcctcgtgttgttcatggtcatcgtcatgtttgaacctggagagtgtttttttagattgctttttcacggggtccaccatggtagggtcccggaagtaatttgattattacatgccaaatattttaatgaaacataccttatgagtagttgtattatttggacaagtagaatctctatgatttcagtagtaattagaataacaatcaaaaaggcctttgattagcatgtttcttcaatgaaatggacattgaggtttattttgattctcacatgctacagcaacaattgaaatcaaatttttcgcagaagaaacttaattaacattgttgtgcaatagtgcttaaaaagtgttaccatggaatggtgtgcgtttaggcactcaataaatttggttatcaaaattaaattaaaaaaattaatatttaaaatattaatattaaatcataactttaattgtttaaagttctcgcggggaaccacccttcttctgaaggtaaaggatagccaatttattgattaagatcagtctcatttagatctagttcaaatagaaatctcaatattttaccatcgaagattttataatgaacagtgaaggttatggagttctaaacagtgtaacagttggcaaagttcactattgcaatattaatgacagaccatttgtgaaagaagaacatttattatgagcataataaagtatgaaagcacgaattactaaacaatcaaagctaactaacaaggacgtgtgtgggtgtgtgtgtgaatgtaaataaggggggggctcaaactggtggcctacaagaagagccttaagatagcaaccacaagggctgtaccataaatgttgtagtaaaaagagttattaaaatgagttagaataactaatgactaattagtagacaaactagtagacaaactaaacaactaacaataacaaggaagtgtgtgtgagtgtgtttgtgtgtaaatgttaattaggggctctcaaactggtgtcttaccagaagagtaagataacaattcccccccttcttctgaggttgttttacgactgttgctttatggccgtgagggaaggtttaactcggtgacatgctatacgtgtctgtgtagatgttaatcagagaatgccagagtcagagagacctacggaggaagtctgtgaagggcctatctaacattagctttcctttaacttataacacaatatcagaaacacatatcaaccttataaacacacacagaatcaaataaacagtcttgcttagcatgtataattattaagcccagattatgttaccagtccgagggaaagagttcagttgcagttctgtgacgtctcctggctttgtggtcgtagagttctgcattcgcgattctgtcgagccgtgtgctcagatgcaggttgaagttctcctgcaggacatcgcgtcgctgcgaggattttgtagagcttgaagggcgaggagatttccttgagtggtgagctggaagctggacctctgacctctggttgttggttggaagagaagaaagctggagcggcgtggtttctccctctagccgatgcaggaggagaagccggcagccccactccttgaagagttgtggagagagatgggagcaaagagctagattttggggagacctctccttatattggccccgatgacctcacaggccttggaacggagtgaccaataggagttgaccctggtaattcttgacacctttgtgggacattgtcaagaccccaggacatgcagcatcctgttacaatctgggagacggagttccttgactgtctcagaacaatgagaacctgtggcatcttgggggattgagtccactcgagcacatgcggcatgtttgttccaagtttgactgaaaggaggcctgtggtttgcacaaaaaccatgtcccaacaacattttctaggcgatttaatctttacataaattggatttgttttaaaaaatatatagaataactcgatctttctgcgtaaataataaaaaataaattcaaatttgaccagtcaagattgaacactaatgaaaagtacctataaaacataatctgtatgtatagttgtttgactgttaaatagtagtcctaacaattgtgtaatggaaatgtattcattgtcttttaatactatttgcttatcataatgtgtttgtttgttttttagcaggtggaggttatctcaatgcgtaaggacttctaccatcattactgtgtaattgtattagttttatcatcagtactgttattgacaacgtctcttgtcttgctgacttgactctcttcatcagattaaacccagggccggttacaatgagcagcaggagctcgacaagcgcgcagtcgatgacaacctcagtgctattgttttttactgaagaagtcgacctgaagaatcttttgaaatgatatgaaatgtttgcctttcaatgaaataaatcaaacatgactggatatttgttcttttgcattgatgtattgttgagtgacagttgaataattttggaaaacttataacagatctcaattttaggatgtcaaatcatttctctgtgtcttattcaaatatgagatttaacaatgacaat

SEQ ID NO: 90 - DNA, 1003 bp, Hippoglossoides platessoides
gcccactttgtattcgcaaggtaagatcaatatttttcaaattcatttagacgagaccaaccgtttgcgaaatgtgctcagcttgttattgtataataacaaagttaacgatctttatttttctgtttttttgtagaatgaagttcactgccaccttcctgatgttgttcatcttcgtcctcatggttgaacctggagagtgtggatggaaaagtgtgtttcgtaaggctaagaaaggtagagtcacggaattaattagctttttacattgcaaatagattttttataacagctggaaaatcacaaaaataaatagtcgatatatttggccaattagaatcactttaatttcaataataatctaaataacaacctaaaaggcctttgattagcatgtttcttcaatgaaatggacattgaggtttattttgattctcacatgcaccgacctgtgcggcaaccattgaattcagatttgtcccagaagaattcaaagtacatttttccaggcgattaaatctttccattactcagattcaaaaataaataaatggaataattgaagcactatgataaaataattacacattcactctgactttacaagtcaagattgaacactattaaaaagtgtgtataaaacaacatctgtatgcataattgtttaactgttaatagtcctaataattgttttatggaaatgtattaatttacatttaatattatttgcttgagtttaccatcatgtgtttttgtttgtttttacacagttggcaagactgttggcggcttggcccttgagtaaggacttctaccatcattactgtataattttgatagtattatcaccagtactgttattaactacttctcttgtctgctgactctctccatccgactcatctgcagtcattaccttggcgagcagcaggagcttgacagcgcgcagtcgatgaggaccccagtgctattgtctttgactgaagaagtcgccttgaaggag

SEQ ID NO: 91 - DNA, 1007 bp, Hippoglossoides platessoides
actttgtattcgcaaggtaagatcaatatttttcaaattcatttagacgagaccaaccgttggcgaaatgtgctcaacttgttattgtataataacaaagttaacgatctttatttttctgtttttttgtagaatgaagttcactgccaccttcctgatgttgttcatcttcgtcctcatggttgaacctggagagtgtggatggaaaaaatggtttaatagggctaagaaaggtagagtcacggaattaattagctttttacattgcaaatagattttttataacagctggaaaatcacaaaaataaatagtcgatatatttggccaattagaatcactttaatttcaataatctaaataacaacctaaaaggcctttgattagcatgtttcttcaatgaaatggacattgaggtttattttgattctcacatgcaccgacctgtgcggcaaccattgaattcagatttgtcccagaagaattcaaagtacatttttccaggcgattaaatctttccattactcagattcaaaaataaataaatagaataattgaagcactatgataaaataattacacattcactctgattttacaagtcaagattgaacactattaaaaactgtgtatagaacatcatctgtatgtgtaattgtttaactgttaatagtcctaataattgttttatggaaatgtattaatttacatttaatattatttgcttgagtttaccatcatgtggttttgtttgtttttacacagttggcaagactgttggcggcttggccgttgagtaaggacttctaccatcattactgtataattttgatagtattatcaccagtactgttattaactacttctcttgtctcgctgactctctccatccgactcctctgcagtcattaccttggcaagcagccggagctcgacaagcgcgcagtcgatgaggaccccagtgctattgtctttgactgaagaagtcgccttgaaggagccttcagaa

SEQ ID NO: 92 - DNA, 999 bp, Hippoglossoides platessoides
ttgcccactttgtattcgcaaggtaagatcaatatttttcaaattcatttagacgagaccaaccatttgggaaatgtgctcagcttgttactgtataatgcaaaagttaagtatctttatttttctgtttttttttgtagaatgaagttcactgccaacttcctcatgttgttcatcttcgtcctcatgtttgaacctggagagtgtggttggcgaacattgcttaaaaaagctggtcacggaattaatacgctttttacattgcaaatagattttttataacagctggaaaatgacaaaaataaatagtcgatatatttggccaattagaattattttgatttcaataataatctaaataacaacctaaaaggtctttgattagcatgtttcttcaatgaaatggacattgaggtttattttgattctcacatgaccgacctgctgcggcaacaattgaattcagatttgtcccagaagaattcaaagtaaattttccaggggattaaatctttccattactcggatttaaaaaaaaaaaaaatagaataactgaattgccatgaaaaaataattacacatactgtctgattttacaagtcaagattgaacactacttaaaagtatgtataaaacatcatctgtatgtataattgtttaactgttaacaaatagtccaaataattgtgttatggaaatgtattaattgtcattaaatataatttgcttgagtttatcatcatgtgttttttttttttttttacacagaggttaagactgttggcaagttggcccttaagtaaggacttctaccatcattactgtataattttgatagtattatcaccagtactgtagtactgacaacttctctctccacccaactcatccgcagacattaccttggcaagcagccggagctcgacaagcgcgcaattgatgacgaccccagtattattgtttttgactgaagaagtcgccttgaaggagccttcagaa

SEQ ID NO: 93 - DNA, 179 bp, Glyptocephalus cynoglossus
atgaagttcactgccaccttcctcatgatgttcatggtcgtcctcatggctgaacccggagaggctggttggggaagtattttcaaacatattttcaaagctggaaagttcatccatggtgcgatccaggcacacaatgacggcgaggagcaggatctcgacaagcgcgcagtcgatga

SEQ ID NO: 94 - DNA, 224 bp, Glyptocephalus cynoglossus
atgaagttcactgccaccttcctggtgttgttcatggtcgtcctcatggctgaacctggagagggtttttggggaaagcttttgaaattgggcatgcatggaatcgggctgctccatcagcatttgggtgctgacgagcagcaggagctcgacgagcgctcagaggaggacgagcccaatgttattgtttttgaatgaagaagtcgcattgaaggagccttcag

SEQ ID NO: 95 - DNA, 862 bp, Glyptocephalus cynoglossus
atgaagttcactgccaccttcctggtgttgttcatggtcgtcctcatggctggatccggagagtgtggttggaaaaagtggctccgtaaaggtagagtcatggatttaatttgctttttacattgcaaatactttaatataacatagttggaaaaccacaaaaataagtagtcgatatatttggccatatagaatcactttgatttcaataataatcaaaacaacaatcaaaaagcccattgattagcatgtcccttcactaaaatggacattgtaatttattttgattctcacaggcaccaacctgctgcggcaacaattgaaatcaaatttgtctcagaagaattcaaagtacattgttctaggcgatttaatctttccattcatcggatctgtttttaaaaatatagaataactggatctctatgttaaaataataaaacacacattctgattttacctgtcaagattgaacacgacttaaaagtatgtataaaacatcatctgtatgtataattgtttaactgtcaactaatagtccaaataattgtgttatggaaatgtattcattgtcatataatatcatttgcttgaatttatcaccatgtgtttttgtttgtttttacacaggtgccaagcaccttggccaggcggccattaagtaaggacttctaccatcattactgtgtaattttaacagtattatcatcagtactgttattgacaactactcttgtctctgttactctctccaggggtttggcctcttgcgaagagcagcaggagctcgacaagcgctcaatggatgacgagcccagtgctattgtttttgactgaagaagtcgccttgaaggagccttca

SEQ ID NO: 96 - DNA, 849 bp, Glyptocephalus cynoglossus
atgaagttcactgccaccttcctggtgttgttcatggtcgtcctcatggctggatccggagagtgtggttggaaaaagtggttcactaaaggtagagtcatggatttaatttgctttttacattgcaaatactttaatataacatagctggaaaatcacaaaaataagtagtcgatatatttggccatatagaatcactttgatttcaataataatcaaaacaataatcaaaaagcctattgattagcatgttccttcactaaaatggacattgtaatttattttgattctcacaggcaccaacctgctgtggcaacaattgaaatcaaatttgtctcagaagaattcaaagtacattgttctaggcgatttaatctttccattcatcggatttgttttcaaaaatatagaataactggatctctatgttaaaataataaaacacattctgattttatctgtcaagattgaacacgacttaaaagtatgaataaaacatcatctgtatgtataattttttaactgtcaactaatagtccaaataattgtgttatggaaatgtattcattgtcatataatatcatttgcttgaatttatcaccatgtgtctttgtttgtttttacacaggtgaaaggttatcccagaggtaaggacttctaccatcattactgtataattttaatagtattatcatcagtactgttattgataacttctcttgtctcgctgactctctccatcaggcatttcgctgacgtcgagcagcaggagctcgacaagcgctcagtggatgacgagcccagttctattgcttttgactgaagaagtcgccttgaaggagccttcag

SEQ ID NO: 97 - DNA, 678 bp, Hippoglossus hippoglossus
ttatgaagttcactgccaccttcctggtgttgttcatggtcgtcctcatggctgagcctggagagtgttttttgggattgctttttcacggggtccaccatggtagggtcacggaagtaattcgatttttacatggcaaatattttaagataacacaccatatgagtagtcgatatatttgaccaattagaatcactttaatttcaataataatcacaataacaatctctaggccatttaatctttccattaatcggatttgtttttttaaatatagaataactggatctctatgttaaaataataaaacatacattctgattttaccagtcaagattgtacgctacttaaaagtatgtataaaacatcatctgtatgtataattgtttaactgttaactaatagtccaaataattgtgtaatggaaatgtattaattgtcatttaatatcatttgcttgaatttatcaccatgtgtttttgtttgtttttacacagttggaaagtggatccatgggtaaggacttctaccatcattactgtgtatttttaatagtattatcatcagtactgttattgatattttctcttgtctcgctgactctctccatcagactcatccatgggcatcacggttacgacgagcagcaggagctcgacaagcgcgcagtcgatgaaa

SEQ ID NO: 98 - DNA, 690 bp, Hippoglossus hippoglossus
ttatgaagttcactgccaccttcctggtgttgttcatggtcgtcctcatggctgaacctggagagggttttttgggaattctttttcacggggtccaccatggtagagtcacggaattaattcgatttttacatggcaaatattttaagataacacaccatatgagtagtcgatatatttgaccaattagaatcactttaatttcaataataatcacaataacaatctctaggccatttaatctttccattaatcggatttgtttttttaaatatagaataactggatctctatgttaaaataataaaacatacattctgattttaccagtcaagattgaacactacttaaaagtatgtataaaacatcatctgtatgtataattgtttaactgttaacaatagtccaaataattgtgttatggaaatgtattaattgtcatttaatatcatttgcttgaatttatcaccatgagttttttgtttgtttttacacaggtagaaagaaggccttgcagtaaggacttctaccatcattactttgtaatttttatagtattatcatcagtactgttattgacaacttctcttgtctcgctgactctctccatcaggatgaactcagagcgtcgcagttacgacgagcggcagcagcagcagcaggagctcgacaagcgcgcagtcgatgaaa

SEQ ID NO: 99 - DNA, 847 bp, Pleuronectes ferruginea
gcccactttgtattcgcaaggtaagatcgatatttttcaaactcatttagacgagaccaagcatttgttgaaatgtgataagcttctaactttataatgcaaatgttaacaatctttttgttctgttgtttttgtaggatgaagttggctgccgccttcctggtgctgttcctggtcgtcctcatggctgaacctggagagggtttcttgggatttctttttcacggtatccaccatggtaaagtcactcatttaatacatttttacatggcaaatatttgaatataacatactatatgagttgtcaatatatgtggccaagtagaagcactttgatttcaataataatcaaaataacaatcactaagccatttaataattgaattaattacatttgttttaaaaaaatatagaataactggatctttatgctaaaataattaaacctaaattcagattttaccactcaagattgaacactacttaaaagtatgtaaaaaaaacatcatctgtatgtataattaaatactagtccagttaattgttttatggaaatgtgttaattgacatatatcatttgcttgaacttataatgtgctttgtttgtttttacacaggtatcagggcgatccatcagtaaggacttctaccatcatgactgtgtatttttaatagtattatcatcagtacttttattaacaacttctcttgtctcgctgactctctccatcagtctcatccatggtcaaagatacgacgagcagcaggagcttgacaagcgctcagtcgatgacaaccccggtgctattgtttttgactgaagacgtcgccttgaaggagccttcag

SEQ ID NO: 100 - DNA, 191 bp, Pleuronectes ferruginea
atgaagttcactgccaccttcctggtgttgtccatggtcgtcctcatggctgaacctggagagggtttctttggagcccttatcaaaggggccatccatggtggcaagttgctccataaactcatcaaaaaaaaacatgaacatcacggttatggcaagcattgggggcttgacaagcgcgcagtcgatga

SEQ ID NO: 101 - DNA, 1100 bp, Pleuronectes americanus
ttgaaagtgaggaagtgagaggaggactaggtcctgtgttttcagtcgttgaattatctaacactatctgagcccctcctgcaataactctaaatgttacacagtgactaggaagtcagtcctgtgtatataaagagttgcatctgttgttatcagtagacaacagattacacctttgaatctcacaaagctcattttgtattcgacaggtaagatcgatatgtttcaaactcatttagatgagaccaagcatttgggaaatgtgctcagcttctaactgtatgatgcaaatgttaacaatctttttgttctgttgttttgtaggatgaagttggctgccgccttcctggtgctgttcctggtcgtcctcatggctgaacctggagagagttttttgggatttctttttcatggtatccgccatggtagggtcactgaattgatacatttttacatggcaaatatttgaatgtaacatactatatgagttgtcaatatatgtggccaagtagaagcactttgatttcagtaataatcaaaataacaatcactaggccatttaataattgcattaattacacttgtttttatatagaatatagaataactggatctttatgctaaaattaataaacatgaattcagattttaagatttttcaagattgaaaactacttaaaagtatgtaaaaaaacatcatctgtatgtataattaaatacttgtccagataattgtgttgtggaaatgtgttaattgacatatatcatttgcttgaatttatcattatctgctttgtttgtttttacacaggtatcaaggcgatccatgggtaaggacttctaccttcatgactgtgtatttttaatagtattatattcagtactgttattgaaaacttctcttgtctcgctgactctctccatcagaatgatccatggtaacagtttagacgagatgcaggagctcgacaagcgctcattcgatgacaaccccaacgcaattgtttttgactgaagaagtcgccctgaaggagccttcagatgatatataatgcttcttgcttttcaatgaaataaattgaataattacccgcaacagc

SEQ ID NO: 102 - DNA, 1300 bp, Pleuronectes americanus
tacttttatctaccactatgtgagctcctcctgttataactctaaatgttacacaatgaagatgaggtcaattctgtgtatataaagagttgcctctgtatagtagacaacatatttcacctttgaatcccacaaagctcactttgtactcaacaggtaagatcgatatttaaaaactaatttagacgaaaccaagcattttggggaatttgctcaacttctaaatgtatgatacaaatgttaacaatcttttatttctgttgttgttttttgtaggatgaagttcactgccaccctcctcctgttgttcatcttcgtcctcatggttgatctcggagagggtcgtcgtaagaaaaaggggtcgaagagaaaggggtccaagggaaaggggtccaagggaaagggcaggtggttggacaggattggtaaaggtagagtcacggaattaatttgctttttacattgcaaatatttttcatataacattgctggaaaatcacaaaaataagtagtcaatatatttggccaaatagaatcactttgatttcaataataatcaaaataacaacctaaaaggcctttgattagcatgttccttcaatgaaatggacattgtaatttactttgattctcacatgctacgacctgctgcagcaacatttgaaaataaatttgtcccagaagattttaaagtacattgttataggcgatttatctttctattactcagatatttgttcaaaccaatagaataactggatctctatgctaaaataataaaacacacattcagatgttaccagtcaagattgaacgctgtttaaaagtaagtatgaaacatcctctgtatgtataattgtttaactggtaacttatagtcctaataattgcgttatggaaatgtattaattgtcatttaatataatttgctggaatttatcactgtgtgtttttgtttgtttttacacagctggcgggataattatcgggggggcccttgagtaaggacttctaccatcattactgtgtaatatttatagttatgatcagtacagttattaacaacttctcttgtctcgctgaacttctccatcagtcacctcgggcaggggcaggtgcaggggccggattacgactaccaggagggggaggagctcaacaagcgctcagacgatgatgacagccccagtcttattttttttgactgaagaagtcgccctgaaggagccttcagatgatatataatgcttctggcttttcattgaaataaataatacgtttacctgcaacagcaaccatg

SEQ ID NO: 103 - DNA, 824 bp, Hippoglossus hippoglossus
ttatgaagttcactgccaccttcctggtgttgttcatggtcgtcctcatggctgaacctggagagggtttgggaaattggatggggccccatatcagcggtagagtcacggaattaatttgctttttccattgcaaatattttaatattgcatagctggaaaatcacgaaataagtagtcgatatatttggccaaatagaatcactttgatttcaataataatcaaaataacaatcaaaaaggcctttgattagcatgttccttcaataaaatggacattgaagtttattttgatgctcacatgcaccgacctgctgcggcaacaattgaaatcaaatttgtctcagaatttaaagtacatttttctaggtgatttaatctttccattaacttgatttgtttttataaatatagaataactggatctttatgccaaaataataaaacacacattctgattttaccagtcaagattgaacactacttaaaagtaatataaaacatcatctgtatgtataattgtttaactgttaacaaaagtccaaataattgtgttatggaaatgtattaattgtcatttaatatcatttgcttgaattcatcaccatgtgttttttgtttgtttttacacaggtgaaaagaaggccttgcagtaaggacttctaccatcattactttgtaatttttatagtattatcatcagtactgttattgacaacttctcttgtctcgctgactctctccatcaggatgaactcagagcgtcgcagttacgacgagcggcagcagcagcagcaggagctcgacaagcgcgcagtcgatga

SEQ ID NO: 104 - DNA, 193 bp, Hippoglossus hippoglossus
atgaagttcactgccaccttcctggtgttgttcatggtcgtcctcatggctgaacctggagagggtttgggaaattggatcgtgcgccctatcggaggtgaaaagaaggccttgcagatgaactcagagcgtcgcagttacgacgagcggcagcagcagcagcaggagctcgacaagcgcgcagtcgatgaaa

SEQ ID NO: 105 - DNA, 281 bp, Hippoglossus hippoglossus
atgaagttcactgccaccttcctggtgttgttcatggtcgtcctcatagctgaacctggagagagtctttttggaaagttcctcaagaaagttgtccatgctggcacgtcaattggcgagacagccttgcatgtcgccgcagagcatcacgggcttcatgcgcatcacgggtgtcacgggcgtcacgggggtcacaggcgtcacgggggtcacaggcgtcacgggcgtcgcggttacgacgagcagcagcaggaggagctcgacaagcgcgcattcgatga

SEQ ID NO: 106 - DNA, 194 bp, Hippoglossus hippoglossus
tatgaagttcactgccaccttcctggtgttgttcatggtcgtcctcatggctgaacctggagagggtttgggaaattggatggggccccatatcagcggtagaaagaaggccttgcacatgaactcagagcgtcgcagttacgacgagcggcagcagcagcagcaggagctcgacaagcgcgcagtcgatgaaa

SEQ ID NO: 107 - DNA, 669 bp, Hippoglossus hippoglossus
atgaagttcactgccaccttcctggtgttgttcatggtcgtcctcatggctgaacctggagagtgttttttgggattgctttttcacggggtccaccatggtagggtcacggaagtaattcgatttttacatggcaaatattttaagataacacaccatatgagtagtcgatatatttggccaattagaatcactttgatttcaataataatcaaaataacaatctctaggcgatttaatatttgcattaattggatttgtttttaaaaatatagaataactggatctttatggtaaaataattaaacatacattctgattttaccagtcaagattgaacactacttagaagtatgtataaaacatcatctgtatgtataattgtttaactgttaacgaatagtccaaataattgtgttatggaaatgtattaattgtcatttaatatcatttgcttgaatttatcaccatgtgtttttgtttgtttttacacagttggaaagttgatccatgggtaaggacttctaccatcattactgtgtatttttaatagtattatcatcagtactattattgacaacttctcttgtctcgctgactctctccatcagactcatccatggcggttacgacgagcagcaggagctcgacaagcgcgcagtcgatgaa

SEQ ID NO: 108 - DNA, 1006 bp, Glyptocephalus cynoglossus
gcccactttgtattcgcaaggtaagagcgatatatttcaaattcattcggatgagaccaagcatttgggaaatgtgctcagcttgttactgtttaatgcaaatgttaacaatatcctttttctgttgtttttgtagaatgaagttcgctgccgccttcctcatgatgttcatggtcgtcctcatggctgaacccggagaggctcgttggggaacgttcttcaaacatattttcaaaggtagagtcacagaattaatttgctttttacattgcaaatattttcatataacatagctggaaaatcacaaaaataagggcttgatatatttggcaaagtagaatccctttgatttcaataataatcaaaataaaaatcagaaaggcctttgattagcatgttccttcaataaaatggacattgtagtttattttgattctcaaatgcaccaacctgctgcggcaacaattgaaatcaaatttgtctccgaaacatttaaagtacatttttcgaggcaatttaatctttcctttgatcgaattcgtttttaaaaatatagaataactggatctttatgctaaaataataaatcatacattctgattttaccagtcaagattgaacgctacttaaaagtatgtataaaacatcatctgtatgtataattgtttaacttttaactaatagtcctaataattgtgttatggaaatgtattcattgtcatttaatatcatttgcttgaatttatcaccatgtgtttttgtttgtttttacacagctggaaggttcatccatgggtaaggacttctaccatcattactgtgtatttttaatagtattatcatcagtactgttattgataacttctcttgtctcgctgactctctccatcagtgcgatccaggcacacaatgacggcgagcagcaggatctcgacaagcgctcagtggatgatgagcccagtgttattgtttttgaatgaagaagtcgccttgaaggagccttcag

SEQ ID NO: 109 - DNA, 1007 bp, Glyptocephalus cynoglossus
gcccactttgtattcgcaaggtaagagcaatatatttcaaattcatttagacgagaccaagcatttgggatctgtgctcaacttgtaactgtataatgcaaatgttaacaatattctttttctgttgtttttgtagaatgaagttcgctgccgccttcctcatgatgttcatggtcgtcctcatggctgaacccggagagggtgcttggatacctgccttgaataggatctatcatggtagagtcacagagttaatttgctttttacattgcaaatattttaatataacatggctggaaaatcacaaaaatgagtactcgatatatttggcaaagtagaatccctttgatttcaataataatcaaaaacacaatcaaaaaggccattgattagcatgttccttcaatgaaatggacattgtagtttattttgattctgacatgcaccaacttgctgcggcaacaattgaattcaaatttgtctcagaaaaatttaaagtacatttttctttccattagtcggatttgttttaaaaaatacagaataactggatctttatgctaaaataataaatcatacattctgattttaccagtcaagattgaacgctacttaaaagtatgtataaaacatcatctgtattgataattgtttaacttttaactaatagtcctaataattgtgttatggaaatgtattcattgtcatttaatatcatttgcttgaatttatcaccatgtgtttttgtttgtttttacacagctctactgaggatcaatcggtaaggacttctaccatcattactgtgtaattttaatagtattatcatcagtactgttattgataacttctcttgtcttgctggctctctccatcagccaaatggtgtattatcgtcggcactggcacggtgacgtcgagcagcaggctctcgacaagcgctcagtggaggaccagcccagttctattgcttctgcctgaagaagtcgccttgaaggagccttcag

SEQ ID NO: 110 - DNA, 1007 bp, Glyptocephalus cynoglossus
gcccactttgtattcgcaaggtaagagcaatatatttcaaattcatttagacgagaccaagcatttgggatctgtgctcaacttgtaactgtataatgcaaatgttaacaatattcttcttctgttgtttttgtagaatgaagttcgctgccgccttcctcatgatgttcatggtcgtcctcatggctgaacccggagagggtgcttggatgcctgccttgaataggatctatcatggtagagtcacagagttaatttgctttttacattgcaaatattttaatataacatggctggaaaatcacaaaaatgagtactcgatatatttggcaaagtagaatccctttgatttcaataataatcaaaaacacaatcaaaaaggccattgattagcatgttccttcaatgaaatggacattgtagtttattttgattctgacatgcaccaacttgctgcggcaacaattgaattcaaatttgtctcagaaaaatttaaagtacatttttctttccattaatcggatttgttttaaaaaatacagaataactggatctttatgctaaaataataaatcatacattctgattttaccagtcaagattgaacgctacttaaaagtatgtataaaacatcatctgtattgataattgtttaacttttaactaatagtcctaataattgtgttatggaaatgtattcattgtcatttaatatcatttgcttgaatttatcaccatgtgtttttgtttgtttttacacagctctactgaggatcaatcggtaaggacttctaccatcattactgtgtaattttaatagtattatcatcagtactgttattgataacttctcttgtcttgctgactctctccatcagccaaatggtgtattatcgtaggcactggcacggtgacgtcgagcagcaggctctcgacaagcgctcagtggaggaccagcccagttctattgcttctgcctgaagaagtcgccttgaaggagccttcag

SEQ ID NO: 111 - DNA, 201 bp, Glyptocephalus cynoglossus
atgaagttcactgccaccttcctggtgttgttcatggtcgtcctcatggctggatccggagagtgtggttggaaaaagtggttcactaaaggtgccaagcaccttggccaggcggccattaacggtttggcctcttgcgaagagcagcaagagctcgacaagcgctcagaggatgacgagcccagtgctattgtttttgaa

SEQ ID NO: 112 - DNA, 862 bp, Glyptocephalus cynoglossus
atgaagttcactgccaccttcctggtgttgttcatggtcgtcctcatggctggatccggagagtgtggttggaaaaagtggctccgtaaaggtagagtcatggatttaatttgctttttacattgcaaatactttaatataacatagttggaaaatcacaaaaataagtagtcgatatatttggccatatagaatcactttgatttcaataataatcaaaacaacaatcaaaaagcccattgattagcatgttccttcactaaaatggacattgtcatttattttgattctcacaggcaccaacctgctgcggcaacaattgaaatcaaatttgtctcagaagaattcaaagtacattgttctaggcgatttaatctttccattcatcggatttgtttttaaaaatatagaataactggatctctatgttaaaataataaaacacacattctgattttacctgtcaagattgaacacgacttaaaagtatgtataaaacatcatctgtatgtataattgtttaactgtcaactaatagtccaaataattgtgttatggaaatgtattcattgtcatataatatcatttgcttgaatttatcaccatgtgtttttgtttgtttttacacaggtgccaagcaccttggccaggcggccattaagtaaggacttctaccatcattactgtgtaattttaacagtattatcatcagtactgttattgacaactactcttgtctctgtgactctctccaggggtttggcctcttgcgaagagcagcaggagctcgacaagcgctcaatggatgacgagcccagtgctattgtttttgactgaagaagtcgccttgaagagccttcag

SEQ ID NO: 113 - DNA, 782 bp, Glyptocephalus cynoglossus
gcccactttgtattcgcaaggtaagagcgatatatttcaaactcatatagacgagaccaagcatttgggaaatgtgctcagcttgttactgtataatgcaaatgttaacaatgtttttgttctgttgtttttgcagaatgaagctcgctgctgccttcctggtgttgttcatggtcgtcctcatggctgaacatggagagggttttggggatttctatatgaagcctggtagagtcacggaattaattcgattttaacatggcaaatattttactataacataccatatgagtagtcgattaattaattggatttgtttttaaaaatatagaataattggatctttatgctaaaataattaaacatacattctgattttaccagttaagattgaacgctacttaaaagtatgtataaaacatcatctgtacatataattgtttaactgttaaccaatagtccaaataattgtgttgtggaaatgtattaattgtcatttaatatcatttgcttgaatttgtcaccatgtgttgttgtttgtttttacacaggtagaaagatttcccatgggtaaggacttctaccatcattactgtgtatttttagcagtattatcatcagtactgttattgataacttctcttgtctcgctgactctctacaggtacatcagaagtccttatggttacgacgagcagcaggaggtcgacaagcgctcagtcgatgacaaccccagtgccattgcttctgactgaagaagtcgccttgaaggagccttcaga

SEQ ID NO: 114 - DNA, 185 bp, Glyptocephalus cynoglossus
atgaagttcactgccaccttcctggtgttgttcatggtcgtcctcatggctgaacctggcgagggttattggcgcttccgcaaccaccgtggtgaaaggttatcccagaggcatttcgctgacgtcgagcagcaggagctcgacaagcgctcagtggatgacgagcccagttctattgcttttga

SEQ ID NO: 115 - DNA, 837 bp, Glyptocephalus cynoglossus
atgaagttcactgccaccttcctcgtgttgttcatcgtcatgtttgaacctggagagtgtttttggaatgctttttcaccgggtccaccatggtcgggtcacggaagtagttcgatttttacatggcaaatatttaaatgaaacataccatatgagtagtcgatatatttggccaagtagaatcactttgacttcaataataatcaaaaacataatcaaaaagcccattgattagcatgttccttcaatgaaatggacattgaggtttattttgattctcacaggcaccaacctgctgcggcaacaattgcattcaaatttgtcccaaagaaacttaattaacattttctggcgatttaatctttgcataaattggatttgtttttaaaaatatagaataactggatctttatgctcaaataattaatcatacattcttattttatcagtcaagattgaacgctacttaaaagtatgtataaaacatcatctgtatgtataattgtttaacttttaactaaaagtcctaataattgtgttatggaaatgtattaattgtcatttaatatcatttccttgaatttatcaccatgtgtttttgtttggtttttacacagctggaaggttgatccataggtaaggacttctaccatcattactgtataatgttaataatagcattatcatcagtactgttattgataacttctcttgtctcgctgactctctccatcagattcatcaaacgtcacggtgacgtcgagcagcaggagctcgacaagcgctcagtggatgacgagcccagttctattgcttttgcctgaagaagtcgccttg

SEQ ID NO: 116 - DNA, 748 bp, Glyptocephalus cynoglossus
atgaagttcactgccaccttcctggtgttgttcatggtcgtcctcatggctgaacctggagactgtatttttggattgattgcgactgcggtccacaatggtaagtcaaggaattaattcgatttttacgtggcaaatattttagtataacataccttatgagtagtcgatatatttgaccaagtagaatcattttgacttcaataataatcaaaataacaatctctaggcaatttaatatttgcattaattggatttgtttttaaaaatatagaataactggatcttaatgctaaaataattaaacatacattctgatattaccagtcaagattgaacgctacttaaaagtatgtataaaacatcatctgtatgtataattgtttaactgtcgactaatagtcctaataattgtgttatggaaatgtattcattgtcatataatatcatttgcttgaatttatcaccatgtgtttttgtttgtttttacacagctggaaggttgatccataggtaaggacttctaccatcattactgtataattttaagagcattatcatcagtactgttattgataacttctcttgtctcgctgactctctccatcagactactcggctttcatcatgggcctcccgggttctggcacggtgacgtcgagcagcaggagctcgacaagcgctcagtggatgaggagcccagttctattgcttttgactgaagaagtcgccttgaaggagccttcag

SEQ ID NO: 117 - DNA, 748 bp, Glyptocephalus cynoglossus
atgaagttcactgccaccttcctggtgttgttcatggtcgtcctcatggctgaacctggagactgtatttttggattgattgcgactgcggtccacaatggtaagtcaaggaattaattcgatttttacttggcaaatattttagtataacataccttatgagtagtcgatatatttgaccaagcagaatcattttgatttcaataataatcaaaataacaatctctaggcaatttaatatttgcattaattggatttgtttttaaaaatatagaataactggatcttaatgctaaaataattaaacatacattctgatattaccagtcaagattgaacgctacttaaaagtatgtataaaacatcatctgtatgtataattgtttaactgtcgactaatagtcctaataattgtgttatggaaatgtattcattgtcatataatatcatttgcttgaatttatcaccatgtgtttttgtttgtttttacacagttggaaggttggtccatgggtaaggacttctaccatcattactgtataattttaagagcattatcatcagtactgttattgataacttctcttgtctcgctgactctctccatcagactactcggctttcatcatgggcctcccgggttctggcacggtgacgtcgtgcagcaggagctcgacaagcgctcagtggatgaggagcccagtgctattgtttttgaatgaagaagtcgccttgaaggagccttcag

SEQ ID NO: 118 - DNA, 748 bp, Glyptocephalus cynoglossus
atgaagttcactgccaccttcctggtgttgttcatggtcgtcctcatggctgaacctggagactgtatttttggattgattgcgactgcggtccacaatggtaagtcaaggaattaattcgatttttacgtggcaaatattttagtataacataccttatgagtagtcgatatatttgaccaagtagaatcattttggtttcaataataatcaaaataacaatctctaggcaatttaatatttgcattaattggatttgtttttaaaaatatagaataactggatcttaatgctaaaataattaaacatacattctgatattaccagtcaagattgaacgctacttaaaagtatgtataaaacatcatctgtatgtataattgtttaactgtcgactaatagtcctaataattgtgttatggaaatgtattcattgtcatataatatcatttgcttgaatttatcaccatgtgtttttgtttgtttttacacagttggaaggttggtccatgggtaaggacttctaccatcattactgtataattttaagagcattatcatcagtactgttattgataacttctcttgtctcgctgactctctccatcagactactcggctttcatcatgggcctcccaggttctggcacggtgacgtcgagcagcaggagctcgacaagcgctcagtggatgaggagcccagtgctattgtttttgaatgaagaagtcgccttgaaggagccttcag

SEQ ID NO: 119 - DNA, 802 bp, Eopsetta jordani
atgaagttcactgccaccttcctcgtgttgttcatggtcatcgtcatgtttgaacctggagagtgtttttttggaatgcgttttcacggggtccaccatggtagggtcacaaaagtgatttgattattacatgccaaatatgttaatgaaacataccatatgagcagtcgtattatttggacaagtagaatcactttgatttcaatagtaattaaaataacaatcaaaaaggcctttgattagcatgttccttcaatgaaatggacattgaggtttattttgattctcacctgcatcgacctgctgcggcaactattgaaatcaaatttgtcccagaagaaactaaattaacattttctaggccatctaatctttgcatgaattggatttgctttcaaaaatatagaataactggatatttatgctaaaataataaaaacacacattctgattttaccagtcaagattgaacactacttaaaagtacgtataaaacatcatctgtatgtataattgtttgacttttaacaaatagtcaaaatgattgttatggaaatgcattaattgtcatttaatatcatttacttgaatttatcaccatgtgtttgtttgttttttagcaggtggaggttttctcaatgcgcaaggacttctaccatcattactgtgtaattttaatagtattatcatcagtactcttattgacaacgtctcttgtctcgctgactctctctatcagattaaacccagggtatcgcggttacgacgagcagcaggagctcgacaagcgcgcagtcgatga

SEQ ID NO: 120 - DNA, 661 bp, Eopsetta jordani
atgaagttcactgccaccttcctggtgttgtccttggtcgtcctcatggctgaacctggagagggtttctttggagcccttctcaaaggtagagtcacggaattaatttgattgttacatggcaaataattttgtataacatatcatatgagcagtcgatgtatttgaccaagaagaatcattttgatttcaataataatcaaaataacaatctcttggagattatatatttgcaataattggattttataaaatatagaacaactggatcttaatgctaaaataattaaacatacattctgattttaccagtcaaaattaaccactactttaaagtatgtataaaacatcatctgtatgtttaattgtttaacttttaacaaatagtccaaataattgtgtaatggaaatgtattcattgtcatataatatagtttgcttgactttatcaccgtgtgtttttgtttgttttttcacaggtgcccaggcgctccatgggtaaggacttctaccatcatgactgtgtaagtttaataatattatcatcagtactgttattaacgacttctcttgtctcgctgactctctccatcagaatcatccacaatgctcgtcacggttacgacgagcagcaggaactcaacaagcgcgcagtcgatga

SEQ ID NO: 121 - DNA, 1011 bp, Eopsetta jordani
gcccactttgtattcgcaaggtaagatcaatatttttcaaattcatttagacgagaccaaccgtttgcgaaatgtgctcagcttgttattgtataataacaaagttaacgatctttatttttctgtttttttgtagaatgaagttcactgccaccttcctgatgttgttcatcttcgtcctcatggttgaacctggagagtgtggttggaaagattggtttcgtaaggctaagaaaggtagaatcacggaattaattagctttttacattgcaaatagattttttataacagctggaaatcacaaaaataaatagtcgatatatttggccaattagaatcactttaatttcaataataatctaaataacaacctaaaaggcctttgattagcatgttccttcaatgaaaaggacattgaggtttattttgattctcacatgcaccgacctgtgcggcaacaattgaattcagatttgtcccagaagaattcaaagtacatttttccaggcgattaaatctttccattactcggatttaaaaataaataaatagaataactgaagcgctatgataaaataattacacattcattctgattttacaagtcaagattgaacactattaaaaagtgtgtataaaacatcatctgtatgtataattgtttaactgttaatagtcttaataattgtgttatggaaatgtattaatttacatttaatatcatttgcttgagtttaccatcatgtgtttttgtttgtttttacacagttggcaagactgttggcggcttggcccttaagtaagaacttctaccatcattactgtataattttgatagtattatcaccagtactgttattaactacttctcttgtctcgctgactctctccatccgactcatccgcagtcattaccttggcgagcagcaggagcttgccaagcgcgcagtcgatgacgaccccagtgttattgtctttgactgaagaagtcgccttgaaggagccttcag

SEQ ID NO: 122 - DNA, 826 bp, Pleuronectes vetulus
atgaagttcactgccaccttcctcatgattttaatcttcgtcctcatggtcgaacctggagagtgtggtattaggaaatggtttaaaaaggctgctcacggtaaagtcacggaattaatttgctttttgctttacaaatatttttttatagcagctggaaaatcacaaaaataaatagtcgatgtatttggccaattagaatcactttgatttcaaataataatctaaatagcaacctaaaaggcctttgattagcatgttccttcaatgaaatggatgttgaggtttattttgattctcacatgcaccgacctgctgcggcaacaattgaattcaaatttgtcccaaaggaattcaaagtaaacttttctagatgatttaatctttccataactcggctttgtttttaaaaatatataataactcaatcactatgataaaataataacacatacattctgatttatacaagacaagattgaaaacttcttaaaagtatgtataaaacatcatctgtttgtataattgtttatcatttcacaaaaagtccaactaattgtgttatggaattgtataaattgtcatttaatataatttttttgagtttatcaatatgtgtttttgtttgttttacacagttggcaaggaagttggcaaggtggcccttaagtaaggacttctaccattattactgtataattttgatagtattatcacccgtactgttattgacaacttctcttttcctgctgactctctccatctgactcatctgcagtgcttgccttgacaagcagcagcagctcgacaagcgcgcagtcgatga

SEQ ID NO: 123 - DNA, 1017 bp, Pleuronectes vetulus
gcccactttgtattcgcaaggtaatatcgatatttttcaaactcatttagacgagaccaagcatttgggaaatgtgctaaggttgttactgtataatgcaaaattaatgatctttatttttctgtttttttttgcagaatgaagttcactgccaccttcctcatgattttaatcttcgtcctcatggtcgaacctggagagtgtggtttgaagaaatggtttaaaaaggctgttcacggtagagtcacggaattaatttgctttttgctttacaaatatttttttatagcagctggaaaatcacaaaaataaatagtcgatgtatttggccaattagaatcactttgatttcaataataatctaaatagcaacctaaaaggcctttgattagcatgttccttcaatgaaatggatgttgaggtttattttgattctcacatgcaccgacctgctgcggcaacaattgaattccaatttgtcccaaaggaattcaaagtaaacttttctaggcgatttaatctttccataactcggctttgtttttaaaaatatataataactcaatccctatgataaaataataacacatacattctgatttatacaagacaagattgaaaacttcttgaaagtatgtatcaaacatcatctgtttgtataattgtttaacagttcacaaaaagtccaactaattgtgttatggaattgtataaattgtcatttaatataatttttttgagtttatcaatatgtgtttttgtttgttttacacagttggcaagaaagttggcaaggtggcccttaagtaaggacttctaccattattactgtgtaattttgatagtattatcaccagtactgttattgacaacttctcttttcctgctgactctctccatccgactcatctgcagtgcttaccttggcgagcagcagcagctcgacaagcgtgcagtcgatgaagagcccagtgttattgcttttgactgaagaagtcgccttgaaggagccttcag

SEQ ID NO: 124 - DNA, 814 bp, Platichthys stellatus
atgaagttcactgccaccttcctcatgatgttcatcttcgtcctcatggttgaacctggagagtgtggttggaggaaatggattaaaaaggctactcacggtaaagtcacggaattaattcgttttttgctttgcaaatattttttttataacagctggaaagtcacaaaaataaatagtcaatatatttggccaattagaatcactttgagttcaataataatctaaataacaaccaaaaaggcctttcctttaatgaaatgtacgttgaagtttattttgaatctcacatgcaccgacctgctgcggcaacaattgaattcaaatttctcccagaggaattcaaagtaaatttttctaggcgatttaatctttccattactctgatttgttttaaatatatagaatgactcaattgctatgataaaataataagccatacattctgatttttacaagacaagattgaaaacttcttaaaagtacgtataaaacatcatctgtatttataattgtttaacatttaacaaattgtcctactaattgtgttatggaaatgtataaattgtcatttaatatcatttgcttgagtttatcattatttgtttttgtttgtttttacacagttggcaagcatattggcaaggcggcccttgagtaagaacttctaccatcattactgtataattttgatagtattatcaccagtactgttattgacaacttctcttgtcctgatgactctgttcatccaactcatctgcagtgcttacattggcgggaagcaagaactcgacaagcgcgcagtcgatga

SEQ ID NO: 125 - DNA, 670 bp, Reinhardtius hippoglossoides
atgaagttcactgccaccttcctggtgttgttcatggtcgtcctcatggctgaacctggagagggttttttcggattgctttttcacgggatccaccatggtagggtcacggaattaattagatgtttacatggcaaatattttaagataacacaccatatgagtagtcgatatatttgaccaattagaatcactttaatttcaataataatcacaataacaatctctaggccatttaatctttccattaatcggatttgtttttttaaatatagaataactggatctttatgctaaaataatgaaacatacattctgattttaccagtcaagattgaacgttacttaaaagtatgtttaaaacatcatctgtatgtataattgtttagctgtaaacaaatagtccaaataattgtgttatggaaatgtattaattgtcatataatataatttgcttgaatttatcaccatgtgtttttgtttgttttttaacacagctggaaagttgatccatgggtaaggacttctaccatcattactgtgtatttttaatagtattatcatcagtactgttattaacaacttctcttctatcgctgactctctccatcagactcatccatcatggttacgacgagcagcaggagctcgacaagcgcgcagtcgatga

SEQ ID NO: 126 - DNA, 813 bp, Hippoglossus stenolepis
atgaagttcactgccaccttcctggtgttgttcatggtcgtcctcatggctgaacctggagagggtttgggaaattggatggggccccatatcagcggtagagtcacggaattaatttgctttttccattgcaaatattttaatattgcatagctggaaaatcacgaaataagtagtcgatatatttggccaaatagaataactttgatttcaataataatcaaaattacaatcaaaaaggcctttgattagcatgttccttcaataaaatggacattgaagtttattttgatgctcacatgcaccgacctgctgcggcaacaattgaaatcaaatttgtctcagaatttaaagtacatttttctaggtgatttaatctttccattcatctgatttattttataaatatagaataactggatctttctgctaaaataataaaacacacattctgattttaccagtcaagattgaacactacttaaaagtatgtataaaacatcatctgtatgtataattgtttaactgttaacaatagtccaaataattgtgttaaggaaatgtattaattgtcatttaatatcatttgcttgaatttatcaccatgagttttttgtttgtttttacacaggtagaaagaaggccttgcagtaaggacttctaccatcattactttgtaatttttatagtattatcatcagtactgttattgacaacttctcttgtctcgctgactctctccatcaggatgaactcagagcgtcgcagttacgacgagtagcagcagaagctcgacaagcgcgcagtcgatga

SEQ ID NO: 127 - DNA, 668 bp, Hippoglossus stenolepis
atgaagttcactgccaccttcctggtgttgttcatggtcgtcctcatggctgaacctggagagtgttttttgggattgctttttcacggggtccaccatggtagggtcacggaagtaattcgatttttacatggcaaatattttaagataacacaccatatgagtagtcgatatatttgatatattagaatcactttgatttcaataataatcaaaataacaatctctaggcgatttaatatttgcattaattggatttgtttttaaaaatatagaataactggatctttatggtaaaataattaaacatacattctgattttaccagtcaagattgaacactacttagaagtatgtataaaacatcatctgtatgtataattgtttaactgttaactaatagtccaaataattgtgttatggaaatgtattaattgtcatttaatatcatttgcttgaatttatcaccatgtgtttttgtttgtttttacacagttggaaatttgatccatgggtaaggacttctaccatcattactgtgtatttttaatagtattatcatcagtactgttattgacaacttctcttgtctcgctgactctctccatcagactcatccatcacggttacgacgagcagcaggagctcgacaagcgcgcagtcgatga

SEQ ID NO: 128 - DNA, 1015 bp, Pleuronichthys coenosus
gcccactttgtattcgcaaggtaatatcgatatttttcaaactcatttagacgagaccaggcatttgggaaacgtgctaaggttgttactgtataatgcaaaattaatgatctttatttttctgtttttttttgcagaatgaagttcactgccaccttcctcatgattttaatcttcgtcctcatggtcgaacctggagagtgtggtattaggaaatggtttaaaaaggctgctcacggtaaagtcacggaattaatttgctttttgctttacaaatatttttttacagcagctggaaaatcacaaaaataaatagtcgatgtatttggccaattagaatcactttgatttcaataataatctaaatagcaacctaaaaggcctttgattagcatgttccttcaatgaaatgggtgttgaggtttattttgattctcacatgcaccgacctgctgcggcaacaattgaattcaaatttgtcccaaaggaattcaaagtaaacttttctaggcgatttaatctttccataactcggctttgtttttaaaaatatataataactcaatcgctatgataaaataataacacatacattctgatttatacaagacaagattgaaaacttcttgaaagtatgtatcaaacatcatctgtttatataattgtttaacatttcacaaaaagtccaactaattgtgttatggaattgtataaattgtcatttaatataatttttttgagtttatcaatatgtgtttttgtttgttttacacagttggcaagaaagttggcaaggtggcccttaagtaaggacttctaccattattactgtataattttgatagtattatcaccagtactgttattgacaacttctcttttcctgctgactctctccatccgactcatctgcagtgcttaccttggcgagcagcagcagctcgacaagcgtgcagtcgatgaagagcccagtgttattgcttttgactgaaggagtcgccttgaaggagccttc

SEQ ID NO: 129 - DNA, 1019 bp, Unknown Organism
  Description of Unknown Organism Nucleotide sequence of pleurocidin-like gene
gcccactttgtattcgcaaggtaatatcgatatttttcaaactcatttagacgagaccaagcatttgggaaacgtgctaaggttgttactgtataatgcaaaattaatgatctttatttttctgtttttttttgcagaatgaagttcactgccaccttcctcatgattttaatcttcgtcctcatggtcgaacctggagagtgtggtattaggaaatggtttaaaaaggctgctcacggtaaagtcacggaattaatttgctttttgctttacaaaatatttttttatagcagctggaaaatcacaaaaataaatagtcgatgtatttggccaattagaatcactttgatttcaataataatctaaatagcaacctaaaaggcctttgattagcatgttccttcaatgaaatggatgttgaggtttattttgattctcacatgcaccgacctgctgcggcaacaattgaattcaaatttgtcccaaaggaattcaaagtaaacttttctaggcgatttaatctttccataactcgggctttgtttttaaaaatatataataactcaatccctatgataaaataataacacatacattctgatttatacaagacaagattgaaaacttcttgaaagtatgtatcaaacatcatctgtttgtataattgtttaacatttcacaaaaagtccaactagttgtgttatggaattgtataaattgtcatttaatataatttttttgagtttatcaatatgtgtttttgtttgttttacacagttggcaagaaagttggcaaggtggcccttaagtaaggacttctaccattattactgtataattttgatagtattatcaccagtactgttattgacaacttctcttttcctgctgactctctccatccgactcatctgcagtgcttaccttggcgagcagcagcagctcgacaagcgtgcagtcgatgaagagcccagtgttattgcttttgactgaagaagtcgccttgaaggagccttcag

SEQ ID NO: 130 - DNA, 832 bp, Unknown Organism
  Description of Unknown Organism Nucleotide sequence of pleurocidin-like gene
aatgaagttcactgccaccttcctcatagaatggttcatcttcgtcctcaatgggttgaaacctgaagaagtgtggttggaaagaaagtggtttaaaaaggctactcacggtaaagtcacggaattaattagcatttttctttgcaaatattttttttatacagctcgaaaattcacaaaaataaatagtcgatatatttggccaattagaatcactttgatttcaataataatctaaataacaacctaaaaggcctttgattagcatgttccttcaatgaaatggacgttgaggtttatattgattctcacatgcaccgacctgctgcgtcaacaattgaattcaaatttgagaggaattcagcgtaaatttttctaggcgatttaatctttccattactcggatttgtttttaaatatatagaataactcaattgctatgataaaataataacacatacattcagatttttacaagacaagattgaaaacttcttaaaggtacgtataaaacatcatctgtatttataattgtttaacatttaacaaataatcctactaattgtgttatggaaatgtataaattgtaatttaatataatttgctttagtttatcattatttgtttttgtttgtttttacacagttggcaagcatgttggcaaggcggcccttgagtaagaacttctaccatcattactgtataattttgatagtgttatcaccagtactgttattgacaacttctcttgtcctgctgactctctccatccgactcatccgcagtgcttacctcggcgagaagcaagaactcgacaagcgcgcagtcgatg

SEQ ID NO: 131 - DNA, 670 bp, Unknown Organism; identical to SEQ ID NO: 125
  Description of Unknown Organism Nucleotide sequence of pleurocidin-like gene

SEQ ID NO: 132 - DNA, 813 bp, Unknown Organism; identical to SEQ ID NO: 126
  Description of Unknown Organism Nucleotide sequence of pleurocidin-like gene

SEQ ID NO: 133 - DNA, 668 bp, Unknown Organism; identical to SEQ ID NO: 127
  Description of Unknown Organism Nucleotide sequence of pleurocidin-like gene

SEQ ID NO: 134 - DNA, 1015 bp, Unknown Organism; identical to SEQ ID NO: 128
  Description of Unknown Organism Nucleotide sequence of pleurocidin-like gene

SEQ ID NO: 135 - DNA, 557 bp, Unknown Organism
  Description of Unknown Organism Nucleotide sequence for hepcidin-like gene
cgcccttaagatgaagacattcagtgttgcagttgcagtggtggtcgtcctcgcatgtatgttcatccttgaaagcaccgctgttcctttctccgaggtgcgaacggaggaggttgaaagcattgacagtccagttggggaacatcaacagccgggcggcacgtccatgaatctgccggtacgttcaatttagtgaatgaattaagtaattacctttagcaaattaacatctaagtggttgcgtttcacccttggaattgaattagcccactagcgctagttgttaaccatttgattgtgagccggtagagagggcttcagggcgagtagtgtgaatacttgtgaagtggagacttggacaaaaatacttaccatgtgcttgttcccacctttttcattttcttttcttggctgagatacagatgcatttcaggttcaagcgtcagagccacctctccctgtgccgttggtgctgcaactgctgtcacaacaagggctgtggcttctgctgcaaattctgaggacctgccagcaaagggcgaattcgtttaaaacac

SEQ ID NO: 136 - DNA, 282 bp, Unknown Organism
  Description of Unknown Organism Nucleotide sequence for hepcidin-like gene
agatgaagacattcagtgttgcagttgcagtggtggtcgtcctcgcatgtatgttcatccttgaaagcaccgctgttcctttctccgaggtgcgaacggaggaggttgaaagcattgacagtccagttggggaacatcaacagccgggcggcacgtccatgaatctgccgatgcatttcaggttcaagcgtcagagccacctctccctgtgccgttggtgctgcaactgctgtcacaacaagggctgtggcttctgctgcaaattctgaggacctgccagca

SEQ ID NO: 137 - DNA, 623 bp, Unknown Organism
  Description of Unknown Organism Nucleotide sequence for hepcidin-like gene
acgaggtccctcatccgctgacaccaaaagaacaatcaatcaactttggactcgtcttagtgcattgaaaattgtgcgttggagagcgtcgctttttgggaacattgaagagttctgatcttcctcataaactgtcacttcaatttcaactgatttcaacaggacttttaaataggctataaacttcctaaaaaaaacgagaatgaaggcctttagtgttgcagtggtactcgtcattgcatgtatgttcatccttgaaagcaccgctgttcctttctccgaggtgcgaacggaggaggttggaagctttgacagtccagttggggaacatcaacagccgggcggcgagtccatgcatctgccggagcctttcaggttcaagcgtcagatccacctctccctgtgcggtttgtgctgcaactgctgtcacaacattggctgtggcttctgctgcaaattctaaggacctgcccgcaacattttctagtttgtacatgtttgcaatgttttctttctgagatgttgtttttgtgactatgataatgatttataaaatcacttcttattgtgacactttaaaaaaaataaacacattctttgaatacaaaaaaaaaaaaaaaaaa

SEQ ID NO: 138 - DNA, 312 bp, Unknown Organism
  Description of Unknown Organism Nucleotide sequence for hepcidin-like gene
cgaacggaggaggttgaaagcattgacagtccagttggggaacatcaacagccgggcggcacgtccatgaatctgccgatgcatttcaggttcaaacgtcagagccacctctccctgtgccgttggtgctgcaactgctgtcacaacaagggctgtggcttctgctgcaaattctgaggacctgccagcactaaagccattttattaacttatcgcctttaatttgcccctattcttctatgtttcttttggactctgtggagaagatgcaatctcattgacgtctttatcactgcacaacctcaatcttgt

SEQ ID NO: 139 - DNA, 277 bp, Unknown Organism
  Description of Unknown Organism Nucleotide sequence for hepcidin-like gene
aagatgaagacattcagtgttgcagtggtacccgtcattgcatgtatgttcatccttgaaagcaccgctgttcctttctccgaggtgcgaacggaggaggttggaagctttgacagtccagttggggaacatcaacagccgggcggcacgtccatgaatctgccgatgcatttcaggttcaagcgtcagagccacctctccctgtgccgttggtgcttcaactgctgtcacaacaaaggctgtggcttctgctgcaaattctgaggacctgccagca

SEQ ID NO: 140 - DNA, 276 bp, Unknown Organism
  Description of Unknown Organism Nucleotide sequence for hepcidin-like gene
taagatgaagcaattcagtgtggcagtggtactcgtcatggcatgtatgttcatcgtggaaagcaccgctgttcctttctccgaggtgcgaacggaggaggttggaagcttggacagtccagttggggaacatcaacagccgggcggcgagtccatgcatctgccggagcctttcaggttcaagcgtcagatccacctctccctgtgcggtttgtgctgcaactgctgtcacaacattggctgtggcttctgctgcaaattctgagactgccagca

SEQ ID NO: 141 - DNA, 647 bp, Unknown Organism
  Description of Unknown Organism Nucleotide sequence for hepcidin-like gene
acgaggcacacgctgaccagggggtcaccacaacttctgaagagacccaggttcctagagagccactagagaatcacccgggagcccgaagaacacaggacgctgcggtgctcgtcggtggccggacacccatgagacagaagacctacaagcctctcagcttcagaaggatttcctgactcagcatctaaaacctccctcaaaatgaaggcattcagcattgcagttgcagtgacactcgtgctcgcctttgtttgcattcagtgcagctctgccgtcccattccaaggggtgcaggagctggaggaggccgggggcaatgacactccagttgcggaacatcaagtgatgtcaatggaatcctggatggagaatcccaccaggcagaagcgccacatcagccacatctccctgtgccgctggtgctgcaactgctgcaaggccaacaagggctgtggcttctgctgcaagttctgaggattcccgcaacacaacctcacaatgtattaatttattacactttttgtcgagaaatgtcctttttcttgacctcttttgtaattttgtataatcttttaaataaaacggggtacgattcatggaaaaaaccctttgaataaaataaaaaaaaaaaaaaaaaaaaaaac

SEQ ID NO: 142 - DNA, 521 bp, Unknown Organism
  Description of Unknown Organism Nucleotide sequence for hepcidin-like gene
aagatgaagacattcagtgttgcagttgcagtgacactcgtgctcgcctttgtttgcattcaggacagctctgccgtcccattccagggggtaagaacgcaactttaactcgcttcatttgcttattagccataaatgttttgtcaggatgctgagacacggctcctaaatgtgtataattcattaacaggtgcaggagctggaggaggcagggggcaatgacactccagttgcggcacatcaaatgatgtcaatggaatcgtggatggtatgttcaatctgttcaatcgactggatgaattaagccaattactgtgagcgcgttaacatttaagtggctgtgttccagcccggtgctgtagggaataaaacccctcgttcatgtgtcttgtccgtccacaggagagtcccgtcaggcagaagcgtcacatcagccacatctccatgtgccgctggtgctgcaactgctgcaaggccaagggctgtggcccctgctgcaaattctgaggacctgcccagca

SEQ ID NO: 143 - DNA, 543 bp, Unknown Organism
  Description of Unknown Organism Nucleotide sequence for hepcidin-like gene
aagatgaagacattcagtgttgcagtcacagtggccgtcgtgctcgtctttatttgtatccagcagagctctggcacctttcctgaggtaagctcctgacttcagatcgtttcattttgcttgttatccatgaatctctcatcaacagactgagacttgattccttctttatcaggtacaagagctggaggaggcagtgagcaatgacaatgcagctgctgaacatcaggagacatcagtggactcatggatggtaggttcagttcactgaatggatcaaaccaattcacatcagacctttcagatggaagtgaatgtgttttagtctcaaaggtgccctgaagctcagtttacacaagcagtgaaaacaaacacagaaagttatgatgatgctgatgaacttctcctcatgtctcatgtctctcacacagatgccatacaacagacagaagcgtgccttcaagtgtaagttctgctgcggctgctgcagagctggtgtctgtggactgtgctgcaagttctgaggattcctgctccaacaac

SEQ ID NO: 144 - DNA, 581 bp, Unknown Organism
  Description of Unknown Organism Nucleotide sequence for hepcidin-like gene
acgagctgacaggagctgacaggagtcaccagcagagtcaaagaactaaacaacttaactcagtcaaactctcaaagatgaagacattcagtgttgcagtcacagtggccgtcgtcctcgtctttatttgtatccagcagagctctgcctcctttcctgaggcacaagagctggaggaggcagtgagcaatgacaatgcagctgctgagcatcaggagacaccagtggactcgtggatgatgccatacaacagacagaagcgtagctttaagtgtaagttctgctgcggctgctgcagagctggtgtctgtggactgtgctgcaagttctgaggattcctgctccaacaaccatcaaatattcatttgttttgccttttgtcttaaagttcattgaactataaacatatttctggttgagcatgtgatagtttaatggtgttactcattggttcatggtatagtcaagtgttcagagatgtgattgtatcacccacatattttctctgttaggtgtattttcaataaatgccaatgatcctttgaaaaaaaaaaaaaaaaaaaaaaaaaaaaaaaaaaaaa

SEQ ID NO: 145 - DNA, 579 bp, Unknown Organism
  Description of Unknown Organism Nucleotide sequence for hepcidin-like gene
acgagcggcacgaggtgaactgacaggagctgacaggagtcaccagcagagtcaaagaactaaacaacttaactcagtcaaactctcaaagatgaagacattcagtgttgcagtcacagtggccgtcgtgctcgtctttatttgtatccagcagagctctgcctcctttcctgaggcacaagagctggaggaggcagtgagcaatgacaatgcagctgctgaacatcaggagacaccagttgactcgtggatgatgccaaacaacagacagaagcgtggctttaagtgtaagttctgctgcggctgctgcagagctggtgtctgtggactgtgctgcaagttctgaggattcctgctccaacaaccatcaaatattcatttgttttgccttttgttttaaagttcattgaactatatacatatttctggtagagcatgtgatagtttaatggtgctactccttggttcatggtgtagttaagtgttcagagatgtgattgtatcacccacatatttctctgttaaggtgtattttcaataaatgttaatgctcctttgaaaaaaaaaaaaaaaaaaaaa

SEQ ID NO: 146 - DNA, 477 bp, Unknown Organism
  Description of Unknown Organism Nucleotide sequence for hepcidin-like gene
acgagactgacaggagctgacaggagtcaccagcagagtcaaagaactaaacaacttaactcagtcaaactctcaaagatgaagacattcagtgttgcagtcacagtggccgtcgtgctcgtctttatttgtatccagcagagctctgccacctttcctgagatgccatacaacagacagaagcgtggctttaagtgtaagttctgctgcggctgctgcggagctggtgtctgtggaatgtgctgcaagttctgaggattcctgctccaacaaccatcaaatattcatttgttttgccttttgtcttaaagttcattgaactataaacatatttctggttgagcatgtgatagtttaatggtgttactcattggttcatggtatagtcaagtgttcagagatgtgattgtatcacccacatattttctctgttaggtgtattttcaataaatgccaatgatcctttgaaaaaaaaaa

SEQ ID NO: 147 - DNA, 483 bp, Unknown Organism
  Description of Unknown Organism Nucleotide sequence for hepcidin-like gene
aagatgaagacattcagtgttgcagtcacagtggccgtcgtgctcgtctttatttgtatccagcagagctctgcctcctttcctgaggtaagcacctgacttcagatcgtttcatttgcttgttatccatgaatctctcatcatcatactgagacttgattccttctttatcaggcacaagagctggaggaggcagtgagcaatgacaatgcagctgctgagcatcaggagacaccagtggactccaggagtgaatgtgttttagtcacaaaagtgccctgaagctcagtttacacaagcagagaaaacaaacagagtaagttatgatgatgctgatgaaggtctcctcatgtctcatgtctctcacacagattccatacaacagacagaagcgtagctttaagtgtaagttctgctgcggctgctgcagagctggtgtctgtggactgtgctgcaagttctgaggattcctgctccaacaac

SEQ ID NO: 148 - DNA, 542 bp, Unknown Organism
  Description of Unknown Organism Nucleotide sequence for hepcidin-like gene
agatgaagacatgcagtgttgcagtcacagtggccgtcgtgctcgtctttatttgtatccagcagagctctgcctcctttcctgaggtaagcacctgacttcagatcgtttcatttgcttgttatccatgaatctctcatcatcatactgagacttgattccttctttatcaggtacaagagctggaggaggcagtgagcaatgacaatgcagctgctgaacatcaggagacaccagttgactcgtggatggtaggttcagttcactgaatggatcaatccatttcacatcagatctttcagatggaagtgaatgtgttttagtcacaaaagtgcccctgaagctcagtttacacaagcagagaaaacaaacagagtaagttatgatgatgctgatgaaggtctcctcatgtctcatgtctctcacacagatgccaaacaacagacagaagcgtggctttaagtgtaagttctgctgcggctgctgcagagctggtgtctgtggactgtgctgcaagttctgaggattcctgctccggacaa

SEQ ID NO: 149 - DNA, 536 bp, Unknown Organism
  Description of Unknown Organism Nucleotide sequence for hepcidin-like gene
aagatgaagacaatcagtgttgcagtcacagtggccgtcgtcctcgtctttatttgtatccagcagagctctgcctcctttcctgaggtaagcacctgacttcagatcgtttaatttgcttgttatccatgaatctctcatcaacatactgagacttgattccttctttatcaggcacaagagctggaggaggcagtgagcaatgacaatgcagctgctgagcatcaggagacaccagtggactcagggatggtaggttcagttcactgaatggatcaatccatttcacatcagatctttcagattgaagtgaatgtgttttagtcacaaaagtgccctgaagctcagtttacacaagcagagaaaacaaacagagtaagttatgatgatgctgatgaaggtctcctcatgtctcatgtctctcacacagattccatacaacagacagaagcgtagctttaagtgtaagttctgctgcggctgctgcagagctggtgtctgtggactgtgctgcaaattctgaggacctgccagca

SEQ ID NO: 150 - DNA, 536 bp, Unknown Organism
  Description of Unknown Organism Nucleotide sequence for hepcidin-like gene
aagatgaagacattcagtggtgcagtcacagtggccgtcgtgctcgtctttatttgtatccagcagagctctgcctcctttcctgaggtaagcacctgacttcagatcgtttcatttgcttgttatccatgaatctctcatcatcatactgagacttgattccttctttatcaggtacaagagctggaggaggcagtgagcaatgacaatgcagctgctgaacatcaggagacaccagttgactcgtggatggtaggttcagttcactgaatggatcaatccatttcacatcagatctttcagatggaagtgaatgtgttttagtcacaaaagtgccctgaagctcagtttacacaagcagagaaaacaaacagagtaagttatgatgatgctgatgaaggtctcctcatgtctcatgtctctcacacagatgccaaacaacagacagaagcgtggctttaagtgtaagttctgctgcggctgctgcagagctggtgtctgtggactgtgctgcaaattctgaggacctgccagca

SEQ ID NO: 151 - DNA, 542 bp, Unknown Organism
  Description of Unknown Organism Nucleotide sequence for hepcidin-like gene
aagatgaagacatcagtggttgcagtcacagtggccgtcgtgctcgtctttatttgtatccagcagagctctgccacctttcctgaggtaagcacctgacttcagatagcttcatttgcttgttatccatgaatctctcatcaacatactgagactttattccttctttatcaggtacaagagctggaggaggcagtgagcaatgacaatgcagctgctgcgcatcaggagacatcagtggactcgtggatggtaggttcagttcactcaatggatcaaaccaattcacatcagatctttcagatggaagcgaatgtgttttagtcaaaaaagtgacctgatgctcagtttacacaagcagagaaaacaagcagagtaagttatgatgatgctgatgaacgtgtcctcatgtctcatgtctctcacacagatgccatacaacagaccgaagcgtagctttaagtgtaagttctgctgcggctgctgcagagctggtgtctgtggactgtgctgcaaattctgaggattcctgctccaacaac

SEQ ID NO: 152 - DNA, 542 bp, Unknown Organism
  Description of Unknown Organism Nucleotide sequence for hepcidin-like gene
aagatgaagacattcagtgtggcagtcacagtggccgtcgtgctcgtctttatttgtatccagcagagctctgccacctttcctgaggtaagcacctgacttcagatagcttcatttgcttgttatccatgaatctctcatcaacatactgagacttgatttcttctttatcaggtacaagagctggaggaggcagtgagcaatgacaatgcagccgctgaacatcaggagacatcagtggactcgtggatggtaggttcagttcactcaatggatcaaaccaattcacatcagatctttcagatggaagtgaatgtgttttagtcacagaagtgccctgatgctcagtttacacaagcagagaaaacaagcagagtaagttatgatgatgctgatgaacgtgtcctcatgtctcatgtctctcacacagatgccatacaacagaccgaagcgtagctttaagtgtaagttctgctgcggctgctgtagagctggtgtctgtggactgtgctgcaaattctgaggattcctgctccaacaac

SEQ ID NO: 153 - DNA, 542 bp, Unknown Organism
  Description of Unknown Organism Nucleotide sequence for hepcidin-like gene
aagatgaagacattcgtggttgcagtcacagtggccgtcgtgctcgtctttatttgtatccagcagagctctgccacctttcctgaggtaagcacctgacttcagatagcttcatttgcttgttatccatgaatctctcatcaacatactgagacttgattccttctttatcaggtacaagagctggaggaggcagtgagcaatgacaatgcagccgctgaacatcaggagacatcagtggactcgtggatggtaggttcagttcactcaatggatcaaaccaattcacatcagatctttcagatggaagtgactgtgttttagtcacaaaagtgccctgatgctcagtttacacaagcagagaaaacaagcagagtaagttatgatgatgctgatgaacgtctcctcatgtctcatgtctctcacacagatgccatacaacagacagaagcgtagctttaagtgtaagttctgctgcggctgctgcagagctggtgtctgtggactgtgctgcaaattctgaggattcctgctccaacaac

SEQ ID NO: 154 - DNA, 533 bp, Unknown Organism
  Description of Unknown Organism Nucleotide sequence for hepcidin-like gene
aagatgaagacatcagtggttgcagtcacagtggccgtcgtgctcgtctttatttgtatccagcagagctctgccacctttcctgaggtaagcacctgacttcagatagcttcatttgcttgttatccatgaatctctcatcaacatactgagactttattccttctttatcaggtacaagagctggaggaggcagtgagcaatgacaatgcagctgctgcacatcaggagacatcagtggactcgtggatggtaggttcagttcactcaatggatcaaaccaattcacatcagatctttcagatgaagtgactgtgttttagtcacaaaagtgccctgatgctcagtttacacaagcagagaaaacaagcagagtaagttatgatgatgctgatgaacgtgtcctcatgtctcatgtctctcacacagatgccatacaacagacataagcgtagctttaagtgtaagttctgctgcggctgctgcagagctggtgtctgtggactgtgctgcaaattctgaggattcctgct

SEQ ID NO: 155 - DNA, 541 bp, Unknown Organism
  Description of Unknown Organism Nucleotide sequence for hepcidin-like gene
aagataagacattcagtgttgcagtcacagtggccgtcgtgctcgtctttatttgtatccagcagagctctgccacctttcctgaggtaagcacctgacttcagatcgtttcatttgcttgttagccttgaatctctcatcaacatactgagacttgatttcttctttatcaggtacaagagctggaggaggcagtgagcaatgacaatgcagctgctgaacatcaggagacatcagtggacttgtggatggtaggttcagttcactgaatggatcaaaccaattcacatcagatctttcagatggaagtgaatgtgttttagtcacaaaagtgccctgaagctcagtttacacgagcagagaaaaccaacacagtaagttatgatgatgctgatgaacgtctcctcatgtctcatgtctctcacacagatgccatacaacagacagaagcgtggctttaagtgtaagttctgctgcggctgctgcagccctggtgtctgtggactttgctgcagattctgaggattcctgctccaacaac

SEQ ID NO: 156 - DNA, 536 bp, Unknown Organism
  Description of Unknown Organism Nucleotide sequence for hepcidin-like gene
aagatgaagacattcagtgttgcagtcgcagtggccgtcgtgctcatctttatttgtatccagcagagctctgccacctttcctgaggtaagcacctgacttcagatagtttcatttgcttgttatccatgaatctctcatcaacatactgagactttattccttctttatcaggtacaagagctggaggaggcagtgagcaatgacaatgcagctgctgaacatcaggagacatcattggactcatggatggtaggttcagttcactcaatggatcaaaccaattcacatcagatctttcagatggaagtgactgtgttttagtcacaaaagtgccctgatgctcagtttacacaagcagagaaaacaagcagagtaagttatgatgatgctgatgaacgtgtcctcatgtctcatgtctctcacacagatgccatacaacagacagaagcgtggctttaagtgtaagttctgctgcggctgctgcagagctggtgtctgtggactgtgctgcaaattctgaggacctgccagca

SEQ ID NO: 157 - DNA, 536 bp, Unknown Organism
  Description of Unknown Organism Nucleotide sequence for hepcidin-like gene
aagatgaagacattcagtgttgcagtcacagtggccgtcgtgctcgtctttatttgtatccagcagagctctgccacctttcctgaggtaagcacctgacttcagatagtttcatttgcttgttatccatgaatctctcatcaacatactgagactttattccttctttatcaggtacaagagctggaggaggcagtgagcaatgacaatgcagctgctgaacatcaggagacatcattggactcatggatggtaggttcagttcactcaatggatcaaaccaattcacatcagatctttcagatggaagtgaatgtgttttagtcacaaaagtgccctgatgctcagtttacacaagcagagaaaacaagcagagtaagttatgatgatgctgatgaacgtgtcctcatgtctcatgtctctcacacagatgccatacaacagacataagcgtagctttaagtgtaagttctgctgcggctgctgcagagctggtgtctgtggactgtgctgcaaattctgaggacctgccagca

SEQ ID NO: 158 - DNA, 535 bp, Unknown Organism
  Description of Unknown Organism Nucleotide sequence for hepcidin-like gene
agatgaagacattcagtgttgcagtcacagtggccgtcgtgctcgtctttatttgtatccagcagagctctgccacctttcctgaggtaagcacctgacttcagatagtttcatttgcttgttatccatgaatctctcatcaacatactgagacttgatttcttctttatcaggtacaagagctgggggaggcagtgagcaatgacaatgcagccgctgaacatcaggagacatcagtggactcgtggatggtaggttcagttcactcaatggatcaaaccaattcacatcagatctttcagatggaagtgaatgtgttttagtcacaaaagtgccctgatgctcagtttacacaagcagagaaaacaagcagagtaagttatgatgatgctgatgaacgtgtcctcatgtctcatgtctctcacacagatgccatacaacagaccgaagcgtagctttaagtgtaagttctgctgcggctgctgcagagctggtgtctgtggactgtgctgcaaattctgaggacctgccagca

SEQ ID NO: 159 - DNA, 277 bp, Unknown Organism
  Description of Unknown Organism Nucleotide sequence for hepcidin-like gene
aagatgaagacattcagtgttgcagtcacagtggccgtcgtgctcatctttatttgtatccagcagagctctgccacctctcctgaggtacaagggctggaggaggcagtgagcaatgacaatgcagctgctgaacatcaggagacatcagtggactcgtggatgatgccatacaacagacagaagcgtggctttaagtgtaagttctgctgcggctgctgcaggcctggtgtctgtggactttgctgcagatcctgaggattcctgctccaacaac

SEQ ID NO: 160 - DNA, 542 bp, Unknown Organism
  Description of Unknown Organism Nucleotide sequence for hepcidin-like gene
aagatgaagacattcagtgttgcagtcacagtggccgtcgtgctcgtctttatttgtatccagcagagctctgccacctttcctgaggtaagcacctgacttcagatcgtttcatttgcttgttagccttgaatctctcatcaacatactgagacttgatttcttctttatcaggtacaagagctggaggaggcagtgagcaatgacaatgcagctgctgaacatcaggagacatcagtggacttgtggatggtaggttcagttcactgaatggatcaaaccaattcacatcagatctttcagatggaagtgaatgtgttttagtcacaaaagtgccctgaagctcagtttacacgagcagagaaaaccaacacagtaagttatgatgatgctgatgaacgtctcctcatgtctcatgtctctcacacagatgccatacaacagacagaagcgtggctttaagtgtaagttctgctgcggctgctgcagacctggtgtctgtggactttgctgcagattctgaggattcctgctccaacaac

SEQ ID NO: 161 - DNA, 539 bp, Unknown Organism
  Description of Unknown Organism Nucleotide sequence for hepcidin-like gene
aagatgaagacattcagtgttgcagtcacagtggccgtcgtgctcgtctttatttgtatccagcagagctctgccacctttcctgaggtaagcacctgacttcagatcgtttcatttgcttgttagccttgaatctctcatcaacatactgagacttgatttcttctttatcaggtacaagagctggaggaggcagtgagcaatgacaatgcagctgctgaacatcaggagacatcagtggacttgtggatggtaggttcagttcactgaatggatcaaaccaattcacatcagatctttcagatggaagtgaatgtgttttagtcacaaaagtgccctgaagctcagtttacacgagcagagaaaaccaacacagtaagttatgatgatgctgatgaacgtctcctcatgtctcatgtctctcacacagatgccatacaacagacagaagcgtggctttaagtgtaagttctgctgcggctgctgcagtcctggtgtctgtggactttgctgcagattctgaggattcctgctccaac

SEQ ID NO: 162 - DNA, 536 bp, Unknown Organism
  Description of Unknown Organism Nucleotide sequence for hepcidin-like gene
aagatgaagacattcagtgttgcagtcacagtggccgtcgtgctcgtctttatttgtatccagcagagctctgccacctttcctgaggtaagcacctgacttcagatcgtttcatttgcttgttagccttgaatctctcatcaacatactgagacttgatttcttctttatcaggtacaagagctggaggaggcagtgagcaatgacaatgcagctgctgaacatcaggagacatcagtggactcgtggatggtaggttcagttcactgaatggatcaaaccaattcacatcagatccttcagatggaagtgaatgtgttttagtcacaaaagtgccctgaagctcagtttacacgagcagagaaaacaaacacagtaagttatgatgatgctgatgaacgtctcctcatgtctcatgtctctcacacagatgccatacaacagacagaagcgtggctttaagtgtaagttctgctgcggctgctgcagtcctggtgtctgtggactttgctgcaaattctgaggacctgccagca

SEQ ID NO: 163 - DNA, 536 bp, Unknown Organism
  Description of Unknown Organism Nucleotide sequence for hepcidin-like gene
aagatgaagacattcagtgttgcagtcacagtggccgtcgtgctcgtctttatttgtatccagcagagctctgccacctttcctgaggtaagcacctgacttcagatcgtttcatttgcttgttagccttgaatctctcatcaacatactgagacttgatttcttctttatcaggtacaagagctggaggaggcagtgagcaatgacaatgcagctgctgaacatcaggagacatcagtggactcgtggatggtaggttcagttcactgaatggatcaaaccaattcacatcagatctttcagatggaagtgaatgtgttttagtcacaaaagtgccctgaagctcagtttacacgagcagagaaaacaaacacagtaagttatgatgatgctgatgaacgtctcctcatgtctcatgtctctcacacagatgccatacaacagacagaagcgtggctttaagtgtaagttctgctgcggctgctgcagacctggtgtctgtggactttgctgcaaattctgaggacctgccagca

SEQ ID NO: 164 - DNA, 271 bp, Unknown Organism
  Description of Unknown Organism Nucleotide sequence for hepcidin-like gene
aagatgaagacattcagtgttgcagtcacagtggccgtcgtgctcgtctttatttgtatccagcagagctctgccacctttcctgaggtacaagagctggaggaggcagtgagcaatgacaatgcagctgctgaacatcaggagacatcagtggactcgtggatgatgccatacaacagacagaagcgtggctttaagtgtaagttctgctgcggctgctgcagacctggtgtctgtggactttgctgcaaattctgaggacctgccagca

SEQ ID NO: 165 - DNA, 277 bp, Unknown Organism
  Description of Unknown Organism Nucleotide sequence for hepcidin-like gene
aagatgaagacattcagtgttgcagtcacagtggccgtcgtgctcgtctttatttgtatccagcagagctctgccacctttcctgaggtacaagagctggaggaggcagtgagcaatgacaatgcagctgctgaacatcaggagacatcagtggactcgtggatgatgccatacaacagacagaagcgtggctttaagtgtaagttctgctgcggctgctgcaggcctggtgtctgtggactttgctgcagattctgaggattcctgctccaacaac

SEQ ID NO: 166 - DNA, 499 bp, Unknown Organism
  Description of Unknown Organism Nucleotide sequence for hepcidin-like gene
aagatgaagacattcagtgttgcagtcacagtggccgtcgtgctcgtcttcatttgtatccagcagagctctgccacctttcctgaggtaagcacctgacttcagatcgtttcatttgcttgttagccttgaatctctcatcaacatactgagacttgatttcttctttatcaggtacaagagctggaggaggcagtgagcagtgacaatgcagctgctgaacatcaggagacatcagtggactcgtggatggtaggttcagttcactgaatgtgttttagtcacaaaagtgccctgaagctcagtttacacaagcagagaaaacaaacagagtaagttatgatgatgctgatgaacgtctcctcatgtctcatgtctctcacacagatgccatacaacagacagaagcgtagctttaagtgcaagttctgctgcggctgctgcagacgtggtgtctgtggactgtgctgcaaattctgaggattcctgctccaacaac

SEQ ID NO: 167 - DNA, 549 bp, Unknown Organism
  Description of Unknown Organism Nucleotide sequence for hepcidin-like gene
aagatgaagactatcagtgttgcagtcacagtggccgtcgtgctcctcttcatttgtacccagcagagctctgccacctttcctgaggtaagcacctgacttcagatcgtttcatttgcttgttagccttgaatctctcatcaacatactgagacttgatttcttctttatcaggtacaagagctggaggaggcagtgagcagtgacaatgcggctgctgaacatcaggagacatcagtggactcgtggatggtaggttcagttcactgaatggatcaaaccaattcacatcagatctttcagatggaagtgaatgtgttttagtcacaaaagtgccctgaagctcagtttacacaagcagagaaaacaaacacagtaagttatgatgatgctgatgaacgtctcctcatgtctcatgtctcatgtctctcacacagatgccatacaacagacagaagcgtggctttaagtgcaagttctgctgcggctgccgctgtggtgctctctgtggactgtgctgcaaattctgaggattcctgctccaacaac

SEQ ID NO: 168 - DNA, 543 bp, Unknown Organism
  Description of Unknown Organism Nucleotide sequence for hepcidin-like gene
aagatgaagacattcagtgttgcagtcacagtggccgtcgtgctcgtcttcatttgtatccagcagagctctgccacctttcctgaggtaagcacctgacttcagatcgtttcatttgcttgttagccttgaatctctcatcaacgtactgagacttgatttcttctttatcaggtacaagagctggaggagccagtgagcagtgacaatgcagctgctgaacatcaggagacatcggtggactcgtggatggtaggttcagttcactgaatggatcaaaccaattcacatcagatctttcagatggaagtgaatgtgttttagtcacaaaagtgccctgaagctcagtttacacaagcagagaaaacaaacacagtaagttatgatgatgctgatgaacgtctcctcatgtctcatgtctcatgtctctcacacagatgccatacaacagacagaagcgtggctttaagtgcaagttctgctgcggctgccgctgtggtgctctctgtggactgtgctgcaaattctgaggacctgccagca

SEQ ID NO: 169 - DNA, 542 bp, Unknown Organism
  Description of Unknown Organism Nucleotide sequence for hepcidin-like gene
aagatgaagacattcagtgttgcagtcacagtggccgtcgtgctcgtctttatttgttccagcagagctctgccacctttcctgaggtaagcacctgacttcagatcgtttcatttgcttgttagccttgaatctctcatcaacatactgagacttgatttcttctttatcaggtacaagagctggaggaggcagtgagcagtgacaatgcagctgctgaacatcaggagacatcagtggactcgtggatggtaggttcagttccctgaatggatcaaaccaattcacatcagatctttcagatggaagtgaatgtgttttagtcacaaaagtgccctgaagctcagtttacacaagcagagaaaacaaacacagtaagttatgatgatgctgatgaacatctcctcatgtctcatgtctcatgtctctcacacagatgccatacaacagacagaagcgtggctttaagtgcaagttctgctgcggctgccgctgtggtgctctctgtggactgtgctgcaaattctgaggacctgccagca

SEQ ID NO: 170 - DNA, 655 bp, Unknown Organism
  Description of Unknown Organism Nucleotide sequence for hepcidin-like gene
acgagctgacaggagctgacaggagtcaccagcagagtcaaagaactaaacaacttaactcagtcaaactctcaaagatgaagacattcagtgttgcagtcacagtggccgtcgtgctcgtctttatttgtatccagcagagctctgccacctttcctgaggtacaagagctggaggaggcagtgagcaatgacaatgcagctgctgagcatcaggagacaccagtggactcagggatgatgccaaacaacagacagaagcgcagcgccgattgttggccatgttgcaatcaaaatggctgtggaacttgctgcaaggtctaaacagactcttgggcagatcaatccaggttcgtctttcgttgtctctccgtggagtcgaaccagagaccttctcagcccatagtccaagtttctgccactagaccaccgcctctccctcatcaaatactcaatgtttttcattttgtcttaaagttcattgaactataaacatatttctggtagagcatgtgatagtttaatggtgttactcattggttcatggtatagtcagatgttcagagatgtgattatatcatccacatattttctctgttaaggtgtactgtcaataaatgtcaatgctcctttgaaaaaaaaaaaaaaaaaaaaac

SEQ ID NO: 171 - DNA, 510 bp, Unknown Organism
  Description of Unknown Organism Nucleotide sequence for hepcidin-like gene
cgtgctcgtctttatttgtatccagcagagctctgccacctttcctgaggtgagctcctgacttcagatcgtttcatttagcttgttatccatgaatctctcatcaacatactgagacttgaatccttctttatcaggtacaggagctggaggaggcagtgagcaatgacaatgcagctgctgaacatcaggagacatcagtggactcatggatggtatgttcagttcactgaatggatcaaaccaattcacatcagatctttcagatggaagtgaatttgttttagtcccaaaagtgccctgaagctcagtttacacaagcagagaaaaacaaaacacagtaagttatgatgatgctgatgaacgtctcctcatgtctcatgtctctcacacagatgccatacaacagacagaagcgcagcgccgagtgtagcttctgctgcaatgaatctggctgtggaatttgctgcaaattctgaggattcctgctccaacaacaagggcgaattc

SEQ ID NO: 172 - DNA, 530 bp, Unknown Organism
  Description of Unknown Organism Nucleotide sequence for hepcidin-like gene
aagatgaagacattcagtgttgcagtcacagtggccgtcgtgctcgtctttatttgtatccagcagagctctgccacctttcctgaggtgagctcctgacttcagatcgtttcatttagcttgttatccatgaatctctcatcaacatactgagacttgaatccttctttatcaggtacaggagctggaggaggcagtgagcaatgacaatgcagctgctgaacatcaggagacatcagtggactcatggatggtatgttcagttcactgaatggatcaaaccaattcacatcagatctttcagatggaagtgaatttgttttagtcccaaaagtgccctgaagctcagtttacacaagcagagaaaaacaaaacacagtaagttatgatgatgctgatgaacgtctcctcatgtctcatgtctctcacacagatgccatacaacagacagaagcgcagcgccgagtgtagcttctgctgcaatgaatctggctgtggaatttgctgcaaattctgaggacctgccagca

SEQ ID NO: 173 - DNA, 348 bp, Unknown Organism
  Description of Unknown Organism Nucleotide sequence for hepcidin-like gene
gtggaggagccagtgagcagtgagaatggagcaaatgaacacacataagatctttcggatggaagtgtatgtgttttagtcacatgagtggctcgaagctcagtacacacgagcagagagaacgaacacagtgtgttttattctgcttgtgtaaactgagcttcagtttacacaagcagagaaaacaaacacagtaagttatgatgatgctgatgaacgtctcctcatgtctcatatctctcacacagatgccaaacaacagacagaagcgtggctctaattgcaaaccatgctgcaatcataatggctgtggaacgtgctgcgaagtctgaggattcctgctccaca

SEQ ID NO: 174 - protein, 88 aa, Unknown Organism
  Description of Unknown Organism Hepcidin peptide sequence
MKTFSVAVAVVVVLACMFILESTAVPFSEVRTEEVESIDSPVGEHQQPGGTSMNLPMHFRFKRQSHLSLCRWCCNCCHNKGCGFCCKF

SEQ ID NO: 175 - protein, 88 aa, Unknown Organism; identical to SEQ ID NO: 174
  Description of Unknown Organism Hepcidin peptide sequence

SEQ ID NO: 176 - protein, 86 aa, Unknown Organism
  Description of Unknown Organism Hepcidin peptide sequence
MKAFSVAVVLVIACMFILESTAVPFSEVRTEEVGSFDSPVGEHQQPGGESMHLPEPFRFKRQIHLSLCGLCCNCCHNIGCGFCCKF

SEQ ID NO: 177 - protein, 58 aa, Unknown Organism
  Description of Unknown Organism Hepcidin peptide sequence
RTEEVESIDSPVGEHQQPGGTSMNLPMHFRFKRQSHLSLCRWCCNCCHNKGCGFCCKF

SEQ ID NO: 178 - protein, 86 aa, Unknown Organism
  Description of Unknown Organism Hepcidin peptide sequence
MKTFSVAVVPVIACMFILESTAVPFSEVRTEEVGSFDSPVGEHQQPGGTSMNLPMHFRFKRQSHLSLCRWCFNCCHNKGCGFCCKF

SEQ ID NO: 179 - protein, 86 aa, Unknown Organism
  Description of Unknown Organism Hepcidin peptide sequence
MKQFSVAVVLVMACMFIVESTAVPFSEVRTEEVGSLDSPVGEHQQPGGESMHLPEPFRFKRQIHLSLCGLCCNCCHNIGCGFCCKF

SEQ ID NO: 180 - protein, 90 aa, Unknown Organism
  Description of Unknown Organism Hepcidin peptide sequence
MKAFSIAVAVTLVLAFVCIQCSSAVPFQGVQELEEAGGNDTPVAEHQVMSMESWMENPTRQKRHISHISLCRWCCNCCKANKGCGFCCKF

SEQ ID NO: 181 - protein, 89 aa, Unknown Organism
  Description of Unknown Organism Hepcidin peptide sequence
MKTFSVAVAVTLVLAFVCIQDSSAVPFQGVQELEEAGGNDTPVAAHQMMSMESWMESPVRQKRHISHISMCRWCCNCCKAKGCGPCCKF

SEQ ID NO: 182 - protein, 84 aa, Unknown Organism
  Description of Unknown Organism Hepcidin peptide sequence
MKTFSVAVTVAVVLVFICIQQSSGTFPEVQELEEAVSNDNAAAEHQETSVDSWMMPYNRQKRAFKCKFCCGCCRAGVCGLCCKF

SEQ ID NO: 183 - protein, 84 aa, Unknown Organism
  Description of Unknown Organism Hepcidin peptide sequence
MKTFSVAVTVAVVLVFICIQQSSASFPEAQELEEAVSNDNAAAEHQETPVDSWMMPYNRQKRSFKCKFCCGCCRAGVCGLCCKF

SEQ ID NO: 184 - protein, 84 aa, Unknown Organism
  Description of Unknown Organism Hepcidin peptide sequence
MKTFSVAVTVAVVLVFICIQQSSASFPEAQELEEAVSNDNAAAEHQETPVDSWMMPNNRQKRGFKCKFCCGCCRAGVCGLCCKF

SEQ ID NO: 185 - protein, 58 aa, Unknown Organism
  Description of Unknown Organism Hepcidin peptide sequence
MKTFSVAVTVAVVLVFICIQQSSATFPEMPYNRQKRGFKCKFCCGCCGAGVCGMCCKF

SEQ ID NO: 186 - protein, 83 aa, Unknown Organism
  Description of Unknown Organism Hepcidin peptide sequence
MKTFSVAVTVAVVLVFICIQQSSASFPEAQELEEAVSNDNAAAEHQETPVDSRIPYNRQKRSFKCKFCCGCCRAGVCGLCCKF

SEQ ID NO: 187 - protein, 84 aa, Unknown Organism
  Description of Unknown Organism Hepcidin peptide sequence
MKTCSVAVTVAVVLVFICIQQSSASFPEVQELEEAVSNDNAAAEHQETPVDSWMMPNNRQKRGFKCKFCCGCCRAGVCGLCCKF

SEQ ID NO: 188 - protein, 84 aa, Unknown Organism
  Description of Unknown Organism Hepcidin peptide sequence
MKTISVAVTVAVVLVFICIQQSSASFPEAQELEEAVSNDNAAAEHQETPVDSGMIPYNRQKRSFKCKFCCGCCRAGVCGLCCKF

SEQ ID NO: 189 - protein, 84 aa, Unknown Organism
  Description of Unknown Organism Hepcidin peptide sequence
MKTFSGAVTVAVVLVFICIQQSSASFPEVQELEEAVSNDNAAAEHQETPVDSWMMPNNRQKRGFKCKFCCGCCRAGVCGLCCKF

SEQ ID NO: 190 - protein, 84 aa, Unknown Organism
  Description of Unknown Organism Hepcidin peptide sequence
MKTSVVAVTVAVVLVFICIQQSSATFPEVQELEEAVSNDNAAAAHQETSVDSWMMPYNRPKRSFKCKFCCGCCRAGVCGLCCKF

SEQ ID NO: 191 - protein, 84 aa, Unknown Organism
  Description of Unknown Organism Hepcidin peptide sequence
MKTFSVAVTVAVVLVFICIQQSSATFPEVQELEEAVSNDNAAAEHQETSVDSWMMPYNRPKRSFKCKFCCGCCRAGVCGLCCKF

SEQ ID NO: 192 - protein, 84 aa, Unknown Organism
  Description of Unknown Organism Hepcidin peptide sequence
MKTFVVAVTVAVVLVFICIQQSSATFPEVQELEEAVSNDNAAAEHQETSVDSWMMPYNRQKRSFKCKFCCGCCRAGVCGLCCKF

SEQ ID NO: 193 - protein, 84 aa, Unknown Organism
  Description of Unknown Organism Hepcidin peptide sequence
MKTSVVAVTVAVVLVFICIQQSSATFPEVQELEEAVSNDNAAAAHQETSVDSWMMPYNRQKRSFKCKFCCGCCRAGVCGLCCKF

SEQ ID NO: 194 - protein, 84 aa, Unknown Organism
  Description of Unknown Organism Hepcidin peptide sequence
MKTFSVAVTVAVVLVFICIQQSSATFPEVQELEEAVSNDNAAAEHQETSVDLWMMPYNRQKRGFKCKFCCGCCSPGVCGLCCRF

SEQ ID NO: 195 - protein, 84 aa, Unknown Organism
  Description of Unknown Organism Hepcidin peptide sequence
MKTFSVAVAVAVVLIFICIQQSSATFPEVQELEEAVSNDNAAAEHQETSLDSWMMPYNRQKRGFKCKFCCGCCRAGVCGLCCKF

SEQ ID NO: 196 - protein, 84 aa, Unknown Organism
  Description of Unknown Organism Hepcidin peptide sequence
MKTFSVAVTVAVVLVFICIQQSSATFPEVQELEEAVSNDNAAAEHQETSLDSWMMPYNRHKRSFKCKFCCGCCRAGVCGLCCKF

SEQ ID NO: 197 - protein, 84 aa, Unknown Organism
  Description of Unknown Organism Hepcidin peptide sequence
MKTFSVAVTVAVVLVFICIQQSSATFPEVQELGEAVSNDNAAAEHQETSVDSWMMPYNRPKRSFKCKFCCGCCRAGVCGLCCKF

SEQ ID NO: 198 - protein, 84 aa, Unknown Organism
  Description of Unknown Organism Hepcidin peptide sequence
MKTFSVAVTVAVVLIFICIQQSSATSPEVQGLEEAVSNDNAAAEHQETSVDSWMMPYNRQKRGFKCKFCCGCCRPGVCGLCCRS

SEQ ID NO: 199 - protein, 84 aa, Unknown Organism
  Description of Unknown Organism Hepcidin peptide sequence
MKTFSVAVTVAVVLVFICIQQSSATFPEVQELEEAVSNDNAAAEHQETSVDLWMMPYNRQKRGFKCKFCCGCCRPGVCGLCCRF

SEQ ID NO: 200 - protein, 84 aa, Unknown Organism; identical to SEQ ID NO: 194
  Description of Unknown Organism Hepcidin peptide sequence

SEQ ID NO: 201 - protein, 84 aa, Unknown Organism
  Description of Unknown Organism Hepcidin peptide sequence
MKTFSVAVTVAVVLVFICIQQSSATFPEVQELEEAVSNDNAAAEHQETSVDSWMMPYNRQKRGFKCKFCCGCCSPGVCGLCCKF

SEQ ID NO: 202 - protein, 84 aa, Unknown Organism
  Description of Unknown Organism Hepcidin peptide sequence
MKTFSVAVTVAVVLVFICIQQSSATFPEVQELEEAVSNDNAAAEHQETSVDSWMMPYNRQKRGFKCKFCCGCCRPGVCGLCCKF

SEQ ID NO: 203 - protein, 84 aa, Unknown Organism; identical to SEQ ID NO: 202
  Description of Unknown Organism Hepcidin peptide sequence

SEQ ID NO: 204 - protein, 84 aa, Unknown Organism
  Description of Unknown Organism Hepcidin peptide sequence
MKTFSVAVTVAVVLVFICIQQSSATFPEVQELEEAVSNDNAAAEHQETSVDSWMMPYNRQKRGFKCKFCCGCCRPGVCGLCCRF

SEQ ID NO: 205 - protein, 84 aa, Unknown Organism
  Description of Unknown Organism Hepcidin peptide sequence
MKTFSVAVTVAVVLVFICIQQSSATFPEVQELEEAVSSDNAAAEHQETSVDSWMMPYNRQKRSFKCKFCCGCCRRGVCGLCCKF

SEQ ID NO: 206 - protein, 84 aa, Unknown Organism
  Description of Unknown Organism Hepcidin peptide sequence
MKTISVAVTVAVVLLFICTQQSSATFPEVQELEEAVSSDNAAAEHQETSVDSWMMPYNRQKRGFKCKFCCGCRCGALCGLCCKF

SEQ ID NO: 207 - protein, 84 aa, Unknown Organism
  Description of Unknown Organism Hepcidin peptide sequence
MKTFSVAVTVAVVLVFICIQQSSATFPEVQELEEPVSSDNAAAEHQETSVDSWMMPYNRQKRGFKCKFCCGCRCGALCGLCCKF

SEQ ID NO: 208 - protein, 84 aa, Unknown Organism
  Description of Unknown Organism Hepcidin peptide sequence
MKTFSVAVTVAVVLVFICIQQSSATFPEVQELEEAVSSDNAAAEHQETSVDSWMMPYNRQKRGFKCKFCCGCRCGALCGLCCKF

SEQ ID NO: 209 - protein, 81 aa, Unknown Organism
  Description of Unknown Organism Hepcidin peptide sequence
MKTFSVAVTVAVVLVFICIQQSSATFPEVQELEEAVSNDNAAAEHQETPVDSGMMPNNRQKRSADCWPCCNQNGCGTCCKV

SEQ ID NO: 210 - protein, 81 aa, Unknown Organism
  Description of Unknown Organism Hepcidin peptide sequence
MKTFSVAVTVAVVLVFICIQQSSATFPEVQELEEAVSNDNAAAEHQETSVDSWMMPYNRQKRSAECSFCCNESGCGICCKF

SEQ ID NO: 211 - protein, 81 aa, Unknown Organism; identical to SEQ ID NO: 210
  Description of Unknown Organism Hepcidin peptide sequence

SEQ ID NO: 212 - protein, 88 aa, Hippoglossus hippoglossus; identical to SEQ ID NO: 174

SEQ ID NO: 213 - protein, 88 aa, Salmo salar; identical to SEQ ID NO: 174

SEQ ID NO: 214 - protein, 86 aa, Salmo salar; identical to SEQ ID NO: 176

SEQ ID NO: 215 - protein, 58 aa, Salmo salar; identical to SEQ ID NO: 177

SEQ ID NO: 216 - protein, 86 aa, Salmo salar; identical to SEQ ID NO: 178

SEQ ID NO: 217 - protein, 86 aa, Salmo salar; identical to SEQ ID NO: 179

SEQ ID NO: 218 - protein, 90 aa, Pleuronectes americanus; identical to SEQ ID NO: 180

SEQ ID NO: 219 - protein, 89 aa, Paralichthys olivaceus; identical to SEQ ID NO: 181

SEQ ID NO: 220 - protein, 84 aa, Pleuronectes americanus; identical to SEQ ID NO: 183

SEQ ID NO: 221 - protein, 84 aa, Pleuronectes americanus; identical to SEQ ID NO: 184

SEQ ID NO: 222 - protein, 58 aa, Pleuronectes americanus; identical to SEQ ID NO: 185

SEQ ID NO: 223 - protein, 83 aa, Pleuronectes americanus; identical to SEQ ID NO: 186

SEQ ID NO: 224 - protein, 84 aa, Pleuronectes americanus; identical to SEQ ID NO: 187

SEQ ID NO: 225 - protein, 84 aa, Hippoglossoides platessoides; identical to SEQ ID NO: 190

SEQ ID NO: 226 - protein, 84 aa, Hippoglossoides platessoides; identical to SEQ ID NO: 191

SEQ ID NO: 227 - protein, 84 aa, Hippoglossoides platessoides; identical to SEQ ID NO: 192

SEQ ID NO: 228 - protein, 84 aa, Hippoglossoides platessoides; identical to SEQ ID NO: 193

SEQ ID NO: 229 - protein, 84 aa, Hippoglossoides platessoides; identical to SEQ ID NO: 194

SEQ ID NO: 230 - protein, 84 aa, Hippoglossoides platessoides; identical to SEQ ID NO: 195

SEQ ID NO: 231 - protein, 84 aa, Hippoglossoides platessoides; identical to SEQ ID NO: 196

SEQ ID NO: 232 - protein, 84 aa, Hippoglossoides platessoides; identical to SEQ ID NO: 197

SEQ ID NO: 233 - protein, 84 aa, Salmo salar; identical to SEQ ID NO: 198

SEQ ID NO: 234 - protein, 84 aa, Hippoglossus hippoglossus; identical to SEQ ID NO: 199

SEQ ID NO: 235 - protein, 84 aa, Hippoglossus hippoglossus; identical to SEQ ID NO: 194

SEQ ID NO: 236 - protein, 84 aa, Hippoglossus hippoglossus; identical to SEQ ID NO: 201

SEQ ID NO: 237 - protein, 84 aa, Hippoglossus hippoglossus; identical to SEQ ID NO: 202

SEQ ID NO: 238 - protein, 84 aa, Hippoglossus hippoglossus; identical to SEQ ID NO: 202

SEQ ID NO: 239 - protein, 84 aa, Hippoglossus hippoglossus; identical to SEQ ID NO: 204

SEQ ID NO: 240 - protein, 81 aa, Pleuronectes americanus; identical to SEQ ID NO: 209

SEQ ID NO: 241 - protein, 81 aa, Pleuronectes ferruginea; identical to SEQ ID NO: 210

SEQ ID NO: 242 - protein, 81 aa, Pleuronectes ferruginea; identical to SEQ ID NO: 210

SEQ ID NO: 243 - protein, 27 aa, Glyptocephalus cynoglossus
MPNNRQKRGSNCKPCCNHNGCGTCCEV

SEQ ID NO: 244 - protein, 67 aa, Hippoglossoides platessoides
MKFTATFLMLFIFVLMVEPGECGWKSVFRKAKKVGKTVGGLALDHYLGEQQELDKRAVDEDPSIVFD

SEQ ID NO: 245 - protein, 67 aa, Hippoglossoides platessoides
MKFTATFLMLFIFVLMVEPGECGWKKWFNRAKKVGKTVGGLAVDHYLGKQPELDKRAVDEDPSIVFD

SEQ ID NO: 246 - protein, 66 aa, Hippoglossoides platessoides
MKFTANFLMLFIFVLMFEPGECGWRTLLKKAEVKTVGKLALKHYLGKQPELDKRAIDDDPSIIVFD

SEQ ID NO: 247 - protein, 68 aa, Pleuronectes americanus
MKFTATFLMIAIFVLMVEPGECGWGSFFKKAAHVGKHVGKAALTHYLGDKQELNKRAVDEDPNVIVFE

SEQ ID NO: 248 - protein, 56 aa, Pleuronectes ferruginea
MKFTATFLMMCIFVLMVEPGECRWGKWFKKATHVGKHVGKAALTAYLGDKQELDKR

SEQ ID NO: 249 - protein, 68 aa, Pleuronectes americanus
MKFTATFLMMFIFVLMVEPGECGWGSIFKHGRHAAKHIGHAAVNHYLGEQQDLDKRAVDEDPNVIVFE

SEQ ID NO: 250 - protein, 56 aa, Glyptocephalus cynoglossus
MKFTATFLMMFMVVLMAEPGEAGWGSIFKHIFKAGKFIHGAIQAHNDGEEQDLDKR

SEQ ID NO: 251 - protein, 67 aa, Glyptocephalus cynoglossus
MKFTATFLVLFMVVLMAGSGECGWKKWFTKGAKHLGQAAINGLASCEEQQELDKRSEDDEPSAIVFE

SEQ ID NO: 252 - protein, 67 aa, Glyptocephalus cynoglossus
MKFTATFLVLFMVVLMAGSGECGWKKWLRKGAKHLGQAAIKGLASCEEQQELDKRSMDDEPSAIVFD

SEQ ID NO: 253 - protein, 67 aa, Glyptocephalus cynoglossus; identical to SEQ ID NO: 252

SEQ ID NO: 254 - protein, 67 aa, Glyptocephalus cynoglossus; identical to SEQ ID NO: 251

SEQ ID NO: 255 - protein, 62 aa, Glyptocephalus cynoglossus
MKFTATFLVLFMVVLMAGSGECGWKKWFTKGERLSQRHFADVEQQELDKRSVDDEPSSIAFD

SEQ ID NO: 256 - protein, 62 aa, Glyptocephalus cynoglossus
MKFTATFLVLFMVVLMAEPGEGYWRFRNHRGERLSQRHFADVEQQELDKRSVDDEPSSIAFD

SEQ ID NO: 257 - protein, 65 aa, Glyptocephalus cynoglossus
MKFTATFLVLVIVMFEPGECFGMLFHRVHHAGRLIHRFIKRHGDVEQQELDKRSVDDEPSSIAFA

SEQ ID NO: 258 - protein, 76 aa, Glyptocephalus cynoglossus
MKFTATFLVLFMVVLMAEPGDCIFGLIATAVHNAGRLIHRLLGFHHGPPGFWHGDVEQQELDKRSVDEEPSAIVFE

SEQ ID NO: 259 - protein, 76 aa, Glyptocephalus cynoglossus
MKFTATFLVLFMVVLMAEPGDCIFGLIATAVHNVGRLVHRLLGFHHGPPGFWHGDVEQQELDKRSVDEEPSAIVFE

SEQ ID NO: 260 - protein, 69 aa, Pleuronectes americanus
MKFTATFLVLSLVVLMAEPGECFLGALIKGAIHGGRFIHGMIQNHHGYDEQQELDKRSVDDNPGAIVFD

SEQ ID NO: 261 - protein, 68 aa, Pleuronectes americanus
MKLAAAFLVLFLVVLMAEPGESFLGFLFHGIRHGIKAIHGMIHGNSLDEMQELDKRSFDDNPNAIVFD

SEQ ID NO: 262 - protein, 68 aa, Pleuronectes ferruginea
MKLAAAFLVLFLVVLMAEPGEGFLGFLFHGIHHGIRAIHHLIHGQRYDEQQELDKRSVDDNPGAIVFD

SEQ ID NO: 263 - protein, 55 aa, Hippoglossus hippoglossus
MKFTATFLVLFMVVLMAEPGECFLGLLFHGVHHVGKLIHGLIHGGYDEQQELDKR

SEQ ID NO: 264 - protein, 57 aa, Hippoglossus hippoglossus
MKFTATFLVLFMVVLMAEPGECFLGLLFHGVHHVGKWIHGLIHGHHGYDEQQELDKR

SEQ ID NO: 265 - protein, 61 aa, Hippoglossus hippoglossus
MKFTATFLVLFMVVLMAEPGEGFLGILFHGVHHGRKKALHMNSERRSYDERQQQQQELDKR

SEQ ID NO: 266 - protein, 60 aa, Hippoglossus hippoglossus
MKFTATFLVLFMVVLMAEPGEGLGNWMGPHISGEKKALHMNSERRSYDERQQQQQELDKR

SEQ ID NO: 267 - protein, 43 aa, Glyptocephalus cynoglossus
MKLTATFLVLFMVVLMAEPGEGFWGKLFKLGLHGIGLLHLHLG

SEQ ID NO: 268 - protein, 67 aa, Pleuronectes americanus
MKFATAFLMLSMVVLMAEPGECRSTEDIIKSISGGGFLNAMNAGYNEQQELNKRSDDDDSPSLIVFD

SEQ ID NO: 269 - protein, 68 aa, Pleuronectes americanus
MKFTATFLLLFIFVLMVDLGEGRRKRKWLRRIGKGVKIIGGAALDHLGQGQVQGQDYDYQEGQELNKR

SEQ ID NO: 270 - protein, 98 aa, Pleuronectes americanus
MKFTATLLLLFIFVLMVDLGEGRRKKKGSKRKGSKGKGSKGKGRWLDRIGKAGGIIIGGALDHLGQGQVQGPDYDYQEGEELNKRSDDDDSPSLIFFD

SEQ ID NO: 271 - protein, 85 aa, Pleuronectes americanus
MKFTATFLLLFIFVLMVDLGEGRRKKKGSKRKGSKGKGSKGKGRWLERIGKAGGIIIGGALDHLGQGQVQGPDYDYQEGEELNKR

SEQ ID NO: 272 - DNA, 728 bp, Pleuronectes americanus
  CDS (153)..(252)
  CDS (483)..(513)
  CDS (615)..(687)
gaattcgcccttgcccactttgtattcgcaaggtaatatcaatatttttcaaattcatttagacgagaccaaccttttgggaaatctgctcagcttattactgtataatgcaaatgttaatgatctttatttttctgttttttttttgtagaatgaagttcactgccaccttcctcatgatgttcatcttcgtcctcatggttgaacctggagagtgtggttggggaagcatttttaagcatggtcgtcatggtaaagtcacggaattaattagcttttaactttgcaaatattgtttttttttttaacagctggaaactcacaaaaataaatagccgatatatttggccaattataatcactttgatctaaataacaacctaaaaggcctttgattagcatgtttcttcaataaaatgattgaacactacttaaaggtatgtataaaacatcatcatgtgtttttgtttgtttttacacagctgccaagcatattggccatgcagccgttaagtaaggacttctaccattattactgtataattttgatagtattatcaccagtattgttattgacaacttctctttttcctgctgatccgactcatccgcagtcattaccttggcgagcagcaagatctcgacaagcgcgcagtcgatgaagacccaaatgttattgtttttgaatgaagaaatcgccttgaaggagccttcagaagggcgaattc

SEQ ID NO: 273 - protein, 68 aa, Pleuronectes americanus; identical to SEQ ID NO: 249

SEQ ID NO: 274 - protein, 60 aa, Pleuronectes americanus
MKFTATFLMIAIFVLMVEPGECGWGSFFKKAAHVGKHVGKAALTHYLGDKQELNKRAVDE

SEQ ID NO: 275 - protein, 60 aa, Pleuronectes americanus
MKFTATFLMMFIFVLMVEPGECGWGSIFKHGRHAAKHIGHAAVNHYLGEQQDLDKRAVDE

SEQ ID NO: 276 - protein, 61 aa, Pleuronectes americanus
MKFTATFLVLSLVVLMAEPGECFLGALIKGAIHGGRFIHGMIQNHHGYDEQQELNKRAVDE

SEQ ID NO: 277 - protein, 72 aa, Pleuronectes americanus
MKFTATFLLLFIFVLMVDLGEGRRKRKWLRRIGKGVKIIGGAALDHLGQGQVQGQDYDYQEGQELNKRAVDE

SEQ ID NO: 278 - protein, 89 aa, Pleuronectes americanus
MKFTATFLLLFIFVLMVDLGEGRRKKKGSKRKGSKGKGSKGKGRWLERIGKAGGIIIGGALDHLGQGQVQGPDYDYQEGEELNKRAVDE

SEQ ID NO: 279 - protein, 24 aa, Artificial Sequence
  Description of Artificial Sequence Synthetic peptide
MWKDVLKKIGTVALHAGKAALGAV

SEQ ID NO: 280 - protein, 25 aa, Artificial Sequence
  Description of Artificial Sequence Synthetic peptide
SIGSAFKKALPVAKKIGKAALPIAK

SEQ ID NO: 281 - DNA, 934 bp, Unknown Organism
  CDS (186)..(266)
  CDS (676)..(771)
  Description of Unknown Organism Type 1 salmonid hepcidin sequence
ctgacaccaaaagaacaatcaatcaactttggactcgtcttagtgcattgaaaattgtgcgttggagagcgtcgctttttgggaacattgaagagttctgatcttcctcataaactgtcacttcaatttcaactgatttcaacaggacttttaaataggctataaacttcctaaaaaaaacgagaatgaaggcctttagtgttgcagtggtactcgtcattgcatgtatgttcatccttgaaagcaccgctgttcctttctccgaggtatgtcaaattctccaacaccaacccactacaaacatgtgtgcatcgattttagaggttggtcatgactcatttgtgcctaatgtctttgcaggtgcgaacggaggaggttggaagctttgacagtccagttggggaacatcaacagccgggcggcgagtccatgcatctgccggtacgttcaattgaatgaatgaattacgctaattacctttagcaaattaacattttagtggttgcgttttaccctcggaatagaattagatcagtagcgctagctgttaaccatttgattgtgagccgttagagggcttcagggcgagcagtgtgcaacgtggttgtgaagtggagatatacttacttgcttgttccctccttttttcatattattttcttggcggggatacaggagcctttcaggttcaagcgtcagatccacctctccctgtgcggtttgtgctgcaactgctgtcacaacattggctgtggcttttgctgcaaattctaaggacctgcccgcaacattttctagtttgtacatgtttgcaatgttttctttctgagatgttgtttttgtgactatgataatgatttataaaatcacttcttattgtgacactttaaaaaaaataaacacattctttgaataacaaaaaaaaaaaaaaaaa

SEQ ID NO: 282 - protein, 86 aa, Unknown Organism; identical to SEQ ID NO: 176
  Description of Unknown Organism Type 1 salmonid hepcidin sequence

SEQ ID NO: 283 - DNA, 557 bp, Unknown Organism; identical to SEQ ID NO: 135
  CDS (11)..(178)
  CDS (423)..(518)
  Description of Unknown Organism Type1 Hepcidin sequence

SEQ ID NO: 284 - protein, 88 aa, Unknown Organism; identical to SEQ ID NO: 174
  Description of Unknown Organism Type1 Hepcidin sequence

SEQ ID NO: 285 - protein, 81 aa, Pleuronectes americanus; identical to SEQ ID NO: 209

SEQ ID NO: 286 - protein, 81 aa, Paralichthys olivaceus
MKTFSAAVTVAVVLVFICIQQSSATSPEVQELEEAVSSDNAAAEHQEQSADSWMMPQNRQKRDVKCGFCCKDGGCGVCCNF

SEQ ID NO: 287 - protein, 84 aa, Pleuronectes americanus; identical to SEQ ID NO: 183

SEQ ID NO: 288 - protein, 84 aa, Pleuronectes americanus; identical to SEQ ID NO: 184

SEQ ID NO: 289 - protein, 58 aa, Pleuronectes americanus; identical to SEQ ID NO: 185

SEQ ID NO: 290 - protein, 84 aa, Pleuronectes americanus; identical to SEQ ID NO: 202

SEQ ID NO: 291 - protein, 84 aa, Pleuronectes americanus; identical to SEQ ID NO: 198

SEQ ID NO: 292 - protein, 84 aa, Hippoglossus hippoglossus; identical to SEQ ID NO: 199

SEQ ID NO: 293 - protein, 84 aa, Hippoglossus hippoglossus; identical to SEQ ID NO: 204

SEQ ID NO: 294 - protein, 86 aa, Hippoglossus hippoglossus
MKTCGFAAAVAVLLTFICIQEGCAVSVAEEQVLKDPMGNGDPQEVPAESSGRQWMMPFHFRQRRGSGPMPCRQCCHCCPENGRVYV

SEQ ID NO: 295 - protein, 85 aa, Morone saxatilis
MKTFSVAVAVAVVLAFICLQESSAVPVTEVQELEEPMSNEYQEMPVESWKMPYNNRHKRHSSPGGCRFCCNCCPNMSGCGVCCRF

SEQ ID NO: 296 - protein, 90 aa, Oryzias latipes
MKAFSIAVAVTLVLAFICILQSSAIPVNGVKELEEAASNDTPVAARHEMSMQPWMLPNHIREKRQSHISMCTMCCNCCKNYKGCGFCCRF

SEQ ID NO: 297 - protein, 90 aa, Pleuronectes americanus; identical to SEQ ID NO: 180

SEQ ID NO: 298 - protein, 89 aa, Paralichthys olivaceus
  MOD_RES (85): Variable amino acid
MKAFSIAVAVTLVLAFVCIQDSSAIPFQGVQELEEAGGNDTPVAAHQMMSMESWMESPVRQKRHISHISMCRWCCNCCKAKGCGXCCKF

SEQ ID NO: 299 - protein, 88 aa, Hippoglossus hippoglossus; identical to SEQ ID NO: 174

SEQ ID NO: 300 - protein, 88 aa, Salmo salar; identical to SEQ ID NO: 174

SEQ ID NO: 301 - protein, 86 aa, Salmo salar; identical to SEQ ID NO: 178

SEQ ID NO: 302 - protein, 61 aa, Oncorhynchus mykiss
  MOD_RES (37): Variable amino acid
LQVLTEEVGSIDSPVGEHQQPGGESMRLPEHFRFKRXSHLSLCRWCCNCCHNKGXGFCCKF

SEQ ID NO: 303 - protein, 86 aa, Salmo salar; identical to SEQ ID NO: 179

SEQ ID NO: 304 - protein, 84 aa, Homo sapiens
MALSSQIWAACLLLLLLLASLTSGSVFPQQTGQLAELQPQDRAGARASWMPMFQRRRRRDTHFPICIFCCGCCHRSKCGMCCKT

SEQ ID NO: 305 - protein, 30 aa, Artificial Sequence
  Description of Artificial Sequence Formula peptide
MKFTATFLXXXLLFIFXVLMXVEDPLGECG

SEQ ID NO: 306 - protein, 18 aa, Artificial Sequence
  Description of Artificial Sequence Formula peptide
YXXXEXXXQELXKRXVDX

SEQ ID NO: 307 - protein, 14 aa, Artificial Sequence
  Description of Artificial Sequence Synthetic signal peptide II
MKXXXXAXXVXXVL

SEQ ID NO: 308 - protein, 8 aa, Artificial Sequence
  Description of Artificial Sequence Synthetic signal peptide III
MKTFSVAV

SEQ ID NO: 309 - protein, 24 aa, Artificial Sequence
  Description of Artificial Sequence Synthetic signal peptide IV
MKTFSVAVTVAVVLXFICIQQSSA

SEQ ID NO: 310 - protein, 31 aa, Artificial Sequence
  Description of Artificial Sequence Synthetic signal peptide V
MKTFSVAVAVXXVLAFXXXXXXSXAVPFXXV

SEQ ID NO: 311 - protein, 21 aa, Artificial Sequence
  Description of Artificial Sequence Synthetic prosequence I peptide
PEVQXLEEAXSXDNAAAEHQE

SEQ ID NO: 312 - protein, 22 aa, Artificial Sequence
  Description of Artificial Sequence Synthetic prosequence II peptide
PFXXVXXXEEVXXXDXPVXXHQ

SEQ ID NO: 313 - DNA, 17 bp, Artificial Sequence
  Description of Artificial Sequence Primer
ggattcctgctccaaca

SEQ ID NO: 314 - DNA, 16 bp, Artificial Sequence
  Description of Artificial Sequence Primer
taaggacctgcccgca

SEQ ID NO: 315 - protein, 8 aa, Artificial Sequence
  Description of Artificial Sequence Synthetic peptide
GWXXXFXK

SEQ ID NO: 316 - protein, 15 aa, Artificial Sequence
  Description of Artificial Sequence Synthetic peptide
GXXXXXXXHXGXXIH

SEQ ID NO: 317 - protein, 19 aa, Artificial Sequence
  Description of Artificial Sequence Synthetic peptide
FKCKFCCGCCXXGVCGXCC

SEQ ID NO: 318 - protein, 19 aa, Artificial Sequence
  Description of Artificial Sequence Synthetic peptide
CXXCCNCCXXKGCGFCCKF

SEQ ID NO: 319 - protein, 21 aa, Artificial Sequence
  Description of Artificial Sequence Synthetic peptide
FKCKFCCGCRCGXXCGLCCKF

SEQ ID NO: 320 - protein, 19 aa, Artificial Sequence
  Description of Artificial Sequence Synthetic peptide
XXXCXXCCNXXGCGXCCKX

SEQ ID NO: 321 - protein, 6 aa, Pleuronectes americanus
WMENPT

SEQ ID NO: 322 - protein, 6 aa, Pleuronectes americanus
GCGFCC

SEQ ID NO: 323 - protein, 6 aa, Pleuronectes americanus
GRRKRK

SEQ ID NO: 324 - DNA, 24 bp, Artificial Sequence
  Description of Artificial Sequence Primer
aagatgaagacattcagtgttgca

SEQ ID NO: 325 - DNA, 19 bp, Artificial Sequence
  Description of Artificial Sequence Primer
gttgttggagcaggaatcc

SEQ ID NO: 326 - DNA, 24 bp, Artificial Sequence
  Description of Artificial Sequence Primer
tgctggcaggtcctcagaatttgc

SEQ ID NO: 327 - DNA, 879 bp, Unknown Organism
  Description of Unknown Organism Nucleotide sequence of pleurocidin-like gene
atgaagttcactgccaccttcctcatgattttaatcttcgtcctcatggtcgaacctggagagtgtggttgtaagaaatggtttaaaaaggctgctcacggtagagtcacggaattaatttgctttttgctttacaaatatttttttatagcagctggaaaatcacaaaaataaatagtcgatgtatttggccaattagaatcactttcatttcaataataatctaaatagcaacctaaaaggcctttgattagcatgttccttcaatgaaatggatgttgaggtttattttgattctcacatgcaccgacctgctgcggcaacaattgaattccaatttgtcccaaaggaattcaaagtaaacttttctaggcgatttaatctttccataactcggctttgtttttaaaaatatataataactcaatccctatgataaaataataacacatacattctgatttatacaagacaagattgaaaacttcttgaaagtatgtatcaaacatcatctgtttgtataattgtttaacatttcacaaaaagtccaactaattgtgttatggaattgtataaattgtcatttaatataatttttttgagtttatcaatatgtgtttttgtttgttttacacagttggcaagaacgttggcaaggtggcccttaagtaaggacttctaccattattactgtataattttgatagtattatcaccagtactgttattgacaacttctcttttcctgctgactctctccatccgactcatctgcagtgcttaccttggtgagcagcagcagctcgacaagcgtgcagtcgatgaagagcccagtgttattgcttttgactgaagaagtcgccttgaaggagccttcag

SEQ ID NO: 328 - DNA, 335 bp, Pleuronectes americanus
  CDS (27)..(230)
acaaagcccactttgtattcgcaagaatgaagttcactgccaccttcctcatgatggccatcttcgtcctcatggttgaacctggagagtgtggctggggaagcttttttaaaaaggctgctcacgttggcaagcatgttggcaaggcggcccttactcattaccttggcgataagcaggagctcaacaagcgtgcagtcgatgaagacccaaatgttattgtttttgaatgaagaaatcgccttgaaggagccttcagatgatatataatccttcttgcttttaatgaaataaatcagacttttacctgcaacagcaaaaaaaaaaaaaaaaaa

SEQ ID NO: 329 - protein, 68 aa, Pleuronectes americanus
MKFTATFLMMAIFVLMVEPGECGWGSFFKKAAHVGKHVGKAALTHYLGDKQELNKRAVDEDPNVIVFE